(19) United States
(12) Patent Application Publication
(10) Pub. No.: US 2024/0299493 A1
(43) Pub. Date: Sep. 12, 2024

(54) COMPOSITIONS AND METHODS FOR TREATING PULMONARY HYPERTENSION

(71) Applicant: Acceleron Pharma Inc., Cambridge, MA (US)

(72) Inventor: Ravindra Kumar, Cambridge, MA (US)

(21) Appl. No.: 18/572,091

(22) PCT Filed: Jun. 17, 2022
(86) PCT No.: PCT/US2022/034090

§ 371 Date: Dec. 19, 2023

Related U.S. Application Data
(60) Provisional application No. 63/213,073, filed on Jun. 21, 2021.

(51) Int. Cl. A61K 38/17; A61P 9/12

(52) U.S. Cl. CPC A61K 38/179; A61P 9/12

(57) ABSTRACT

In some aspects, the disclosure relates to compositions and methods comprising ActRII antagonists to treat, prevent, or reduce the progression rate and/or severity of post-capillary pulmonary hypertension (PcPH), particularly treating, preventing or reducing the progression rate and/or severity of one or more PcPH-associated complications.

### CROSS-REFERENCE TO RELATED APPLICATIONS

This application claims the benefit of and priority to U.S. Provisional Application No. 63/213,073, filed Jun. 21, 2021. The foregoing application is incorporated herein by reference in its entirety.

### FIELD OF INVENTION

This application relates to ActRII antagonists, compositions, and methods comprising ActRII antagonists to treat, prevent, or reduce the progression rate and/or severity of post-capillary pulmonary hypertension (PcPH), particularly treating, preventing or reducing the progression rate and/or severity of one or more PcPH-associated complications.

### BACKGROUND OF THE INVENTION

Pulmonary hypertension (PH) is a disease characterized by high blood pressure in lung vasculature, including pulmonary arteries, pulmonary veins, and pulmonary capillaries. In general, PH is defined as a mean pulmonary arterial pressure (mPAP) 20 mm Hg at rest or ≥30 mm Hg with exercise [Hill et al., Respiratory Care 54(7):958-68 (2009)]. One of the main PH symptoms is difficulty in breathing or shortness of breath, and other symptoms include fatigue, dizziness, fainting, peripheral edema (swelling in foot, legs or ankles), bluish lips and skin, chest pain, angina pectoris, light-headedness during exercise, non-productive cough, racing pulse and palpitations. PH can be a severe disease causing heart failure, which is one of the most common causes of death in people who have pulmonary hypertension. Postoperative pulmonary hypertension may complicate many types of surgeries or procedures, and present a challenge associated with a high mortality.

PH may be grouped based on different manifestations of the disease sharing similarities in pathophysiologic mechanisms, clinical presentation, and therapeutic approaches [Simonneau et al., JACC 54(1):S44-54 (2009)]. Clinical classification of PH was first proposed in 1973, and a recent updated clinical classification was endorsed by the World Health Organization (WHO) in 2018. According to the updated PH clinical classification, there are five main groups of PH: pulmonary arterial hypertension (PAH), characterized by a pulmonary arterial wedge pressure (PAWP) ≤15 mm Hg; PH due to left heart disease (also known as pulmonary venous hypertension or congestive heart failure), characterized by a PAWP >15 mm Hg; PH due to lung diseases and/or hypoxia; PH due to pulmonary artery obstructions; and PH with unclear and/or multifactorial mechanisms [Simonneau (2019) Eur Respir J: 53:1801913]. PH due to left heart disease is further classified into PH due to heart failure with preserved left ventricular ejection fraction; PH due to heart failure with reduced left ventricular ejection fraction; valvular heart disease; and congenital/acquired cardiovascular conditions leading to post-capillary PH [Simonneau (2019) Eur Respir J: 53:1801913]. Diagnosis of various types of PH typically requires a series of tests.

In general, PH treatment depends on the cause or classification of PH. Where PH is caused by a known medicine or medical condition, it is known as a secondary PH, and its treatment is usually directed at the underlying disease. Treatment of Group 2 pulmonary hypertension (e.g., venous hypertension) generally involves optimizing left ventricular function by administering diuretics, beta blockers, and ACE inhibitors, or repairing or replacing a mitral valve or aortic valve.

There is a high, unmet need for effective therapies for treating pulmonary hypertension. Accordingly, it is an object of the present disclosure to provide methods for treating, preventing, or reducing the progression rate and/or severity of PH, particularly treating, preventing or reducing the progression rate and/or severity of one or more PH-associated complications.

### SUMMARY OF THE INVENTION

In part, the data presented herein demonstrates that ActRII antagonists (e.g., ActRII polypeptides) can be used to treat post-capillary pulmonary hypertension (PcPH) (e.g., WHO Group 2 and/or Group 5 PH). For example, data presented herein show that a soluble ActRIIA polypeptide can be used, individually, to ameliorate various complications of Group 2 PH (e.g., reduce cardiac hypertrophy, elevate fractional shortening, restore left ventricular ejection fraction, reduce E/E′ ratio, reduce IVRT, reduce elevated right ventricle free wall thickness (RVFWT), increase tricuspid annular plane systolic excursion (TAPSE)) in a left heart failure-induced PH model (TAC-PH). Thus, the disclosure establishes that antagonists of the ActRII (ActRIIA and ActRIIB) signaling pathways may be used to reduce the severity of PcPH (e.g., WHO Group 2 and/or Group 5 PH). While soluble ActRIIA polypeptides may affect PcPH through a mechanism other than ActRIIA/B ligand antagonisms, the disclosure nonetheless demonstrates that desirable therapeutic agents may be selected on the basis of ActRII signaling antagonist activity. Therefore, in some embodiments, the disclosure provides methods for using various ActRII signaling antagonists for treating PcPH, particularly WHO Group 2 and/or Group 5 PH, including, for example, antagonists that inhibit one or more TGF-beta family ligands [e.g., activin A, activin B, activin AB, activin AC, BMP6, BMP7, BMP9, BMP10, GDF3, GDF8, and GDF11]; antagonists that inhibit ActRIIA or ActRIIB; and antagonists that inhibit one or more downstream signaling components (e.g., Smad proteins). As used herein, such signaling antagonists are collectively referred to as “ActRII antagonists” or “ActRII inhibitors”. Accordingly, the disclosure provides, in part, ActRII antagonist compositions and methods for treating PcPH (e.g., WHO Group 2 and/or Group 5 PH), particularly treating one or more complications of PcPH (e.g., smooth muscle and/or endothelial cell proliferation in the pulmonary artery, angiogenesis in the pulmonary artery, dyspnea, chest pain, pulmonary vascular remodeling, right ventricular hypertrophy, left ventricular hypertrophy, left atrium dilation, and pulmonary fibrosis). ActRII antagonists to be used in accordance with the methods and uses of the disclosure include, for example, ligand traps (e.g., soluble ActRIIA polypeptides or ActRIIB polypeptides). Optionally, ActRII antagonists may be used in combination with one or more supportive therapies and/or additional active agents for treating PcPH.

In certain aspects, the disclosure relates to methods of treating post-capillary pulmonary hypertension (PcPH). In certain aspects, the disclosure provides methods of treating post-capillary pulmonary hypertension (PcPH), comprising administering to a patient in need thereof an effective amount of an effective amount of an ActRIIA variant polypeptide. In other aspects, the disclosure provides methods of treating, preventing, or reducing the progression rate and/or severity of one or more complications of post-capillary pulmonary hypertension, comprising administering to a patient in need thereof an effective amount of an ActRIIA variant polypeptide. In other aspects, the disclosure provides methods of treating post-capillary pulmonary hypertension (PcPH), comprising administering to a patient in need thereof an effective amount of an effective amount an ActRIIB variant polypeptide. In other aspects, the disclosure provides methods of treating, preventing, or reducing the progression rate and/or severity of one or more complications of post-capillary pulmonary hypertension, comprising administering to a patient in need thereof an effective amount of an ActRIIB variant polypeptide. In some embodiments, the one or more complications of post-capillary pulmonary hypertension is selected from the group consisting of: smooth muscle and/or endothelial cell proliferation in the pulmonary artery, angiogenesis in the pulmonary artery, dyspnea, chest pain, pulmonary vascular remodeling, right ventricular hypertrophy, left ventricular hypertrophy, left atrium dilation, and pulmonary fibrosis. In some embodiments, the PcPH is isolated post-capillary pulmonary hypertension (IpcPH). In some embodiments, the PcPH is combined post- and pre-capillary PH (CpcPH). In some embodiments, the patient has Group 2 pulmonary hypertension as recognized by the World Health Organization (WHO). In some embodiments, the patient has pulmonary hypertension due to heart failure with preserved left ventricular ejection fraction (LVEF). In some embodiments, the patient has pulmonary hypertension due to heart failure with reduced left ventricular ejection fraction (LVEF). In some embodiments, the patient has valvular heart disease. In some embodiments, the patient has congenital/acquired cardiovascular conditions leading to post-capillary PH. In some embodiments, the patient has Group 5 pulmonary hypertension as recognized by the WHO. In some embodiments, the patient has pulmonary hypertension with unclear and/or multifactorial mechanisms. In some embodiments, the patient has a mean pulmonary arterial pressure (mPAP) selected from the group consisting of: an mPAP of at least 20 mmHg; an mPAP of at least 25 mmHg; an mPAP of at least 30 mmHg; an mPAP of at least 35 mmHg; an mPAP of at least 40 mmHg; an mPAP of at least 45 mmHg; and an mPAP of at least 50 mmHg. In some embodiments, the method reduces mPAP in the patient. In some embodiments, the method reduces the mPAP in the patient by at least 10% (e.g., 10%, 15%, 20%, 25%, 30%, 35%, 40%, 45%, or at least 50%). In some embodiments, the method reduces the mPAP by at least 3 mmHg (e.g., at least 3, 5, 7, 10, 12, 15, 20, or 25 mm Hg) in the patient. In some embodiments, the patient has a pulmonary arterial wedge pressure (PAWP) of greater than 15 mmHg. In some embodiments, the method decreases the PAWP in the patient. In some embodiments, the method reduces the PAWP in the patient by at least 10% (e.g., 10%, 15%, 20%, 25%, 30%, 35%, 40%, 45%, or at least 50%). In some embodiments, the patient has a left ventricular end diastolic pressure (LVEDP) of greater than 15 mmHg prior to treatment. In some embodiments, the method decreases the LVEDP in the patient. In some embodiments, the method reduces the LVEDP in the patient by at least 10% (e.g., 10%, 15%, 20%, 25%, 30%, 35%, 40%, 45%, or at least 50%). In some embodiments, the patient has a diastolic pressure gradient (DPG) of less than 7 mmHg prior to treatment. In some embodiments, the patient has a DPG of at least 7 mmHg prior to treatment. In some embodiments, the method decreases the DPG in the patient. In some embodiments, the method reduces the DPG in the patient by at least 10% (e.g., 10%, 15%, 20%, 25%, 30%, 35%, 40%, 45%, or at least 50%). In some embodiments, the patient has a transpulmonary pressure gradient (TPG) of less than or equal to 12 mm Hg. In some embodiments, the patient has a TPG of greater than 12 mm Hg. In some embodiments, the method decreases the TPG in the patient. In some embodiments, the method reduces the TPG in the patient by at least 10% (e.g., 10%, 15%, 20%, 25%, 30%, 35%, 40%, 45%, or at least 50%). In some embodiments, the patient has a pulmonary vascular resistance (PVR) greater than or equal to 3 Wood Units. In some embodiments, the method decreases the PVR in the patient. In some embodiments, the method reduces the PVR in the patient by at least 10% (e.g., 10%, 15%, 20%, 25%, 30%, 35%, 40%, 45%, or at least 50%). In some embodiments, the method prevents the progression of IpcPH to CpcPH. In some embodiments, the method reduces the development of a pre-capillary component of PH. In some embodiments, the patient has preserved left ventricular ejection fraction. In some embodiments, the preserved left ventricular fraction is greater than 45%. In some embodiments, the preserved left ventricular fraction is measured using echocardiography. In some embodiments, the patient has diastolic dysfunction of the left ventricle. In some embodiments, the patient has systolic dysfunction of the left ventricle. In some embodiments, the method decreases right ventricular hypertrophy in the patient. In some embodiments, the method decreases right ventricular hypertrophy in the patient by at least 10% (e.g., 10%, 15%, 20%, 25%, 30%, 35%, 40%, 45%, or at least 50%). In some embodiments, the method decreases left ventricular hypertrophy in the patient. In some embodiments, the method decreases left ventricular hypertrophy in the patient by at least 10% (e.g., 10%, 15%, 20%, 25%, 30%, 35%, 40%, 45%, or at least 50%). In some embodiments, the method decreases smooth muscle hypertrophy in the patient. In some embodiments, the method decreases smooth muscle hypertrophy in the patient by at least 10% (e.g., 10%, 15%, 20%, 25%, 30%, 35%, 40%, 45%, or at least 50%). In some embodiments, the method decreases pulmonary arteriole muscularity in the patient. In some embodiments, the method decreases pulmonary arteriole muscularity in the patient by at least 10% (e.g., 10%, 15%, 20%, 25%, 30%, 35%, 40%, 45%, or at least 50%). In some embodiments, the patient has a comorbidity selected from the group consisting of systemic hypertension, diabetes mellitus, obesity, coronary artery disease (CAD), heart failure, and anemia. In some embodiments, the patient has elevated brain natriuretic peptide (BNP) levels as compared to a healthy patient. In some embodiments, the patient has a BNP level of at least 100 pg/mL (e.g., 100, 150, 200, 300, 400, 500, 1000, 3000, 5000, 10,000, 15,000, or 20,000 pg/mL). In some embodiments, the method decreases BNP levels in the patient by at least 10% (e.g., 10%, 15%, 20%, 25%, 30%, 35%, 40%, 45%, 0%, 5%, 60%, 65%, 70%, 75%, or at least 80%). In some embodiments, the method decreases BNP levels to normal levels (i.e., <100 pg/ml). In some embodiments, the method increases exercise capacity of the patient. In some embodiments, the patient has a 6-minute walk distance from 150 to 400 meters. In some embodiments, the method increases the patient's 6-minute walk distance. In some embodiments, the method increases the patient's 6-minute walk distance by at least 10 meters (e.g., at least 10, 20, 30, 40, 50, 60, 70, 80, 90, 100, 125, 150, 175, 200, 250, 300, or more than 400 meters). In some embodiments, the method reduces the patient's Borg dyspnea index (BDI). In some embodiments, the method reduces the patient's BDI by at least 0.5 index points (e.g., at least 0.5, 1, 1.5, 2, 2.5, 3, 3.5, 4, 4.5, 5, 5.5, 6, 6.5, 7, 7.5, 8, 8.5, 9, 9.5, or 10 index points). In some embodiments, the patient has decreased renal function. In some embodiments, the method further improves renal function. In some embodiments, the patient has Functional Class II or Class III pulmonary hypertension in accordance with the World Health Organization's functional classification system for pulmonary hypertension. In some embodiments, the patient has Functional Class I, Class II, Class III, or Class IV pulmonary hypertension as recognized by the World Health Organization. In some embodiments, the method prevents or delays pulmonary hypertension Functional Class progression (e.g., prevents or delays progression from Functional Class I to Class II, Class II to Class III, or Class III to Class IV pulmonary hypertension as recognized by the World Health Organization). In some embodiments, the method promotes or increases pulmonary hypertension Functional Class regression (e.g., promotes or increases regression from Class IV to Class III, Class III to Class II, or Class II to Class I pulmonary hypertension as recognized by the World Health Organization). In some embodiments, the method delays clinical worsening of PcPH. In some embodiments, the method delays clinical worsening of PcPH in accordance with the World Health Organization's functional classification system for pulmonary hypertension. In some embodiments, the method reduces the risk of hospitalization for one or more complications associated with PcPH. In some embodiments, the patient has a hemoglobin level from >8 and <15 g/dl. In some embodiments, the patient has been treated with one or more vasodilators. In some embodiments, the patient has been treated with one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, treprostinil, iloprost, ambrisentan, and tadalafil. In some embodiments, the method further comprises administration of one or more vasodilators. In some embodiments, the method further comprises administration of one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, treprostinil, iloprost, ambrisentan, and tadalafil.

In certain aspects, an ActRIIA variant polypeptide, to be used in accordance with methods and uses described herein, is a ActRIIA variant polypeptide that comprises the sequence of GAILGRSETQECLX₁X₂NANWX₃X₄X₅X₆TNQTGVEX₇CX₈GX₉X₁₀X₁₁X₁₂X₁₃X₁₄HCX₁₅A TWX₁₆NISGSIEIVX₁₇X₁₈GCX₁₉X₂₀X₂₁DX₂₂NCYDRTDCVEX₂₃X₂₄X₂₅X₂₆PX₂₇VYFCCCE GNMCNEKFSYFPEMEVTQPTS (SEQ ID NO: 139), wherein X₁ is F or Y; X₂ is F or Y; X₃ is E or A; X₄ is K or L; X₅ is D or E; X₆ is R or A; X₇ is P or R; X₈ is Y or E; X₉ is D or E; X₁₀ is K or Q; X₁₁ is D or A; X₁₂ is K or A; X₁₃ is R or A; X₁₄ is R or L; X₁₅ is F or Y; X₁₆ is K, R, or A; X₁₇ is K, A, Y, F, or I; X₁₈ is Q or K; X₁₉ is W or A; X₂₀ is L or A; X₂₁ is D, K, R, A, F, G, M, N, or I; X₂₂ is I, F, or A; X₂₃ is K or T; X₂₄ is K or E; X₂₅ is D or E; X₂₆ is S or N; and X₂₇ is E or Q, and wherein the ActRIIA variant polypeptide has at least one amino acid substitution relative to a wild-type extracellular ActRIIA having the sequence of SEQ ID NO: 211 or an extracellular ActRIIA having any one of the sequences of SEQ ID NOs: 212-232. In some embodiments, the ActRIIA variant polypeptide has a sequence of GAILGRSETQECLFX₂NANWX₃X₄X₅X₆TNQTGVEX₇CX₈GX₉KX₁₁X₁₂X₁₃X₁₄HCX₁₅AT WX₁₆NISGSIEIVX₁₇X₁₈GCX₁₉X₂₀X₂₁DX₂₂NCYDRTDCVEX₂₃X₂₄X₂₅X₂₆PX₂₇VYFCCCEG NMCNEKFSYFPEMEVTQPTS (SEQ ID NO: 140). In some embodiments, the ActRIIA variant polypeptide has a sequence of GAILGRSETQECLFX₂NANWEX₄X₅RTNQTGVEX₇CX₈GX₉KDKRX₁₄HCX₁₅ATWX₁₆NI SGSIEIVKX₁₈GCWLDDX₂₂NCYDRTDCVEX₂₃X₂₄X₂₅X₂₆PX₂₇VYFCCCEGNMCNEKFSY FPEMEVTQPTS (SEQ ID NO: 141). In some embodiments, the ActRIIA variant polypeptide has a sequence of GAILGRSETQECLFX₂NANWEX₄DRTNQTGVEX₇CXGX₉KDKRX₄HCX₁₅ATWX₁₆NIS GSIEIVKX₁₈GCWLDDX₂₂NCYDRTDCVEX₂₃KX₂₅X₂₆PX₂₇VYFCCCEGNMCNEKFSYFP EMEVTQPTS (SEQ ID NO: 142). In some embodiments, the ActRIIA variant polypeptide has a sequence of GAILGRSETQECLFX₂NANWEX₄DRTNQTGVEPCX₈GX₉KDKRX₁₄HCFATWKNISGSI EIVKX₁₈GCWLDDINCYDRTDCVEX₂₃KX₂₅X₂₆PX₂₇VYFCCCEGNMCNEKFSYFPEMEV TQPTS (SEQ ID NO: 143). In some embodiments, X₁ is F. In some embodiments, X₁ is Y. In some embodiments, X₁₀ is K. In some embodiments, X₁₀ is Q. In some embodiments, X₂ is F. In some embodiments, X₂ is Y. In some embodiments, X₃ is E. In some embodiments, X₃ is A. In some embodiments, X₄ is K. In some embodiments, X₄ is L. In some embodiments, X₅ is D. In some embodiments, X₅ is E. In some embodiments, X₆ is R. In some embodiments, X₆ is A. In some embodiments, X₇ is P. In some embodiments, X₇ is R. In some embodiments, X₈ is Y. In some embodiments, X₈ is E. In some embodiments, X₉ is D. In some embodiments, X₉ is E. In some embodiments, X₁₁ is D. In some embodiments, X₁₁ is A. In some embodiments, X₁₂ is K. In some embodiments, X₁₂ is A. In some embodiments, X₁₃ is R. In some embodiments, X₁₃ is A. In some embodiments, X₁₄ is R. In some embodiments, X₁₄ is L. In some embodiments, X₁₅ is F. In some embodiments, X₁₅ is Y. In some embodiments, X₁₁ is K. In some embodiments, X₁₆ is R. In some embodiments, X₁₆ is A. In some embodiments, X₁₇ is K. In some embodiments, X₁₇ is A. In some embodiments, X₁₇ is Y. In some embodiments, X₁₇ is F. In some embodiments, X₁₇ is I. In some embodiments, X₁₈ is Q. In some embodiments, X₁₈ is K. In some embodiments, X₁₉ is W. In some embodiments, X₁₉ is A. In some embodiments, X₂₀ is L. In some embodiments, X₂₀ is A. In some embodiments, X₂₁ is D. In some embodiments, X₂₁ is K. In some embodiments, X₂₁ is R. In some embodiments, X₂₁ is A. In some embodiments, X₂₁ is F. In some embodiments, X₂₁ is G. In some embodiments, X₂₁ is M. In some embodiments, X₂₁ is N. In some embodiments, X₂₁ is I. In some embodiments, X₂₂ is I. In some embodiments, X₂₂ is F. In some embodiments, X₂₂ is A. In some embodiments, X₂₃ is K. In some embodiments, X₂₃ is T. In some embodiments, X₂₄ is K. In some embodiments, X₂₄ is E. In some embodiments, X₂₅ is D. In some embodiments, X₂₅ is E. In some embodiments, X₂₆ is S. In some embodiments, X₂₆ is N. In some embodiments, X₂₇ is E. In some embodiments, X₂₇ is Q. In some embodiments, X₂₃ is T, X₂₄ is E, X₂₅ is E, and X₂₆ is N. In some embodiments, X₂₃ is T, X₂₄ is E, X₂₅ is E, and X₂₆ is N. In some embodiments, X₁₇ is K. In some embodiments, the ActRIIA variant polypeptide has the sequence of any one of SEQ ID NOs: 145-210. In some embodiments, the amino acid at position X₂₄ is replaced with the amino acid K. In some embodiments, the amino acid at position X₂₄ is replaced with the amino acid E. In some embodiments, the ActRIIA variant polypeptide further comprises a C-terminal extension of one or more amino acids. In some embodiments, the C-terminal extension is NP. In some embodiments, the C-terminal extension is NPVTPK. In some embodiments, the ActRIIA variant polypeptide binds to one or more ligands selected from the group consisting of: activin A, activin B, GDF11, GDF8, and BMP6. In some embodiments, the ActRIIA variant polypeptide does not bind or does not substantially bind to one or more ligands selected from the group consisting of: BMP10, BMP9, and GDF3. In some embodiments, the ActRIIA variant polypeptide is lyophilized. In some embodiments, the ActRIIA variant polypeptide is soluble. In some embodiments, the ActRIIA variant polypeptide is administered using subcutaneous injection. In some embodiments, ActRIIA variant polypeptide is administered every 4 weeks.

In some embodiments, the ActRIIA variant polypeptide comprises an amino acid sequence selected from the group consisting of SEQ ID NO: 196 and SEQ ID NO: 207. In some embodiments, the ActRIIA polypeptide is a fusion protein further comprising a heterologous domain. In some embodiments, the heterologous domain is an Fc immunoglobulin domain. In some embodiments, the ActRIIA variant polypeptide and/or fusion protein further comprises a linker domain positioned between the ActRIIA variant polypeptide and the heterologous domain. In some embodiments, the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24). In some embodiments, the ActRIIA variant polypeptide or fusion protein comprises one or more amino acid modifications selected from the group consisting of: a glycosylated amino acid, a PEGylated amino acid, a farnesylated amino acid, an acetylated amino acid, a biotinylated amino acid, and an amino acid conjugated to a lipid moiety. In some embodiments, the ActRIIA variant polypeptide or fusion protein is glycosylated and has a mammalian glycosylation pattern. In some embodiments, the ActRIIA variant polypeptide or fusion protein has a glycosylation pattern obtainable from a Chinese hamster ovary cell line. In some embodiments, the ActRIIA variant polypeptide or fusion protein binds to one or more ligands selected from the group consisting of: activin A, activin B, GDF11, GDF8, and BMP6. In some embodiments, the ActRIIA variant polypeptide or fusion protein binds to activin A. In some embodiments, the ActRIIA variant polypeptide or fusion protein inhibits one or more TGFβ superfamily ligands selected from the group consisting of: activin A, activin B, GDF11, GDF8, and BMP6. In some embodiments, the ActRIIA variant polypeptide or fusion protein inhibits activin A. In some embodiments, the ActRIIA variant polypeptide or fusion protein does not bind or does not substantially bind to one or more ligands selected from the group consisting of: BMP10, BMP9, and GDF3. In some embodiments, the ActRIIA variant polypeptide or fusion protein binds to one or more of BMP10, BMP9, or GDF3 with lower affinity compared to a corresponding wild-type ActRIIA polypeptide. In some embodiments, the ActRIIA variant polypeptide is in a pharmaceutical preparation.

In certain aspects, a ActRIIB variant polypeptide, to be used in accordance with methods and uses described herein, is a ActRIIB variant polypeptide that comprises the amino acid sequence of SEQ ID NO: 303, wherein at least one of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 303 is substituted with another amino acid, and wherein said ActRIIB variant polypeptide is capable of binding myostatin and activin A, but demonstrates a decreased binding affinity for BMP9 relative to a wild-type ActRIIB polypeptide. In some embodiments, at least one of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 303 is substituted with the amino acid at the corresponding position of SEQ ID NO: 304, and wherein the ActRIIB variant polypeptide is capable of binding myostatin and activin A, but demonstrates a decreased binding affinity for BMP9 relative to a wild-type ActRIIB polypeptide. In some embodiments, the ActRIIB variant polypeptide comprises the amino acid sequence selected from the group consisting of: SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338 and SEQ ID NO: 339. In some embodiments, the ActRIIB variant polypeptide comprises the amino acid sequence selected from the group consisting of: SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, and SEQ ID NO: 407. In some embodiments, the ActRIIB variant polypeptide comprises an amino acid sequence selected from the group consisting of SEQ ID NO: 318 and SEQ ID NO: 331. In some embodiments, the ActRIIB variant polypeptide is selected from the group consisting of: a polypeptide comprising an amino acid sequence that is at least 90% identical to SEQ ID NO: 318; a polypeptide comprising an amino acid sequence that is at least 95% identical to SEQ ID NO: 318; a polypeptide comprising an amino acid sequence that is at least 99% identical to SEQ ID NO: 318; and a polypeptide comprising the amino acid sequence of SEQ ID NO: 318. In some embodiments, the ActRIIB variant polypeptide is selected from the group consisting of: a polypeptide comprising an amino acid sequence that is at least 90% identical to SEQ ID NO: 331; a polypeptide comprising an amino acid sequence that is at least 95% identical to SEQ ID NO: 331; a polypeptide comprising an amino acid sequence that is at least 99% identical to SEQ ID NO: 331; and a polypeptide comprising the amino acid sequence of SEQ ID NO: 331. In some embodiments, the ActRIIB variant polypeptide binds to one or more ligands selected from the group consisting of: activin A, activin B, GDF11, GDF8, and BMP6. In some embodiments, the ActRIIB variant polypeptide does not bind or does not substantially bind to one or more ligands selected from the group consisting of: BMP10, BMP9, and GDF3. In some embodiments, the ActRIIB variant polypeptide is lyophilized. In some embodiments, the ActRIIB variant polypeptide is soluble. In some embodiments, the ActRIIB variant polypeptide is administered using subcutaneous injection. In some embodiments, the ActRIIB variant polypeptide is administered every 4 weeks.

As described herein, in certain aspects, ActRIIB variant polypeptides may be homomultimers. In some embodiments, the ActRIIB variant polypeptide is part of a homodimer protein complex. In some embodiments, the ActRIIB variant polypeptide does not comprise an acidic amino acid at the position corresponding to L79 of SEQ ID NO: 1. In some embodiments, the ActRIIB variant polypeptide is a fusion protein further comprises a heterologous domain. In some embodiments, the heterologous domain is an Fc immunoglobulin domain. In some embodiments, the fusion protein further comprises a linker domain positioned between the ActRIIB variant polypeptide domain and the heterologous domain. In some embodiments, the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24). In some embodiments, the ActRIIB variant polypeptide or fusion protein comprises one or more amino acid modifications selected from the group consisting of: a glycosylated amino acid, a PEGylated amino acid, a farnesylated amino acid, an acetylated amino acid, a biotinylated amino acid, and an amino acid conjugated to a lipid moiety. In some embodiments, the ActRIIB variant polypeptide or fusion protein is glycosylated and has a mammalian glycosylation pattern. In some embodiments, the ActRIIB variant polypeptide or fusion protein has a glycosylation pattern obtainable from a Chinese hamster ovary cell line. In some embodiments, the ActRIIB variant polypeptide or fusion protein binds to one or more ligands selected from the group consisting of: activin A, activin B, GDF11, GDF8, and BMP6. In some embodiments, the ActRIIB variant polypeptide or fusion protein binds to activin A. In some embodiments, the ActRIIB variant polypeptide or fusion protein inhibits one or more TGFβ superfamily ligands selected from the group consisting of: activin A, activin B, GDF11, GDF8, and BMP6. In some embodiments, the ActRIIB variant polypeptide or fusion protein inhibits activin A. In some embodiments, the ActRIIB variant polypeptide or fusion protein does not bind or does not substantially bind to one or more ligands selected from the group consisting of: BMP10, BMP9, and GDF3. In some embodiments, the ActRIIB variant polypeptide or fusion protein binds to one or more of BMP10, BMP9, or GDF3 with lower affinity compared to a corresponding wild-type ActRIIB polypeptide. In some embodiments, the ActRIIB variant polypeptide and/or fusion protein is in a pharmaceutical preparation. In some embodiments, the pharmaceutical preparation is administered using subcutaneous injection. In some embodiments, the pharmaceutical preparation is administered every 4 weeks.

In certain aspects, the methods disclosed herein comprise further administering to the patient an additional active agent and/or supportive therapy. In some embodiments, the additional active agent and/or supportive therapy is selected from the group consisting of: beta-blockers, angiotensin-converting enzyme inhibitors (ACE inhibitors), angiotensin receptor blockers (ARBs), diuretic agents, lipid-lowering medications, endothelin blockers, PDE5 inhibitors, prostacyclins, or a left ventricular assist device (LVAD). In some embodiments, the additional active agent and/or supportive therapy is selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1-->3)-beta-D-glucopyranosyl] oleanolic acid; 3-O-[beta-D-glucopyranosyl-(1-->2)-beta-D-glucopyranosyl] oleanolic acid; 3-O-[beta-D-glucopyranosyl-(1-->3)-beta-D-glucopyranosyl] oleanolic acid 28-O-beta-D-glucopyranosyl ester; 3-O-[beta-D-glucopyranosyl-(1-->2)-beta-D-glucopyranosyl] oleanolic acid 28-O-beta-D-glucopyranosyl ester; 3-O-[a-L-rhamnopyranosyl-(1-->3)-beta-D-glucuronopyranosyl]oleanolic acid; 3-O-[alpha-L-rhamnopyranosyl-(1-->3)-beta-D-glucuronopyranosyl]oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate); a left ventricular assist device (LVAD), or lung and/or heart transplantation.

In certain aspects, the disclosure provides a kit comprising a lyophilized polypeptide and an injection device. In certain aspects the disclosure provides a kit comprising a lyophilized polypeptide and an injection device, wherein the polypeptide is an ActRIIA polypeptide comprising an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 9, 10, 11, 36, 39, 139, 140, 141, 142, 143, 144, 145, 146, 147, 148, 149, 150, 151, 152, 153, 154,155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 283, 304, 408, and 409. In certain embodiments, the disclosure provides a kit comprising a lyophilized polypeptide and an injection device, wherein the polypeptide is an ActRIIB polypeptide comprising an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 4, 5, 6, 40, 42, 45, 46, 47, 48, 69, 74, 77, 78, 79, 138, 282, 289, 290, 291, 292, 293, 294, 295, 296, 297, 298, 299, 300, 301, 302, 303, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, and 407. In some embodiments, the polypeptide is glycosylated. In some embodiments, the polypeptide binds to one or more ligands selected from the group consisting of: activin A, activin B, and GDF11. In some embodiments, the polypeptide further binds to one or more ligands selected from the group consisting of: BMP10, GDF8, and BMP6. In some embodiments, the polypeptide binds to activin and/or GDF11. In some embodiments, the kit comprises one or more vials containing the lyophilized polypeptide. In some embodiments, the injection device comprises a pre-filled syringe. In some embodiments, the injection device comprises a pump apparatus. In some embodiments, the pump apparatus comprises an electromechanical pumping assembly. In some embodiments, the pump apparatus is a wearable pump apparatus. In some embodiments, the pre-filled syringe comprises a reconstitution solution. In some embodiments, the reconstitution solution comprises a pharmaceutically acceptable carrier and/or excipient. In some embodiments, the pharmaceutically acceptable carrier is selected from saline solution, purified water, or sterile water for injection. In some embodiments, the pharmaceutically acceptable excipient is selected from a buffering agent [e.g., citric acid (monohydrate) and/or trisodium citrate (dehydrate)], a surfactant (e.g., polysorbate 80), a stabilizer (e.g., sucrose), and a lyoprotectant (e.g., sucrose). In some embodiments, the injection device comprises a vial adapter. In some embodiments, the vial adapter is capable of attaching to a vial. In some embodiments, the vial adapter is capable of attaching to a pre-filled syringe. In some embodiments, the pre-filled syringe and the vial are attached to opposite ends of the vial adapter. In some embodiments, the reconstitution solution is transferred from the pre-filled syringe to the vial. In some embodiments, the lyophilized polypeptide is reconstituted into a sterile injectable solution. In some embodiments, the lyophilized polypeptide is reconstituted into a sterile injectable solution prior to use. In some embodiments, the sterile injectable solution is sterile water for injection. In some embodiments, the sterile injectable solution is administered parenterally. In some embodiments, the injection device is used to administer the sterile injectable solution parenterally. In some embodiments, the sterile injectable solution is administered via subcutaneous injection. In some embodiments, the sterile injectable solution is administered via intradermal injection. In some embodiments, the sterile injectable solution is administered via intramuscular injection. In some embodiments, the sterile injectable solution is administered via intravenous injection. In some embodiments, the sterile injectable solution is self-administered. In some embodiments, the sterile injectable solution comprises a therapeutically effective dose. In some embodiments, the therapeutically effective dose comprises a weight based dose. In some embodiments, the lyophilized polypeptide is administered every 4 weeks. In some embodiments, the kit is used to treat post-capillary pulmonary hypertension (PcPH). In some embodiments, the PcPH is isolated post-capillary pulmonary hypertension (IpcPH). In some embodiments, the PcPH is combined post- and pre-capillary PH (CpcPH). In some embodiments, the patient has Group 2 pulmonary hypertension as recognized by the WHO. In some embodiments, the patient has pulmonary hypertension due to heart failure with preserved left ventricular ejection fraction (LVEF). In some embodiments, the patient has pulmonary hypertension due to heart failure with reduced left ventricular ejection fraction (LVEF). In some embodiments, the patient has valvular heart disease. In some embodiments, the patient has congenital/acquired cardiovascular conditions leading to post-capillary PH. In some embodiments, the patient has Group 5 pulmonary hypertension as recognized by the WHO. In some embodiments, the patient has pulmonary hypertension with unclear and/or multifactorial mechanisms. In some embodiments, the shelf life of the lyophilized polypeptide is at least 1, 1.5, 2, 2.5, or 3 years. In some embodiments, the lyophilized polypeptide is reconstituted. In some embodiments, the reconstituted polypeptide has a shelf life of at least 2 hrs, 3 hrs, or 4 hrs.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

FIG. 1 shows an alignment of extracellular domains of human ActRIIB (SEQ ID NO: 2) and human ActRIIA (SEQ ID NO: 10) with the residues that are deduced herein, based on composite analysis of multiple ActRIIB and ActRIIA crystal structures, to directly contact ligand indicated with boxes.

FIG. 2 shows a multiple sequence alignment of various vertebrate ActRIIB proteins (SEQ ID NOs: 53-58) and human ActRIIA (SEQ ID NO: 59) as well as a consensus ActRII sequence derived from the alignment (SEQ ID NO: 60).

FIG. 3 shows a multiple sequence alignment of various vertebrate ActRIIA proteins and human ActRIIA (SEQ ID NOs: 10 and 62-68).

FIG. 4 shows multiple sequence alignment of Fc domains from human IgG isotypes using Clustal 2.1. Hinge regions are indicated by dotted underline. Double underline indicates examples of positions engineered in IgG1 (SEQ ID NO: 133) Fc to promote asymmetric chain pairing and the corresponding positions with respect to other isotypes IgG2 (SEQ ID NO: 135), IgG3 (SEQ ID NO: 136) and IgG4 (SEQ ID NO: 134).

FIG. 5A and FIG. 5B show the purification of ActRIIA-hFc expressed in CHO cells. The protein purifies as a single, well-defined peak as visualized by sizing column (FIG. 5A) and Coomassie stained SDS-PAGE (FIG. 5B) (left lane: molecular weight standards; right lane: ActRIIA-hFc).

FIG. 6A and FIG. 6B shows the binding of ActRIIA-hFc to activin (FIG. 6A) and GDF-11 (FIG. 6B), as measured by Biacore™ assay.

FIG. 7 shows the full, unprocessed amino acid sequence for ActRIIB(25-131)-hFc (SEQ ID NO: 69). The TPA leader (residues 1-22) and double-truncated ActRIIB extracellular domain (residues 24-131, using numbering based on the native sequence in SEQ ID NO: 1) are each underlined. Boxed is the glutamate revealed by sequencing to be the N-terminal amino acid of the mature fusion protein, which is at position 25 relative to SEQ ID NO: 1.

FIGS. 8A and 8B show a nucleotide sequence encoding ActRIIB(25-131)-hFc (the coding strand is shown at top, SEQ ID NO: 70, and the complement shown at bottom 3′-5′, SEQ ID NO: 71). Sequences encoding the TPA leader (nucleotides 1-66) and ActRIIB extracellular domain (nucleotides 73-396) are underlined. The corresponding amino acid sequence for ActRIIB(25-131) (SEQ ID NO: 138) is also shown.

FIGS. 9A and 9B show an alternative nucleotide sequence encoding ActRIIB(25-131)-hFc (the coding strand is shown at top, SEQ ID NO: 72, and the complement shown at bottom 3′-5′, SEQ ID NO: 73). This sequence confers a greater level of protein expression in initial transformants, making cell line development a more rapid process. Sequences encoding the TPA leader (nucleotides 1-66) and ActRIIB extracellular domain (nucleotides 73-396) are underlined, and substitutions in the wild type nucleotide sequence of the ECD (see FIG. 8) are boxed. The corresponding amino acid sequence for ActRIIB(25-131) (SEQ ID NO: 138) is also shown.

FIG. 10 shows the full amino acid sequence for the ActRIIB(L79D 20-134)-hFc (SEQ ID NO: 74), including the TPA leader sequence (double underline), ActRIIB extracellular domain (residues 20-134 in SEQ ID NO: 1; single underline), and hFc domain. The aspartate substituted at position 79 in the native sequence is double underlined and boxed, as is the glycine revealed by sequencing to be the N-terminal residue in the mature fusion protein.

FIGS. 11A and 11B show a nucleotide sequence encoding ActRIIB(L79D 20-134)-hFc. SEQ ID NO: 75 corresponds to the sense strand, and SEQ ID NO: 76 corresponds to the antisense strand. The TPA leader (nucleotides 1-66) is double underlined, and the ActRIIB extracellular domain (nucleotides 76-420) is single underlined.

FIG. 12 shows the full amino acid sequence for the truncated ActRIIB(L79D 25-131)-hFc (SEQ ID NO: 77), including the TPA leader (double underline), truncated ActRIIB extracellular domain (residues 25-131 in SEQ ID NO:1; single underline), and hFec domain. The aspartate substituted at position 79 in the native sequence is double underlined and boxed, as is the glutamate revealed by sequencing to be the N-terminal residue in the mature fusion protein.

FIG. 13 shows the amino acid sequence for the truncated ActRIIB(L79D 25-131)-hFc without a leader (SEQ ID NO: 78). The truncated ActRIIB extracellular domain (residues 25-131 in SEQ ID NO: 1) is underlined. The aspartate substituted at position 79 in the native sequence is double underlined and boxed, as is the glutamate revealed by sequencing to be the N-terminal residue in the mature fusion protein.

FIG. 14 shows the amino acid sequence for the truncated ActRIIB(L79D 25-131) without the leader, hFc domain, and linker (SEQ ID NO: 79). The aspartate substituted at position 79 in the native sequence is underlined and boxed, as is the glutamate revealed by sequencing to be the N-terminal residue in the mature fusion protein.

FIGS. 15A and 15B show a nucleotide sequence encoding ActRIIB(L79D 25-131)-hFc. SEQ ID NO: 80 corresponds to the sense strand, and SEQ ID NO: 81 corresponds to the antisense strand. The TPA leader (nucleotides 1-66) is double underlined, and the truncated ActRIIB extracellular domain (nucleotides 76-396) is single underlined. The amino acid sequence for the ActRIIB extracellular domain (SEQ ID NO: 79) is also shown.

FIGS. 16A and 16B show an alternative nucleotide sequence encoding ActRIIB(L79D 25-131)-hFc. SEQ ID NO: 82 corresponds to the sense strand, and SEQ ID NO: 83 corresponds to the antisense strand. The TPA leader (nucleotides 1-66) is double underlined, the truncated ActRIIB extracellular domain (nucleotides 76-396) is underlined, and substitutions in the wild-type nucleotide sequence of the extracellular domain are double underlined and boxed (compare with SEQ ID NO: 81, FIG. 15). The amino acid sequence for the ActRIIB extracellular domain (SEQ ID NO: 79) is also shown.

FIG. 17 shows nucleotides 76-396 (SEQ ID NO: 84) of the alternative nucleotide sequence shown in FIG. 16 (SEQ ID NO: 82). The same nucleotide substitutions indicated in FIG. 16 are also underlined and boxed here. SEQ ID NO: 84 encodes only the truncated ActRIIB extracellular domain (corresponding to residues 25-131 in SEQ ID NO: 1) with a L79D substitution, e.g., ActRIIB(L79D 25-131).

FIG. 18 shows a schematic image of a linearized version of cardiopulmonary circulation and the regions associated with various types of PH.

FIG. 19 shows a schematic image of a linearized version of cardiopulmonary circulation and the hemodynamic parameters associated with pre-capillary PH.

FIG. 20 shows a schematic image of a linearized version of cardiopulmonary circulation and the hemodynamic parameters associated with isolated post-capillary PH (IpcPH).

FIG. 21 shows a schematic image of a linearized version of cardiopulmonary circulation and the hemodynamic parameters associated with combined post- and pre-capillary PH (CpcPH).

FIGS. 22-26 shows the therapeutic effect of ActRIIA-mFc in a TAC-PH model based on endpoints for left ventricle function. Twenty-six C57/B6 male mice (10 wks old) underwent TAC pulmonary hypertension surgery (TAC-PH) and ten age-matched animals underwent a mock surgical procedure (Sham) at day 0. Two weeks after the surgery, TAC-PH mice were randomized into two groups. i) fourteen mice were injected subcutaneously with vehicle control (phosphate buffered saline (PBS)), twice weekly for 4 weeks starting from day 14 after surgery, “TAC-PH/PBS”; and a ii) twelve mice were injected subcutaneously with ActRIIA-mFc at a dose of 10 mg/kg twice weekly for 4 weeks starting from day 14 after TAC surgery, “TAC-PH/ActRIIA-mFc”. FIGS. 22-26 show endpoints for left ventricle function, including changes in cardiac hypertrophy heart weight/body weight (HW/BW) (FIG. 22), LV function parameters fractional shorting (FIG. 23) and LV ejection fraction (FIG. 24); and LV diastolic function parameters E/E′ [Ratio of mitrial inflow velocity (E) to mitrial annular velocity (E′)] (FIG. 25) and isovolumetric relaxation time (IVRT) (FIG. 26). Relative to “TAC-PH/PBS” treated mice, “TAC-PH/ActRIIA-mFc” treated mice demonstrated a significant effect of ActRIIA-mFc in reducing cardiac hypertrophy and improving cardiac function. Statistical significance (p value) is depicted as * p<0.05, **p<0.01, ***p<0.001, and ****p<0.0001 for comparison between “Sham” and sample “TAC-PH/PBS”. Statistical significance (p value) is depicted as #p<0.05, ##p<0.01, ###p<0.001, and ####p<0.0001 for comparison between “Sham” and sample “TAC-PH/ActRIIA-mFc”. Statistical significance (p value) is depicted as @ p<0.05, @@p<0.01, @@@p<0.001, and @@@@p<0.0001 for comparison between sample “TAC-PH/PBS” and sample “TAC-PH/ActRIIA-mFc”.

FIGS. 27-30 show the therapeutic effect of ActRIIA-mFc in a TAC-PH model based on endpoints for right ventricle function. Twenty-six C57/B6 male mice (10 wks old) underwent TAC pulmonary hypertension surgery (TAC-PH) and ten age-matched animals underwent a mock surgical procedure (Sham) at day 0. Two weeks after the surgery, TAC-PH mice were randomized into two groups. i) fourteen mice were injected subcutaneously with vehicle control (phosphate buffered saline (PBS)), twice weekly for 4 weeks starting from day 14 after surgery, “TAC-PH/PBS”; and a ii) twelve mice were injected subcutaneously with ActRIIA-mFc at a dose of 10 mg/kg twice weekly for 4 weeks starting from day 14 after TAC surgery, “TAC-PH/ActRIIA-mFc”. FIGS. 27-30 show endpoints for right ventricle function, including RV remodeling parameter right ventricular free wall thickness (RVFWT) (FIG. 27), RV remodeling and function parameter tricuspid annular plane systolic excursion (TAPSE) (FIG. 28), and RV function parameters RV stroke work (FIG. 29) and RV contractility (dP/dT) (FIG. 30). Relative to “TAC-PH/PBS” treated mice, “TAC-PH/ActRIIA-mFc” treated mice demonstrated a significant effect of ActRIIA-mFc in improving right heart remodeling and function. Statistical significance (p value) is depicted as * p<0.05, **p<0.01, ***p<0.001, and ****p<0.0001 for comparison between “Sham” and sample “TAC-PH/PBS”. Statistical significance (p value) is depicted as #p<0.05, ##p<0.01, ###p<0.001, and ####p<0.0001 for comparison between “Sham” and sample “TAC-PH/ActRIIA-mFc”. Statistical significance (p value) is depicted as @ p<0.05, @@p<0.01, @@@p<0.001, and @@@@p<0.0001 for comparison between sample “TAC-PH/PBS” and sample “TAC-PH/ActRIIA-mFc”.

FIGS. 31 and 32 show the therapeutic effect of ActRIIA-mFc in a TAC-PH model based on endpoints for lung remodeling. Twenty-six C57/B6 male mice (10 wks old) underwent TAC pulmonary hypertension surgery (TAC-PH) and ten age-matched animals underwent a mock surgical procedure (Sham) at day 0. Two weeks after the surgery, TAC-PH mice were randomized into two groups. i) fourteen mice were injected subcutaneously with vehicle control (phosphate buffered saline (PBS)), twice weekly for 4 weeks starting from day 14 after surgery, “TAC-PH/PBS”; and a ii) twelve mice were injected subcutaneously with ActRIIA-mFc at a dose of 10 mg/kg twice weekly for 4 weeks starting from day 14 after TAC surgery, “TAC-PH/ActRIIA-mFc”. FIGS. 31 and 32 show endpoints for lung remodeling, including ratio of lung weight to tibia length (LW/TL) (FIG. 31) and lung fibrosis percentage (FIG. 32). Relative to “TAC-PH/PBS” treated mice, “TAC-PH/ActRIIA-mFc” treated mice demonstrated a significant effect of ActRIIA-mFc in reducing pulmonary remodeling and fibrosis. Statistical significance (p value) is depicted as * p<0.05, **p<0.01, ***p<0.001, and ****p<0.0001 for comparison between “Sham” and sample “TAC-PH/PBS”. Statistical significance (p value) is depicted as #p<0.05, ##p<0.01, ###p<0.001, and ####p<0.0001 for comparison between “Sham” and sample “TAC-PH/ActRIIA-mFc”. Statistical significance (p value) is depicted as @ p<0.05, @@p<0.01, @@@p<0.001, and @@@@p<0.0001 for comparison between sample “TAC-PH/PBS” and sample “TAC-PH/ActRIIA-mFc”.

FIG. 33 shows components of a kit comprising a lyophilized polypeptide and an injection device. A vial (1) holds lyophilized polypeptide, reconstituted sterile injectable solution, or sterile injectable solution. A prefilled syringe (2) containing a reconstitution solution is used to reconstitute lyophilized polypeptide from (1) into a sterile injectable solution. A vial adapter (3) couples the vial (1) to the pre-filled syringe (2) via attachment to the vial at one end, and attachment to the pre-filled syringe at an opposite end. A syringe (4) and needle (5) are provided for administration of sterile injectable solution. Swab wipes (6) are provided for sterilization of individual kit components.

FIGS. 34-37 show that treatment with an ActRIIA-mFc fusion protein improves diastolic dysfunction in a rat model of left ventricular diastolic dysfunction (also referred to as HEpEF) group 2 (subgroup 2.2) pulmonary hypertension (PH). The experimental strategy used to test the preventative effects of ActRIIA-mFc in the rat model of HEpEF is shown in FIG. 34. FIGS. 35-37 show endpoints for left ventricular function, including the left ventricular ejection fraction (FIG. 35); LV diastolic function parameters E/E′ [Ratio of mitrial inflow velocity (E) to mitrial annular velocity (E′)] (FIG. 36); and isovolumetric relaxation time (IVRT) (FIG. 37). Statistical significance (p value) is depicted as * p<0.05, **p<0.01, and ***p<0.001.

FIGS. 38-40 show that treatment with an ActRIIA-mFc fusion protein reduces left heart remodeling in a rat model of left ventricular diastolic dysfunction (also referred to as HEpEF) group 2 (subgroup 2.2) pulmonary hypertension (PH). FIGS. 38-40 show endpoints for left heart remodeling, including changes in ratio of heart weight to tibia length (HW/TL) (FIG. 38); interventricular septal dimension at diastole (IVSd) (FIG. 39); and left ventricular mass (LVM) (FIG. 40). Statistical significance (p value) is depicted as * p<0.05, **p<0.01, and ***p<0.001.

FIGS. 41-43 show that treatment with an ActRIIA-mFc fusion protein reduces right ventricular systolic pressure (RVSP) and improves right ventricular function in a rat model of left ventricular diastolic dysfunction (also referred to as HEpEF) group 2 (subgroup 2.2) pulmonary hypertension (PH). FIGS. 41-43 show endpoints for right ventricular function, including changes in right ventricular free wall thickness (FIG. 41); pulmonary artery acceleration time (PAAT) (FIG. 42); and right ventricular systolic pressure (RVSP) (FIG. 43). Statistical significance (p value) is depicted as * p<0.05 and **p<0.01.

FIGS. 44-46 show that treatment with an ActRIIA-mFc fusion protein significantly reduced the fibrosis in LV, RV and lung in a rat model of left ventricular diastolic dysfunction (also referred to as HEpEF) group 2 (subgroup 2.2) pulmonary hypertension (PH). FIGS. 44-46 show a reduction in fibrosis, including changes in left ventricular fibrosis (FIG. 44); right ventricular fibrosis (FIG. 45); and lung fibrosis (FIG. 46). Statistical significance (p value) is depicted as * p<0.05 and **p<0.01.

FIGS. 47-50 show that treatment with an ActRIIA-mFc fusion protein significantly improves hyperglycemia and glucose intolerance in a rat model of left ventricular diastolic dysfunction (also referred to as HEpEF) group 2 (subgroup 2.2) pulmonary hypertension (PH). FIGS. 47-50 show endpoints for hyperglycemia and glucose intolerance, including changes in body weight (FIG. 47); fasting glucose (FIG. 48); blood glucose (FIG. 49); and glucose/creatine ratio (FIG. 50). Statistical significance (p value) is depicted as * p<0.05, **p<0.01, and ***p<0.001.

FIGS. 51-55 show that treatment with an ActRIIA-mFc fusion protein inhibits cardiac remodeling and improves LV function in a mouse model of PH due to heart failure with reduced LVEF (also referred to as HErEF) group 2 (subgroup 2.1) pulmonary hypertension (PH) and valvular heart disease (subgroup 2.3). The experimental strategy used to test the preventative effects of ActRIIA-mFc in the rat model of HErEF is shown in FIG. 51. FIGS. 52-55 show endpoints for left ventricle function, including changes in cardiac hypertrophy heart weight/tibia length (HW/TL) (FIG. 53), LV function parameters such as LV ejection fraction (FIG. 52), LV diastolic function parameters E/E′ [Ratio of mitral inflow velocity (E) to mitral annular velocity (E′)] (FIG. 54) and isovolumetric relaxation time (IVRT) (FIG. 55). Relative to “TAC PBS” treated mice, “TAC ActRIIA-mFc” treated mice demonstrated a significant effect of ActRIIA-mFc in inhibiting cardiac remodeling and improving LV function. Statistical significance (p value) is depicted as * p<0.05, **p<0.01, ***p<0.001, and ****p<0.0001 for comparison between “TAC PBS” and sample “TAC ActRIIA-mFc”. Statistical significance (p value) is depicted as #p<0.05, ##p<0.01, ###p<0.001, and ####p<0.0001 for comparison between “Sham” and sample “TAC PBS.”

FIGS. 56-58 show the therapeutic effect of ActRIIA-mFc in a TAC-PH model based on endpoints for right ventricle function. FIGS. 56-58 show endpoints for right ventricle function including right ventricular systolic pressure (RVSP) (FIG. 56), right ventricular free wall thickness (RVFWT) (FIG. 57), and pulmonary artery acceleration time (PAAT) (FIG. 58). Relative to “TAC PBS” treated mice, “TAC ActRIIA-mFc” mice treated with either 3 mpk and 10 mpk demonstrated a significant effect of ActRIIA-mFc in reducing RVSP and improving RV function. Statistical significance (p value) is depicted as * p<0.05, **p<0.01, ***p<0.001, and ****p<0.0001 for comparison between “TAC PBS” and sample “TAC ActRIIA-mFc.” Statistical significance (p value) is depicted as #p<0.05, ##p<0.01, ###p<0.001, and ####p<0.0001 for comparison between “Sham” and sample “TAC PBS.”

FIGS. 59-61 show the therapeutic effect of ActRIIA-mFc in a TAC-PH model based on endpoints for fibrosis in the left ventricle (LV), right ventricle (RV), and lung. FIGS. 59-61 show endpoints for fibrosis in the left ventricle (LV) (FIG. 59), right ventricle (RV) (FIG. 60), and lung (FIG. 61). Relative to “TAC PBS” treated mice, “TAC ActRIIA-mFc” mice treated with either 3 mpk or 10 mpk demonstrated a significant effect of ActRIIA-mFc in reducing fibrosis in the LV (FIG. 59), RV (FIG. 60), and lung (FIG. 61). Statistical significance (p value) is depicted as * p<0.05, **p<0.01, ***p<0.001, and ****p<0.0001 for comparison between “TAC PBS” and sample “TAC ActRIIA-mFc.” Statistical significance (p value) is depicted as #p<0.05, ##p<0.01, ###p<0.001, and ####p<0.0001 for comparison between “Sham” and sample “TAC PBS.”

FIGS. 62-67 show that treatment with an ActRIIA-mFc fusion protein reduces right ventricular systolic pressure (RVSP) and improves cardiopulmonary function in a rat model of left ventricular diastolic dysfunction (also referred to as HEpEF) group 2 (subgroup 2.2) pulmonary hypertension (PH). The experimental strategy used to test the preventative effects of an ActRIIA-mFc fusion protein in the rat model of HEpEF is shown in FIG. 62. FIGS. 63-67 show endpoints for right ventricular function, including changes in pulmonary artery acceleration time (PAAT) (FIG. 63); right ventricular systolic pressure (RVSP) (FIG. 64); right ventricular wall thickness (RVWT) (FIG. 65); tricuspid annular plane systolic excursion (TAPSE) (FIG. 66); and Fulton index, calculated as the ratio of right ventricular weight (RV) to weight of the combined left ventricle and septum (LV+S) (FIG. 67). Statistical significance (p value) is depicted as * p<0.05, **p<0.01, ***p<0.001, and ****p<0.0001.

### DETAILED DESCRIPTION

### 1. Overview

The present disclosure relates to compositions and methods of treating post-capillary pulmonary hypertension (e.g., WHO Group 2 and/or Group 5 PH) comprising administering to a patient in need thereof an effective amount of an ActRII polypeptide as described herein. In certain embodiments, the present disclosure provides methods of treating or preventing post-capillary pulmonary hypertension (PcPH) in an individual in need thereof through administering to the individual a therapeutically effective amount of an ActRII polypeptide as described herein. In certain embodiments, the present disclosure provides methods of treating or preventing combined post- and pre-capillary PH in an individual in need thereof through administering to the individual a therapeutically effective amount of an ActRII polypeptide as described herein.

Pulmonary hypertension due to left heart disease (PH-LHD) (also known as WHO Group 2 PH) is a complex pathophenotype that, when present, may result in an increased susceptibility to adverse events and a worse clinical outcome. Among those patients with PH-LHD, two phenotypes have been described: 1) a group of isolated post-capillary (IpcPH) or “passive” PH in which elevated pulmonary pressures are reversible and in proportion to increases in left atrial pressure, and 2) a group with “pre-capillary” component [combined post-capillary and pre-capillary PH (CpcPH)] whose pulmonary hypertension is worse than can be fully explained by passive elevation secondary to elevated left atrial pressure. This latter group, CpcPH, may have comorbid pulmonary vascular remodeling and therefore may demonstrate persistent PH after interventions to lower left sided filling pressures.

PH-LHD is sometimes defined as patients having a pulmonary capillary wedge pressure (PCWP) >15 mmHg and a mean pulmonary arterial pressure (mPAP) ≥25 mmHg (or a mean pulmonary arterial pressure (mPAP) ≥20 mmHg under updated guidelines). PH-LHD occurs as a consequence of the backward transmission of high left sided filling pressures, mainly driven by left ventricular diastolic function, directly to the post-capillary pulmonary vessels and, thereby, to the rest of the pulmonary circulation. In some embodiments, PH-LHD is driven by both systolic and diastolic dysfunction. PH-LHD may be associated with or caused by PH due to heart failure with preserved left ventricular ejection fraction (LVEF) [also known as HFpEF], PH due to heart failure with reduced LVEF (also known as HFrEF), valvular heart disease, or congenital/acquired cardiovascular conditions leading to post-capillary PH. Compared with PAH, patients with PH-LHD are often older, female, with a higher prevalence of cardiovascular co-morbidities and most, if not all, of the features of metabolic syndrome.

For WHO Group 2 (PH-LHD) and Group 5 PH patients, there are no approved specific therapies available beyond treatment of the underlying disease. Most PH-LHD therapies target the underlying condition (e.g., repair of valvular heart disease) rather than specifically treating PH. The lack of specific therapies is particularly problematic because PH-LHD is the most common cause of PH in western countries and its presence commonly results in adverse course of the disease. Specifically, the presence of PH-LHD can result in more severe symptoms in LHD, worse exercise tolerance, and a negative impact on outcome. Accordingly, there is a high unmet need for new treatments for post-capillary pulmonary hypertension (e.g., WHO Group 2 and/or Group 5 PH) and these treatments would have the potential to positively affect large numbers of patients.

As demonstrated herein, an ActRIIA polypeptide, which binds to various ActRIIA-interacting ligands, is effective in ameliorating various complications of Group 2 PH (e.g., reduce cardiac hypertrophy, elevate fractional shortening, restore left ventricular ejection fraction, reduce E/E′ ratio, reduce IVRT, reduce elevated right ventricle free wall thickness (RVFWT), increase tricuspid annular plane systolic excursion (TAPSE)) in a left heart failure-induced PH model (TAC-PH). While not wishing to be bound to any particular mechanism, it is expected that the effects of these agents is caused primarily by an ActRII (ActRIIA and/or ActRIIB) signaling antagonist effect. Regardless of the mechanism, it is apparent from the data presented herein that ActRII antagonists decrease cardiac hypertrophy, restore left ventricular ejection fraction, and have other positivity effects in treating post-capillary pulmonary hypertension.

The animal models for PcPH (e.g., WHO Group 2) that were used in the studies described herein are considered to be predicative of efficacy in humans, and therefore, this disclosure provides methods for using ActRIIA polypeptides or ActRIIB polypeptides and other ActRII antagonists to treat PcPH (e.g., WHO Group 2 and/or Group 5 PH), particularly treating, preventing, or reducing the severity or duration of one or more complications of PcPH, in humans. As disclosed herein, the term ActRII antagonists refers a variety of agents that may be used to antagonize ActRII signaling including, for example, antagonists that inhibit one or more TGF-beta family ligands [e.g., activin A, activin B, activin AB, activin AC, BMP6, BMP7, BMP9, BMP10, GDF3, GDF8, and GDF11]; antagonists that inhibit ActRIIA or ActRIIB; and antagonists that inhibit one or more downstream signaling components (e.g., Smad proteins).

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art. In general, such interval of accuracy is ±10%. Alternatively, and particularly in biological systems, the terms “about” and “approximately” may mean values that are within an order of magnitude, preferably ≤5-fold and more preferably ≤2-fold of a given value.

Numeric ranges disclosed herein are inclusive of the numbers defining the ranges. The term “between” as used in the present application is inclusive of the numbers defining the ranges. Moreover, all ranges disclosed herein are to be understood to encompass any and all subranges subsumed therein. For example, a stated range of “1 to 10” or “between 1 to 10” should be considered to include any and all subranges between and inclusive of the minimum value of 1 or more, e.g., 1 to 6.1, and ending with a maximum value of 10 or less, e.g., 5.5 to 10.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers.

### 2. ActRII Antagonists

In certain aspects, the disclosure relates to ActRII antagonists (e.g., ActRII polypeptides) and uses thereof (e.g., of treating, preventing, or reducing the progression rate and/or severity of post-capillary pulmonary hypertension (PcPH) or one or more complications of PcPH). As used herein, the term “ActRII” refers to the family of type II activin receptors. This family includes activin receptor type IIA (ActRIIA) and activin receptor type IIB (ActRIIB).

As used herein, the term “ActRIIB” refers to a family of activin receptor type IIB (ActRIIB) proteins from any species and variants derived from such ActRIIB proteins by mutagenesis or other modification. Reference to ActRIIB herein is understood to be a reference to any one of the currently identified forms. Members of the ActRIIB family are generally transmembrane proteins, composed of a ligand-binding extracellular domain comprising a cysteine-rich region, a transmembrane domain, and a cytoplasmic domain with predicted serine/threonine kinase activity.

The term “ActRIIB polypeptide” includes polypeptides comprising any naturally occurring polypeptide of an ActRIIB family member as well as any variants thereof (including mutants, fragments, fusions, and peptidomimetic forms) that retain a useful activity. Examples of such variant ActRIIB polypeptides are provided throughout the present disclosure as well as in International Patent Application Publication Nos. WO 2006/012627, WO 2008/097541, WO 2010/151426, WO 2011/020045, WO2019140283, WO2018/089706, WO2018/089715 WO2019/094751, WO2016/171948, and WO2018/075747 which are incorporated herein by reference in their entirety. Numbering of amino acids for all ActRIIB-related polypeptides described herein is based on the numbering of the human ActRIIB precursor protein sequence provided below (SEQ ID NO: 1), unless specifically designated otherwise.

The human ActRIIB precursor protein sequence is as follows:

| (SEQ ID NO: 1) | |
|---|---|
| 1 | MTAPWVALAL LWGSLCAGSG RGEAETRECI YYNANWELER T [symbol] QSGLERCE |
| 51 | GEQDKRLHCY ASWR [symbol] SSGTI ELVKKGCWLD DFNCYDRQEC VATEENPQVY |
| 101 | FCCCEGNFCN ERFTHLPEAG GPEVTYEPPP TAPTLLTVLA YSLLPIGGLS |
| 151 | LIVLLAFWMY RHRKPPYGHV DIHEDPGPPP PSPLVGLKPL QLLEIKARGR |
| 201 | FGCVWKAQLM NDEVAVKIFP LQDKQSWQSE REIFSTPGMK HENLLQFIAA |
| 251 | EKRGSNLEVE LWLITAFHDK GSLTDYLKGN IITWNELCHV AETMSRGLSY |
| 301 | LHEDVPWCRG EGHKPSIAHR DFKSKNVLLK SDLTAVLADF GLAVRFEPGK |
| 351 | PPGDTHGQVG TRRYMAPEVL EGAINFQRDA FLRIDMYAMG LVLWELVSRC |
| 401 | KAADGPVDEY MLPFEEEIGQ HPSLEELQEV VVHKKMRPTI KDHWLKHPGL |
| 451 | AQLCVTIEEC WDHDAEARLS AGCVEERVSL IRRSVNGTTS DCLVSLVTSV |
| 501 | TNVDLPPKES SI |

The signal peptide is indicated with a single underline; the extracellular domain is indicated in bold font; and the potential, endogenous N-linked glycosylation sites are indicated with a double underline.

The processed (mature) extracellular ActRIIB polypeptide sequence is as follows:

(SEQ ID NO: 2)
GRGEAETRECIYYNANWELERTNQSGLERCEGEQDKRLHCYASWRNSSG
TIELVKKGCWLDDFNCYDRQECVATEENPQVYFCCCEGNFCNERFTHLP
EAGGPEVTYEPPPTAPT.

In some embodiments, the protein may be produced with an “SGR . . . ” sequence at the N-terminus. The C-terminal “tail” of the extracellular domain is indicated by a single underline. The sequence with the “tail” deleted (a Δ15 sequence) is as follows:

(SEQ ID NO: 3)
GRGEAETRECIYYNANWELERTNQSGLERCEGEQDKRLHCYASWRNSSG
TIELVKKGCWLDDFNCYDRQECVATEENPQVYFCCCEGNFCNERFTHLP
EA.

A form of ActRIIB with an alanine at position 64 of SEQ ID NO: 1 (A64) is also reported in the literature. See, e.g., Hilden et al. (1994) Blood, 83(8): 2163-2170. Applicants have ascertained that an ActRIIB-Fc fusion protein comprising an extracellular domain of ActRIIB with the A64 substitution has a relatively low affinity for activin and GDF11. By contrast, the same ActRIIB-Fc fusion protein with an arginine at position 64 (R64) has an affinity for activin and GDF11 in the low nanomolar to high picomolar range. Therefore, sequences with an R64 are used as the “wild-type” reference sequence for human ActRIIB in this disclosure.

The form of ActRIIB with an alanine at position 64 is as follows:

| (SEQ ID NO: 4) | |
|---|---|
| 1 | MTAPWVALAL LWGSLCAGSG RGEAETRECI YYNANWELER TNQSGLERCE |
| 51 | GEQDKRLHCY ASWANSSGTI ELVKKGCWLD DFNCYDRQEC VATEENPQVY |
| 101 | FCCCEGNFCN ERFTHLPEAG GPEVTYEPPP TAPTLLTVLA YSLLPIGGLS |
| 151 | LIVLLAFWMY RHRKPPYGHV DIHEDPGPPP PSPLVGLKPL QLLEIKARGR |
| 201 | FGCVWKAQLM NDFVAVKIFP LQDKQSWQSE REIFSTPGMK HENLLQFIAA |
| 251 | EKRGSNLEVE LWLITAFHDK GSLTDYLKGN IITWNELCHV AETMSRGLSY |
| 301 | LHEDVPWCRG EGHKPSIAHR DFKSKNVLLK SDLTAVLADF GLAVRFEPGK |
| 351 | PPGDTHGQVG TRRYMAPEVL EGAINFQRDA FLRIDMYAMG LVLWELVSRC |
| 401 | KAADGPVDEY MLPFEEEIGQ HPSLEELQEV VVHKKMRPTI KDHWLKHPGL |
| 451 | AQLCVTIEEC WDHDAEARLS AGCVEERVSL IRRSVNGTTS DCLVSLVTSV |
| 501 | TNVDLPPKES SI |

The signal peptide is indicated by single underline and the extracellular domain is indicated by bold font.

The processed (mature) extracellular ActRIIB polypeptide sequence of the alternative A64 form is as follows:

(SEQ ID NO: 5)
GRGEAETRECIYYNANWELERTNQSGLERCEGEQDKRLHCYASWANSSG
TIELVKKGCWLDDFNCYDRQECVATEENPQVYFCCCEGNFCNERFTHLP
EAGGPEVTYEPPPTAPT

In some embodiments, the protein may be produced with an “SGR . . . ” sequence at the N-terminus. The C-terminal “tail” of the extracellular domain is indicated by single underline. The sequence with the “tail” deleted (a Δ15 sequence) is as follows:

(SEQ ID NO: 6)
GRGEAETRECIYYNANWELERTNQSGLERCEGEQDKRLHCYASWANSSG
TIELVKKGCWLDDFNCYDRQECVATEENPQVYFCCCEGNFCNERFTHLP
EA

A nucleic acid sequence encoding the human ActRIIB precursor protein is shown below (SEQ ID NO: 7), representing nucleotides 25-1560 of Genbank Reference Sequence NM_001106.3, which encode amino acids 1-513 of the ActRIIB precursor. The sequence as shown provides an arginine at position 64 and may be modified to provide an alanine instead.

The signal sequence is underlined.

| (SEQ ID NO: 7) | |
|---|---|
| 1 | ATGACGGCGC CCTGGGTGGC CCTCGCCCTC CTCTGGGGAT CGCTGTGCGC |
| 51 | CGGCTCTGGG CGTGGGGAGG CTGAGACACG GGAGTGCATC TACTACAACG |
| 101 | CCAACTGGGA GCTGGAGCGC ACCAACCAGA GCGGCCTGGA GCGCTGCGAA |
| 151 | GGCGAGCAGG ACAAGCGGCT GCACTGCTAC GCCTCCTGGC GCAACAGCTC |
| 201 | TGGCACCATC GAGCTCGTGA AGAAGGGCTG CTGGCTAGAT GACTTCAACT |
| 251 | GCTACGATAG GCAGGAGTGT GTGGCCACTG AGGAGAACCC CCAGGTGTAC |
| 301 | TTCTGCTGCT GTGAAGGCAA CTTCTGCAAC GAACGCTTCA CTCATTTGCC |
| 351 | AGAGGCTGGG GGCCCGGAAG TCACGTACGA GCCACCCCCG ACAGCCCCCA |
| 401 | CCCTGCTCAC GGTGCTGGCC TACTCACTGC TGCCCATCGG GGGCCTTTCC |
| 451 | CTCATCGTCC TGCTGGCCTT TTGGATGTAC CGGCATCGCA AGCCCCCCTA |
| 501 | CGGTCATGTG GACATCCATG AGGACCCTGG GCCTCCACCA CCATCCCCTC |
| 551 | TGGTGGGCCT GAAGCCACTG CAGCTGCTGG AGATCAAGGC TCGGGGGCGC |
| 601 | TTTGGCTGTG TCTGGAAGGC CCAGCTCATG AATGACTTTG TAGCTGTCAA |
| 651 | GATCTTCCCA CTCCAGGACA AGCAGTCGTG GCAGAGTGAA CGGGAGATCT |
| 701 | TCAGCACACC TGGCATGAAG CACGAGAACC TGCTACAGTT CATTGCTGCC |
| 751 | GAGAAGCGAG GCTCCAACCT CGAAGTAGAG CTGTGGCTCA TCACGGCCTT |
| 801 | CCATGACAAG GGCTCCCTCA CGGATTACCT CAAGGGGAAC ATCATCACAT |
| 851 | GGAACGAACT GTGTCATGTA GCAGAGACGA TGTCACGAGG CCTCTCATAC |
| 901 | CTGCATGAGG ATGTGCCCTG GTGCCGTGGC GAGGGCCACA AGCCGTCTAT |
| 951 | TGCCCACAGG GACTTTAAAA GTAAGAATGT ATTGCTGAAG AGCGACCTCA |
| 1001 | CAGCCGTGCT GGCTGACTTT GGCTTGGCTG TTCGATTTGA GCCAGGGAAA |
| 1051 | CCTCCAGGGG ACACCCACGG ACAGGTAGGC ACGAGACGGT ACATGGCTCC |
| 1101 | TGAGGTGCTC GAGGGAGCCA TCAACTTCCA GAGAGATGCC TTCCTGCGCA |
| 1151 | TTGACATGTA TGCCATGGGG TTGGTGCTGT GGGAGCTTGT GTCTCGCTGC |
| 1201 | AAGGCTGCAG ACGGACCCGT GGATGAGTAC ATGCTGCCCT TTGAGGAAGA |
| 1251 | GATTGGCCAG CACCCTTCGT TGGAGGAGCT GCAGGAGGTG GTGGTGCACA |
| 1301 | AGAAGATGAG GCCCACCATT AAAGATCACT GGTTGAAACA CCCGGGCCTG |
| 1351 | GCCCAGCTTT GTGTGACCAT CGAGGAGTGC TGGGACCATG ATGCAGAGGC |
| 1401 | TCGCTTGTCC GCGGGCTGTG TGGAGGAGCG GGTGTCCCTG ATTCGGAGGT |
| 1451 | CGGTCAACGG CACTACCTCG GACTGTCTCG TTTCCCTGGT GACCTCTGTC |
| 1501 | ACCAATGTGG ACCTGCCCCC TAAAGAGTCA AGCATC |

A nucleic acid sequence encoding processed extracellular human ActRIIB polypeptide is as follows (SEQ ID NO: 8). The sequence as shown provides an arginine at position 64, and may be modified to provide an alanine instead.

| (SEQ ID NO: 8) | |
|---|---|
| 1 | GGGCGTGGGG AGGCTGAGAC ACGGGAGTGC ATCTACTACA ACGCCAACTG |
| 51 | GGAGCTGGAG CGCACCAACC AGAGCGGCCT GGAGCGCTGC GAAGGCGAGC |
| 101 | AGGACAAGCG GCTGCACTGC TACGCCTCCT GGCGCAACAG CTCTGGCACC |
| 151 | ATCGAGCTCG TGAAGAAGGG CTGCTGGCTA GATGACTTCA ACTGCTACGA |
| 201 | TAGGCAGGAG TGTGTGGCCA CTGAGGAGAA CCCCCAGGTG TACTTCTGCT |
| 251 | GCTGTGAAGG CAACTTCTGC AACGAACGCT TCACTCATTT GCCAGAGGCT |
| 301 | GGGGGCCCGG AAGTCACGTA CGAGCCACCC CCGACAGCCC CCACC |

In some embodiments the ActRIIB polypeptide comprises the accession number NP_001097.2 (SEQ ID NO: 1 herein), and variants thereof. In some embodiments, the term “wild-type ActRIIB” refers to the extracellular domain of ActRIIB, amino acids 1 to 134 (with signal sequence), or amino acids 19 through 134 of SEQ ID NO: 1 (without signal sequence) (referred to herein as SEQ ID NO: 407).

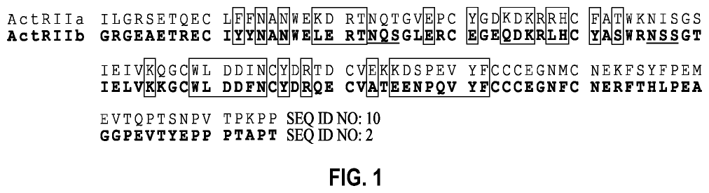

An alignment of the amino acid sequences of human ActRIIB extracellular domain and human ActRIIA extracellular domain are illustrated in FIG. 1. This alignment indicates amino acid residues within both receptors that are believed to directly contact ActRII ligands. For example, the composite ActRII structures indicated that the ActRIIB-ligand binding pocket is defined, in part, by residues Y31, N33, N35, L38 through T41, E47, E50, Q53 through K55, L57, H58, Y60, S62, K74, W78 through N83, Y85, R87, A92, and E94 through F101 (based on the numbering of SEQ ID NO: 1). At these positions, it is expected that conservative mutations will be tolerated.

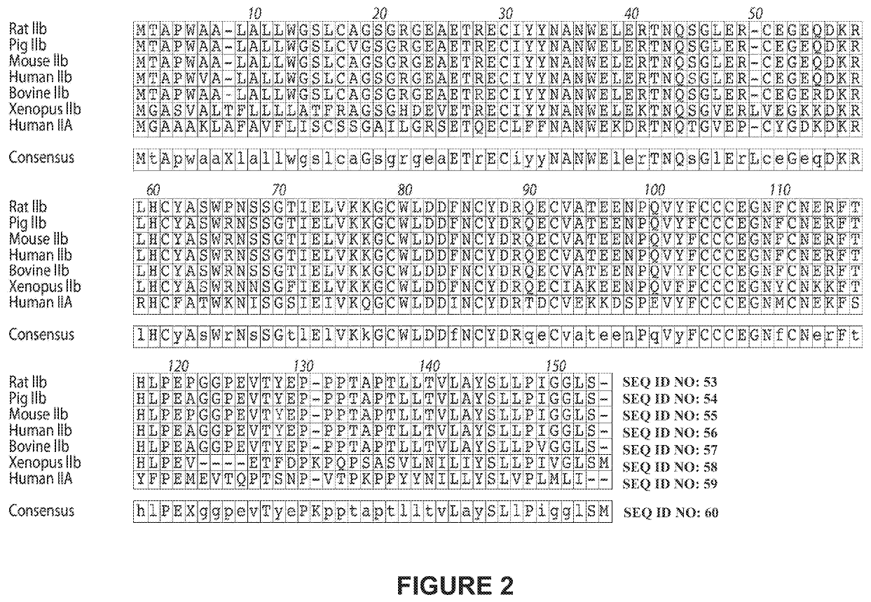

In addition, ActRIIB is well-conserved among vertebrates, with large stretches of the extracellular domain completely conserved. For example, FIG. 2 depicts a multi-sequence alignment of a human ActRIIB extracellular domain compared to various ActRIIB orthologs. Many of the ligands that bind to ActRIIB are also highly conserved. Accordingly, from these alignments, it is possible to predict key amino acid positions within the ligand-binding domain that are important for normal ActRIIB-ligand binding activities as well as to predict amino acid positions that are likely to be tolerant to substitution without significantly altering normal ActRIIB-ligand binding activities. Therefore, an active, human ActRIIB variant polypeptide useful in accordance with the presently disclosed methods may include one or more amino acids at corresponding positions from the sequence of another vertebrate ActRIIB, or may include a residue that is similar to that in the human or other vertebrate sequences. Without meaning to be limiting, the following examples illustrate this approach to defining an active ActRIIB variant. L46 in the human extracellular domain (SEQ ID NO: 2) is a valine in Xenopus ActRIIB (SEQ ID NO: 58), and so this position may be altered, and optionally may be altered to another hydrophobic residue, such as V, I or F, or a non-polar residue such as A. E52 in the human extracellular domain is a K in Xenopus, indicating that this site may be tolerant of a wide variety of changes, including polar residues, such as E, D, K, R, H, S, T, P, G, Y and probably A. T93 in the human extracellular domain is a K in Xenopus, indicating that a wide structural variation is tolerated at this position, with polar residues favored, such as S, K, R, E, D, H, G, P, G and Y. F108 in the human extracellular domain is a Y in Xenopus, and therefore Y or other hydrophobic group, such as I, V or L should be tolerated. E111 in the human extracellular domain is K in Xenopus, indicating that charged residues will be tolerated at this position, including D, R, K and H, as well as Q and N. R112 in the human extracellular domain is K in Xenopus, indicating that basic residues are tolerated at this position, including R and H. A at position 119 in the human extracellular domain is relatively poorly conserved, and appears as P in rodents and V in Xenopus, thus essentially any amino acid should be tolerated at this position.

Moreover, ActRII proteins have been characterized in the art in terms of structural and functional characteristics, particularly with respect to ligand binding [Attisano et al. (1992) Cell 68(1):97-108; Greenwald et al. (1999) Nature Structural Biology 6(1): 18-22; Allendorph et al. (2006) PNAS 103(20: 7643-7648; Thompson et al. (2003) The EMBO Journal 22(7): 1555-1566; as well as U.S. Pat. Nos. 7,709,605, 7,612,041, and 7,842,663]. In addition to the teachings herein, these references provide amply guidance for how to generate ActRIIB variants that retain one or more normal activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRIIB, as demarcated by the outermost of these conserved cysteines, corresponds to positions 29-109 of SEQ ID NO: 1 (ActRIIB precursor). The structurally less-ordered amino acids flanking these cysteine-demarcated core sequences can be truncated by 1, 2, 3, 4, 5, 6, 7, 8, 9, 10, 11, 12, 13, 14, 15, 16, 17, 18, 19, 20, 21, 22, 23, 24, 25, 26, 27, or 28 residues at the N-terminus and/or 1, 2, 3, 4, 5, 6, 7, 8, 9, 10, 11, 12, 13, 14, 15, 16, 17, 18, 19, 20, 21, or 22 residues a the C-terminus without necessarily altering ligand binding. Exemplary ActRIIB extracellular domains for N-terminal and/or C-terminal truncation include SEQ ID NOs: 2, 3, 5, 6, 318, and 331.

Attisano et al. showed that a deletion of the proline knot at the C-terminus of the extracellular domain of ActRIIB reduced the affinity of the receptor for activin. An ActRIIB-Fc fusion protein containing amino acids 20-119 of present SEQ ID NO: 1, “ActRIIB(20-119)-Fc”, has reduced binding to GDF11 and activin relative to an ActRIIB(20-134)-Fc, which includes the proline knot region and the complete juxtamembrane domain (see, e.g., U.S. Pat. No. 7,842,663). However, an ActRIIB(20-129)-Fc protein retains similar, but somewhat reduced activity, relative to the wild-type, even though the proline knot region is disrupted.

Thus, ActRIIB extracellular domains that stop at amino acid 134, 133, 132, 131, 130 and 129 (with respect to SEQ ID NO: 1) are all expected to be active, but constructs stopping at 134 or 133 may be most active. Similarly, mutations at any of residues 129-134 (with respect to SEQ ID NO: 1) are not expected to alter ligand-binding affinity by large margins. In support of this, it is known in the art that mutations of P129 and P130 (with respect to SEQ ID NO: 1) do not substantially decrease ligand binding. Therefore, an ActRIIB polypeptide of the present disclosure may end as early as amino acid 109 (the final cysteine), however, forms ending at or between 109 and 119 (e.g., 109, 110, 111, 112, 113, 114, 115, 116, 117, 118, or 119) are expected to have reduced ligand binding. Amino acid 119 (with respect to present SEQ ID NO:1) is poorly conserved and so is readily altered or truncated. ActRIIB polypeptides ending at 128 (with respect to SEQ ID NO: 1) or later should retain ligand-binding activity. ActRIIB polypeptides ending at or between 119 and 127 (e.g., 119, 120, 121, 122, 123, 124, 125, 126, or 127), with respect to SEQ ID NO: 1, will have an intermediate binding ability. Any of these forms may be desirable to use, depending on the clinical or experimental setting.

At the N-terminus of ActRIIB, it is expected that a protein beginning at amino acid 29 or before (with respect to SEQ ID NO: 1) will retain ligand-binding activity. Amino acid 29 represents the initial cysteine. An alanine-to-asparagine mutation at position 24 (with respect to SEQ ID NO: 1) introduces an N-linked glycosylation sequence without substantially affecting ligand binding [U.S. Pat. No. 7,842,663]. This confirms that mutations in the region between the signal cleavage peptide and the cysteine cross-linked region, corresponding to amino acids 20-29, are well tolerated. In particular, ActRIIB polypeptides beginning at position 20, 21, 22, 23, and 24 (with respect to SEQ ID NO: 1) should retain general ligand-biding activity, and ActRIIB polypeptides beginning at positions 25, 26, 27, 28, and 29 (with respect to SEQ ID NO: 1) are also expected to retain ligand-biding activity. It has been demonstrated, e.g., U.S. Pat. No. 7,842,663, that, surprisingly, an ActRIIB construct beginning at 22, 23, 24, or 25 will have the most activity.

Taken together, a general formula for an active portion (e.g., ligand-binding portion) of ActRIIB comprises amino acids 29-109 of SEQ ID NO: 1. Therefore ActRIIB polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIB beginning at a residue corresponding to any one of amino acids 20-29 (e.g., beginning at any one of amino acids 20, 21, 22, 23, 24, 25, 26, 27, 28, or 29) of SEQ ID NO: 1 and ending at a position corresponding to any one amino acids 109-134 (e.g., ending at any one of amino acids 109, 110, 111, 112, 113, 114, 115, 116, 117, 118, 119, 120,121, 122, 123, 124, 125, 126, 127, 128, 129, 130, 131, 132, 133, or 134) of SEQ ID NO: 1. Other examples include polypeptides that begin at a position from 20-29 (e.g., any one of positions 20, 21, 22, 23, 24, 25, 26, 27, 28, or 29) or 21-29 (e.g., any one of positions 21, 22, 23, 24, 25, 26, 27, 28, or 29) of SEQ ID NO: 1 and end at a position from 119-134 (e.g., any one of positions 119, 120, 121, 122, 123, 124, 125, 126, 127, 128, 129, 130, 131, 132, 133, or 134), 119-133 (e.g., any one of positions 119, 120, 121, 122, 123, 124, 125, 126, 127, 128, 129, 130, 131, 132, or 133), 129-134 (e.g., any one of positions 129, 130, 131, 132, 133, or 134), or 129-133 (e.g., any one of positions 129, 130, 131, 132, or 133) of SEQ ID NO: 1. Other examples include constructs that begin at a position from 20-24 (e.g., any one of positions 20, 21, 22, 23, or 24), 21-24 (e.g., any one of positions 21, 22, 23, or 24), or 22-25 (e.g., any one of positions 22, 22, 23, or 25) of SEQ ID NO: 1 and end at a position from 109-134 (e.g., any one of positions 109, 110, 111, 112, 113, 114, 115, 116, 117, 118, 119, 120, 121, 122, 123, 124, 125, 126, 127, 128, 129, 130, 131, 132, 133, or 134), 119-134 (e.g., any one of positions 119, 120, 121, 122, 123, 124, 125, 126, 127, 128, 129, 130, 131, 132, 133, or 134) or 129-134 (e.g., any one of positions 129, 130, 131, 132, 133, or 134) of SEQ ID NO: 1. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 1.

The variations described herein may be combined in various ways. In some embodiments, ActRIIB variants comprise no more than 1, 2, 5, 6, 7, 8, 9, 10 or 15 conservative amino acid changes in the ligand-binding pocket, optionally zero, one or more non-conservative alterations at positions 40, 53, 55, 74, 79 and/or 82 in the ligand-binding pocket. Sites outside the binding pocket, at which variability may be particularly well tolerated, include the amino and carboxy termini of the extracellular domain (as noted above), and positions 42-46 and 65-73 (with respect to SEQ ID NO: 1). An asparagine-to-alanine alteration at position 65 (N65A) does not appear to decrease ligand binding in the R64 background [U.S. Pat. No. 7,842,663]. This change probably eliminates glycosylation at N65 in the A64 background, thus demonstrating that a significant change in this region is likely to be tolerated. While an R64A change is poorly tolerated, R64K is well-tolerated, and thus another basic residue, such as H may be tolerated at position 64 [U.S. Pat. No. 7,842,663]. Additionally, the results of the mutagenesis program described in the art indicate that there are amino acid positions in ActRIIB that are often beneficial to conserve. With respect to SEQ ID NO: 1, these include position 80 (acidic or hydrophobic amino acid), position 78 (hydrophobic, and particularly tryptophan), position 37 (acidic, and particularly aspartic or glutamic acid), position 56 (basic amino acid), position 60 (hydrophobic amino acid, particularly phenylalanine or tyrosine). Thus, the disclosure provides a framework of amino acids that may be conserved in ActRIIB polypeptides. Other positions that may be desirable to conserve are as follows: position 52 (acidic amino acid), position 55 (basic amino acid), position 81 (acidic), 98 (polar or charged, particularly E, D, R or K), all with respect to SEQ ID NO: 1.

It has been previously demonstrated that the addition of a further N-linked glycosylation site (N-X-S/T) into the ActRIIB extracellular domain is well-tolerated (see, e.g., U.S. Pat. No. 7,842,663). Therefore, N-X-S/T sequences may be generally introduced at positions outside the ligand binding pocket defined in FIG. 1 in ActRIIB polypeptide of the present disclosure. Particularly suitable sites for the introduction of non-endogenous N-X-S/T sequences include amino acids 20-29, 20-24, 22-25, 109-134, 120-134 or 129-134 (with respect to SEQ ID NO: 1). N-X-S/T sequences may also be introduced into the linker between the ActRIIB sequence and an Fc domain or other fusion component as well as optionally into the fusion component itself. Such a site may be introduced with minimal effort by introducing an N in the correct position with respect to a pre-existing S or T, or by introducing an S or T at a position corresponding to a pre-existing N. Thus, desirable alterations that would create an N-linked glycosylation site are: A24N, R64N, S67N (possibly combined with an N65A alteration), E105N, R112N, G120N, E123N, P129N, A132N, R112S and R112T (with respect to SEQ ID NO: 1). Any S that is predicted to be glycosylated may be altered to a T without creating an immunogenic site, because of the protection afforded by the glycosylation. Likewise, any T that is predicted to be glycosylated may be altered to an S. Thus the alterations S67T and S44T (with respect to SEQ ID NO: 1) are contemplated. Likewise, in an A24N variant, an S26T alteration may be used. Accordingly, an ActRIIB polypeptide of the present disclosure may be a variant having one or more additional, non-endogenous N-linked glycosylation consensus sequences as described above.

In certain embodiments, the disclosure relates to ActRII antagonists (inhibitors) that comprise a ActRIIB polypeptide, which includes fragments, functional variants, and modified forms thereof as well as uses thereof (e.g., treating or preventing PcPH or one or more PcPH-associated complication). Preferably, ActRIIB polypeptides are soluble (e.g., comprise an extracellular domain of ActRIIB). In some embodiments, ActRIIB polypeptides antagonize activity (e.g., Smad signaling) of one or more TGF-beta family ligands [e.g., activin A, activin B, BMP6, BMP9, BMP10, GDF3, GDF8, and/or GDF11]. Therefore, in some embodiments, ActRIIB polypeptides bind to one or more TGF-beta family ligands [e.g., activin A, activin B, BMP6, BMP9, BMP10, GDF3, GDF8, and/or GDF11]. In some embodiments, ActRIIB polypeptides of the disclosure demonstrate a decreased binding affinity for BMP9. In some embodiments, ActRIIB polypeptides of the disclosure do not bind BMP9. In some embodiments, ActRIIB polypeptides of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIB beginning at a residue corresponding to amino acids 20-29 (e.g., beginning at any one of amino acids 20, 21, 22, 23, 24, 25, 26, 27, 28, or 29) of SEQ ID NO: 1 and ending at a position corresponding to amino acids 109-134 (e.g., ending at any one of amino acids 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1. In some embodiments, ActRIIB polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 29-109 of SEQ ID NO: 1. In some embodiments, ActRIIB polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 29-109 of SEQ ID NO: 1, wherein the position corresponding to L79 of SEQ ID NO: 1 is an acidic amino acid (naturally occurring acidic amino acids D and E or an artificial acidic amino acid). In certain embodiments, ActRIIB polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 25-131 of SEQ ID NO: 1. In certain embodiments, ActRIIB polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 25-131 of SEQ ID NO: 1, wherein the position corresponding to L79 of SEQ ID NO: 1 is an acidic amino acid. In some embodiments, ActRIIB polypeptide of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 4, 5, 6, 40, 42, 45, 46, 47, 48, 69, 74, 77, 78, 79, 138, 282, 289, 290, 291, 292, 293, 294, 295, 296, 297, 298, 299, 300, 301, 302, 303, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, and 407. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 1. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 2. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 3. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 4. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 5. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 6. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 40. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 42. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 45. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 46. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 47. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 48. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 69. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 74. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 77. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 78. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 79. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 138. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 282. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 289. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 290. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 291. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 292. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 293. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 294. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 295. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 296. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 297. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 298. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 299. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 300. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 301. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 302. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 303. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 305. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 306. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 307. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 308. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 309. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 310. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 311. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 312. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 313. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 314. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 315. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 316. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 317. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 318. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 319. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 320. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 321. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 322. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 323. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 324. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 325. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 326. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 327. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 328. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 329. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 330. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 331. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 332. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 333. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 334. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 335. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 336. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 337. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 338. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 339. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 340. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 341. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 342. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 343. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 344. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 345. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 346. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 347. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 348. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 349. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 350. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 351. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 352. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 353. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 354. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 355. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 356. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 357. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 358. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 359. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 360. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 361. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 362. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 363. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 364. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 365. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 366. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 367. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 368. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 369. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 370. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 371. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 372. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 373. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 374. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 375. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 376. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 377. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 378. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 379. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 380. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 381. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 382. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 383. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 384. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 385. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 386. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 387. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 388. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 389. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 390. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 391. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 392. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 393. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 394. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 395. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 396. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 397. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 398. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 399. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 400. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 401. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 402. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 403. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 404. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 405. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 406. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 407. In some embodiments, ActRIIB polypeptide of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 4, 5, 6, 40, 42, 45, 46, 47, 48, 69, 74, 77, 78, 79, 138, 282, 289, 290, 291, 292, 293, 294, 295, 296, 297, 298, 299, 300, 301, 302, 303, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, and 407, wherein the position corresponding to L79 of SEQ ID NO: 1 is an acidic amino acid. In some embodiments, ActRIIB polypeptides of the disclosure comprise, consist, or consist essentially of, at least one ActRIIB polypeptide wherein the position corresponding to L79 of SEQ ID NO: 1 is not an acidic amino acid (i.e., is not naturally occurring acid amino acids D or E or an artificial acidic amino acid residue).

In some embodiments, the ActRIIB polypeptide of the disclosure comprises an alternate, soluble form of ActRIIB (designated ActRIIB5), in which exon 4, including the ActRIIB transmembrane domain, has been replaced by a different C-terminal sequence (see, e.g., WO 2007/053775). In some embodiments, ActRIIB5 polypeptides of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a polypeptide selected from the group consisting of SEQ ID NOs: 50, 51, or 52.

In some embodiments, ActRIIB polypeptides of the disclosure comprise, consist, or consist essentially of, at least one extracellular ActRIIB variant polypeptide having the sequence of SEQ ID NO: 282 shown below:

(SEQ ID NO: 282)
GRGEAETRECIFYNANWEKDRTNQSGLEPCYGDQDKRRHCFASWKNSSG
TIELVKQGCWLDDINCYDRQECVAKKDSPEVYFCCCEGNFCNERFTHLP
EAGGPEVTYEPPPTAPT.

In some embodiments, ActRIIB polypeptides of the disclosure comprise, consist, or consist essentially of, at least one extracellular ActRIIB variant polypeptide having the sequence of any one of SEQ ID NOs: 282, 289, or 290-302. In some embodiments, ActRIIB polypeptides of the disclosure comprise, consist, or consist essentially of, at least one extracellular ActRIIB variant polypeptide having the sequence of any one of SEQ ID NOs: 282 or 290-302 (Table 3).

In some embodiments, ActRIIB polypeptides of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the processed (mature) extracellular ActRIIB polypeptide sequence (SEQ ID NO: 2).

Polypeptides described herein include an extracellular ActRIIB variant having at least one amino acid substitution relative to the processed (mature) extracellular ActRIIB polypeptide sequence having the sequence of SEQ ID NO: 2. Possible amino acid substitutions at 28 different positions may be introduced to an extracellular ActRIIB variant (Table 1). An extracellular ActRIIB variant may have one or more (e.g., 1-28, 1-25, 1-23, 1-21, 1-19, 1-17, 1-15, 1-13, 1-11, 1-9, 1-7, 1-5, 1-3, or 1-2; e.g., 1, 2, 3, 4, 5, 6, 7, 8, 9, 10, 11, 12, 13, 14, 15, 16, 17, 18, 19, 20, 21, 22, 23, 24, 25, 26, or 27) amino acid substitutions relative the sequence of a processed (mature) extracellular ActRIIB polypeptide sequence (SEQ ID NO: 2). In some embodiments, an extracellular ActRIIB variant (e.g., an extracellular ActRIIB variant having a sequence of SEQ ID NO: 289) may include amino acid substitutions at all of the 28 positions as listed in Table 1. In some embodiments, an extracellular ActRIIB variant may include amino acid substitutions at a number of positions, e.g., at 3, 4, 5, 6, 7, 8, 9, 10, 12, 15, 16, 18, 20, 22, 24, 26, or 27 out of the 28 positions, as listed in Table 1. In some embodiments, the substitutions are substitutions of an amino acid from an ActRIIA polypeptide sequence into the same position in an ActRIIB polypeptide sequence. In some embodiments, the substitutions are novel changes (e.g., substitutions of amino acids that are not in the corresponding position of ActRIIA, e.g., S48T, I51L, Q69D, or E70T).

Amino acid substitutions can worsen or improve the activity and/or binding affinity of the ActRIIB variants disclosed herein (e.g., an extracellular ActRIIB variant having the sequence of any one of SEQ ID NOs: 282, 289, and 290-30 (e.g., SEQ ID NOs: 282 and 290-302)). In some embodiments, the amino acid substitutions worsen the binding affinity of the ActRIIB variants to BMP9 (e.g., the variants have reduced binding to BMP9 relative to wild-type extracellular ActRIIB, or have lower binding to BMP9 than to other ActRIIB ligands (e.g., activin A or B, myostatin, or GDF-11)). In some embodiments, the ActRIIB variants have reduced or no substantial binding to BMP9. In some embodiments, the amino acid substitutions improve the binding affinity of ActRIIB to myostatin, activin A or B, and/or GDF-11 (e.g., the variants have improved binding affinity relative to wild-type extracellular ActRIIB, or bind more strongly to myostatin, activin A or B, or GDF-11 than to BMP9). In some embodiments, the amino acid substitutions reduce the binding affinity of ActRIIB to myostatin, activin A or B, and/or GDF-11 (e.g., the variants have decreased binding affinity relative to wild-type extracellular ActRIIB, or have reduced binding to myostatin, activin A or B, or GDF-11 as compared to BMP9). In some embodiments, the amino acid substitutions do not substantially change extracellular ActRIIB function (e.g., the ActRIIB variants increase lean mass, muscle, mass, or bone mineral density, or reduce or prevent fibrosis, by a similar amount as wild-type extracellular ActRIIB, e.g., the ActRIIB variants are functionally equivalent to the wild-type extracellular ActRIIB). In some embodiments, the amino acid substitutions confer a property or activity of an ActRIIA polypeptide on an ActRIIB variant polypeptide (e.g., the ActRIIB variant polypeptide has a longer half-life than wild-type extracellular ActRIIB). In some embodiments, the ActRIIB variant polypeptides have one or more, two or more, or three or more of the above properties (e.g., reduced BMP9 binding and improved binding to activin A or B, myostatin, and/or GDF-11, or reduced BMP9 binding and functional equivalence to wild-type ActRIIB).

In some embodiments, ActRIIB polypeptides of the disclosure (e.g., an extracellular ActRIIB variant having the sequence of any one of SEQ ID NOs: 282, 289, and 290-30 (e.g., SEQ ID NOs: 282 and 290-302)) have one or more amino acid substitutions that reduce BMP9 binding. In some embodiments, the amino acid substitution that reduces BMP9 binding is E75K (e.g., X₂₄ is K in SEQ ID NO: 289). In some embodiments, the amino acid substitutions that reduce BMP9 binding are Q69T and E70D (e.g., X₂₁ is T and X₂₂ is D in SEQ ID NO: 289). In some embodiments, the amino acid substitutions that reduce BMP9 binding are Q69D and E70T (e.g., X₂₁ is D and X₂₂ is T in SEQ ID NO: 289). In some embodiments, the amino acid substitutions that reduce BMP9 binding are T74K, E75K, E76D, N77S, and Q79E (e.g., X₂₃, X₂₄, X₂₅, X₂₆, and X₂₈ are K, K, D, S, and E, respectively, in SEQ ID NO: 289). In some embodiments, the ActRIIB variants have more than one of the aforementioned amino acid substitutions that reduce BMP9 binding (e.g., substitution E75K and substitutions Q69D and E70T, or substitution E75K and substitutions Q69T and E70D). In some embodiments, the ActRIIB variants disclosed herein have one or more amino acid substitutions that reduce BMP9 binding, and one or more additional amino acid substitutions. The additional amino acid substitutions may confer other beneficial properties, such as altered binding to activins or myostatin or improved activity. For example, amino acid substitutions T74K, E75K, E76D, N77S, and Q79E lead to a reduction in ActRIIB variant activity, but including additional substitutions S25T and S47I; E31Y, E33D, and Q34K; or Y41F, R45K, and K56Q improves the ActRIIB variant activity. The additional amino acid substitutions may include one or more of substitutions I11L, Y12F, L19K, E20D, S25T, L27V, R29P, E31Y, E33D, Q34K, L38R, Y41F, R45K, S47I, S48T, T50S, I51L, L53I, K56Q, F63I, T74K, E76D, N77S, Q79E, or F89M.

In some embodiments, variant ActRIIB polypeptides of the disclosure comprise one or more amino acid substitutions relative to the sequence of SEQ ID NO: 2, in which the variant contains one or more amino acid substitutions that impart reduced BMP9 binding relative to wild type extracellular ActRIIB, and one or more additional amino acid substitutions, wherein the substitutions that reduce BMP9 binding are one or more of: (a) amino acid substitution E75K; (b) amino acid substitutions Q69T and E70D; or (c) amino acid substitutions Q69D and E70T. In some embodiments, the one or more additional amino acid substitutions are selected from the group consisting of I11L, Y12F, L19K, E20D, S25T, L27V, R29P, E31Y, E33D, Q34K, L38R, Y41F, R45K, 5471, S48T, T50S, I51L, L53I, K56Q, F63I, T74K, E76D, N77S, Q79E, and F89M. In some embodiments, the variant contains amino acid substitution E75K and additional amino acid substitutions E20D and F63I. In some embodiments, the variant polypeptide further comprises amino acid substitution E75K. In some embodiments, the variant contains amino acid substitution E75K and additional amino acid substitutions that reduce BMP9 binding. In some embodiments of any of the above embodiments, the additional amino acid substitutions that reduce BMP9 binding are T74K, E76D, N77S, and Q79E. In some embodiments, the variant further contains one or more additional amino acid substitutions. In some embodiments, the variant contains additional amino acid substitutions Y41F, R45K, and K56Q. In some embodiments, the variant further contains additional amino acid substitutions Y12F, L19K, E20D, R29P, E31Y, E33D, L38R, and F63I. In some embodiments, the variant contains additional amino acid substitutions S25T and S47I. In some embodiments, the variant contains additional amino acid substitution S48T. In some embodiments, the variant contains additional amino acid substitution R29P. In some embodiments, the variant contains additional amino acid substitutions E31Y, E33D, and Q34K. In some embodiments, the variant contains additional amino acid substitutions Y12F, L19K, and E20D. In some embodiments, the variant contains additional amino acid substitutions E31Y, E33D, and L38R. In some embodiments, the variant contains amino acid substitutions Q69T and E70D, and additional amino acid substitutions I11L, L27V, Q34K, T50S, I51L, L53I, and F89M. In some embodiments, the variant contains amino acid substitutions Q69D and E70T, and additional amino acid substitutions I11L, L27V, Q34K, T50S, I51L, L53I, and F89M. In some embodiments, the variant further contains amino acid substitution E75K. In some embodiments, the variant polypeptide comprises the sequence of any one of SEQ ID NOs: 282 or 290-302. See, e.g., Table 3.

In some embodiments, a polypeptide described herein includes an extracellular ActRIIB variant having the sequence of SEQ ID NO: 289.

TABLE 1
Amino acid substitutions in an extracellular ActRIIB variant having a
sequence of SEQ ID NO: 289

GRGEAETRECX₁X₂YNANWEX₃X₄RTNQX₅GX₆EX₇CX₈GX₉X₁₀DKRX₁₁HCX₁₂ASWX
₁₃NX₁₄X₁₅GX₁₆X₁₇EX₁₈VKX₁₉GCWLDDX₂₀NCYDRX₂₁X₂₂CVAX₂₃X₂₄X₂₅X₂₆PX₂₇VYF
CCCEGNX₂₈CNERFTHLPEAGGPEVTYEPPPTAPT
(SEQ ID NO: 289)

| | | | |
|---|---|---|---|
| X₁ | I or L | X₁₅ | S, N, Q, or T |
| X₂ | F, Y, A, V, I, L, M, or W | X₁₆ | S, N, Q, or T |
| X₃ | L or K | X₁₇ | L, V, A, I, M, F, Y, or W |
| X₄ | D, E, or A | X₁₈ | L, V, A, I, M, F, Y, or W |
| X₅ | T, S, N, or Q | X₁₉ | K or Q |
| X₆ | L, V, A, I, M, F, Y, | X₂₀ | L, V, A, I, M, F, Y, |
| | or W | | or W |
| X₇ | P or R | X₂₁ | Q, T, S, N or D |
| X₈ | Y or E | X₂₂ | E, D, A, or T |
| X₉ | D, E, or A | X₂₃ | K or T |
| X₁₀ | K or Q | X₂₄ | K or E |
| X₁₁ | R or L | X₂₅ | D, E, or A |
| X₁₂ | A, V, I, L, M, Y, W or F | X₂₆ | T, S, N, or Q |
| X₁₃ | R, H, or K | X₂₇ | E or Q |
| X₁₄ | S or I | X₂₈ | L, V, A, I, M, F, Y, or W |

TABLE 2
Compositions that can be administered to a subject according to the methods
described herein.

| Row | Composition |
|---|---|
| 1 | A polypeptide containing an ActRIIB variant, the variant having a sequence of |
| | GRGEAETRECX₁X₂YNANWEX₃X₄RTNQX₅GX₆EX₇CX₈GX₉X₁₀DKRX₁₁HCX₁₂AS |
| | WX₁₃NX₁₄X₁₅GX₁₆X₁₇EX₁₈VKX₁₉GCWLDDX₂₀NCYDRX₂₁X₂₂CVAX₂₃X₂₄X₂₅X₂₆PX₂₇ |
| | VYFCCCEGNX₂₈CNERFTHLPEAGGPEVTYEPPPTAPT (SEQ ID NO: 289), |
| | wherein X₁ is I or L; X₂ is F or Y; X₃ is L or K; X₄ is D or E; X₅ is T or S; X₆ is L or V; |
| | X₇ is P or R; X₈ is Y or E; X₉ is D or E; X₁₀ is K or Q; X₁₁ is R or L; X₁₂ is Y or F; X₁₃ |
| | is R or K; X₁₄ is S or I; X₁₅ is S or T; X₁₆ is S or T; X₁₇ is I or L; X₁₈ is I or L; X₁₉ is |
| | K or Q; X₂₀ is F or I; X₂₁ is Q, T, or D; X₂₂ is E, D, or T; X₂₃ is K or T; X₂₄ is K or E; |
| | X₂₅ is D or E; X₂₆ is S or N; X₂₇ is E or Q; and X₂₈ is F or M, and wherein X₂₄ is E and/or |
| | either X₂₁ is T and X₂₂ is D or X₂₁ is D and X₂₂ is T. |
| 2 | A polypeptide containing an ActRIIB variant, the variant having a sequence of |
| | GRGEAETRECX₁X₂YNANWEX₃X₄RTNQX₅GX₆EX₇CX₈GX₉X₁₀DKRX₁₁HCX₁₂AS |
| | WX₁₃NX₁₄X₁₅GX₁₆X₁₇EX₁₈VKX₁₉GCWLDDX₂₀NCYDRX₂₁X₂₂CVAX₂₃X₂₄X₂₅X₂₆PX₂₇ |
| | VYFCCCEGNX₂₈CNERFTHLPEAGGPEVTYEPPPTAPT (SEQ ID NO: 289), |
| | wherein X₁ is I or L; X₂ is F, Y, A, V, I, L, M, or W; X₃ is L or K; X₄ is D, E, or A; X₅ |
| | is T, S, N, or Q; X₆ is L, V, A, I, M, F, Y, or W; X₇ is P or R; X₈ is Y or E; X₉ is D, E, |
| | or A; X₁₀ is K or Q; X₁₁ is R or L; X₁₂ is A, V, I, L, M, Y, W or F; X₁₃ is R, H, or K; |
| | X₁₄ is S or I; X₁₅ is S, N, Q, or T; X₁₆ is S, N, Q, or T; X₁₇ is L, V, A, I, M, F, Y, or |
| | W; X₁₈ is L, V, A, I, M, F, Y, or W; X₁₉ is K or Q; X₂₀ is L, V, A, I, M, F, Y, or W; X₂₁ |
| | is Q, T, S, N or D; X₂₂ is E, D, A, or T; X₂₃ is K or T; X₂₄ is K or E; X₂₅ is D, A, or E; |
| | X₂₆ is T, S, N, or Q; X₂₇ is E or Q; and X₂₈ is L, V, A, I, M, F, Y, or W, and wherein X₂₄ |
| | is E and/or either X₂₁ is T and X₂₂ is D or X₂₁ is D and X₂₂ is T. |
| 3 | The variant of row 1 or 2, wherein X₁ is I. |
| 4 | The variant of row 1 or 2, wherein X₁ is L. |
| 5 | The variant of any one of rows 1-4, wherein X₂ is F. |
| 6 | The variant of any one of rows 1-4, wherein X₂ is Y. |
| 7 | The variant of any one of rows 1-6, wherein X₃ is L. |
| 8 | The variant of any one of rows 1-6, wherein X₃ is K. |
| 9 | The variant of any one of rows 1-8, wherein X₄ is D. |
| 10 | The variant of any one of rows 1-8, wherein X₄ is E. |
| 11 | The variant of any one of rows 1-10, wherein X₅ is T. |
| 12 | The variant of any one of rows 1-10, wherein X₅ is S. |
| 13 | The variant of any one of rows 1-12, wherein X₆ is L. |
| 14 | The variant of any one of rows 1-12, wherein X₆ is V. |
| 15 | The variant of any one of rows 1-14, wherein X₇ is P. |
| 16 | The variant of any one of rows 1-14, wherein X₇ is R. |
| 17 | The variant of any one of rows 1-16, wherein X₈ is Y. |
| 18 | The variant of any one of rows 1-16, wherein X₈ is E. |
| 19 | The variant of any one of rows 1-18, wherein X₉ is D. |
| 20 | The variant of any one of rows 1-18, wherein X₉ is E. |
| 21 | The variant of any one of rows 1-20, wherein X₁₀ is K. |
| 22 | The variant of any one of rows 1-20, wherein X₁₀ is Q. |
| 23 | The variant of any one of rows 1-22, wherein X₁₁ is R. |
| 24 | The variant of any one of rows 1-22, wherein X₁₁ is L. |
| 25 | The variant of any one of rows 1-24, wherein X₁₂ is Y. |
| 26 | The variant of any one of rows 1-24, wherein X₁₂ is F. |
| 27 | The variant of any one of rows 1-26, wherein X₁₃ is R. |
| 28 | The variant of any one of rows 1-26, wherein X₁₃ is K. |
| 29 | The variant of any one of rows 1-28, wherein X₁₄ is S. |
| 30 | The variant of any one of rows 1-28, wherein X₁₄ is I. |
| 31 | The variant of any one of rows 1-30, wherein X₁₅ is S. |
| 32 | The variant of any one of rows 1-30, wherein X₁₅ is T. |
| 33 | The variant of any one of rows 1-32, wherein X₁₆ is S. |
| 34 | The variant of any one of rows 1-32, wherein X₁₆ is T. |
| 35 | The variant of any one of rows 1-34, wherein X₁₇ is I. |
| 36 | The variant of any one of rows 1-34, wherein X₁₇ is L. |
| 37 | The variant of any one of rows 1-36, wherein X₁₈ is I. |
| 38 | The variant of any one of rows 1-36, wherein X₁₈ is L. |
| 39 | The variant of any one of rows 1-38, wherein X₁₉ is K. |
| 40 | The variant of any one of rows 1-38, wherein X₁₉ is Q. |
| 41 | The variant of any one of rows 1-40, wherein X₂₀ is F. |
| 42 | The variant of any one of rows 1-40, wherein X₂₀ is I. |
| 43 | The variant of any one of rows 1-42, wherein X₂₁ is Q. |
| 44 | The variant of any one of rows 1-42, wherein X₂₁ is T. |
| 45 | The variant of any one of rows 1-42, wherein X₂₁ is D. |
| 46 | The variant of any one of rows 1-43, wherein X₂₂ is E. |
| 47 | The variant of any one of rows 1-42 and row 45, wherein X₂₂ is D. |
| 48 | The variant of any one of rows 1-42 and row 45, wherein X₂₂ is T. |
| 49 | The variant of any one of rows 1-48, wherein X₂₃ is K. |
| 50 | The variant of any one of rows 1-48, wherein X₂₃ is T. |
| 51 | The variant of any one of rows 1-50, wherein X₂₄ is K. |
| 52 | The variant of any one of rows 1-42, row 44, row 45, row 47, and row 48-50, wherein |
| | X₂₄ is E. |
| 53 | The variant of any one of rows 1-52, wherein X₂₅ is D. |
| 54 | The variant of any one of rows 1-52, wherein X₂₅ is E. |
| 55 | The variant of any one of rows 1-54, wherein X₂₆ is S. |
| 56 | The variant of any one of rows 1-54, wherein X₂₆ is N. |
| 57 | The variant of any one of rows 1-56, wherein X₂₇ is E. |
| 58 | The variant of any one of rows 1-56, wherein X₂₇ is Q. |
| 59 | The variant of any one of rows 1-58, wherein X₂₈ is F. |
| 60 | The variant of any one of rows 1-58, wherein X₂₈ is M. |

In some embodiments, a polypeptide described herein includes an extracellular ActRIIB variant having a sequence of any one of SEQ ID NOs: 282 and 290-302 (Table 3).

TABLE 3
Extracellular ActRIIB variants having the sequences of SEQ ID NOs: 282
and 290-302

| SEQ ID | |
|---|---|
| NO | Amino Acid Sequence |
| 282 | GRGEAETRECIFYNANWEKDRTNQSGLEPCYGDQDKRRHCFASWKNSSGTI |
| | ELVKQGCWLDDINCYDRQECVAKKDSPEVYFCCCEGNFCNERFTHLPEAG |
| | GPEVTYEPPPTAPT |
| 290 | GRGEAETRECIYYNANWELDRTNQSGLERCEGEQDKRLHCYASWRNSSGTI |
| | ELVKKGCWLDDINCYDRQECVATKENPQVYFCCCEGNFCNERFTHLPEAG |
| | GPEVTYEPPPTAPT |
| 291 | GRGEAETRECIYYNANWELERTNQSGLERCEGEQDKRLHCYASWRNSSGTI |
| | ELVKKGCWLDDFNCYDRQECVAKKDSPEVYFCCCEGNFCNERFTHLPEAG |
| | GPEVTYEPPPTAPT |
| 292 | GRGEAETRECIYYNANWELERTNQTGLERCEGEQDKRLHCYASWRNISGTI |
| | ELVKKGCWLDDFNCYDRQECVAKKDSPEVYFCCCEGNFCNERFTHLPEAG |
| | GPEVTYEPPPTAPT |
| 293 | GRGEAETRECIYYNANWELERTNQTGLERCEGEQDKRLHCYASWRNITGTI |
| | ELVKKGCWLDDFNCYDRQECVAKKDSPEVYFCCCEGNFCNERFTHLPEAG |
| | GPEVTYEPPPTAPT |
| 294 | GRGEAETRECIYYNANWELERTNQSGLEPCEGEQDKRLHCYASWRNSSGTI |
| | ELVKKGCWLDDFNCYDRQECVAKKDSPEVYFCCCEGNFCNERFTHLPEAG |
| | GPEVTYEPPPTAPT |
| 295 | GRGEAETRECIYYNANWELERTNQSGLERCYGDKDKRLHCFASWKNSSGTI |
| | ELVKQGCWLDDFNCYDRQECVAKKDSPEVYFCCCEGNFCNERFTHLPEAG |
| | GPEVTYEPPPTAPT |
| 296 | GRGEAETRECIYYNANWELERTNQSGLERCEGEQDKRLHCYASWRNSSGTI |
| | ELVKKGCWLDDFNCYDRQECVAKKDSPEVYFCCCEGNFCNERFTHLPEAG |
| | GPEVTYEPPPTAPT |
| 297 | GRGEAETRECIFYNANWEKDRTNQSGLERCYGDQDKRRHCYASWRNSSGT |
| | IELVKKGCWLDDFNCYDRQECVAKKDSPEVYFCCCEGNFCNERFTHLPEAG |
| | GPEVTYEPPPTAPT |
| 298 | GRGEAETRECIYYNANWELERTNQSGLERCEGEQDKRLHCYASWRNSSGTI |
| | ELVKKGCWLDDFNCYDRQECVAKKDSPEVYFCCCEGNFCNERFTHLPEAG |
| | GPEVTYEPPPTAPT |
| 299 | GRGEAETRECLYYNANWELERTNQSGVERCEGEKDKRLHCYASWRNSSGS |
| | LEIVKKGCWLDDFNCYDRTDCVATEENPQVYFCCCEGNMCNERFTHLPEA |
| | GGPEVTYEPPPTAPT |
| 300 | GRGEAETRECLYYNANWELERTNQSGVERCEGEKDKRLHCYASWRNSSGS |
| | LEIVKKGCWLDDENCYDRDTCVATEENPQVYFCCCEGNMCNERFTHLPEA |
| | GGPEVTYEPPPTAPT |
| 301 | GRGEAETRECLYYNANWELERTNQSGVERCEGEKDKRLHCYASWRNSSGS |
| | LEIVKKGCWLDDFNCYDRTDCVATKENPQVYFCCCEGNMCNERFTHLPEA |
| | GGPEVTYEPPPTAPT |
| 302 | GRGEAETRECLYYNANWELERTNQSGVERCEGEKDKRLHCYASWRNSSGS |
| | LEIVKKGCWLDDFNCYDRDTCVATKENPQVYFCCCEGNMCNERFTHLPEA |
| | GGPEVTYEPPPTAPT |

In one aspect, the present disclosure provides isolated variant ActRIIB polypeptides comprising hybrid soluble ActRIIB polypeptides which retain myostatin- and activin A-neutralizing activities, but demonstrate dramatically reduced BMP9-neutralization. In various embodiments, the variant ActRIIB polypeptides comprise a hybrid soluble ActRIIB polypeptide having the amino acid sequence of SEQ ID NO: 303 wherein at least one of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or Ti 10 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least two of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 are substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least three of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 are substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least four of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 are substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least five of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 are substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least six of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 are substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least seven of amino acid residues R3, I6, Y7, Y8, L14, E15, S20, L22, R24, E26, E28, Q29, L33, L48, Y36, 538, R40, 542, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 are substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least eight of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 are substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least nine of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 are substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least ten of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 are substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least fifteen of amino acid residues R3, I6, Y7, Y8, L14, E15, S20, L22, R24, E26, E28, Q29, L33, L48, Y36, S38, R40, 542, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 are substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least twenty of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 are substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least twenty-five of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 are substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least thirty of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 are substituted with another amino acid, and wherein the hybrid ActRIIB polypeptide is capable of binding myostatin and activin A, but demonstrates a decreased binding affinity for BMP9 relative to a wild-type ActRIIB polypeptide.

In various embodiments, the variant ActRIIB polypeptides comprise hybrid soluble ActRIIB polypeptides having an amino acid sequence set forth in any one of SEQ ID NOs: 305-339 (see, e.g., Table 11), wherein the hybrid ActRIIB polypeptide is capable of binding myostatin and activin A, but demonstrates a decreased binding affinity for BMP9 relative to a wild-type ActRIIB polypeptide. In various embodiments, the hybrid soluble ActRIIB polypeptides are hybrid soluble ActRIIB polypeptides having an amino acid sequence that is at least 70%, 75%, 80%, 85%, 90%, 91%, 92%, 93%, 94%, 95%, 96%, 97%, 98%, 99% or 100% identical to an amino acid sequence selected from SEQ ID NOs: 305-339 (see, e.g., Table 11), wherein the hybrid ActRIIB polypeptide is capable of binding myostatin and activin A, but demonstrates a decreased binding affinity for BMP9 relative to a wild-type ActRIIB polypeptide.

In various embodiments, the variant ActRIIB polypeptide comprises an amino acid sequence set forth in any one of SEQ ID NOs: 340-406, wherein the variant ActRIIB polypeptide is capable of binding myostatin and activin A, but demonstrates a decreased binding affinity for BMP9 relative to a wild-type ActRIIB polypeptide. In various embodiments, the variant ActRIIB polypeptides are hybrid soluble ActRIIB polypeptides having an amino acid sequence that is at least 70%, 75%, 80%, 85%, 90%, 91%, 92%, 93%, 94%, 95%, 96%, 97%, 98%, 99% or 100% identical to an amino acid sequence selected from SEQ ID NOs: 340-406, wherein the hybrid ActRIIB polypeptide is capable of binding myostatin and activin A, but demonstrates a decreased binding affinity for BMP9 relative to a wild-type ActRIIB polypeptide.

In another aspect, the present disclosure provides isolated nucleic acid molecules comprising a polynucleotide encoding a hybrid soluble ActRIIB polypeptide of the present disclosure. In various embodiments, the polynucleotides encodes one of the polypeptide sequences set forth in SEQ ID NOs: 305-406, wherein the hybrid ActRIIB polypeptide is capable of binding myostatin and activin A, but demonstrates a decreased binding affinity for BMP9 relative to a wild-type ActRIIB polypeptide. In various embodiments, the polynucleotides encode a polypeptide having an amino acid sequence at least 70%, 75%, 80%, 85%, 90%, 91%, 92%, 93%, 94%, 95%, 96%, 97%, 98%, 99% or 100% identity to any one of the polypeptides sequences set forth in SEQ ID NOs: 305-406, wherein the hybrid ActRIIB polypeptide is capable of binding myostatin and activin A, but demonstrates a decreased binding affinity for BMP9 relative to a wild-type ActRIIB polypeptide. In various embodiments, the polynucleotides encode a polypeptide having at least 90% identity to any one of the polypeptides sequences set forth in SEQ ID NOs: 305-406, wherein the hybrid ActRIIB polypeptide is capable of binding myostatin and activin A, but demonstrates a decreased binding affinity for BMP9 relative to a wild-type ActRIIB polypeptide. In various embodiments, the polynucleotides encode a polypeptide having an amino acid sequence at least 95% identity to any one of the polypeptides sequences set forth in SEQ ID NOs: 305-406, wherein the hybrid ActRIIB polypeptide is capable of binding myostatin and activin A, but demonstrates a decreased binding affinity for BMP9 relative to a wild-type ActRIIB polypeptide.

In some embodiments, an ActRIIB polypeptide of the disclosure comprises a hybrid soluble ActRIIB polypeptide that is derived from wild-type ActRIIB and wild-type ActRIIA. The hybrid soluble ActRIIB polypeptides are specifically engineered by replacing one or more amino acids of a truncated wild-type ActRIIB polypeptide with the amino acids from a truncated wild-type ActRIIA polypeptide at corresponding positions based on sequence alignment between the two truncated ActRII polypeptide extracellular domains at the amino acid level. The one or more amino acid replacements are specifically selected for purposes of providing hybrid soluble ActRIIB polypeptides which demonstrate a reduction of BMP9-neutralization as compared to wild-type ActRIIB polypeptide, while retaining myostatin- and activin A-neutralization.

In various embodiments, the truncated extracellular domain of ActRIIB used to prepare the hybrid soluble ActRIIB polypeptides has the 110 amino acid sequence set forth in SEQ ID NO: 303:

(SEQ ID NO: 303)
ETRECIYYNANWELERTNQSGLERCEGEQDKRLHCYASWRNSSGTIELV
KKGCWLDDFNCYDRQECVATEENPQVYFCCCEGNFCNERFTHLPEAGGP
EVTYEPPPTAPT

In various embodiments, the truncated extracellular domain of ActRIIA used to prepare the hybrid soluble ActRIIB polypeptides has the 110 amino acid sequence set forth in SEQ ID NO: 304:

(SEQ ID NO: 304)
ETQECLFFNANWEKDRTNQTGVEPCYGDKDKRRHCFATWKNISGSIEIV
KQGCWLDDINCYDRTDCVEKKDSPEVYFCCCEGNMCNEKFSYFPEMEVT
QPTSNPVTPKPP

In various embodiments, the variant ActRIIB polypeptides comprise a hybrid soluble ActRIIB polypeptide having the amino acid sequence of SEQ ID NO: 303 wherein at least one of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with the amino acid at the corresponding position of wild-type ActRIIA sequence (SEQ ID NO: 304), and wherein the hybrid soluble ActRIIB polypeptide is capable of binding myostatin and activin A, but demonstrates a decreased binding affinity for BMP9 relative to a wild-type ActRIIB polypeptide.

In various embodiments, the hybrid soluble ActRIIB polypeptide comprises the amino acid sequence of SEQ ID NO: 305, wherein amino acid residues E26, E28, Q29, L33, F58, Q64, E65, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 306, wherein amino acid residues E26, E28, Q29, L33, Q64, E65, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 307, wherein amino acid residues F58, Q64, E65, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 308, wherein amino acid residues F58, Q64, E65, A68, T69, E70, E71, and N72 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 309, wherein amino acid residues Q64, E65, A68, T69, E70, E71, and N72 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 310, wherein amino acid residues Q64, E65, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 311, wherein amino acid residues A68, T69, E70, E71, N72 and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 312, wherein amino acid residues A68, T69, E70, E71, and N72 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 313, wherein amino acid residues F58, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 314, wherein amino acid residues Q64, E65, A68, T69, E70, E71, N72, Q74, and F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 315, wherein amino acid residues A68, T69, E70, E71, N72, Q74, and F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 316, wherein amino acid residues R3, L14, E15, S20, L22, R24, E26, E28, Q29, and L33 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 317, wherein amino acid residues R3, L14, E15, S20, L22, and R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 318, wherein amino acid residues E26, E28, Q29, and L33 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 319, wherein amino acid residues L14, E15, S20, L22, and R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 320, wherein amino acid residues R3, L14, E15, 520, L22, and R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 321, wherein amino acid residues R3, L14, E15, and S20 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 322, wherein amino acid residues R3, L14, and E15 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 323, wherein amino acid residues L14 and E15 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 324, wherein amino acid residue R3 of SEQ ID NO: 303 has been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 325, wherein amino acid residues Y36, S38, and K51 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 326, wherein amino acid residues E26, E28, Q29, L33, and F58 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 327, wherein amino acid residue E70 of SEQ ID NO: 303 has been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 328, wherein amino acid residue F58 of SEQ ID NO: 303 has been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 329, wherein amino acid residues F58 and E70 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 330, wherein amino acid residues E28, Q29, F58, and E70 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 331, wherein amino acid residues E28, F58, and E70 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 332, wherein amino acid residues E28 and E70 of SEQ ID NO: 303 have been replaced by the amino acid residues in the corresponding positions of SEQ ID NO: 304. In some embodiments, the hybrid soluble ActRIIB polypeptide has decreased binding affinity for BMP9.

In some embodiments, the hybrid soluble ActRIIB polypeptide binds myostatin and/or activin A. In various embodiments, the hybrid soluble ActRIIB polypeptide comprises the amino acid sequence of SEQ ID NO: 333, wherein amino acid residue E28 of SEQ ID NO: 303 has been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 334, wherein amino acid residues E26, E28, Q29, L33, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 335, wherein amino acid residues Y7, Y8, L14, E15, S20, L22, and R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 336, wherein amino acid residues Y36, S38, R40, S42, T45, and K51 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 337, wherein amino acid residues Q64 and E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 338, wherein amino acid residue F84 of SEQ ID NO: 303 have been replaced by the amino acid residue in the corresponding position of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 339, wherein amino acid residues E28 and F58 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 340, wherein amino acid residues R3, I6, Y7, Y8, L14, E15, L22, R24, E26, E28, Q29, L33 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 341, wherein amino acid residues R3, I6, Y7, Y8, L14, E15, L22, R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 342, wherein amino acid residues 16, Y7, Y8, L14, E15, L22, R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 343, wherein amino acid residues 16, Y7, Y8, L14, E15, L22, R24, E26 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 344, wherein amino acid residues 16, Y7, Y8, L14, E15, L22, R24, E26, E28, Q29, L33 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 345, wherein amino acid residues 16, Y7, Y8, L14, E15, L22, R24, E26, E28, Q29, L33, Y36, S38, R40, S42, T45, L48, K51 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 346, wherein amino acid residues 16, Y7, Y8, L14, E15, L22, R24, E26, E28, Q29, L33, Y36, S38, R40, S42, T45, L48, K51, F58 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 347, wherein amino acid residues 16, Y7, Y8, L14, E15, L22, R24, E26, E28, Q29, L33, Y36, S38, R40, S42, T45, L48, K51, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 348, wherein amino acid residues R3, E26, E28, Q29, L33, Y36, S38, R40, S42, T45, L48, K51, F58 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 349, wherein amino acid residues E26, E28, Q29, L33, Y36, S38, R40, S42, T45, L48, K51, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 350, wherein amino acid residues E26, E28, Q29, L33, Y36, S38, R40, S42, T45, L48, K51, F58, Q64, E65, A68, T69, E70, E71, N72, Q74, F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 351, wherein amino acid residues Y36, S38, R40, S42, T45, L48, K51, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 352, wherein amino acid residues Y36, S38, R40, S42, T45, L48, K51, F58, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 353, wherein amino acid residues Y36, S38, R40, S42, T45, L48, K51, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 354, wherein amino acid residues Y36, S38, R40, S42, T45, L48, K51 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 355, wherein amino acid residues R3, E26, E28, Q29, L33, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 356, wherein amino acid residues R3, E26, E28, Q29, L33, F58, Q64, E65, A68, T69, E70, E71, N72, Q74, F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 357, wherein amino acid residues R3, E26, E28, Q29, L33, Y36, S38, R40, S42, T45, L48, K51, F58, Q64, E65, A68, T69, E70, E71, N72, Q74, F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 358, wherein amino acid residues R3, E26, E28, Q29, L33, Y36, S38, R40, S42, T45, L48, K51, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 359, wherein amino acid residues 16, Y7, Y8, L14, E15, L22, R24, Y36, S38, R40, S42, T45, L48, K51, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 360, wherein amino acid residues 16, Y7, Y8, L14, E15, L22, R24, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 361, wherein amino acid residues 16, Y7, Y8, L14, E15, L22, R24, E26, E28, Q29, L33, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 362, wherein amino acid residues E26, E28, Q29, L33, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 363, wherein amino acid residues E26, E28, Q29, L33, K51, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 364, wherein amino acid residues E26, E28, Q29, L33, L48, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 365, wherein amino acid residues E26, E28, Q29, L33, T45, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 366, wherein amino acid residues E26, E28, Q29, L33, T45, L48, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 367, wherein amino acid residues E26, E28, Q29, L33, T45, L48, K51, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 368, wherein amino acid residues Q64, E65, F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 369, wherein amino acid residues R88, T90, H91, L92, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 370, wherein amino acid residues R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 371, wherein amino acid residues E26, E28, Q29, L33, F58, Q64, E65, A68, T69, E70, E71, N72, Q74, R88, T90, H91, L92, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 372, wherein amino acid residues E26, E28, Q29, L33, Q64, E65, A68, T69, E70, E71, N72, Q74, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 373, wherein amino acid residues E26, E28, Q29, L33, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 374, wherein amino acid residues E26, E28, Q29, L33, K51, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 375, wherein amino acid residues E26, E28, Q29, L33, L48, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 376, wherein amino acid residues E26, E28, Q29, L33, T45, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 377, wherein amino acid residues T45, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 378, wherein amino acid residues L48, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 379, wherein amino acid residues K51, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 380, wherein amino acid residues A68, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 381, wherein amino acid residues A68, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 382, wherein amino acid residues E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 383, wherein amino acid residues E71, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 384, wherein amino acid residues N72, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 385, wherein amino acid residues Q74, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 386, wherein amino acid residues E28, Q29, A68, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 387, wherein amino acid residues Q29, T69, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 388, wherein amino acid residues E28, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 389, wherein amino acid residues E28, Q29, K51, T69, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 390, wherein amino acid residues E28, Q29, L48, K51, T69E, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 391, wherein amino acid residues E26, E28, T45, L48, K51, T69, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 392, wherein amino acid residues Q29, L48, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 393, wherein amino acid residues E26, E28, L33, Q70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 394, wherein amino acid residues L33, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 395, wherein amino acid residues E26, T45, L48, Q64, E65, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 396, wherein amino acid residues L33, T45, T69, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 397, wherein amino acid residues L33, L48, T69, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 398, wherein amino acid residues L33, T45, L48, E70, R88, T90, H91, L92, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 399, wherein amino acid residues E28, L48, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 400, wherein amino acid residues E28, T45, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 401, wherein amino acid residues E28, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 402, wherein amino acid residues L48, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 403, wherein amino acid residues E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 404, wherein amino acid residues E28, L48, T79, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 405, wherein amino acid residues R3, I6, Y7, Y8, L14, E15, S20, L22, R24, E26, E28, Q29, L33, Y36, S38, R40, S42, T45, L48, K51, F58, Q64, E65, A68, T69, E71, N72, Q74, F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 406, wherein amino acid residues E26, E28, Q29, L33, F56, E68 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In certain embodiments, the present disclosure relates to ActRIIA polypeptides. As used herein, the term “ActRIIA” refers to a family of activin receptor type IIA (ActRIIA) proteins from any species and variants derived from such ActRIIA proteins by mutagenesis or other modification. Reference to ActRIIA herein is understood to be a reference to any one of the currently identified forms. Members of the ActRIIA family are generally transmembrane proteins, composed of a ligand-binding extracellular domain comprising a cysteine-rich region, a transmembrane domain, and a cytoplasmic domain with predicted serine/threonine kinase activity.

The term “ActRIIA polypeptide” includes polypeptides comprising any naturally occurring polypeptide of an ActRIIA family member as well as any variants thereof (including mutants, fragments, fusions, and peptidomimetic forms) that retain a useful activity. Examples of such variant ActRIIA polypeptides are provided throughout the present disclosure as well as in International Patent Application Publication Nos. WO 2006/012627, WO 2007/062188, WO2018/089706, WO2018/089715, and WO2019/094751 which are incorporated herein by reference in their entirety. Numbering of amino acids for all ActRIIA-related polypeptides described herein is based on the numbering of the human ActRIIA precursor protein sequence provided below (SEQ ID NO: 9), unless specifically designated otherwise.

The canonical human ActRIIA precursor protein sequence is as follows:

| (SEQ ID NO: 9) | |
|---|---|
| 1 | MGAAAKLAFA VFLISCSSGA ILGRSETQEC LFFNANWEKD RT [symbol] QTGVEPC |
| 51 | YGDKDKRRHC FATWK [symbol] ISGS IEIVKQGCWL DDINCYDRTD CVEKKDSPEV |
| 101 | YFCCCEGNMC NEKFSYFPEM EVTQPTSNPV TPKPPYYNIL LYSLVPLMLI |
| 151 | AGIVICAFWV YRHHKMAYPP VLVPTQDPGP PPPSPLLGLK PLQLLEVKAR |
| 201 | GRFGCVWKAQ LLNEYVAVKI FPIQDKQSWQ NEYEVYSLPG MKHENILQFI |
| 251 | GAEKRGTSVD VDLWLITAFH EKGSLSDFLK ANVVSWNELC HIAETMARGL |
| 301 | AYLHEDIPGL KDGHKPAISH RDIKSKNVLL KNNLTACIAD FGLALKFEAG |
| 351 | KSAGDTHGQV GTRRYMAPEV LEGAINFQRD AFLRIDMYAM GLVLWELASR |
| 401 | CTAADGPVDE YMLPFEEEIG QHPSLEDMQE VVVHKKKRPV LRDYWQKHAG |
| 451 | MAMLCETIEE CWDHDAEARL SAGCVGERIT QMQRLINIIT TEDIVTVVTM |
| 501 | VTNVDEPPKE SSL |

The signal peptide is indicated by a single underline; the extracellular domain is indicated in bold font; and the potential, endogenous N-linked glycosylation sites are indicated by a double underline.

A processed (mature) extracellular human ActRIIA polypeptide sequence is as follows:

(SEQ ID NO: 10)
ILGRSETQECLFFNANWEKDRINQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPP

The C-terminal “tail” of the extracellular domain is indicated by single underline. The sequence with the “tail” deleted (a Δ15 sequence) is as follows:

(SEQ ID NO: 11)
ILGRSETQECLFFNANWEKDRINQTGVEPCYGDKDKRRHCFATWKNISG
SIEIVKQGCWLDDINCYDRTDCVEKKDSPEVYFCCCEGNMCNEKFSYFP
EM

A nucleic acid sequence encoding the human ActRIIA precursor protein (SEQ ID NO: 9) is shown below (SEQ ID NO: 12), as follows nucleotides 159-1700 of Genbank Reference Sequence NM 001616.4. The signal sequence is underlined.

| (SEQ ID NO: 12) | |
|---|---|
| 1 | ATGGGAGCTG CTGCAAAGTT GGCGTTTGCC GTCTTTCTTA TCTCCTGTTC |
| 51 | TTCAGGTGCT ATACTTGGTA GATCAGAAAC TCAGGAGTGT CTTTTCTTTA |
| 101 | ATGCTAATTG GGAAAAAGAC AGAACCAATC AAACTGGTGT TGAACCGTGT |
| 151 | TATGGTGACA AAGATAAACG GCGGCATTGT TTTGCTACCT GGAAGAATAT |
| 201 | TTCTGGTTCC ATTGAAATAG TGAAACAAGG TTGTTGGCTG GATGATATCA |
| 251 | ACTGCTATGA CAGGACTGAT TGTGTAGAAA AAAAAGACAG CCCTGAAGTA |
| 301 | TATTTTTGTT GCTGTGAGGG CAATATGTGT AATGAAAAGT TTTCTTATTT |
| 351 | TCCGGAGATG GAAGTCACAC AGCCCACTTC AAATCCAGTT ACACCTAAGC |
| 401 | CACCCTATTA CAACATCCTG CTCTATTCCT TGGTGCCACT TATGTTAATT |
| 451 | GCGGGGATTG TCATTTGTGC ATTTTGGGTG TACAGGCATC ACAAGATGGC |
| 501 | CTACCCTCCT GTACTTGTTC CAACTCAAGA CCCAGGACCA CCCCCACCTT |
| 551 | CTCCATTACT AGGTTTGAAA CCACTGCAGT TATTAGAAGT GAAAGCAAGG |
| 601 | GGAAGATTTG GTTGTGTCTG GAAAGCCCAG TTGCTTAACG AATATGTGGC |
| 651 | TGTCAAAATA TTTCCAATAC AGGACAAACA GTCATGGCAA AATGAATACG |
| 701 | AAGTCTACAG TTTGCCTGGA ATGAAGCATG AGAACATATT ACAGTTCATT |
| 751 | GGTGCAGAAA AACGAGGCAC CAGTGTTGAT GTGGATCTTT GGCTGATCAC |
| 801 | AGCATTTCAT GAAAAGGGTT CACTATCAGA CTTTCTTAAG GCTAATGTGG |
| 851 | TCTCTTGGAA TGAACTGTGT CATATTGCAG AAACCATGGC TAGAGGATTG |
| 901 | GCATATTTAC ATGAGGATAT ACCTGGCCTA AAAGATGGCC ACAAACCTGC |
| 951 | CATATCTCAC AGGGACATCA AAAGTAAAAA TGTGCTGTTG AAAAACAACC |
| 1001 | TGACAGCTTG CATTGCTGAC TTTGGGTTGG CCTTAAAATT TGAGGCTGGC |
| 1051 | AAGTCTGCAG GCGATACCCA TGGACAGGTT GGTACCCGGA GGTACATGGC |
| 1101 | TCCAGAGGTA TTAGAGGGTG CTATAAACTT CCAAAGGGAT GCATTTTTGA |
| 1151 | GGATAGATAT GTATGCCATG GGATTAGTCC TATGGGAACT GGCTTCTCGC |
| 1201 | TGTACTGCTG CAGATGGACC TGTAGATGAA TACATGTTGC CATTTGAGGA |
| 1251 | GGAAATTGGC CAGCATCCAT CTCTTGAAGA CATGCAGGAA GTTGTTGTGC |
| 1301 | ATAAAAAAAA GAGGCCTGTT TTAAGAGATT ATTGGCAGAA ACATGCTGGA |
| 1351 | ATGGCAATGC TCTGTGAAAC CATTGAAGAA TGTTGGGATC ACGACGCAGA |
| 1401 | AGCCAGGTTA TCAGCTGGAT GTGTAGGTGA AAGAATTACC CAGATGCAGA |
| 1451 | GACTAACAAA TATTATTACC ACAGAGGACA TTGTAACAGT GGTCACAATG |
| 1501 | GTGACAAATG TTGACTTTCC TCCCAAAGAA TCTAGTCTA |

A nucleic acid sequence encoding the processed soluble (extracellular) human ActRIIA polypeptide (SEQ ID NO: 10) is as follows:

| (SEQ ID NO: 13) | |
|---|---|
| 1 | ATACTTGGTA GATCAGAAAC TCAGGAGTGT CTTTTCTTTA ATGCTAATTG |
| 51 | GGAAAAAGAC AGAACCAATC AAACTGGTGT TGAACCGTGT TATGGTGACA |
| 101 | AAGATAAACG GCGGCATTGT TTTGCTACCT GGAAGAATAT TTCTGGTTCC |
| 151 | ATTGAAATAG TGAAACAAGG TTGTTGGCTG GATGATATCA ACTGCTATGA |
| 201 | CAGGACTGAT TGTGTAGAAA AAAAAGACAG CCCTGAAGTA TATTTTTGTT |
| 251 | GCTGTGAGGG CAATATGTGT AATGAAAAGT TTTCTTATTT TCCGGAGATG |
| 301 | GAAGTCACAC AGCCCACTTC AAATCCAGTT ACACCTAAGC CACCC |

In some embodiments, the ActRIIA polypeptide sequence comprises accession number UniProtKB/Swiss-Prot P27037.1 (SEQ ID NO: 408 herein), and variants thereof. In some embodiments, the term “wild-type ActRIIA polypeptide” refers to the extracellular domain of ActRIIA, amino acids 1 to 135 (with signal sequence), or amino acids 20 through 135 of SEQ ID NO: 407 (without signal sequence) (referred to herein as SEQ ID NO: 409).

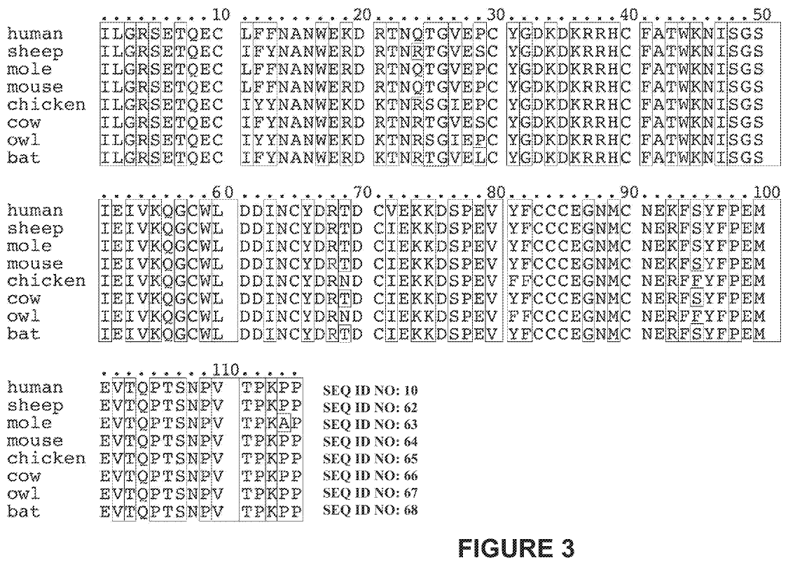

ActRIIA is well-conserved among vertebrates, with large stretches of the extracellular domain completely conserved. For example, FIG. 3 depicts a multi-sequence alignment of a human ActRIIA extracellular domain compared to various ActRIIA orthologs. Many of the ligands that bind to ActRIIA are also highly conserved. Accordingly, from these alignments, it is possible to predict key amino acid positions within the ligand-binding domain that are important for normal ActRIIA-ligand binding activities as well as to predict amino acid positions that are likely to be tolerant to substitution without significantly altering normal ActRIIA-ligand binding activities. Therefore, an active, human ActRIIA variant polypeptide useful in accordance with the presently disclosed methods may include one or more amino acids at corresponding positions from the sequence of another vertebrate ActRIIA, or may include a residue that is similar to that in the human or other vertebrate sequences.

Without meaning to be limiting, the following examples illustrate this approach to defining an active ActRIIA variant. As illustrated in FIG. 3, F13 in the human extracellular domain (SEQ ID NO: 10) is Y in Ovis aries (SEQ ID NO: 62), Gallus gallus (SEQ ID NO: 65), Bos Taurus (SEQ ID NO: 66), Tyto alba (SEQ ID NO: 67), and Myotis davidii (SEQ ID NO: 68) ActRIIA, indicating that aromatic residues are tolerated at this position, including F, W, and Y. Q24 in the human extracellular domain (SEQ ID NO: 10) is R in Bos Taurus ActRIIA, indicating that charged residues will be tolerated at this position, including D, R, K, H, and E. S95 in the human extracellular domain (SEQ ID NO: 10) is F in Gallus gallus and Tyto alba ActRIIA, indicating that this site may be tolerant of a wide variety of changes, including polar residues, such as E, D, K, R, H, S, T, P, G, Y, and probably hydrophobic residue such as L, I, or F. E52 in the human extracellular domain (SEQ ID NO: 10) is D in Ovis aries ActRIIA, indicating that acidic residues are tolerated at this position, including D and E. P29 in the human extracellular (SEQ ID NO: 10) domain is relatively poorly conserved, appearing as S in Ovis aries ActRIIA and L in Myotis davidii ActRIIA, thus essentially any amino acid should be tolerated at this position.

Moreover, as discussed above, ActRII proteins have been characterized in the art in terms of structural/functional characteristics, particularly with respect to ligand binding [Attisano et al. (1992) Cell 68(1):97-108; Greenwald et al. (1999) Nature Structural Biology 6(1): 18-22; Allendorph et al. (2006) PNAS 103(20: 7643-7648; Thompson et al. (2003) The EMBO Journal 22(7): 1555-1566; as well as U.S. Pat. Nos. 7,709,605, 7,612,041, and 7,842,663]. In addition to the teachings herein, these references provide amply guidance for how to generate ActRII variants that retain one or more desired activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRIIA, as demarcated by the outermost of these conserved cysteines, corresponds to positions 30-110 of SEQ ID NO: 9 (ActRIIA precursor). Therefore, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, 28, or 29 residues at the N-terminus and by about 1, 2, 3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding. Exemplary ActRIIA extracellular domains truncations include SEQ ID NOs: 10 and 11.

Accordingly, a general formula for an active portion (e.g., ligand binding) of ActRIIA is a polypeptide that comprises, consists essentially of, or consists of amino acids 30-110 of SEQ ID NO: 9. Therefore ActRIIA polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIA beginning at a residue corresponding to any one of amino acids 21-30 (e.g., beginning at any one of amino acids 21, 22, 23, 24, 25, 26, 27, 28, 29, or 30) of SEQ ID NO: 9 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134, or 135) of SEQ ID NO: 9. Other examples include constructs that begin at a position selected from 21-30 (e.g., beginning at any one of amino acids 21, 22, 23, 24, 25, 26, 27, 28, 29, or 30), 22-30 (e.g., beginning at any one of amino acids 22, 23, 24, 25, 26, 27, 28, 29, or 30), 23-30 (e.g., beginning at any one of amino acids 23, 24, 25, 26, 27, 28, 29, or 30), 24-30 (e.g., beginning at any one of amino acids 24, 25, 26, 27, 28, 29, or 30) of SEQ ID NO: 9, and end at a position selected from 111-135 (e.g., ending at any one of amino acids 111, 112, 113, 114, 115, 116, 117, 118, 119, 120, 121, 122, 123, 124, 125, 126, 127, 128, 129, 130, 131, 132, 133, 134 or 135), 112-135 (e.g., ending at any one of amino acids 112, 113, 114, 115, 116, 117, 118, 119, 120, 121, 122, 123, 124, 125, 126, 127, 128, 129, 130, 131, 132, 133, 134 or 135), 113-135 (e.g., ending at any one of amino acids 113, 114, 115, 116, 117, 118, 119, 120, 121, 122, 123, 124, 125, 126, 127, 128, 129, 130, 131, 132, 133, 134 or 135), 120-135 (e.g., ending at any one of amino acids 120, 121, 122, 123, 124, 125, 126, 127, 128, 129, 130, 131, 132, 133, 134 or 135), 130-135 (e.g., ending at any one of amino acids 130, 131, 132, 133, 134 or 135), 111-134 (e.g., ending at any one of amino acids 110, 111, 112, 113, 114, 115, 116, 117, 118, 119, 120, 121, 122, 123, 124, 125, 126, 127, 128, 129, 130, 131, 132, 133, or 134), 111-133 (e.g., ending at any one of amino acids 110, 111, 112, 113, 114, 115, 116, 117, 118, 119, 120, 121, 122, 123, 124, 125, 126, 127, 128, 129, 130, 131, 132, or 133), 111-132 (e.g., ending at any one of amino acids 110, 111, 112, 113, 114, 115, 116, 117, 118, 119, 120, 121, 122, 123, 124, 125, 126, 127, 128, 129, 130, 131, or 132), or 111-131 (e.g., ending at any one of amino acids 110, 111, 112, 113, 114, 115, 116, 117, 118, 119, 120, 121, 122, 123, 124, 125, 126, 127, 128, 129, 130, or 131) of SEQ ID NO: 9. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 9. Thus, in some embodiments, an ActRIIA polypeptide may comprise, consists essentially of, or consist of a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9. Optionally, ActRIIA polypeptides comprise a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9, and comprising no more than 1, 2, 5, 10 or 15 conservative amino acid changes in the ligand-binding pocket.

In certain embodiments, the disclosure relates to ActRII antagonists (inhibitors) that comprise an ActRIIA polypeptide, which includes fragments, functional variants, and modified forms thereof as well as uses thereof (e.g., increasing an immune response in a patient in need thereof and treating cancer). Preferably, ActRIIA polypeptides are soluble (e.g., an extracellular domain of ActRIIA). In some embodiments, ActRIIA polypeptides inhibit (e.g., Smad signaling) of one or more ligands [e.g., GDF11, GDF8, activin (activin A, activin B, activin AB, activin AC, BMP6, BMP7, BMP10, GDF3, GDF8, and/or GDF11]. In some embodiments, ActRIIA polypeptides bind to one or more ligands [e.g., activin A, activin B, activin AB, activin AC, BMP6, BMP7, BMP10, GDF3, GDF8, and/or GDF11]. In some embodiments, ActRIIA polypeptides of the disclosure demonstrate a decreased binding affinity for BMP9. In some embodiments, ActRIIA polypeptides of the disclosure do not bind BMP9. In some embodiments, ActRIIA polypeptide of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIA beginning at a residue corresponding to amino acids 21-30 (e.g., beginning at any one of amino acids 21, 22, 23, 24, 25, 26, 27, 28, 29, or 30) of SEQ ID NO: 9 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134 or 135) of SEQ ID NO: 9. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 30-110 of SEQ ID NO: 9. In certain embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 21-135 of SEQ ID NO: 9. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 9, 10, 11, 36, 39, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 283, 304, 408, and 409. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 9. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 10. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 11. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 36. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 39. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 139. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 140. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 141. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 142. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 143. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 144. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 145. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 146. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 147. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 148. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 149. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 150. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 151. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 152. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 153. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 154. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 155. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 156. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 157. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 158. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 159. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 160. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 161. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 162. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 163. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 164. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 165. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 166. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 167. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 168. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 169. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 170. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 171. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 172. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 173. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 174. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 175. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 176. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 177. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 178. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 179. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 180. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 181. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 182. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 183. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 184. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 185. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 186. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 187. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 188. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 189. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 190. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 191. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 192. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 193. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 194. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 195. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 196. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 197. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 198. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 199. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 200. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 201. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 202. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 203. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 204. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 205. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 206. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 207. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 208. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 209. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 210. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 211. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 283. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 304. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 408. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 409.

In some embodiments, an extracellular ActRIIA variant polypeptide may have a sequence of any one of SEQ ID NOs: 139-210. In some embodiments, an extracellular ActRIIA variant polypeptide has a sequence of any one of SEQ ID NOs: 144-210 (Table 5).

In some embodiments, an extracellular ActRIIA variant polypeptide may, for example, comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the sequence of a wild-type extracellular ActRIIA polypeptide (SEQ ID NO: 211).

In some embodiments, polypeptides described herein include an extracellular ActRIIA variant having at least one amino acid substitution relative to the wild-type extracellular ActRIIA having the sequence of SEQ ID NO: 211 or the extracellular ActRIIA having any one of the sequences of SEQ ID NOs: 212-232. Possible amino acid substitutions at 27 different positions may be introduced to an extracellular ActRIIA variant (Table 4). An extracellular ActRIIA variant may have one or more (e.g., 1-27, 1-25, 1-23, 1-21, 1-19, 1-17, 1-15, 1-13, 1-11, 1-9, 1-7, 1-5, 1-3, or 1-2; e.g., 1, 2, 3, 4, 5, 6, 7, 8, 9, 10, 11, 12, 13, 14, 15, 16, 17, 18, 19, 20, 21, 22, 23, 24, 25, 26, or 27) amino acid substitutions relative the sequence of a wild-type extracellular ActRIIA (SEQ ID NO: 211). In some embodiments, an extracellular ActRIIA variant (e.g., an extracellular ActRIIA variant having a sequence of SEQ ID NO: 139) may include amino acid substitutions at all of the 27 positions as listed in Table 4. In some embodiments, an extracellular ActRIIA variant may include amino acid substitutions at a number of positions, e.g., at 2, 4, 6, 8, 10, 12, 14, 16, 18, 20, 22, 24, or 26 out of the 27 positions, as listed in Table 4.

Amino acid substitutions can worsen or improve the activity and/or binding affinity of the ActRIIA variants disclosed herein. In some embodiments, to maintain polypeptide function, it is important that the lysine (K) at position X₁₇ in the sequences shown in Tables 3 and 4 (SEQ ID NOs: 139-210 (e.g., SEQ ID NOs: 144-210)) be retained. Substitutions at that position can lead to a loss of activity. For example, an ActRIIA variant having the sequence GAILGRSETQECLFYNANWELERTNQTGVERCEGEKDKRLHCYATWRNISGSIEIVA KGCWLDDFNCYDRTDCVETEENPQVYFCCCEGNMCNEKFSYFPEMEVTQPTS (SEQ ID NO: 283) has reduced activity in vivo, indicating that the substitution of alanine (A) for lysine (K) at X₁₇ is not tolerated. ActRIIA variants disclosed herein, including variants in Tables 3 and 4 (e.g., SEQ ID NOs: 139-210 (e.g., SEQ ID NOs: 144-210), therefore, may retain amino acid K at the position corresponding to X₁₇ in SEQ ID NO: 139 or SEQ ID NO: 140.

In some embodiments, the ActRIIA variants disclosed herein have reduced or no substantial binding to BMP9. In some embodiments, BMP9 binding is reduced in ActRIIA variants containing the amino acid sequence TEEN at positions X₂₃, X₂₄, X₂₅, and X₂₆, as well as in variants that maintain the amino acid K at position X₂₄ and have the amino acid sequence TKEN at positions X₂₃, X₂₄, X₂₅, and X₂₆. The sequences TEEN and TKEN can be employed interchangeably in the ActRIIA variants (e.g., the variants in Tables 3 and 4, e.g., SEQ ID NOs: 139-210 (e.g., SEQ ID NOs: 144-210)) disclosed herein to provide reduced BMP9 binding.

In some embodiments, the ActRIIA variants disclosed herein may further include a C-terminal extension (e.g., additional amino acids at the C-terminus). The C-terminal extension can add one to six additional amino acids at the C-terminus (e.g., 1, 2, 3, 4, 5, 6 or more additional amino acids) to any of the variant polypeptides shown in Tables 3 and 4 (e.g., SEQ ID NOs: 139-208 (e.g., SEQ ID NOs: 144-208)). One potential C-terminal extension that can be included in the ActRIIA variant polypeptides disclosed herein is amino acid sequence NP. For example, the sequence including the C-terminal extension is SEQ ID NO: 209 (e.g., SEQ ID NO: 207 with a C-terminal extension of NP). Another exemplary C-terminal extension that can be included in the ActRIIA variant polypeptides disclosed herein is amino acid sequence NPVTPK (SEQ ID NO: 288). For example, the sequence including the C-terminal extension is SEQ ID NO: 210 (e.g., SEQ ID NO: 207 with a C-terminal extension of

TABLE 4
Amino acid substitutions in an extracellular ActRIIA variant having a
sequence of any one of SEQ ID NOs: 139-143

GAILGRSETQECLX₁X₂NANWX₃X₄X₅X₆TNQTGVEX₇CX₈GX₉X₁₀X₁₁X₁₂X₁₃X₁₄HCX₁₅
ATWX₁₆NISGSIEIVX₁₇X₁₈GCX₁₉X₂₀X₂₁DX₂₂NCYDRTDCVEX₂₃X₂₄X₂₅X₂₆PX₂₇VYFCC
CEGNMCNEKFSYFPEMEVTQPTS (SEQ ID NO: 139)
GAILGRSETQECLFX₂NANWX₃X₄X₅X₆TNQTGVEX₇CX₈GX₉KX₁₁X₁₂X₁₃X₁₄HCX₁₅A
TWX₁₆NISGSIEIVX₁₇X₁₈GCX₁₉X₂₀X₂₁DX₂₂NCYDRTDCVEX₂₃X₂₄X₂₅X₂₆PX₂₇VYFCCC
EGNMCNEKFSYFPEMEVTQPTS (SEQ ID NO: 140)
GAILGRSETQECLFX₂NANWEX₄X₅RTNQTGVEX₇CX₈GX₉KDKRX₁₄HCX₁₅ATWX₁₆
NISGSIEIVKX₁₈GCWLDDX₂₂NCYDRTDCVEX₂₃X₂₄X₂₅X₂₆PX₂₇VYFCCCEGNMCNE
KFSYFPEMEVTQPTS (SEQ ID NO: 141)
GAILGRSETQECLFX₂NANWEX₄DRTNQTGVEX₇CX₈GX₉KDKRX₁₄HCX₁₅ATW
X₁₆NISGSIEIVKX₁₈GCWLDDX₂₂NCYDRTDCVEX₂₃KX₂₅X₂₆PX₂₇VYFCCCEGNMCN
EKFSYFPEMEVTQPTS (SEQ ID NO: 142)
GAILGRSETQECLFX₂NANWEX₄DRTNQTGVEPCX₈GX₉KDKRX₁₄HCFATWKNISG
SIEIVKX₁₈GCWLDDINCYDRTDCVEX₂₃KX₂₅X₂₆PX₂₇VYFCCCEGNMCNEKFSYFPE
MEVTQPTS (SEQ ID NO: 143)

| | | | |
|---|---|---|---|
| X₁ | F, A, V, I, L, M, F, | X₁₅ | F, A, V, I, L, M, F, |
| | Y or W | | Y or W |
| X₂ | F, A, V, I, L, M, F, | X₁₆ | K, R, H, or A |
| | Y or W | | |
| X₃ | D, E or A | X₁₇ | K, A, Y, F, I, V, L, |
| | | | M, or W |
| X₄ | K or L | X₁₈ | Q or K |
| X₅ | D, E, or A | X₁₉ | W or A |
| X₆ | R, H, K, or A | X₂₀ | L or A |
| X₇ | P or R | X₂₁ | D, K, R, H, A, F, G, |
| | | | M, N, or I |
| X₈ | Y or E | X₂₂ | I, F, A, V, L, M, Y |
| | | | or W |
| X₉ | D, E, or A | X₂₃ | K or T |
| X₁₀ | K or Q | X₂₄ | K, R, H, D, or E |
| X₁₁ | D, E, or A | X₂₅ | D, E, or A |
| X₁₂ | K, R, H, or A | X₂₆ | S, N, T, or Q |
| X₁₃ | R, H, K, or A | X₂₇ | E or Q |
| X₁₄ | R or L | | |

In some embodiments, an extracellular ActRIIA variant comprising the sequence of SEQ ID NO: 140 has the following amino acid substitutions: X₃ is, X₆ is R, X₁₁ is D, X₁₂ is K, X₁₃ is R, X₁₆, is K or R, X₁₇ is K, X₁₉ is W, X₂₀ is L, X₂₁ is D, and X₂₂ is I or F. In some embodiments, an extracellular ActRIIA variant comprising the sequence of SEQ ID NO: 139 or 140 has the following amino acid substitutions: X₁₇ is K. In some embodiments, an extracellular ActRIIA variant comprising the sequence of SEQ ID NOs: 139-141 has the following amino acid substitutions: X₁₇ is K, X₂₃ is T, X₂₄ is E, X₂₅ is E, and X₂₆, is N. In some embodiments, an extracellular ActRIIA variant comprising the sequence of any one of SEQ ID NOs: 139-143 has the following amino acid substitutions: X₁₇ is K, X₂₃ is T, X₂₄ is K, X₂₅ is E, and X₂₆, is N.

In some embodiments, a polypeptide described herein includes an extracellular ActRIIA variant having a sequence of any one of SEQ ID NOs: 144-210 (Table 5).

TABLE 5
Extracellular ActRIIA variant polypeptides having the sequences of SEQ ID
NOs: 144-210

| SEQ ID | |
|---|---|
| NO | Amino Acid Sequence |
| 144 | GAILGRSETQECLFYNANWELDRTNQTGVEPCEGEKDKRLHCFATWKNIS |
| | GSIEIVKKGCWLDDINCYDRTDCVETKENPQVYFCCCEGNMCNEKFSYFPE |
| | MEVTQPTS |
| 145 | GAILGRSETQECLFYNANWELERTNQTGVEPCEGEKDKRLHCFATWKNIS |
| | GSIEIVKKGCWLDDINCYDRTDCVETKENPQVYFCCCEGNMCNEKFSYFPE |
| | MEVTQPTS |
| 146 | GAILGRSETQECLFYNANWELDRTNQTGVERCEGEKDKRLHCFATWKNIS |
| | GSIEIVKKGCWLDDINCYDRTDCVETKENPQVYFCCCEGNMCNEKFSYFPE |
| | MEVTQPTS |
| 147 | GAILGRSETQECLFYNANWELDRTNQTGVEPCEGEKDKRLHCYATWKNIS |
| | GSIEIVKKGCWLDDINCYDRTDCVETKENPQVYFCCCEGNMCNEKFSYFPE |
| | MEVTQPTS |
| 148 | GAILGRSETQECLFYNANWELDRTNQTGVEPCEGEKDKRLHCFATWRNIS |
| | GSIEIVKKGCWLDDINCYDRTDCVETKENPQVYFCCCEGNMCNEKFSYFPE |
| | MEVTQPTS |
| 149 | GAILGRSETQECLFYNANWELDRTNQTGVEPCEGEKDKRLHCFATWKNIS |
| | GSIEIVKKGCWLDDFNCYDRTDCVETKENPQVYFCCCEGNMCNEKFSYFP |
| | EMEVTQPTS |
| 150 | GAILGRSETQECLFYNANWELDRTNQTGVEPCEGEKDKRLHCFATWKNIS |
| | GSIEIVKKGCWLDDINCYDRTDCVETEENPQVYFCCCEGNMCNEKFSYFPE |
| | MEVTQPTS |
| 151 | GAILGRSETQECLFYNANWELERTNQTGVERCEGEKDKRLHCFATWKNIS |
| | GSIEIVKKGCWLDDINCYDRTDCVETKENPQVYFCCCEGNMCNEKFSYFPE |
| | MEVTQPTS |
| 152 | GAILGRSETQECLFYNANWELERTNQTGVEPCEGEKDKRLHCYATWKNIS |
| | GSIEIVKKGCWLDDINCYDRTDCVETKENPQVYFCCCEGNMCNEKFSYFPE |
| | MEVTQPTS |
| 153 | GAILGRSETQECLFYNANWELERTNQTGVEPCEGEKDKRLHCFATWRNIS |
| | GSIEIVKKGCWLDDINCYDRTDCVETKENPQVYFCCCEGNMCNEKFSYFPE |
| | MEVTQPTS |
| 154 | GAILGRSETQECLFYNANWELERTNQTGVEPCEGEKDKRLHCFATWKNIS |
| | GSIEIVKKGCWLDDFNCYDRTDCVETKENPQVYFCCCEGNMCNEKFSYFP |
| | EMEVTQPTS |
| 155 | GAILGRSETQECLFYNANWELERTNQTGVEPCEGEKDKRLHCFATWKNIS |
| | GSIEIVKKGCWLDDINCYDRTDCVETEENPQVYFCCCEGNMCNEKFSYFPE |
| | MEVTQPTS |
| 156 | GAILGRSETQECLFYNANWELDRTNQTGVERCEGEKDKRLHCYATWKNIS |
| | GSIEIVKKGCWLDDINCYDRTDCVETKENPQVYFCCCEGNMCNEKFSYFPE |
| | MEVTQPTS |
| 157 | GAILGRSETQECLFYNANWELDRTNQTGVERCEGEKDKRLHCFATWRNIS |
| | GSIEIVKKGCWLDDINCYDRTDCVETKENPQVYFCCCEGNMCNEKFSYFPE |
| | MEVTQPTS |
| 158 | GAILGRSETQECLFYNANWELDRTNQTGVERCEGEKDKRLHCFATWKNIS |
| | GSIEIVKKGCWLDDFNCYDRTDCVETKENPQVYFCCCEGNMCNEKFSYFP |
| | EMEVTQPTS |
| 159 | GAILGRSETQECLFYNANWELDRTNQTGVERCEGEKDKRLHCFATWKNIS |
| | GSIEIVKKGCWLDDINCYDRTDCVETEENPQVYFCCCEGNMCNEKFSYFPE |
| | MEVTQPTS |
| 160 | GAILGRSETQECLFYNANWELDRTNQTGVEPCEGEKDKRLHCYATWRNIS |
| | GSIEIVKKGCWLDDINCYDRTDCVETKENPQVYFCCCEGNMCNEKFSYFPE |
| | MEVTQPTS |
| 161 | GAILGRSETQECLFYNANWELDRTNQTGVEPCEGEKDKRLHCYATWKNIS |
| | GSIEIVKKGCWLDDFNCYDRTDCVETKENPQVYFCCCEGNMCNEKFSYFP |
| | EMEVTQPTS |
| 162 | GAILGRSETQECLFYNANWELDRTNQTGVEPCEGEKDKRLHCYATWKNIS |
| | GSIEIVKKGCWLDDINCYDRTDCVETEENPQVYFCCCEGNMCNEKFSYFPE |
| | MEVTQPTS |
| 163 | GAILGRSETQECLFYNANWELDRTNQTGVEPCEGEKDKRLHCFATWRNIS |
| | GSIEIVKKGCWLDDFNCYDRTDCVETKENPQVYFCCCEGNMCNEKFSYFP |
| | EMEVTQPTS |
| 164 | GAILGRSETQECLFYNANWELDRTNQTGVEPCEGEKDKRLHCFATWRNIS |
| | GSIEIVKKGCWLDDINCYDRTDCVETEENPQVYFCCCEGNMCNEKFSYFPE |
| | MEVTQPTS |
| 165 | GAILGRSETQECLFYNANWELDRTNQTGVEPCEGEKDKRLHCFATWKNIS |
| | GSIEIVKKGCWLDDFNCYDRTDCVETEENPQVYFCCCEGNMCNEKFSYFPE |
| | MEVTQPTS |
| 166 | GAILGRSETQECLFYNANWELERTNQTGVERCEGEKDKRLHCYATWKNIS |
| | GSIEIVKKGCWLDDINCYDRTDCVETKENPQVYFCCCEGNMCNEKFSYFPE |
| | MEVTQPTS |
| 167 | GAILGRSETQECLFYNANWELERTNQTGVERCEGEKDKRLHCFATWRNIS |
| | GSIEIVKKGCWLDDINCYDRTDCVETKENPQVYFCCCEGNMCNEKFSYFPE |
| | MEVTQPTS |
| 168 | GAILGRSETQECLFYNANWELERTNQTGVERCEGEKDKRLHCFATWKNIS |
| | GSIEIVKKGCWLDDFNCYDRTDCVETKENPQVYFCCCEGNMCNEKFSYFP |
| | EMEVTQPTS |
| 169 | GAILGRSETQECLFYNANWELERTNQTGVERCEGEKDKRLHCFATWKNIS |
| | GSIEIVKKGCWLDDINCYDRTDCVETEENPQVYFCCCEGNMCNEKFSYFPE |
| | MEVTQPTS |
| 170 | GAILGRSETQECLFYNANWELERTNQTGVEPCEGEKDKRLHCYATWRNIS |
| | GSIEIVKKGCWLDDINCYDRTDCVETKENPQVYFCCCEGNMCNEKFSYFPE |
| | MEVTQPTS |
| 171 | GAILGRSETQECLFYNANWELERTNQTGVEPCEGEKDKRLHCYATWKNIS |
| | GSIEIVKKGCWLDDFNCYDRTDCVETKENPQVYFCCCEGNMCNEKFSYFP |
| | EMEVTQPTS |
| 172 | GAILGRSETQECLFYNANWELERTNQTGVEPCEGEKDKRLHCYATWKNIS |
| | GSIEIVKKGCWLDDINCYDRTDCVETEENPQVYFCCCEGNMCNEKFSYFPE |
| | MEVTQPTS |
| 173 | GAILGRSETQECLFYNANWELERTNQTGVEPCEGEKDKRLHCFATWRNIS |
| | GSIEIVKKGCWLDDFNCYDRTDCVETKENPQVYFCCCEGNMCNEKFSYFP |
| | EMEVTQPTS |
| 174 | GAILGRSETQECLFYNANWELERTNQTGVEPCEGEKDKRLHCFATWRNIS |
| | GSIEIVKKGCWLDDINCYDRTDCVETEENPQVYFCCCEGNMCNEKFSYFPE |
| | MEVTQPTS |
| 175 | GAILGRSETQECLFYNANWELERTNQTGVEPCEGEKDKRLHCFATWKNIS |
| | GSIEIVKKGCWLDDFNCYDRTDCVETEENPQVYFCCCEGNMCNEKFSYFPE |
| | MEVTQPTS |
| 176 | GAILGRSETQECLFYNANWELDRTNQTGVERCEGEKDKRLHCYATWRNIS |
| | GSIEIVKKGCWLDDINCYDRTDCVETKENPQVYFCCCEGNMCNEKFSYFPE |
| | MEVTQPTS |
| 177 | GAILGRSETQECLFYNANWELDRTNQTGVERCEGEKDKRLHCYATWKNIS |
| | GSIEIVKKGCWLDDENCYDRTDCVETKENPQVYFCCCEGNMCNEKFSYFP |
| | EMEVTQPTS |
| 178 | GAILGRSETQECLFYNANWELDRTNQTGVERCEGEKDKRLHCYATWKNIS |
| | GSIEIVKKGCWLDDINCYDRTDCVETEENPQVYFCCCEGNMCNEKFSYFPE |
| | MEVTQPTS |
| 179 | GAILGRSETQECLFYNANWELDRTNQTGVERCEGEKDKRLHCFATWRNIS |
| | GSIEIVKKGCWLDDFNCYDRTDCVETKENPQVYFCCCEGNMCNEKFSYFP |
| | EMEVTQPTS |
| 180 | GAILGRSETQECLFYNANWELDRTNQTGVERCEGEKDKRLHCFATWRNIS |
| | GSIEIVKKGCWLDDINCYDRTDCVETEENPQVYFCCCEGNMCNEKFSYFPE |
| | MEVTQPTS |
| 181 | GAILGRSETQECLFYNANWELDRTNQTGVERCEGEKDKRLHCFATWKNIS |
| | GSIEIVKKGCWLDDFNCYDRTDCVETEENPQVYFCCCEGNMCNEKFSYFPE |
| | MEVTQPTS |
| 182 | GAILGRSETQECLFYNANWELDRTNQTGVEPCEGEKDKRLHCYATWRNIS |
| | GSIEIVKKGCWLDDFNCYDRTDCVETKENPQVYFCCCEGNMCNEKFSYFP |
| | EMEVTQPTS |
| 183 | GAILGRSETQECLFYNANWELDRTNQTGVEPCEGEKDKRLHCYATWRNIS |
| | GSIEIVKKGCWLDDINCYDRTDCVETEENPQVYFCCCEGNMCNEKFSYFPE |
| | MEVTQPTS |
| 184 | GAILGRSETQECLFYNANWELDRTNQTGVEPCEGEKDKRLHCYATWKNIS |
| | GSIEIVKKGCWLDDFNCYDRTDCVETEENPQVYFCCCEGNMCNEKFSYFPE |
| | MEVTQPTS |
| 185 | GAILGRSETQECLFYNANWELDRTNQTGVEPCEGEKDKRLHCFATWRNIS |
| | GSIEIVKKGCWLDDFNCYDRTDCVETEENPQVYFCCCEGNMCNEKFSYFPE |
| | MEVTQPTS |
| 186 | GAILGRSETQECLFYNANWELERTNQTGVERCEGEKDKRLHCYATWRNIS |
| | GSIEIVKKGCWLDDINCYDRTDCVETKENPQVYFCCCEGNMCNEKFSYFPE |
| | MEVTQPTS |
| 187 | GAILGRSETQECLFYNANWELERTNQTGVERCEGEKDKRLHCYATWKNIS |
| | GSIEIVKKGCWLDDFNCYDRTDCVETKENPQVYFCCCEGNMCNEKFSYFP |
| | EMEVTQPTS |
| 188 | GAILGRSETQECLFYNANWELERTNQTGVERCEGEKDKRLHCYATWKNIS |
| | GSIEIVKKGCWLDDINCYDRTDCVETEENPQVYFCCCEGNMCNEKFSYFPE |
| | MEVTQPTS |
| 189 | GAILGRSETQECLFYNANWELERTNQTGVERCEGEKDKRLHCFATWRNIS |
| | GSIEIVKKGCWLDDFNCYDRTDCVETKENPQVYFCCCEGNMCNEKFSYFP |
| | EMEVTQPTS |
| 190 | GAILGRSETQECLFYNANWELERTNQTGVERCEGEKDKRLHCFATWRNIS |
| | GSIEIVKKGCWLDDINCYDRTDCVETEENPQVYFCCCEGNMCNEKFSYFPE |
| | MEVTQPTS |
| 191 | GAILGRSETQECLFYNANWELERTNQTGVERCEGEKDKRLHCFATWKNIS |
| | GSIEIVKKGCWLDDFNCYDRTDCVETEENPQVYFCCCEGNMCNEKFSYFPE |
| | MEVTQPTS |
| 192 | GAILGRSETQECLFYNANWELERTNQTGVEPCEGEKDKRLHCYATWRNIS |
| | GSIEIVKKGCWLDDFNCYDRTDCVETKENPQVYFCCCEGNMCNEKFSYFP |
| | EMEVTQPTS |
| 193 | GAILGRSETQECLFYNANWELERTNQTGVEPCEGEKDKRLHCYATWRNIS |
| | GSIEIVKKGCWLDDINCYDRTDCVETEENPQVYFCCCEGNMCNEKFSYFPE |
| | MEVTQPTS |
| 194 | GAILGRSETQECLFYNANWELERTNQTGVEPCEGEKDKRLHCYATWKNIS |
| | GSIEIVKKGCWLDDFNCYDRTDCVETEENPQVYFCCCEGNMCNEKFSYFPE |
| | MEVTQPTS |
| 195 | GAILGRSETQECLFYNANWELERTNQTGVEPCEGEKDKRLHCFATWRNIS |
| | GSIEIVKKGCWLDDFNCYDRTDCVETEENPQVYFCCCEGNMCNEKFSYFPE |
| | MEVTQPTS |
| 196 | GAILGRSETQECLFYNANWELDRTNQTGVERCEGEKDKRLHCYATWRNIS |
| | GSIEIVKKGCWLDDFNCYDRTDCVETKENPQVYFCCCEGNMCNEKFSYFP |
| | EMEVTQPTS |
| 197 | GAILGRSETQECLFYNANWELDRTNQTGVERCEGEKDKRLHCYATWRNIS |
| | GSIEIVKKGCWLDDINCYDRTDCVETEENPQVYFCCCEGNMCNEKFSYFPE |
| | MEVTQPTS |
| 198 | GAILGRSETQECLFYNANWELDRTNQTGVERCEGEKDKRLHCYATWKNIS |
| | GSIEIVKKGCWLDDFNCYDRTDCVETEENPQVYFCCCEGNMCNEKFSYFPE |
| | MEVTQPTS |
| 199 | GAILGRSETQECLFYNANWELDRTNQTGVERCEGEKDKRLHCFATWRNIS |
| | GSIEIVKKGCWLDDFNCYDRTDCVETEENPQVYFCCCEGNMCNEKFSYFPE |
| | MEVTQPTS |
| 200 | GAILGRSETQECLFYNANWELDRTNQTGVEPCEGEKDKRLHCYATWRNIS |
| | GSIEIVKKGCWLDDFNCYDRTDCVETEENPQVYFCCCEGNMCNEKFSYFPE |
| | MEVTQPTS |
| 201 | GAILGRSETQECLFYNANWELERTNQTGVERCEGEKDKRLHCYATWRNIS |
| | GSIEIVKKGCWLDDFNCYDRTDCVETKENPQVYFCCCEGNMCNEKFSYFP |
| | EMEVTQPTS |
| 202 | GAILGRSETQECLFYNANWELERTNQTGVERCEGEKDKRLHCYATWRNIS |
| | GSIEIVKKGCWLDDINCYDRTDCVETEENPQVYFCCCEGNMCNEKFSYFPE |
| | MEVTQPTS |
| 203 | GAILGRSETQECLFYNANWELERTNQTGVERCEGEKDKRLHCYATWKNIS |
| | GSIEIVKKGCWLDDFNCYDRTDCVETEENPQVYFCCCEGNMCNEKFSYFPE |
| | MEVTQPTS |
| 204 | GAILGRSETQECLFYNANWELERTNQTGVERCEGEKDKRLHCFATWRNIS |
| | GSIEIVKKGCWLDDFNCYDRTDCVETEENPQVYFCCCEGNMCNEKFSYFPE |
| | MEVTQPTS |
| 205 | GAILGRSETQECLFYNANWELDRTNQTGVERCEGEKDKRLHCYATWRNIS |
| | GSIEIVKKGCWLDDENCYDRTDCVETEENPQVYFCCCEGNMCNEKFSYFPE |
| | MEVTQPTS |
| 206 | GAILGRSETQECLFYNANWELERTNQTGVEPCEGEKDKRLHCYATWRNIS |
| | GSIEIVKKGCWLDDFNCYDRTDCVETEENPQVYFCCCEGNMCNEKFSYFPE |
| | MEVTQPTS |
| 207 | GAILGRSETQECLFYNANWELERTNQTGVERCEGEKDKRLHCYATWRNIS |
| | GSIEIVKKGCWLDDFNCYDRTDCVETEENPQVYFCCCEGNMCNEKFSYFPE |
| | MEVTQPTS |
| 208 | GAILGRSETQECLYYNANWELERTNQTGVERCEGEQDKRLHCYATWRNIS |
| | GSIEIVKKGCWLDDFNCYDRTDCVETEENPQVYFCCCEGNMCNEKFSYFPE |
| | MEVTQPTS |
| 209 | GAILGRSETQECLFYNANWELERTNQTGVERCEGEKDKRLHCYATWRNIS |
| | GSIEIVKKGCWLDDFNCYDRTDCVETEENPQVYFCCCEGNMCNEKFSYFPE |
| | MEVTQPTSNP |
| 210 | GAILGRSETQECLFYNANWELERTNQTGVERCEGEKDKRLHCYATWRNIS |
| | GSIEIVKKGCWLDDFNCYDRTDCVETEENPQVYFCCCEGNMCNEKFSYFPE |
| | MEVTQPTSNPVTPK |

In some embodiments, a polypeptide disclosed herein comprises an extracellular ActRIIA variant polypeptide (e.g., any one of SEQ ID NOs: 139-210 (e.g., SEQ ID NOs: 144-210)) having an amino acid K at the position corresponding to X₁₇ in SEQ ID NO: 139 or SEQ ID NO: 140. In some embodiments, altering the amino acid at position X₁₇ can result in reduced activity. For example, an ActRIIA variant having the sequence

(SEQ ID NO: 283)
GAILGRSETQECLFYNANWELERTNQTGVERCEGEKDKRLHCYATWRNI
SGSIEIVAKGCWLDDFNCYDRTDCVETEENPQVYFCCCEGNMCNEKFSY
FPEMEVTQPTS

has reduced activity in vivo, indicating that the substitution of A for K at X₁₇ is not tolerated.

In some embodiments, a polypeptide disclosed herein including an extracellular ActRIIA variant (e.g., any one of SEQ ID NOs: 139-210 (e.g., SEQ ID NOs: 144-210)) with the sequence TEEN at positions X₂₃, X₂₄, X₂₅, and X₂₆ can have a substitution of the amino acid K for the amino acid E at position X₂₄. In some embodiments, a polypeptide disclosed herein including an extracellular ActRIIA variant (e.g., any one of SEQ ID NOs: 139-210 (e.g., SEQ ID NOs: 144-210)) with the sequence TKEN at positions X₂₃, X₂₄, X₂₅, and X₂₆ can have a substitution of the amino acid E for the amino acid K at position X₂₄. In some embodiments, polypeptides having the sequence TEEN or TKEN at positions X₂₃, X₂₄, X₂₅, and X₂₆ have reduced binding to BMP9.

In some embodiments, a polypeptide disclosed herein including an extracellular ActRIIA variant (e.g., any one of SEQ ID NOs: 139-208 (e.g., SEQ ID NOs: 144-208)) may further include a C-terminal extension (e.g., additional amino acids at the C-terminus). In some embodiments, the C-terminal extension is amino acid sequence NP. For example, the sequence including the C-terminal extension is SEQ ID NO: 209 (e.g., SEQ ID NO: 207 with a C-terminal extension of NP). In some embodiments, the C-terminal extension is amino acid sequence NPVTPK (SEQ ID NO: 288). For example, the sequence including the C-terminal extension is SEQ ID NO: 210 (e.g., SEQ ID NO: 207 with a C-terminal extension of NPVTPK). The C-terminal extension can add one to six additional amino acids at the C-terminus (e.g., 1, 2, 3, 4, 5, 6 or more additional amino acids).

In some embodiments, Compositions that can be administered to a subject according to the methods described herein are provided in Table 6, below.

TABLE 6
Compositions that can be administed to a subject according to the methods
described herein.

| Row | Composition |
|---|---|
| 1 | A polypeptide comprising an extracellular activin receptor type IIa (ActRIIA) variant, |
| | the variant having a sequence of |
| | GAILGRSETQECLX₁X₂NANWX₃X₄X₅X₆TNQTGVEX₇CX₈GX₉X₁₀X₁₁X₁₂X₁₃X₁₄HC |
| | X₁₅ATWX₁₆NISGSIEIVX₁₇X₁₈GCX₁₉X₂₀X₂₁DX₂₂NCYDRTDCVEX₂₃X₂₄X₂₅X₂₆PX₂₇V |
| | YFCCCEGNMCNEKFSYFPEMEVTQPTS (SEQ ID NO: 139), wherein X₁ is F or Y; |
| | X₂ is F or Y; X₃ is E or A; X₄ is K or L; X₅ is D or E; X₆ is R or A; X₇ is P or R; X₈ is Y |
| | or E; X₉ is D or E; X₁₀ is K or Q; X₁₁ is D or A; X₁₂ is K or A; X₁₃ is R or A; X₁₄ is R or |
| | L; X₁₅ is F or Y; X₁₆ is K, R, or A; X₁₇ is K, A, Y, F, or I; X₁₈ is Q or K; X₁₉ is W or A; |
| | X₂₀ is L or A; X₂₁ is D, K, R, A, F, G, M, N, or I; X₂₂ is I, F, or A; X₂₃ is K or T; X₂₄ is |
| | K or E; X₂₅ is D or E; X₂₆ is S or N; and X₂₇ is E or Q, and wherein the variant has at |
| | least one amino acid substitution relative to a wild-type extracellular ActRIIA having |
| | the sequence of SEQ ID NO: 211 or an extracellular ActRIIA having any one of the |
| | sequences of SEQ ID NOs: 212-232. |
| 2 | A polypeptide comprising an extracellular activin receptor type IIa (ActRIIA) variant, |
| | the variant having a sequence of |
| | GAILGRSETQECLX₁X₂NANWX₃X₄X₅X₆TNQTGVEX₇CX₈GX₉X₁₀X₁₁X₁₂X₁₃X₁₄HC |
| | X₁₅ATWX₁₆NISGSIEIVX₁₇X₁₈GCX₁₉X₂₀X₂₁DX₂₂NCYDRTDCVEX₂₃X₂₄X₂₅X₂₆PX₂₇V |
| | YFCCCEGNMCNEKFSYFPEMEVTQPTS (SEQ ID NO: 139), wherein X₁ is F, A, V, |
| | I, L, M, F, Y or W; X₂ is F, A, V, I, L, M, F, Y or W; X₃ is D, E or A; X₄ is K or L; X₅ |
| | is D, E, or A; X₆ is R, H, K, or A; X₇ is P or R; X₈ is Y or E; X₉ is D, E, or A; X₁₀ is K |
| | or Q; X₁₁ is D, E, or A; X₁₂ is K, R, H, or A; X₁₃ is R, H, K, or A; X₁₄ is R or L; X₁₅ is |
| | F, A, V, I, L, M, F, Y or W; X₁₆ is K, R, H, or A; X₁₇ is K, A, Y, F, I, V, L, M, or W; |
| | X₁₈ is Q or K; X₁₉ is W or A; X₂₀ is L or A; X₂₁ is D, K, R, H, A, F, G, M, N, or I; X₂₂ is |
| | I, F, A, V, L, M, Y or W; X₂₃ is K or T; X₂₄ is K, R, H, D, or E; X₂₅ is D, E, or A; X₂₆ is |
| | S, N, T, or Q; and X₂₇ is E or Q, and wherein the variant has at least one amino acid |
| | substitution relative to a wild-type extracellular ActRIIA having the sequence of SEQ |
| | ID NO: 211 or an extracellular ActRIIA having any one of the sequences of SEQ ID |
| | NOs: 212-232. |
| 3 | The polypeptide of row 1 or 2, wherein the variant has a sequence of |
| | GAILGRSETQECLFX₂NANWX₃X₄X₅X₆TNQTGVEX₇CX₈GX₉KX₁₁X₁₂X₁₃X₁₄HCX₁₅ |
| | ATWX₁₆NISGSIEIVX₁₇X₁₈GCX₁₉X₂₀X₂₁DX₂₂NCYDRTDCVEX₂₃X₂₄X₂₅X₂₆PX₂₇VYF |
| | CCCEGNMCNEKFSYFPEMEVTQPTS (SEQ ID NO: 140). |
| 4 | The polypeptide of rows 1-3, wherein the variant has a sequence of |
| | GAILGRSETQECLFX₂NANWEX₄X₅RTNQTGVEX₇CX₈GX₉KDKRX₁₄HCX₁₅ATW |
| | X₁₆NISGSIEIVKX₁₈GCWLDDX₂₂NCYDRTDCVEX₂₃X₂₄X₂₅X₂₆PX₂₇VYFCCCEGNM |
| | CNEKFSYFPEMEVTQPTS (SEQ ID NO: 141). |
| 5 | The polypeptide of any one of rows 1-4, wherein the variant has a sequence of |
| | GAILGRSETQECLFX₂NANWEX₄DRTNQTGVEX₇CX₈GX₉KDKRX₁₄HCX₁₅ATWX₁₆ |
| | NISGSIEIVKX₁₈GCWLDDX₂₂NCYDRTDCVEX₂₃KX₂₅X₂₆PX₂₇VYFCCCEGNMCN |
| | EKFSYFPEMEVTQPTS (SEQ ID NO: 142). |
| 6 | The polypeptide of any one of rows 1-5, wherein the variant has a sequence of |
| | GAILGRSETQECLFX₂NANWEX₄DRTNQTGVEPCX₈GX₉KDKRX₁₄HCFATWKNI |
| | SGSIEIVKX₁₈GCWLDDINCYDRTDCVEX₂₃KX₂₅X₂₆PX₂₇VYFCCCEGNMCNEKFS |
| | YFPEMEVTQPTS (SEQ ID NO: 143). |
| 7 | The polypeptide of row 1 or 2, wherein X₁ is F. |
| 8 | The polypeptide of row 1 or 2, wherein X₁ is Y. |
| 9 | The polypeptide of row 1, 2, 7, or 8 wherein X₁₀ is K. |
| 10 | The polypeptide of row 1, 2, 7, or 8 wherein X₁₀ is Q. |
| 11 | The polypeptide of any one of rows 1-10, wherein X₂ is F. |
| 12 | The polypeptide of any one of rows 1-10, wherein X₂ is Y. |
| 13 | The polypeptide of any one of rows 1, 2, 3, and 7-12, wherein X₃ is E. |
| 14 | The polypeptide of any one of rows 1, 2, 3, and 7-12, wherein X₃ is A. |
| 15 | The polypeptide of any one of rows 1-14, wherein X₄ is K. |
| 16 | The polypeptide of any one of rows 1-14, wherein X₄ is L. |
| 17 | The polypeptide of any one of rows 1, 2, 3, 4, and 7-16, wherein X₅ is D. |
| 18 | The polypeptide of any one of rows 1, 2, 3, 4, and 7-16, wherein X₅ is E. |
| 19 | The polypeptide of any one of rows 1, 2, 3, and 7-18, wherein X₆ is R. |
| 20 | The polypeptide of any one of rows 1, 2, 3, and 7-18, wherein X₆ is A. |
| 21 | The polypeptide of any one of rows 1-5 and 7-20, wherein X₇ is P. |
| 22 | The polypeptide of any one of rows 1-5 and 7-20, wherein X₇ is R. |
| 23 | The polypeptide of any one of rows 1-22, wherein X₈ is Y. |
| 24 | The polypeptide of any one of rows 1-22, wherein X₈ is E. |
| 25 | The polypeptide of any one of rows 1-24, wherein X₉ is D. |
| 26 | The polypeptide of any one of rows 1-24, wherein X₉ is E |
| 27 | The polypeptide of any one of rows 1, 2, 3, and 7-26, wherein X₁₁ is D. |
| 28 | The polypeptide of any one of rows 1, 2, 3, and 7-26, wherein X₁₁ is A. |
| 29 | The polypeptide of any one of rows 1, 2, 3, and 7-28, wherein X₁₂ is K. |
| 30 | The polypeptide of any one of rows 1, 2, 3, and 7-28, wherein X₁₂ is A. |
| 31 | The polypeptide of any one of rows 1, 2, 3, and 7-30, wherein X₁₃ is R. |
| 32 | The polypeptide of any one of rows 1, 2, 3, and 7-30, wherein X₁₃ is A. |
| 33 | The polypeptide of any one of rows 1-32, wherein X₁₄ is R. |
| 34 | The polypeptide of any one of rows 1-32, wherein X₁₄ is L. |
| 35 | The polypeptide of any one of rows 1-5 and 7-34, wherein X₁₅ is F. |
| 36 | The polypeptide of any one of rows 1-5 and 7-34, wherein X₁₅ is Y. |
| 37 | The polypeptide of any one of rows 1-5 and 7-36, wherein X₁₆ is K. |
| 38 | The polypeptide of any one of rows 1-5 and 7-36, wherein X₁₆ is R. |
| 39 | The polypeptide of any one of rows 1-5 and 7-36, wherein X₁₆ is A. |
| 40 | The polypeptide of any one of rows 1, 2, 3, and 7-39, wherein X₁₇ is K. |
| 41 | The polypeptide of any one of rows 1, 2, 3, and 7-39, wherein X₁₇ is A. |
| 42 | The polypeptide of any one of rows 1, 2, 3, and 7-39, wherein X₁₇ is Y. |
| 43 | The polypeptide of any one of rows 1, 2, 3, and 7-39, wherein X₁₇ is F. |
| 44 | The polypeptide of any one of rows 1, 2, 3, and 7-39, wherein X₁₇ is I. |
| 45 | The polypeptide of any one of rows 1-44, wherein X₁₈ is Q. |
| 46 | The polypeptide of any one of rows 1-44, wherein X₁₈ is K. |
| 47 | The polypeptide of any one of rows 1-3 and 7-46, wherein X₁₉ is W. |
| 48 | The polypeptide of any one of rows 1-3 and 7-46, wherein X₁₉ is A. |
| 49 | The polypeptide of any one of rows 1-3 and 7-48, wherein X₂₀ is L. |
| 50 | The polypeptide of any one of rows 1-3 and 7-48, wherein X₂₀ is A. |
| 51 | The polypeptide of any one of rows 1-3 and 7-50, wherein X₂₁ is D. |
| 52 | The polypeptide of any one of rows 1-3 and 7-50, wherein X₂₁ is K. |
| 53 | The polypeptide of any one of rows 1-3 and 7-50, wherein X₂₁ is R. |
| 54 | The polypeptide of any one of rows 1-3 and 7-50, wherein X₂₁ is A. |
| 55 | The polypeptide of any one of rows 1-3 and 7-50, wherein X₂₁ is F. |
| 56 | The polypeptide of any one of rows 1-3 and 7-50, wherein X₂₁ is G. |
| 57 | The polypeptide of any one of rows 1-3 and 7-50, wherein X₂₁ is M. |
| 58 | The polypeptide of any one of rows 1-3 and 7-50, wherein X₂₁ is N. |
| 59 | The polypeptide of any one of rows 1-3 and 7-50, wherein X₂₁ is I. |
| 60 | The polypeptide of any one of rows 1-5 and 7-59, wherein X₂₂ is I. |
| 61 | The polypeptide of any one of rows 1-5 and 7-59, wherein X₂₂ is F. |
| 62 | The polypeptide of any one of rows 1-5 and 7-59, wherein X₂₂ is A. |
| 63 | The polypeptide of any one of rows 1-62, wherein X₂₃ is K. |
| 64 | The polypeptide of any one of rows 1-62, wherein X₂₃ is T. |
| 65 | The polypeptide of any one of rows 1-4 and 7-64, wherein X₂₄ is K. |
| 66 | The polypeptide of any one of rows 1-4 and 7-64, wherein X₂₄ is E. |
| 67 | The polypeptide of any one of rows 1-66, wherein X₂₅ is D. |
| 68 | The polypeptide of any one of rows 1-66, wherein X₂₅ is E. |
| 69 | The polypeptide of any one of rows 1-68, wherein X₂₆ is S. |
| 70 | The polypeptide of any one of rows 1-68, wherein X₂₆ is N. |
| 71 | The polypeptide of any one of rows 1-70, wherein X₂₇ is E. |
| 72 | The polypeptide of any one of rows 1-70, wherein X₂₇ is Q. |
| 73 | The polypeptide of any one of rows 1-72, wherein X₂₃ is T, X₂₄ is E, X₂₅ is E, and X₂₆ is |
| | N. |
| 74 | The polypeptide of any one of rows 1-72, wherein X₂₃ is T, X₂₄ is E, X₂₅ is E, and X₂₆ is |
| | N. |
| 75 | The polypeptide of any one of rows 1-74, wherein X₁₇ is K. |
| 76 | The polypeptide of row 1 or 2, wherein the variant has the sequence of any one of SEQ |
| | ID NOs: 144-210. |
| 77 | The polypeptide of row 76, wherein the variant has the sequence of SEQ ID NO: 207. |
| 78 | The polypeptide of row 76, wherein the variant has the sequence of SEQ ID NO: 196. |
| 79 | The polypeptide of row 76, wherein the variant has the sequence of SEQ ID NO: 144. |
| 80 | The polypeptide of row 76, wherein the variant has the sequence of SEQ ID NO: 176. |
| 81 | The polypeptide of row 76, wherein the variant has the sequence of SEQ ID NO: 179. |
| 82 | The polypeptide of row 76, wherein the variant has the sequence of SEQ ID NO: 182. |
| 83 | The polypeptide of row 76, wherein the variant has the sequence of SEQ ID NO: 208. |
| 84 | The polypeptide of row 76, wherein the variant has the sequence of SEQ ID NO: 209. |
| 85 | The polypeptide of row 76, wherein the variant has the sequence of SEQ ID NO: 210. |
| 86 | The polypeptide of any one of rows 1-85, wherein the amino acid at position X₂₄ is |
| | replaced with the amino acid K. |
| 87 | The polypeptide of any one of rows 1-86, wherein the amino acid at position X₂₄ is |
| | replaced with the amino acid E. |
| 88 | The polypeptide of any one of rows 1-87, further comprising a C-terminal extension of |
| | one or more amino acids. |
| 89 | The polypeptide of row 88, wherein the C-terminal extension is NP. |
| 90 | The polypeptide of row 88, wherein the C-terminal extension is NPVTPK. |
| 91 | The polypeptide of any one of rows 1-90, further comprising an Fc domain monomer |
| | fused to the C-terminus of the polypeptide by way of a linker. |
| 92 | The polypeptide of row 91, wherein the Fc domain monomer comprises the sequence of |
| | SEQ ID NO: 233. |
| 93 | The polypeptide of any one of rows 1-90, further comprising a wild-type Fc domain |
| | fused to the C-terminus of the polypeptide by way of a linker. |
| 94 | The polypeptide of row 93, wherein the wild-type Fc domain comprises the sequence of |
| | SEQ ID NO: 284. |
| 95 | The polypeptide of any one of rows 1-90, further comprising an Fc domain with amino |
| | acid substitutions fused to the C-terminus of the polypeptide by way of a linker. |
| 96 | The polypeptide of row 95, wherein the Fc domain does not form a dimer. |
| 97 | The polypeptide of any one of rows 1-90, further comprising an albumin-binding |
| | peptide fused to the C-terminus of the polypeptide by way of a linker. |
| 98 | The polypeptide of row 97, wherein the albumin-binding peptide comprises the |
| | sequence of SEQ ID NO: 285. |
| 99 | The polypeptide of any one of rows 1-90, further comprising a fibronectin domain fused |
| | to the C-terminus of the polypeptide by way of a linker. |
| 100 | The polypeptide of row 99, wherein the fibronectin domain comprises the sequence of |
| | SEQ ID NO: 286. |
| 101 | The polypeptide of any one of rows 1-90, further comprising a human serum albumin |
| | fused to the C-terminus of the polypeptide by way of a linker. |
| 102 | The polypeptide of row 101, wherein the human serum albumin comprises the sequence |
| | of SEQ ID NO: 287. |
| 103 | The polypeptide of row 91 or 92, wherein the polypeptide forms a dimer. |
| 104 | The polypeptide of any one of rows 91-103, wherein the linker is an amino acid spacer. |
| 105 | The polypeptide of row 104, wherein the amino acid spacer is GGG, GGGA (SEQ ID |
| | NO: 234),GGGG (SEQ ID NO: 20), GGGAG (SEQ ID NO: 263), GGGAGG (SEQ ID |
| | NO: 264), or GGGAGGG (SEQ ID NO: 265). |

In some embodiments, an extracellular ActRIIA variant described herein does not have the sequence of any one of SEQ ID NOs: 212-232 shown in Table 7 below.

TABLE 7
Excluded Extracellular ActRIIA Variant polypeptides.

| SEQ ID NO: | Amino Acid Sequence |
|---|---|
| 212 | GAILGRSETQECLFFNANWEKDRTNQTGVEPCYGDKDKRRHCFATWA |
| | NISGSIEIVKQGCWLDDINCYDRTDCVEKKDSPEVYFCCCEGNMCNEKF |
| | SYFPEMEVTQPTS |
| 213 | GAILGRSETQECLFFNANWAKDRTNQTGVEPCYGDKDKRRHCFATWK |
| | NISGSIEIVKQGCWLDDINCYDRTDCVEKKDSPEVYFCCCEGNMCNEKF |
| | SYFPEMEVTQPTS |
| 214 | GAILGRSETQECLFFNANWEKDATNQTGVEPCYGDKDKRRHCFATWK |
| | NISGSIEIVKQGCWLDDINCYDRTDCVEKKDSPEVYFCCCEGNMCNEKF |
| | SYFPEMEVTQPTS |
| 215 | GAILGRSETQECLFFNANWEKDRTNQTGVEPCYGDKAKRRHCFATWK |
| | NISGSIEIVKQGCWLDDINCYDRTDCVEKKDSPEVYFCCCEGNMCNEKF |
| | SYFPEMEVTQPTS |
| 216 | GAILGRSETQECLFFNANWEKDRTNQTGVEPCYGDKDARRHCFATWK |
| | NISGSIEIVKQGCWLDDINCYDRTDCVEKKDSPEVYFCCCEGNMCNEKF |
| | SYFPEMEVTQPTS |
| 217 | GAILGRSETQECLFFNANWEKDRTNQTGVEPCYGDKDKARHCFATWK |
| | NISGSIEIVKQGCWLDDINCYDRTDCVEKKDSPEVYFCCCEGNMCNEKF |
| | SYFPEMEVTQPTS |
| 218 | GAILGRSETQECLFFNANWEKDRTNQTGVEPCYGDKDKRRHCFATWK |
| | NISGSIEIVAQGCWLDDINCYDRTDCVEKKDSPEVYFCCCEGNMCNEKF |
| | SYFPEMEVTQPTS |
| 219 | GAILGRSETQECLFFNANWEKDRTNQTGVEPCYGDKDKRRHCFATWK |
| | NISGSIEIVYQGCWLDDINCYDRTDCVEKKDSPEVYFCCCEGNMCNEKF |
| | SYFPEMEVTQPTS |
| 220 | GAILGRSETQECLFFNANWEKDRTNQTGVEPCYGDKDKRRHCFATWK |
| | NISGSIEIVFQGCWLDDINCYDRTDCVEKKDSPEVYFCCCEGNMCNEKF |
| | SYFPEMEVTQPTS |
| 221 | GAILGRSETQECLFFNANWEKDRTNQTGVEPCYGDKDKRRHCFATWK |
| | NISGSIEIVIQGCWLDDINCYDRTDCVEKKDSPEVYFCCCEGNMCNEKFS |
| | YFPEMEVTQPTS |
| 222 | GAILGRSETQECLFFNANWEKDRTNQTGVEPCYGDKDKRRHCFATWK |
| | NISGSIEIVKQGCALDDINCYDRTDCVEKKDSPEVYFCCCEGNMCNEKFS |
| | YFPEMEVTQPTS |
| 223 | GAILGRSETQECLFFNANWEKDRTNQTGVEPCYGDKDKRRHCFATWK |
| | NISGSIEIVKQGCWADDINCYDRTDCVEKKDSPEVYFCCCEGNMCNEKF |
| | SYFPEMEVTQPTS |
| 224 | GAILGRSETQECLFFNANWEKDRTNQTGVEPCYGDKDKRRHCFATWK |
| | NISGSIEIVKQGCWLKDINCYDRTDCVEKKDSPEVYFCCCEGNMCNEKF |
| | SYFPEMEVTQPTS |
| 225 | GAILGRSETQECLFFNANWEKDRTNQTGVEPCYGDKDKRRHCFATWK |
| | NISGSIEIVKQGCWLRDINCYDRTDCVEKKDSPEVYFCCCEGNMCNEKF |
| | SYFPEMEVTQPTS |
| 226 | GAILGRSETQECLFFNANWEKDRTNQTGVEPCYGDKDKRRHCFATWK |
| | NISGSIEIVKQGCWLADINCYDRTDCVEKKDSPEVYFCCCEGNMCNEKF |
| | SYFPEMEVTQPTS |
| 227 | GAILGRSETQECLFFNANWEKDRTNQTGVEPCYGDKDKRRHCFATWK |
| | NISGSIEIVKQGCWLFDINCYDRTDCVEKKDSPEVYFCCCEGNMCNEKF |
| | SYFPEMEVTQPTS |
| 228 | GAILGRSETQECLFFNANWEKDRTNQTGVEPCYGDKDKRRHCFATWK |
| | NISGSIEIVKQGCWLGDINCYDRTDCVEKKDSPEVYFCCCEGNMCNEKF |
| | SYFPEMEVTQPTS |
| 229 | GAILGRSETQECLFFNANWEKDRTNQTGVEPCYGDKDKRRHCFATWK |
| | NISGSIEIVKQGCWLMDINCYDRTDCVEKKDSPEVYFCCCEGNMCNEKF |
| | SYFPEMEVTQPTS |
| 230 | GAILGRSETQECLFFNANWEKDRTNQTGVEPCYGDKDKRRHCFATWK |
| | NISGSIEIVKQGCWLNDINCYDRTDCVEKKDSPEVYFCCCEGNMCNEKF |
| | SYFPEMEVTQPTS |
| 231 | GAILGRSETQECLFFNANWEKDRTNQTGVEPCYGDKDKRRHCFATWK |
| | NISGSIEIVKQGCWLIDINCYDRTDCVEKKDSPEVYFCCCEGNMCNEKFS |
| | YFPEMEVTQPTS |
| 232 | GAILGRSETQECLFFNANWEKDRTNQTGVEPCYGDKDKRRHCFATWK |
| | NISGSIEIVKQGCWLDDANCYDRTDCVEKKDSPEVYFCCCEGNMCNEK |
| | FSYFPEMEVTQPTS |

Furthermore, in some embodiments, a polypeptide described herein has a serum half-life of at least 7 days in humans. In some embodiments, the polypeptide may bind to bone morphogenetic protein 9 (BMP9) with a K_{D} of 200 pM or higher. In some embodiments, the polypeptide may bind to activin A with a K_{D} of 10 pM or higher. In some embodiments, the polypeptide does not bind to BMP9 or activin A. In some embodiments, the polypeptide binds to activin and/or myostatin and exhibits reduced binding to BMP9. In some embodiments, the polypeptide that has reduced binding to BMP9 has the sequence TEEN or TKEN at positions X₂₃, X₂₄, X₂₅, and X₂₆.

Additionally, in some embodiments, the polypeptide may bind to human BMP9 with a K_{D} of about 200 pM or higher (e.g., a K_{D} of about 200, 300, 400, 500, 600, 700, 800, or 900 pM or higher, e.g., a K_{D} of about 1, 2, 3, 4, 5, 6, 7, 8, 9, 10, 20, 30, 40, or 50 nM or higher, e.g., a K_{D} of between about 200 pM and about 50 nM). In some embodiments, the polypeptide does not substantially bind to human BMP9. In some embodiments, the polypeptide may bind to human activin A with a K_{D} of about 800 pM or less (e.g., a K_{D} of about 800, 700, 600, 500, 400, 300, 200,100, 90, 80, 70, 60, 50, 40, 30, 20, 10, 9, 8, 7, 6, 5, 4, 3, 2, or 1 pM or less, e.g., a K_{D} of between about 800 pM and about 200 pM). In some embodiments, the polypeptide may bind to human activin B with a K_{D} of 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 200 pM). In some embodiments, the polypeptide may also bind to growth and differentiation factor 11 (GDF-11) with a K_{D} of approximately 5 pM or higher (e.g., a K_{D} of about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 pM or higher).

To illustrate, one or more mutations may be selected that increase the selectivity of the altered ligand-binding domain for GDF11 and/or GDF8 over one or more ActRII-binding ligands such as activins (activin A, activin B, activin AB, activin C, and/or activin E), particularly activin A. Optionally, the altered ligand-binding domain has a ratio of K_{d} for activin binding to K_{d} for GDF11 and/or GDF8 binding that is at least 2-, 5-, 10-, 20-, 50-, 100- or even 1000-fold greater relative to the ratio for the wild-type ligand-binding domain. Optionally, the altered ligand-binding domain has a ratio of IC₅₀ for inhibiting activin to IC₅₀ for inhibiting GDF11 and/or GDF8 that is at least 2-, 5-, 10-, 20-, 50-, 100- or even 1000-fold greater relative to the wild-type ligand-binding domain. Optionally, the altered ligand-binding domain inhibits GDF11 and/or GDF8 with an IC₅₀ at least 2-, 5-, 10-, 20-, 50-, 100- or even 1000-times less than the IC₅₀ for inhibiting activin.

Amino acid residues of the ActRIIB proteins (e.g., E39, K55, Y60, K74, W78, L79, D80, and F101 with respect to SEQ ID NO: 1) are in the ActRIIB ligand-binding pocket and help mediate binding to its ligands including, for example, activin A, GDF11, and GDF8. Thus the present disclosure provides polypeptides comprising an altered-ligand binding domain (e.g., a GDF8/GDF11-binding domain) of an ActRIIB receptor which comprises one or more mutations at those amino acid residues.

As a specific example, the positively-charged amino acid residue Asp (D80) of the ligand-binding domain of ActRIIB can be mutated to a different amino acid residue to produce a polypeptide that preferentially binds to GDF8, but not activin. In some embodiments, the D80 residue with respect to SEQ ID NO: 1 is changed to an amino acid residue selected from the group consisting of: an uncharged amino acid residue, a negative amino acid residue, and a hydrophobic amino acid residue. As a further specific example, the hydrophobic residue L79 of SEQ ID NO: 1 can be altered to confer altered activin-GDF11/GDF8 binding properties. For example, an L79P substitution reduces GDF11 binding to a greater extent than activin binding. In contrast, replacement of L79 with an acidic amino acid [an aspartic acid or glutamic acid; an L79D or an L79E substitution] greatly reduces activin A binding affinity while retaining GDF11 binding affinity. In exemplary embodiments, the methods described herein utilize a polypeptide which is a variant ActRIIB polypeptide comprising an acidic amino acid (e.g., D or E) at the position corresponding to position 79 of SEQ ID NO: 1, optionally in combination with one or more additional amino acid substitutions, additions, or deletions.

In some embodiments, the present disclosure contemplates making functional variants by modifying the structure of an ActRII polypeptide for such purposes as enhancing therapeutic efficacy or stability (e.g., shelf-life and resistance to proteolytic degradation in vivo). Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide or to a reference variant polypeptide, or to bind to one or more TGF-beta ligands including, for example, activin A, activin B, activin AB, activin AC, BMP6, BMP7, BMP9, BMP10, GDF3, GDF8, and GDF11.

In certain embodiments, the present disclosure contemplates specific mutations of an ActRII polypeptide so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, polypeptides of the present disclosure for use in humans may be expressed in a mammalian cell line that provides proper glycosylation, such as HEK293 or CHO cell lines, although other mammalian expression cell lines are expected to be useful as well.

The present disclosure further contemplates a method of generating mutants, particularly sets of combinatorial mutants of an ActRII polypeptide as well as truncation mutants. Pools of combinatorial mutants are especially useful for identifying functionally active (e.g., ligand binding) ActRII sequences. The purpose of screening such combinatorial libraries may be to generate, for example, polypeptides variants which have altered properties, such as altered pharmacokinetic or altered ligand binding. A variety of screening assays are provided below, and such assays may be used to evaluate variants. For example, ActRII variants may be screened for ability to bind to one or more TGF-beta ligands (e.g., activin A, activin B, activin AB, activin AC, BMP6, BMP7, BMP9, BMP10, GDF3, GDF8, and GDF11), to prevent binding of a TGF-beta ligand to an ActRII polypeptide and/or to interfere with signaling caused by a TGF-beta ligand.

The activity of ActRII polypeptides may also be tested in a cell-based or in vivo assay. For example, the effect of an ActRII polypeptide on the expression of genes involved in PcPH pathogenesis (e.g., WHO Group 2 and/or Group 5 PH) may be assessed. This may, as needed, be performed in the presence of one or more recombinant TGF-beta ligand proteins (e.g., activin A, activin B, activin AB, activin AC, BMP6, BMP7, BMP9, BMP10, GDF3, GDF8, and GDF11), and cells may be transfected so as to produce an ActRII polypeptide and optionally, aTGF-beta family ligand. Likewise, an ActRII polypeptide may be administered to a mouse or other animal and effects on PcPH pathogenesis (e.g., WHO Group 2 and/or Group 5 PH) may be assessed using art-recognized methods. Similarly, the activity of an ActRII polypeptide or variant thereof may be tested in blood cell precursor cells for any effect on growth of these cells, for example, by the assays as described herein and those of common knowledge in the art. A SMAD-responsive reporter gene may be used in such cell lines to monitor effects on downstream signaling.

Combinatorial-derived variants can be generated which have increased selectivity or generally increased potency relative to a reference ActRII polypeptide. Such variants, when expressed from recombinant DNA constructs, can be used in gene therapy protocols. Likewise, mutagenesis can give rise to variants which have intracellular half-lives dramatically different than the corresponding unmodified ActRII polypeptide. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system, can allow tighter control of recombinant polypeptide complex levels within the cell. In an Fc fusion protein, mutations may be made in the linker (if any) and/or the Fc portion to alter the half-life of the ActRII polypeptide.

A combinatorial library may be produced by way of a degenerate library of genes encoding a library of polypeptides which each include at least a portion of potential ActRII polypeptide sequences. For instance, a mixture of synthetic oligonucleotides can be enzymatically ligated into gene sequences such that the degenerate set of potential ActRII encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art [Narang, S A (1983) Tetrahedron 39:3; Itakura et al. (1981) Recombinant DNA, Proc. 3rd Cleveland Sympos. Macromolecules, ed. AG Walton, Amsterdam: Elsevier pp 273-289; Itakura et al. (1984) Annu. Rev. Biochem. 53:323; Itakura et al. (1984) Science 198:1056; and Ike et al. (1983) Nucleic Acid Res. 11:477]. Such techniques have been employed in the directed evolution of other proteins [Scott et al., (1990) Science 249:386-390; Roberts et al. (1992) PNAS USA 89:2429-2433; Devlin et al. (1990) Science 249: 404-406; Cwirla et al., (1990) PNAS USA 87: 6378-6382; as well as U.S. Pat. Nos. 5,223,409, 5,198,346, and 5,096,815].

Alternatively, other forms of mutagenesis can be utilized to generate a combinatorial library. For example, ActRII polypeptides of the disclosure can be generated and isolated from a library by screening using, for example, alanine scanning mutagenesis [Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [Gustin et al. (1993) Virology 193:653-660; and Brown et al. (1992) Mol. Cell Biol. 12:2644-2652; McKnight et al. (1982) Science 232:316], by saturation mutagenesis [Meyers et al., (1986) Science 232:613]; by PCR mutagenesis [Leung et al. (1989) Method Cell Mol Biol 1:11-19]; or by random mutagenesis, including chemical mutagenesis [Miller et al. (1992) A Short Course in Bacterial Genetics, CSHL Press, Cold Spring Harbor, NY; and Greener et al. (1994) Strategies in Mol Biol 7:32-34]. Linker scanning mutagenesis, particularly in a combinatorial setting, is an attractive method for identifying truncated (bioactive) forms of ActRII polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of ActRII polypeptides. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include TGF-beta ligand (e.g., activin A, activin B, activin AB, activin AC, BMP6, BMP7, BMP9, BMP10, GDF3, GDF8, and GDF11) binding assays and/or ligand-mediated cell signaling assays.

As will be recognized by one of skill in the art, most of the described mutations, variants or modifications described herein may be made at the nucleic acid level or, in some cases, by post-translational modification or chemical synthesis. Such techniques are well known in the art and some of which are described herein. In part, the present disclosure identifies functionally active portions (fragments) and variants of ActRII polypeptides that can be used as guidance for generating and using other variant ActRII polypeptides within the scope described herein.

In certain embodiments, functionally active fragments of ActRII polypeptides of the present disclosure can be obtained by screening polypeptides recombinantly produced from the corresponding fragment of the nucleic acid encoding an ActRII polypeptide. In addition, fragments can be chemically synthesized using techniques known in the art such as conventional Merrifield solid phase f-Moc or t-Boc chemistry. The fragments can be produced (recombinantly or by chemical synthesis) and tested to identify those peptidyl fragments that can function as antagonists (inhibitors) of ActRII receptors and/or one or more TGF-beta ligands (e.g., activin A, activin B, activin AB, activin AC, BMP6, BMP7, BMP9, BMP10, GDF3, GDF8, and GDF11).

In certain embodiments, ActRII polypeptides of the present disclosure may further comprise post-translational modifications in addition to any that are naturally present in the ActRII polypeptide. Such modifications include, but are not limited to, acetylation, carboxylation, glycosylation, phosphorylation, lipidation, and acylation. As a result, the ActRII polypeptide may contain non-amino acid elements, such as polyethylene glycols, lipids, polysaccharide or monosaccharide, and phosphates. Effects of such non-amino acid elements on the functionality of a ligand trap polypeptide may be tested as described herein for other ActRII variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, WI38, NIH-3T3 or HEK293) have specific cellular machinery and characteristic mechanisms for such post-translational activities and may be chosen to ensure the correct modification and processing of the ActRII polypeptides.

In certain aspects, ActRII polypeptides of the present disclosure include fusion proteins having at least a portion (domain) of an ActRII polypeptide and one or more heterologous portions (domains). Well-known examples of such fusion domains include, but are not limited to, polyhistidine, Glu-Glu, glutathione S-transferase (GST), thioredoxin, protein A, protein G, an immunoglobulin heavy-chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) (SEQ ID NO: 137) fusion partners. As another example, a fusion domain may be selected so as to facilitate detection of the ActRII polypeptide. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagglutinin (HA), and c-myc tags. In some cases, the fusion domains have a protease cleavage site, such as for Factor Xa or thrombin, which allows the relevant protease to partially digest the fusion proteins and thereby liberate the recombinant proteins therefrom. The liberated proteins can then be isolated from the fusion domain by subsequent chromatographic separation. Other types of fusion domains that may be selected include multimerizing (e.g., dimerizing, tetramerizing) domains and functional domains (that confer an additional biological function) including, for example constant domains from immunoglobulins (e.g., Fc domains).

In certain aspects, ActRII polypeptides of the present disclosure contain one or more modifications that are capable of “stabilizing” the polypeptides. By “stabilizing” is meant anything that increases the in vitro half-life, serum half-life, regardless of whether this is because of decreased destruction, decreased clearance by the kidney, or other pharmacokinetic effect of the agent. For example, such modifications enhance the shelf-life of the polypeptides, enhance circulatory half-life of the polypeptides, and/or reduce proteolytic degradation of the polypeptides. Such stabilizing modifications include, but are not limited to, fusion proteins (including, for example, fusion proteins comprising an ActRII polypeptide domain and a stabilizer domain), modifications of a glycosylation site (including, for example, addition of a glycosylation site to a polypeptide of the disclosure), and modifications of carbohydrate moiety (including, for example, removal of carbohydrate moieties from a polypeptide of the disclosure). As used herein, the term “stabilizer domain” not only refers to a fusion domain (e.g., an immunoglobulin Fc domain) as in the case of fusion proteins, but also includes nonproteinaceous modifications such as a carbohydrate moiety, or nonproteinaceous moiety, such as polyethylene glycol. In certain preferred embodiments, an ActRII polypeptide is fused with a heterologous domain that stabilizes the polypeptide (a “stabilizer” domain), preferably a heterologous domain that increases stability of the polypeptide in vivo. Fusions with a constant domain of an immunoglobulin (e.g., a Fc domain) are known to confer desirable pharmacokinetic properties on a wide range of proteins. Likewise, fusions to human serum albumin can confer desirable properties.

An example of a native amino acid sequence that may be used for the Fc portion of human IgG1 (G1Fc) is shown below (SEQ ID NO: 14). Dotted underline indicates the hinge region, and solid underline indicates positions with naturally occurring variants. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 14. Naturally occurring variants in G1Fc would include E134D and M136L according to the numbering system used in SEQ ID NO: 14 (see Uniprot P01857).

(SEQ ID NO: 14)

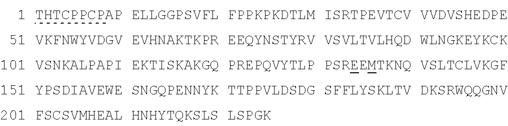

Optionally, the IgG1 Fc domain has one or more mutations at residues such as Asp-265, lysine 322, and Asn-434. In certain cases, the mutant IgG1 Fc domain having one or more of these mutations (e.g., Asp-265 mutation) has reduced ability of binding to the Fcγ receptor relative to a wild-type Fc domain. In other cases, the mutant Fc domain having one or more of these mutations (e.g., Asn-434 mutation) has increased ability of binding to the MHC class I-related Fc-receptor (FcRN) relative to a wild-type IgG1 Fc domain. In certain cases, the mutant IgG1 Fc domain lacks the N-terminal lysine (K).

In some embodiments, the sequence that may be used for the Fc portion of human IgG1 (G1Fc) is shown below:

(SEQ ID NO: 233)
THTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYK
CKVSNKALPVPIEKTISKAKGQPREPQVYTLPPSREEMTKNQVSLTCLV
KGFYPSDIAVEWESNGQPENNYKTTPPVLDSDGPFFLYSKLTVDKSRWQ
QGNVFSCSVMHEALHNHYTQKSLSLSPGK

In some embodiments, an Fc domain is from an IgG1 antibody and includes amino acid substitutions L12A, L13A, and G15A, relative to the sequence of SEQ ID NO: 233. In some embodiments, an Fc domain is from an IgG1 antibody and includes amino acid substitutions D43A, K1ODA, and N212A, relative to the sequence of SEQ ID NO: 233. In certain cases, the mutant IgG1 Fc domain lacks the N-terminal lysine (K). In some embodiments, a polypeptide described herein (e.g., an ActRII polypeptide)) may be fused to the N- or C-terminus of an Fc domain monomer (e.g., SEQ ID NO: 233) through conventional genetic or chemical means, e.g., chemical conjugation. If desired, a linker (e.g., a spacer) can be inserted between the polypeptide and the Fc domain monomer. In some embodiments, the Fc domain monomer can be fused to the N- or C-terminus (e.g., C-terminus) of the polypeptide.

In some embodiments, an Fc domain includes one or more of the following amino acid substitutions: T366W, T366Y, T394W, F405W, Y349T, Y349E, Y349V, L351T, L351H, L351N, L352K, P353S, S354D, D356K, D356R, D356S, E357K, E357R, E357Q, S364A, T366E, L368T, L368Y, L368E, K370E, K370D, K370Q, K392E, K392D, T394N, P395N, P396T, V397T, V397Q, L398T, D399K, D399R, D399N, F405T, F405H, F405R, Y407T, Y407H, Y407I, K409E, K409D, K409T, and K4091, relative to the sequence of human IgG1. In some embodiments, an Fc domain includes the amino acid substitution T366W, relative to the sequence of human IgG1. The sequence of a wild-type Fc domain is shown in SEQ ID NO: 284.

An example of a native amino acid sequence that may be used for the Fc portion of human IgG2 (G2Fc) is shown below (SEQ ID NO: 15). Dotted underline indicates the hinge region and double underline indicates positions where there are data base conflicts in the sequence (according to UniProt P01859). In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 15. In certain cases, the IgG2 Fc domain lacks the N-terminal lysine (K).

(SEQ ID NO: 15)

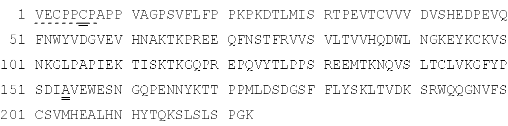

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 16) contains a short hinge region consisting of a single 15-residue segment, whereas the second G3Fc sequence (SEQ ID NO: 17) contains a full-length hinge region. In each case, dotted underline indicates the hinge region, and solid underline indicates positions with naturally occurring variants according to UniProt P01859. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NOs: 16 and 17. In certain cases, the IgG3 Fc domain lacks the N-terminal lysine (K).

(SEQ ID NO: 16)

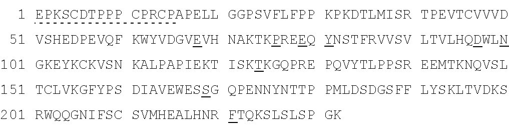

(SEQ ID NO: 17)

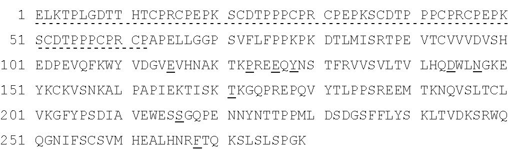

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 16, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 18). Dotted underline indicates the hinge region. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 18. In certain cases, the IgG4 Fc domain lacks the N-terminal lysine (K).

(SEQ IDNO: 18)

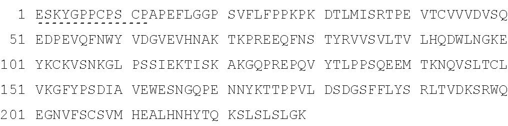

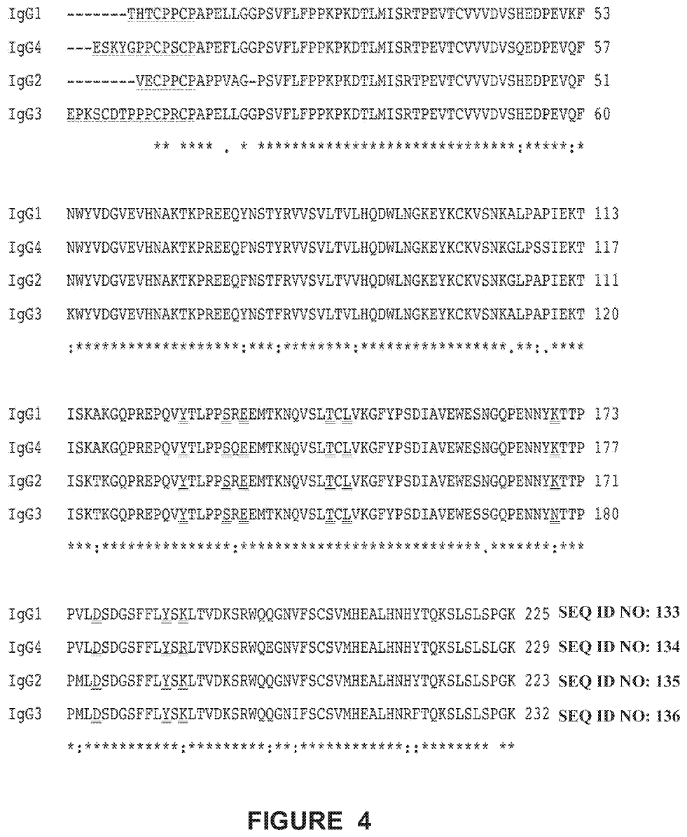

A variety of engineered mutations in the Fc domain are presented herein with respect to the G1Fc sequence (SEQ ID NO: 14), and analogous mutations in G2Fc, G3Fc, and G4Fc can be derived from their alignment with G1Fc in FIG. 4. Due to unequal hinge lengths, analogous Fc positions based on isotype alignment (FIG. 4) possess different amino acid numbers in SEQ ID NOs: 14, 15, 16, 17, and 18. It can also be appreciated that a given amino acid position in an immunoglobulin sequence consisting of hinge, C_{H}2, and C_{H}3 regions (e.g., SEQ ID NOs: 14, 15, 16, 17, and 18) will be identified by a different number than the same position when numbering encompasses the entire IgG1 heavy-chain constant domain (consisting of the C_{H}1, hinge, C_{H}2, and C_{H}3 regions) as in the Uniprot database. For example, correspondence between selected C_{H}3 positions in a human G1Fc sequence (SEQ ID NO: 14), the human IgG1 heavy chain constant domain (Uniprot P01857), and the human IgG1 heavy chain is as follows.

Correspondence of C_{H}3 Positions in Different Numbering Systems

| G1Fc | IgG1 heavy chain | |
|---|---|---|
| (Numbering begins | constant domain | IgG1 heavy chain |
| at first threonine | (Numbering begins at | (EU numbering scheme |
| in hinge region) | C_{H}1) | of Kabat et al., 1991*) |
| Y127 | Y232 | Y349 |
| S132 | S237 | S354 |
| E134 | E239 | E356 |
| T144 | T249 | T366 |
| L146 | L251 | L368 |
| K170 | K275 | K392 |
| D177 | D282 | D399 |
| Y185 | Y290 | Y407 |
| K187 | K292 | K409 |

*Kabat et al. (eds) 1991; pp. 688-696 in Sequences of Proteins of Immunological Interest, 5^{th} ed., Vol. 1, NIH, Bethesda, MD.

The application further provides Fc fusion proteins with engineered or variant Fc regions. Such Fc fusion proteins may be useful, for example, in modulating effector functions, such as, antigen-dependent cytotoxicity (ADCC) and complement-dependent cytotoxicity (CDC). Additionally, the modifications may improve the stability of the Fc fusion proteins. Amino acid sequence variants of the Fc fusion proteins are prepared by introducing appropriate nucleotide changes into the DNA, or by peptide synthesis. Such variants include, for example, deletions from, and/or insertions into and/or substitutions of, residues within the amino acid sequences of the antibodies and Fc fusion proteins disclosed herein. Any combination of deletion, insertion, and substitution is made to arrive at the final construct, provided that the final construct possesses the desired characteristics. The amino acid changes also may alter post-translational processes of the Fc fusion proteins, such as changing the number or position of glycosylation sites. In some embodiments, Fc polypeptide domains of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a polypeptide selected from the group consisting of SEQ ID NOs: 14, 15, 16, 17, 18, 133, 134, 135, 136, 233, and 284.

In some embodiments, a polypeptide disclosed herein (e.g., an ActRIIA variant polypeptide or an ActRIIB variant polypeptide) may further include a moiety (e.g., Fc domain monomer, a wild-type Fc domain, an Fc domain with amino acid substitutions (e.g., one or more substitutions that reduce dimerization), an albumin-binding peptide, a fibronectin domain, or a human serum albumin), which may be fused to the N- or C-terminus (e.g., C-terminus) of the polypeptide by way of a linker or other covalent bonds. A polypeptide (e.g., an ActRIIA variant polypeptide or an ActRIIB variant polypeptide) fused to an Fc domain monomer may form a dimer (e.g., homodimer or heterodimer) through the interaction between two Fc domain monomers, which combine to form an Fc domain in the dimer.

Fc fusion proteins with reduced effector function may be produced by introducing changes in the amino acid sequence, including, but are not limited to, the Ala-Ala mutation described by Bluestone et al. (see WO 94/28027 and WO 98/47531; also see Xu et al. 2000 Cell Immunol 200; 16-26) and the P329G/L234A/L235A (P329G LALA) mutation described by Schlothauer et al. (see Schlothauer T., et al. Protein Eng Des Sel. 2016 October; 29(10):457-466). Thus in certain embodiments, Fc fusion proteins of the disclosure with mutations within the constant region including the Ala-Ala mutation or the P329G LALA mutation may be used to reduce or abolish effector function. According to these embodiments, Fc fusion proteins may comprise a mutation to an alanine at position 234 or a mutation to an alanine at position 235, or a combination thereof. In one embodiment, the Fc fusion protein comprises an IgG4 framework, wherein the Ala-Ala mutation would describe a mutation(s) from phenylalanine to alanine at position 234 and/or a mutation from leucine to alanine at position 235. In some embodiments, Fc fusion proteins may further comprise mutation from proline to glycine at position 329. In another embodiment, the Fc fusion protein comprises an IgG1 framework, wherein the Ala-Ala mutation would describe a mutation(s) from leucine to alanine at position 234 and/or a mutation from leucine to alanine at position 235. In some embodiments, the Fc fusion protein comprising an IgG1 framework further comprises a mutation from proline to glycine at position 329. The Fc fusion protein may alternatively or additionally carry other mutations, including the point mutation K322A in the CH2 domain (Hezareh et al. 2001 J Virol. 75: 12161-8).

In some embodiments, the Fc fusion protein may be modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO99/51642, Duncan & Winter Nature 322: 738-40 (1988); U.S. Pat. Nos. 5,648,260; 5,624,821; and WO94/29351.

In some embodiments, a polypeptide described herein may include an extracellular ActRIIA variant fused to a serum protein-binding peptide. Binding to serum protein peptides can improve the pharmacokinetics of protein pharmaceuticals.

As one example, albumin-binding peptides that can be used in the methods and compositions described here are generally known in the art. In one embodiment, the albumin binding peptide includes the sequence DICLPRWGCLW (SEQ ID NO: 285).

In some embodiments, albumin-binding peptides may be joined to the N- or C-terminus (e.g., C-terminus) of a polypeptide described herein (e.g., an ActRIIA or ActRIIB polypeptide)) to increase the serum half-life of the extracellular ActRIIA variant. In some embodiments, an albumin-binding peptide is joined, either directly or through a linker, to the N- or C-terminus of the polypeptide.

In some embodiments, a polypeptide described herein (e.g., an ActRIIA or ActRIIB polypeptide) may be fused to the N- or C-terminus of albumin-binding peptide (e.g., SEQ ID NO: 285) through conventional genetic or chemical means, e.g., chemical conjugation. If desired, a linker (e.g., a spacer) can be inserted between the polypeptide and the albumin-binding peptide. Without being bound to a theory, it is expected that inclusion of an albumin-binding peptide in an extracellular ActRIIA or ActRIIB variant described herein may lead to prolonged retention of the therapeutic protein through its binding to serum albumin.

In some embodiments, a polypeptide described herein may include a polypeptide (e.g. an ActRIIA or ActRIIB polypeptide) fused to fibronectin domains. Binding to fibronectin domains can improve the pharmacokinetics of protein pharmaceuticals.

A fibronectin domain is a high molecular weight glycoprotein of the extracellular matrix, or a fragment thereof, that binds to, e.g., membrane-spanning receptor proteins such as integrins and extracellular matrix components such as collagens and fibrins. In some embodiments, a fibronectin domain is joined to the N- or C-terminus (e.g., C-terminus) of a polypeptide described herein (e.g., an ActRIIA or ActRIIB polypeptide) to increase the serum half-life of the polypeptide. A fibronectin domain can be joined, either directly or through a linker, to the N- or C-terminus of a polypeptide.

As one example, fibronectin domains that can be used in the methods and compositions described here are generally known in the art. In one embodiment, the fibronectin domain is a fibronectin type III domain (SEQ ID NO: 286) having amino acids 610-702 of the sequence of UniProt ID NO: P02751. In another embodiment, the fibronectin domain is an adnectin protein.

In some embodiments, a polypeptide described herein (e.g., an ActRIIA or ActRIIB polypeptide) may be fused to the N- or C-terminus of a fibronectin domain (e.g., SEQ ID NO: 286) through conventional genetic or chemical means, e.g., chemical conjugation. If desired, a linker (e.g., a spacer) can be inserted between the polypeptide and the fibronectin domain. Without being bound to a theory, it is expected that inclusion of a fibronectin domain in a polypeptide described herein may lead to prolonged retention of the therapeutic protein through its binding to integrins and extracellular matrix components such as collagens and fibrins.

In some embodiments, a polypeptide described herein may include an ActRIIA or ActRIIB polypeptide fused to serum albumin. Binding to serum albumins can improve the pharmacokinetics of protein pharmaceuticals.

Serum albumin is a globular protein that is the most abundant blood protein in mammals. Serum albumin is produced in the liver and constitutes about half of the blood serum proteins. It is monomeric and soluble in the blood. Some of the most crucial functions of serum albumin include transporting hormones, fatty acids, and other proteins in the body, buffering pH, and maintaining osmotic pressure needed for proper distribution of bodily fluids between blood vessels and body tissues. In some embodiments, serum albumin is human serum albumin. In some embodiments, a human serum albumin is joined to the N- or C-terminus (e.g., C-terminus) of a polypeptide described herein (e.g., an ActRIIA or ActRIIB polypeptide) to increase the serum half-life of the polypeptide. In some embodiments, the human serum albumin can be joined, either directly or through a linker, to the N- or C-terminus of a polypeptide disclosed herein.

As one example, serum albumins that can be used in the methods and compositions described herein are generally known in the art. In one embodiment, the serum albumin includes the sequence of UniProt ID NO: P02768 (SEQ ID NO: 287).

In some embodiments, a polypeptide described herein (e.g., an ActRIIA or ActRIIB polypeptide) may be fused to the N- or C-terminus of a human serum albumin (e.g., SEQ ID NO: 287) through conventional genetic or chemical means, e.g., chemical conjugation. If desired, a linker (e.g., a spacer) can be inserted between the polypeptide and the human serum albumin. Without being bound to a theory, it is expected that inclusion of a human serum albumin in an ActRIIA or ActRIIB polypeptide described herein may lead to prolonged retention of the therapeutic protein.

It is understood that different elements of the fusion proteins (e.g., immunoglobulin Fc fusion proteins) may be arranged in any manner that is consistent with desired functionality. For example, an ActRII polypeptide domain may be placed C-terminal to a heterologous domain, or alternatively, a heterologous domain may be placed C-terminal to an ActRII polypeptide domain. The ActRII polypeptide domain and the heterologous domain need not be adjacent in a fusion protein, and additional domains or amino acid sequences may be included C- or N-terminal to either domain or between the domains.

For example, an ActRII receptor fusion protein may comprise an amino acid sequence as set forth in the formula A-B-C. The B portion corresponds to an ActRII polypeptide domain. The A and C portions may be independently zero, one, or more than one amino acid, and both the A and C portions when present are heterologous to B. The A and/or C portions may be attached to the B portion via a linker sequence. In certain embodiments, an ActRII fusion protein comprises an amino acid sequence as set forth in the formula A-B-C, wherein A is a leader (signal) sequence, B consists of an ActRII polypeptide domain, and C is a polypeptide portion that enhances one or more of in vivo stability, in vivo half-life, uptake/administration, tissue localization or distribution, formation of protein complexes, and/or purification. In certain embodiments, an ActRII fusion protein comprises an amino acid sequence as set forth in the formula A-B-C, wherein A is a TPA leader sequence, B consists of an ActRII receptor polypeptide domain, and C is an immunoglobulin Fc domain. Preferred fusion proteins comprise the amino acid sequence set forth in any one of SEQ ID NOs: 36, 39, 40, 42, 45, 46, 48, 69, 74, 77, 78, 139, 140, 141, 142, 143, 144, 318, or 331.

In preferred embodiments, ActRII polypeptides to be used in accordance with the methods described herein are isolated polypeptides. As used herein, an isolated protein or polypeptide is one which has been separated from a component of its natural environment. In some embodiments, a polypeptide of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of purity are well known in the art [see, e.g., Flatman et al., (2007) J. Chromatogr. B 848:79-87]. In some embodiments, ActRII polypeptides to be used in accordance with the methods described herein are recombinant polypeptides.

ActRII polypeptides of the disclosure can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the polypeptides of the disclosure, including fragments or variants thereof, may be recombinantly produced using various expression systems [e.g., E. coli, Chinese Hamster Ovary (CHO) cells, COS cells, baculovirus] as is well known in the art. In a further embodiment, the modified or unmodified polypeptides of the disclosure may be produced by digestion of recombinantly produced full-length ActRII polypeptides by using, for example, a protease, e.g., trypsin, thermolysin, chymotrypsin, pepsin, or paired basic amino acid converting enzyme (PACE). Computer analysis (using commercially available software, e.g., MacVector, Omega, PCGene, Molecular Simulation, Inc.) can be used to identify proteolytic cleavage sites. Alternatively, such polypeptides may be produced from recombinantly generated full-length ActRII polypeptides using chemical cleavage (e.g., cyanogen bromide, hydroxylamine, etc.).

### 3. Linkers

The disclosure provides for ActRII polypeptides and variants thereof (e.g., ActRIIA polypeptides or ActRIIB polypeptides), and in these embodiments, the polypeptide portion (e.g. ActRIIA polypeptide) is connected to the heterologous portion (e.g., Fc portion) by means of a linker. In some embodiments, the linkers are glycine and serine rich linkers. In some embodiments, the linker may be rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 19), GGGG (SEQ ID NO: 20), TGGGG (SEQ ID NO: 21), SGGGG (SEQ ID NO: 22), TGGG (SEQ ID NO: 23), SGGG (SEQ ID NO: 24), or GGGGS (SEQ ID NO: 25) singlets, or repeats. Other near neutral amino acids, such as, but not limited to, Thr, Asn, Pro and Ala, may also be used in the linker sequence. In some embodiments, the linker comprises various permutations of amino acid sequences containing Gly and Ser. In some embodiments, the linker is greater than 10 amino acids in length. In further embodiments, the linkers have a length of at least 12, 15, 20, 21, 25, 30, 35, 40, 45 or 50 amino acids. In some embodiments, the linker is less than 40, 35, 30, 25, 22 or 20 amino acids. In some embodiments, the linker is 10-50, 10-40, 10-30, 10-25, 10-21, 10-15, 10, 15-25, 17-22, 20, or 21 amino acids in length. In preferred embodiments, the linker comprises the amino acid sequence GlyGlyGlyGlySer (GGGGS) (SEQ ID NO: 25), or repetitions thereof (GGGGS)n, where n≥2. In particular embodiments n≥3, or n=3-10. In some embodiments, n≥4, or n=4-10. In some embodiments, n is not greater than 4 in a (GGGGS)n linker. In some embodiments, n=4-10, 4-9, 4-8, 4-7, 4-6, 4-5, 5-8, 5-7, or 5-6. In some embodiments, n=3, 4, 5, 6, or 7. In particular embodiments, n=4. In some embodiments, a linker comprising a (GGGGS). sequence also comprises an N-terminal threonine. In some embodiments, the linker is any one of the following:

(SEQ ID NO: 85)
GGGGSGGGGS
(SEQ ID NO: 86)
TGGGGSGGGGS
(SEQ ID NO: 87)
TGGGGSGGGGSGGGGS
(SEQ ID NO: 88)
TGGGGSGGGGSGGGGSGGGGS
(SEQ ID NO: 89)
TGGGGSGGGGSGGGGSGGGGSGGGGS
(SEQ ID NO: 90)
TGGGGSGGGGSGGGGSGGGGSGGGGSGGGGS
or
(SEQ ID NO: 91)
TGGGGSGGGGSGGGGSGGGGSGGGGSGGGGSGGGGS.

In some embodiments, the linker comprises the amino acid sequence of TGGGPKSCDK (SEQ ID NO: 92). In some embodiments, the linker is any one of SEQ ID NOs: 85-92 lacking the N-terminal threonine. In some embodiments, the linker does not comprise the amino acid sequence of SEQ ID NO: 90 or 91.

In some embodiments, a polypeptide described (e.g., ActRIIA polypeptides or ActRIIB polypeptides) herein may include a polypeptide fused to a moiety by way of a linker. In some embodiments, the moiety increases stability of the polypeptide. In some embodiments, the moiety is selected from the group consisting of an Fc domain monomer, a wild-type Fc domain, an Fc domain with amino acid substitutions (e.g., one or more substitutions that reduce dimerization), an albumin-binding peptide, a fibronectin domain, or a human serum albumin. In some embodiments, a linker between a moiety (e.g., an Fc domain monomer (e.g., the sequence of SEQ ID NO: 233), a wild-type Fc domain (e.g., SEQ ID NO: 284), an Fc domain with amino acid substitutions (e.g., one or more substitutions that reduce dimerization), an albumin-binding peptide (e.g., SEQ ID NO: 285), a fibronectin domain (e.g., SEQ ID NO: 286), or a human serum albumin (e.g., SEQ ID NO: 287)) and a polypeptide (e.g., ActRIIA polypeptides or ActRIIB polypeptides), can be an amino acid linker including 1-200 amino acids. Suitable peptide linkers are known in the art, and include, for example, peptide linkers containing flexible amino acid residues such as glycine, alanine, and serine. In some embodiments, a linker can contain motifs, e.g., multiple or repeating motifs, of GA, GS, GG, GGA, GGS, GGG, GGGA (SEQ ID NO: 234), GGGS (SEQ ID NO: 235), GGGG (SEQ ID NO: 20), GGGGA (SEQ ID NO: 236), GGGGS (SEQ ID NO: 25), GGGGG (SEQ ID NO: 237), GGAG (SEQ ID NO: 238), GGSG (SEQ ID NO: 239), AGGG (SEQ ID NO: 240), or SGGG (SEQ ID NO: 24). In some embodiments, a linker can contain 2 to 12 amino acids including motifs of GA or GS, e.g., GA, GS, GAGA (SEQ ID NO: 241), GSGS (SEQ ID NO: 242), GAGAGA (SEQ ID NO: 243), GSGSGS (SEQ ID NO: 244), GAGAGAGA (SEQ ID NO: 245), GSGSGSGS (SEQ ID NO: 246), GAGAGAGAGA (SEQ ID NO: 247), GSGSGSGSGS (SEQ ID NO: 248), GAGAGAGAGAGA (SEQ ID NO: 249), and GSGSGSGSGSGS (SEQ ID NO: 250). In some embodiments, a linker can contain 3 to 12 amino acids including motifs of GGA or GGS, e.g., GGA, GGS, GGAGGA (SEQ ID NO: 251), GGSGGS (SEQ ID NO: 252), GGAGGAGGA (SEQ ID NO: 253), GGSGGSGGS (SEQ ID NO: 254), GGAGGAGGAGGA (SEQ ID NO: 255), and GGSGGSGGSGGS (SEQ ID NO: 256). In some embodiments, a linker can contain 4 to 12 amino acids including motifs of GGAG (SEQ ID NO: 238), GGSG (SEQ ID NO: 239), GGAGGGAG (SEQ ID NO: 257), GGSGGGSG (SEQ ID NO: 258), GGAGGGAGGGAG (SEQ ID NO: 259), and GGSGGGSGGGSG (SEQ ID NO: 260). In some embodiments, a linker can contain motifs of GGGGA (SEQ ID NO: 236) or GGGGS (SEQ ID NO: 25), e.g., GGGGAGGGGAGGGGA (SEQ ID NO: 261) and GGGGSGGGGSGGGGS (SEQ ID NO: 262). In some embodiments, an amino acid linker between a moiety (e.g., an Fc domain monomer, a wild-type Fc domain, an Fc domain with amino acid substitutions (e.g., one or more substitutions that reduce dimerization), an albumin-binding peptide, a fibronectin domain, or a human serum albumin) and a polypeptide (e.g., ActRIIA polypeptides or ActRIIB polypeptides) may be GGG, GGGA (SEQ ID NO: 234), GGGG (SEQ ID NO: 20), GGGAG (SEQ ID NO: 263), GGGAGG (SEQ ID NO: 264), or GGGAGGG (SEQ ID NO: 265).

In some embodiments, a linker can also contain amino acids other than glycine, alanine, and serine, e.g., AAAL (SEQ ID NO: 266), AAAK (SEQ ID NO: 267), AAAR (SEQ ID NO: 268), EGKSSGSGSESKST (SEQ ID NO: 269), GSAGSAAGSGEF (SEQ ID NO: 270), AEAAAKEAAAKA (SEQ ID NO: 271), KESGSVSSEQLAQFRSLD (SEQ ID NO: 272), GENLYFQSGG (SEQ ID NO: 273), SACYCELS (SEQ ID NO: 274), RSIAT (SEQ ID NO: 275), RPACKIPNDLKQKVMNH (SEQ ID NO: 276), GGSAGGSGSGSSGGSSGASGTGTAGGTGSGSGTGSG (SEQ ID NO: 277), AAANSSIDLISVPVDSR (SEQ ID NO: 278), or GGSGGGSEGGGSEGGGSEGGGSEGGGSEGGGSGGGS (SEQ ID NO: 279). In some embodiments, a linker can contain motifs, e.g., multiple or repeating motifs, of EAAAK (SEQ ID NO: 280). In some embodiments, a linker can contain motifs, e.g., multiple or repeating motifs, of praline-rich sequences such as (XP)n, in which X may be any amino acid (e.g., A, K, or E) and n is from 1-5, and PAPAP (SEQ ID NO: 281).

The length of the peptide linker and the amino acids used can be adjusted depending on the two proteins involved and the degree of flexibility desired in the final protein fusion polypeptide. The length of the linker can be adjusted to ensure proper protein folding and avoid aggregate formation.

### 4. Nucleic Acids Encoding ActRII Polypeptides and Variants Thereof

In certain embodiments, the present disclosure provides isolated and/or recombinant nucleic acids encoding ActRII polypeptides (including fragments, functional variants, and fusion proteins thereof). For example, SEQ ID NO: 7 encodes a naturally occurring human ActRIIB precursor polypeptide (the R64 variant described above), while SEQ ID NO: 8 encodes the processed extracellular domain of ActRIIB (the R64 variant described above). The subject nucleic acids may be single-stranded or double-stranded. Such nucleic acids may be DNA or RNA molecules. These nucleic acids may be used, for example, in methods for making ActRII-based variant polypeptides as described herein.

As used herein, isolated nucleic acid(s) refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

In certain embodiments, nucleic acids encoding ActRII polypeptides of the disclosure are understood to include nucleic acids that are variants of any one of SEQ ID NOs: 7, 8, 12, 13, 37, 43, 49, 70, 71, 72, 73, 75, 76, 80, 81, 82, 83, 84, and 94. Variant nucleotide sequences include sequences that differ by one or more nucleotide substitutions, additions, or deletions including allelic variants, and therefore, will include coding sequence that differ from the nucleotide sequence designated in any one of SEQ ID NOs: 7, 8, 12, 13, 37, 43, 49, 70, 71, 72, 73, 75, 76, 80, 81, 82, 83, 84, and 94.

In certain embodiments, ActRII polypeptides of the disclosure are encoded by isolated and/or recombinant nucleic acid sequences that are at least 70%, 75%, 80%, 85%, 90%, 910%, 92%, 93%, 94% 95%, 96%, 97%, 98%, 99%, or 100% identical to any one of SEQ ID NOs: 7, 8, 12, 13, 37, 43, 49, 70, 71, 72, 73, 75, 76, 80, 81, 82, 83, 84, and 94. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 7. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 8. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 12. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 13. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 37. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 43. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 49. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 70. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 71. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 72. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 73. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 75. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 76. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 80. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 81. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 82. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 83. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 84. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 94. One of ordinary skill in the art will appreciate that nucleic acid sequences that are at least 70%, 75%, 80%, 85%, 90%, 91%, 92%, 93%, 94% 95%, 96%, 97%, 98%, 99%, or 100% identical to the sequences complementary to SEQ ID NOs: 7, 8, 12, 13, 37, 43, 49, 70, 71, 72, 73, 75, 76, 80, 81, 82, 83, and 84, and variants thereof, are also within the scope of the present disclosure. In further embodiments, the nucleic acid sequences of the disclosure can be isolated, recombinant, and/or fused with a heterologous nucleotide sequence, or in a DNA library.

In other embodiments, nucleic acids of the present disclosure also include nucleotide sequences that hybridize under highly stringent conditions to the nucleotide sequence designated in SEQ ID NOs: 7, 8, 12, 13, 37, 43, 49, 70, 71, 72, 73, 75, 76, 80, 81, 82, 83, and 84, complement sequences of SEQ ID NOs: 7, 8, 12, 13, 37, 43, 49, 70, 71, 72, 73, 75, 76, 80, 81, 82, 83, and 84, or fragments thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOs: 7, 8, 12, 13, 37, 43, 49, 70, 71, 72, 73, 75, 76, 80, 81, 82, 83, and 84 to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the present disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art and can be used in a variety of host cells. Typically, one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and can vary with the host cell used.

In certain aspects, the subject nucleic acid disclosed herein is provided in an expression vector comprising a nucleotide sequence encoding an ActRII polypeptide and operably linked to at least one regulatory sequence. Regulatory sequences are art-recognized and are selected to direct expression of the ActRII polypeptide. Accordingly, the term “regulatory sequence” includes promoters, enhancers, and other expression control elements.

Exemplary regulatory sequences are described in Goeddel; Gene Expression Technology: Methods in Enzymology, Academic Press, San Diego, CA (1990). For instance, any of a wide variety of expression control sequences that control the expression of a DNA sequence when operatively linked to it may be used in these vectors to express DNA sequences encoding an ActRII polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant ActRII polypeptide include plasmids and other vectors. For instance, suitable vectors include plasmids of the following types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, e.g., Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the 1-gal containing pBlueBac III).

In a preferred embodiment, a vector will be designed for production of the subject ActRII polypeptides in CHO cells, such as a Pcmv-Script vector (Stratagene, La Jolla, Calif.), pcDNA4 vectors (Invitrogen, Carlsbad, Calif.) and pCI-neo vectors (Promega, Madison, Wisc.). As will be apparent, the subject gene constructs can be used to cause expression of the subject ActRII polypeptides in cells propagated in culture, e.g., to produce proteins, including fusion proteins or variant proteins, for purification.

This disclosure also pertains to a host cell transfected with a recombinant gene including a coding sequence for one or more of the subject ActRII polypeptides. The host cell may be any prokaryotic or eukaryotic cell. For example, an ActRII polypeptide of the disclosure may be expressed in bacterial cells such as E. coli, insect cells (e.g., using a baculovirus expression system), yeast, or mammalian cells [e.g. a Chinese hamster ovary (CHO) cell line]. Other suitable host cells are known to those skilled in the art.

Accordingly, the present disclosure further pertains to methods of producing the subject ActRII polypeptides. For example, a host cell transfected with an expression vector encoding an ActRII polypeptide can be cultured under appropriate conditions to allow expression of the ActRII polypeptide to occur. The polypeptide may be secreted and isolated from a mixture of cells and medium containing the polypeptide. Alternatively, the ActRII polypeptide may be retained cytoplasmically or in a membrane fraction and the cells harvested, lysed and the protein isolated. A cell culture includes host cells, media and other byproducts. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the ActRII polypeptides, and affinity purification with an agent that binds to a domain fused to the ActRII polypeptide (e.g., a protein A column may be used to purify an ActRII-Fc fusion proteins). In some embodiments, the ActRII polypeptide is a fusion protein containing a domain which facilitates its purification.

In some embodiments, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. An ActRII polypeptide may be purified to a purity of >90%, >95%, >96%, >98%, or >99% as determined by size exclusion chromatography and >90%, >95%, >96%, >98%, or >99% as determined by SDS PAGE. The target level of purity should be one that is sufficient to achieve desirable results in mammalian systems, particularly non-human primates, rodents (mice), and humans.

In another embodiment, a fusion gene coding for a purification leader sequence, such as a poly-(His)/enterokinase cleavage site sequence at the N-terminus of the desired portion of the recombinant ActRII polypeptide, can allow purification of the expressed fusion protein by affinity chromatography using a Ni²⁺ metal resin. The purification leader sequence can then be subsequently removed by treatment with enterokinase to provide the purified ActRII polypeptide. See, e.g., Hochuli et al. (1987) J. Chromatography 411:177; and Janknecht et al. (1991) PNAS USA 88:8972.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence. See, e.g., Current Protocols in Molecular Biology, eds. Ausubel et al., John Wiley & Sons: 1992.

### 5. Screening Assays

In certain aspects, the present disclosure relates to the use of the subject ActRII antagonists (e.g., ActRII polypeptides) to identify compounds (agents) which may be used to treat, prevent, or reduce the progression rate and/or severity of post-capillary pulmonary hypertension (PcPH) (e.g., WHO Group 2 and/or Group 5 PH).

There are numerous approaches to screening for therapeutic agents for treating PcPH (e.g., WHO Group 2 and/or Group 5 PH) by targeting signaling (e.g., Smad signaling) of one or more ligands. In certain embodiments, high-throughput screening of compounds can be carried out to identify agents that perturb ligand-mediated effects on a selected cell line. In certain embodiments, the assay is carried out to screen and identify compounds that specifically inhibit or reduce binding of a TGF-beta ligand (e.g., activin A, activin B, activin AB, activin AC, BMP6, BMP7, BMP9, BMP10, GDF3, GDF8, and/or GDF11) to its binding partner, such as an a type II receptor (e.g., ActRIIA and/or ActRIIB). Alternatively, the assay can be used to identify compounds that enhance binding of a ligand to its binding partner such as a type II receptor. In a further embodiment, the compounds can be identified by their ability to interact with a type II receptor.

A variety of assay formats will suffice and, in light of the present disclosure, those not expressly described herein will nevertheless be comprehended by one of ordinary skill in the art. As described herein, the test compounds (agents) disclosed herein may be created by any combinatorial chemical method. Alternatively, the subject compounds may be naturally occurring biomolecules synthesized in vivo or in vitro. Compounds (agents) to be tested for their ability to act as modulators of tissue growth can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules. In certain embodiments, the test agent is a small organic molecule having a molecular weight of less than about 2,000 Daltons.

The test compounds of the disclosure can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S-transferase (GST), photoactivatable crosslinkers or any combinations thereof.

In many drug-screening programs which test libraries of compounds and natural extracts, high-throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between a TGF-beta ligand (e.g., activin A, activin B, activin AB, activin AC, BMP6, BMP7, BMP9, BMP10, GDF3, GDF8, and/or GDF11) to its binding partner, such as an a type II receptor (e.g., ActRIIA and/or ActRIIB).

Merely to illustrate, in an exemplary screening assay of the present disclosure, the compound of interest is contacted with an isolated and purified ActRIIB polypeptide which is ordinarily capable of binding to an ActRIIB ligand, as appropriate for the intention of the assay. To the mixture of the compound and ActRIIB polypeptide is then added to a composition containing an ActRIIB ligand (e.g., GDF11). Detection and quantification of ActRIIB/ActRIIB-ligand complexes provides a means for determining the compound's efficacy at inhibiting (or potentiating) complex formation between the ActRIIB polypeptide and its binding protein. The efficacy of the compound can be assessed by generating dose-response curves from data obtained using various concentrations of the test compound. Moreover, a control assay can also be performed to provide a baseline for comparison. For example, in a control assay, isolated and purified ActRIIB ligand is added to a composition containing the ActRIIB polypeptide, and the formation of ActRIIB/ActRIIB ligand complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Complex formation between a ligand and its binding protein may be detected by a variety of techniques. For instance, modulation of the formation of complexes can be quantitated using, for example, detectably labeled proteins such as radiolabeled (e.g., ³²P, ³⁵S, ¹⁴C or ³H), fluorescently labeled (e.g., FITC), or enzymatically labeled ActRIIB polypeptide and/or its binding protein, by immunoassay, or by chromatographic detection.

In certain embodiments, the present disclosure contemplates the use of fluorescence polarization assays and fluorescence resonance energy transfer (FRET) assays in measuring, either directly or indirectly, the degree of interaction between a ligand and its binding protein. Further, other modes of detection, such as those based on optical waveguides (see, e.g., PCT Publication WO 96/26432 and U.S. Pat. No. 5,677,196), surface plasmon resonance (SPR), surface charge sensors, and surface force sensors, are compatible with many embodiments of the disclosure.

Moreover, the present disclosure contemplates the use of an interaction trap assay, also known as the “two-hybrid assay,” for identifying agents that disrupt or potentiate interaction between a ligand and its binding partner. See, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present disclosure contemplates the use of reverse two-hybrid systems to identify compounds (e.g., peptides) that dissociate interactions between a ligand and its binding protein [see, e.g., Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368].

In certain embodiments, the subject compounds are identified by their ability to interact with a particular ligand. The interaction between the compound and the ligand may be covalent or non-covalent. For example, such interaction can be identified at the protein level using in vitro biochemical methods, including photo-crosslinking, radiolabeled ligand binding, and affinity chromatography [see, e.g., Jakoby W B et al. (1974) Methods in Enzymology 46:1]. In certain cases, the compounds may be screened in a mechanism-based assay, such as an assay to detect compounds which bind to a particular ligand. This may include a solid-phase or fluid-phase binding event. Alternatively, the gene encoding a ligand can be transfected with a reporter system (e.g., β-galactosidase, luciferase, or green fluorescent protein) into a cell and screened against the library preferably by high-throughput screening or with individual members of the library. Other mechanism-based binding assays may be used; for example, binding assays which detect changes in free energy. Binding assays can be performed with the target fixed to a well, bead or chip or captured by an immobilized antibody or resolved by capillary electrophoresis. The bound compounds may be detected usually using colorimetric endpoints or fluorescence or surface plasmon resonance.

### 6. Methods of Use

In part, the present disclosure relates to methods of treating post-capillary pulmonary hypertension (PcPH) (e.g., WHO Group 2 and/or Group 5 PH) comprising administering to a patient in need thereof an effective amount of any of, or any combination of, the ActRII antagonists as described herein. In some embodiments, the patient is administered any of the ActRIIA polypeptides or variants and/or fragments thereof disclosed herein. In some embodiments, the patient is administered any of the ActRIIB polypeptides or variants and/or fragments thereof disclosed herein. In some embodiments, the disclosure contemplates methods of treating one or more complications of PcPH (e.g., smooth muscle and/or endothelial cell proliferation in the pulmonary artery, angiogenesis in the pulmonary artery, dyspnea, chest pain, pulmonary vascular remodeling, right ventricular hypertrophy, left ventricular hypertrophy, left atrium dilation, and pulmonary fibrosis) comprising administering to a patient in need thereof an effective amount of a ActRII antagonist. In some embodiments, the disclosure contemplates methods of preventing one or more complications of PcPH comprising administering to a patient in need thereof an effective amount of an ActRII antagonist. In some embodiments, the disclosure contemplates methods of reducing the progression rate of PcPH comprising administering to a patient in need thereof an effective amount of an ActRII antagonist.

In some embodiments, the PcPH is combined post- and pre-capillary PH. In certain embodiments, the present disclosure provides methods of treating or preventing post-capillary pulmonary hypertension (PcPH) in an individual in need thereof through administering to the individual a therapeutically effective amount of an ActRII antagonist. These methods are particularly aimed at therapeutic and prophylactic treatments of animals, and more particularly, humans. The terms “subject,” an “individual,” or a “patient” are interchangeable throughout the specification and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

The terms “treatment”, “treating”, “alleviating” and the like are used herein to generally mean obtaining a desired pharmacologic and/or physiologic effect, and may also be used to refer to improving, alleviating, and/or decreasing the severity of one or more clinical complication of a condition being treated (e.g., WHO Group 2 and/or Group 5 PH). The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample.

In general, treatment or prevention of a disease or condition as described in the present disclosure (e.g., WHO Group 2 and/or Group 5 PH) is achieved by administering one or more ActRII antagonist of the present disclosure in an “effective amount”. An effective amount of an agent refers to an amount effective, at dosages and for periods of time necessary, to achieve the desired therapeutic or prophylactic result. A “therapeutically effective amount” of an agent of the present disclosure may vary according to factors such as the disease state, age, sex, and weight of the individual, and the ability of the agent to elicit a desired response in the individual. A “prophylactically effective amount” refers to an amount effective, at dosages and for periods of time necessary, to achieve the desired prophylactic result.

In certain aspects, the disclosure contemplates the use of an ActRII antagonist, in combination with one or more additional active agents or other supportive therapy for treating or preventing a disease or condition (e.g., WHO Group 2 and/or Group 5 PH). As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more ActRII antagonist of the present disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of the ActRII antagonist of the present disclosure with the additional active agent or therapy and/or the desired effect.

WHO Classification Outline A pulmonary hypertension condition treated by methods describe herein, can comprise any one or more of the conditions recognized according to the World Health Organization (WHO). See, e.g., Simonneau (2019) Eur Respiri J: 53:1801913.

TABLE 8
Clinical Classification of Pulmonary Hypertension

Group 1: Pulmonary arterial hypertension (PAH)
1.1 Idiopathic PAH
1.2 Heritable PAH
1.2.1 BMPR2
1.2.2 ALK-1, ENG, SMAD9, CAV1, KCNK3
1.2.3 Unknown
1.3 Drug and toxin induced PAH
1.4 Associated with:
1.4.1 Connective tissue disease
1.4.2 HIV infection
1.4.3 Portal hypertension
1.4.4 Congenital heart diseases
1.4.5 Schistosomiasis
1.5 PAH long-term responders to calcium channel blockers
1.6 PAH with overt features of venous/capillaries
(PVOD/PCH) involvement
1.7 Persistent PH of the newborn syndrome
Group 2: Pulmonary hypertension due to left heart disease
2.1 PH due to heart failure with preserved LVEF¹ (HFpEF)
2.2 PH due to heart failure with reduced LVEF (HFrEF)
2.3 Valvular heart disease
2.4 Congenital/acquired cardiovascular conditions leading
to post-capillary PH
Group 3: Pulmonary hypertension due to lung disease and/or hypoxia
3.1 Obstructive lung disease
3.2 Restrictive lung disease
3.3 Other lung disease with mixed restrictive/obstructive pattern
3.4 Hypoxia without lung disease
3.5 Developmental lung disorders
Group 4: Pulmonary hypertension due to pulmonary artery obstructions
4.1 Chronic thromboembolic PH
4.2 Other pulmonary artery obstructions
4.2.1 Sarcoma (high or intermediate grade) or angiosarcoma
4.2.2 Other malignant tumours
Renal carcinoma
Uterine carcinoma
Germ cell tumours of the testis
Other tumours
4.2.3 Non-malignant tumours
Uterine leiomyoma
4.2.4 Arteritis without connective tissue disease
4.2.5 Congenital pulmonary artery stenoses
4.2.6 Parasites
Hydatidosis
Group 5: Pulmonary hypertension with unclear and/or
multifactorial mechanisms.
5.1 Hematological disorders (e.g., Chronic hemolytic
anaemia and myeloproliferative disorders)
5.2 Systemic and metabolic disorders (e.g., Pulmonary
Langerhans cell histiocytosis, Gaucher disease, Glycogen
storage disease, Neurofibromatosis, and Sarcoidosis)
5.3 Others (e.g., Chronic renal failure with or without
haemodialysis and Fibrosing mediastinitis)
5.4 Complex congenital heart disease
¹Left ventricular ejection fraction

The clinical purpose of the classification of PH is to categorize clinical conditions associated with PH into five groups according to their pathophysiological mechanisms, clinical presentation, hemodynamic characteristics, and treatment strategy. This clinical classification may be updated when new data are available on the above features or when additional clinical entities are considered.

Pulmonary hypertension (PH) has been previously classified as primary or secondary PH. The term primary pulmonary hypertension has now been replaced by idiopathic PAH or familial PAH depending on the absence or presence of genetic information; the term secondary pulmonary hypertension has been abandoned.

As used herein, the term “pulmonary hemodynamic parameter” refers to any parameter used to describe or evaluate the blood flow through the heart and pulmonary vasculature. Examples of pulmonary hemodynamic parameters include, but are not limited to, mean pulmonary arterial pressure (mPAP), diastolic pulmonary arterial pressure (dPAP) [also known as pulmonary artery diastolic pressure (PADP)], systolic pulmonary arterial vpressure (sPAP) [also known as pulmonary artery systolic pressure (PASP)], mean right atrial pressure (mRAP), pulmonary capillary wedge pressure (PCWP) [also known as pulmonary artery wedge pressure (PAWP)], left ventricular end-diastolic pressure (LVEDP), diastolic pressure gradient (DPG) [also known as diastolic pressure difference (DPD)], left atrial pressure (LAP), transpulmonary gradient (TPG), pulmonary vascular resistance (PVR) and cardiac output (CO).

Many of the pulmonary hemodynamic parameters described above are interrelated. For example, PCWP is often used as a more convenient, less invasive approximation of LAP.

As another example, PVR is related to mPAP, PCWP and CO according to the following equation:

$$PVR = ( m P AP - PCWP ) / CO [ Woods Units ]$$

The PVR measures the resistance to flow imposed by the pulmonary vasculature without the influence of the left-sided filling pressure. PVR can also be measured according to the following equations:

$$PVR = TPG \times 80 / CO [ unit : dynes - sec - {cm}^{- 5} ] OR PVR = ( mPAP - PCWP ) \times 80 / CO [ unit : dynes - sec - {cm}^{- 5} ]$$

In some embodiments, the total pulmonary resistance (TPR) can be measured using the following equation:

$$TPR = mPAP / CO$$

According to some embodiments, a pre-capillary pulmonary arterial contribution to PH may be reflected by an elevated PVR. In some embodiments, the normal PVR is 20-130 dynes-sec-cm⁻⁵ or 0.5-1.1 Wood units. According to some embodiments, an elevated PVR may refer to a PVR above 2 Wood units, above 2.5 Wood units, above 3 Wood units or above 3.5 Wood units.

As yet another example, TPG is the difference between mPAP and left atrial pressure (PLA; commonly estimated by pulmonary capillary wedge pressure: PCWP) as shown by the following equation: TPG-mPAP-PCWP

The TPG is influenced by all the determinants of mPAP, including flow, resistance and left heart filling pressure. A pre-capillary pulmonary arterial contribution to PH may be reflected by an increased trans-pulmonary gradient (TPG). According to some embodiments, an increased TPG may refer to an mPAP-PCWP that exceeds 12-15 mmHg.

DPG (defined as diastolic PAP—mean PAWP) appears to best approach the characteristics required to determine pulmonary vascular disease. In some embodiments, the DPG is synonymous with diastolic pressure difference (DPD). In normal subjects, DPG generally lies in the 1-3 mmHg range, and in patients evaluated for cardiac disease (excluding shunts), DPG remains ≤5 mmHg in most cases.

As a further example, mPAP is related to dPAP and sPAP according to the following equation: mPAP=(⅔)dPAP+(⅓)sPAP

Furthermore, dPAP and sPAP can be used to calculate the pulse pressure (mmHg) using the following equation: pulse pressure=sPAP−dPAP

Pulse pressure can be used to calculate the pulmonary artery compliance using the following equation: pulmonary artery compliance (mI·mmHg⁻¹)=stroke volume/pulse pressure. In some embodiments, the pulmonary hemodynamic parameters are measured directly, such as during a right heart catheterization. In other embodiments, the pulmonary hemodynamic parameters are estimated and/or evaluated through other techniques such as magnetic resonance imaging (MRI) or echocardiography.

Exemplary pulmonary hemodynamic parameters include mPAP, PAWP, TPG, DPG, and PVR. The one or more pulmonary hemodynamic parameters may be measured by any appropriate procedures, such as by utilizing a right heart catheterization or echocardiography. Various hemodynamic types of PH are shown in Table 9 together with their corresponding clinical classification (Table 8).

TABLE 9
Hemodynamic Types of Pulmonary Hypertension (PH)

| Hemo- | Hemo- | | |
|---|---|---|---|
| dynamic | dynamic | | WHO PH |
| Type | Subtype | Characteristics | Classification |
| Pulmonary | — | mPAP > 20 mmHg | All (Groups 1-5) |
| Hypertension | | | |
| Pre-Capillary | — | mPAP > 20 mmHg | Group 1: Pulmonary |
| PH | | PAWP ≤ 15 mmHg | arterial hypertension |
| | | PVR ≥ 3 Wood units | Group 3: PH due to |
| | | | lung disease and/or |
| | | | hypoxia |
| | | | Group 4: PH due to |
| | | | pulmonary artery |
| | | | obstructions |
| | | | Group 5: PH with |
| | | | unclear and/or |
| | | | multifactorial |
| | | | mechanisms. |
| Post- | Isolated | mPAP > 20 mmHg | Group 2: PH due to |
| Capillary | Post- | PAWP > 15 mmHg | left heart disease |
| PH | Capillary PH | PVR < 3 Wood units | Group 5: PH with |
| | | DPG < 7 mmHg | unclear and/or |
| | Combined | mPAP > 20 mmHg | multifactorial |
| | Pre- | PAWP > 15 mmHg | mechanisms. |
| | and Post- | PVR ≥ 3 Wood units | |
| | Capillary PH | DPG ≥ 7 mmHg | |

The types of PH and the difference between pre-capillary pulmonary hypertension and post-capillary pulmonary hypertension are based on pulmonary hemodynamic parameters. As used herein, the term “pre-capillary pulmonary hypertension” includes WHO clinical Groups 1, 3, 4, and 5. In general, pre-capillary pulmonary hypertension is characterized using the pulmonary hemodynamic parameters shown in Table 9 (i.e., an mPAP >20 mmHg or in some embodiments an mPAP >25 mmHg). As used herein, the term “post-capillary pulmonary hypertension” (PcPH) includes both isolated post-capillary pulmonary hypertension (IpcPH) and combined pre- and post-capillary pulmonary hypertension (CpcPH), both within WHO clinical Groups 2 and 5. In some embodiments, IpcPH is characterized using the pulmonary hemodynamic parameters shown in Table 9 (i.e., one or more of the following pulmonary hemodynamic parameters: mPAP >20 mmHg, PAWP >15 mmHg, PVR <3 Wood units, and/or DPG <7 mmHg). In some embodiments, CpcPH is characterized using the pulmonary hemodynamic parameters shown in Table 9 (i.e., one or more of the following pulmonary hemodynamic parameters: mPAP >20 mmHg, PAWP >15 mmHg, PVR ≥3 Wood units, and/or DPG ≥7 mmHg). In some embodiments, CpcPH is characterized as comprising one or more of the following hemodynamic parameters: mPAP ≥25 mmHg; PAWP >15 mmHg; and PVR >3 WU.

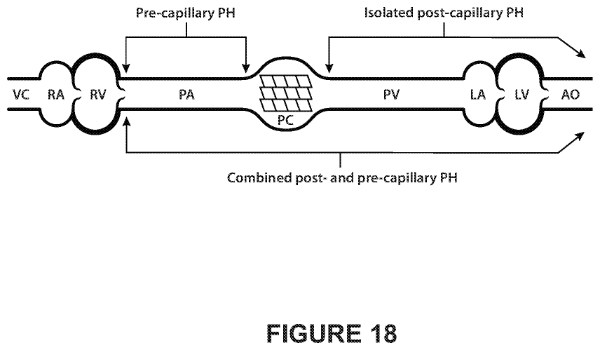

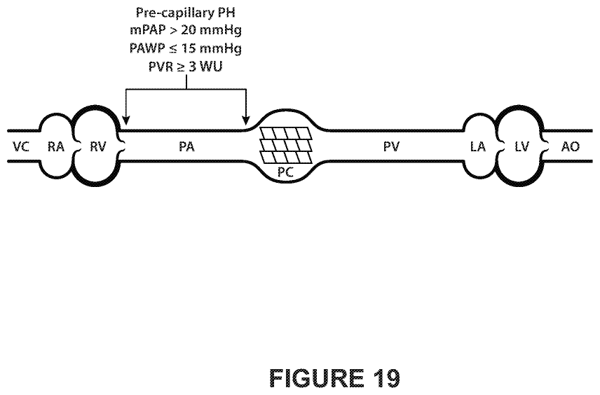

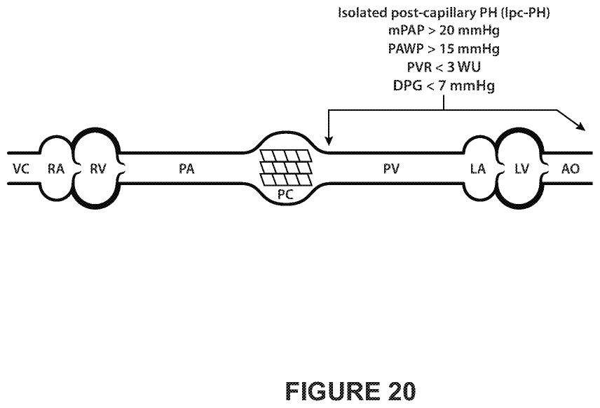

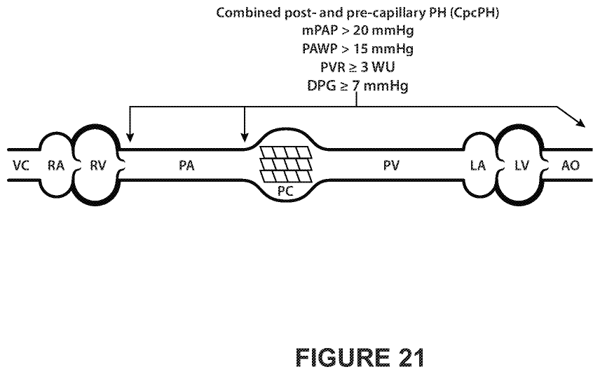

For example, as shown in FIG. 18 the difference between pre-capillary pulmonary hypertension, isolated post-capillary pulmonary hypertension, and combined post- and pre-capillary pulmonary hypertension are based on pulmonary hemodynamic parameters and the involvement of various regions of the cardiopulmonary system (pre and/or post capillary regions). See, e.g., Aras M A, et al. Curr Cardiol Rep. 2019; 21(7):62 and Galiè N. et al. Eur Heart J. 2018; 39(15):1265-1268. Similarly, FIG. 19 shows a schematic image of a linearized version of cardiopulmonary circulation and the hemodynamic parameters associated with pre-capillary PH. Id. A schematic image of a linerarized version of cardiopulmonary circulation and the hemodynamic parameters associated with isolated post-capillary PH (IpcPH) is shown in FIG. 20. Id. Similarly, FIG. 21 shows a schematic image of a linearized version of cardiopulmonary circulation and the hemodynamic parameters associated with combined post- and pre-capillary PH (CpcPH). Id. Abbreviations used in FIGS. 18-21 are as follows: VC—vena cava; RA—right atrium; RV—right ventricle; PA—pulmonary artery; PC—pulmonary capillaries; PV—pulmonary ventricles; LA—left atrium; LV—left ventricle; AO—Aorta; mPAP—mean pulmonary arterial pressure; PAWP—pulmonary arterial wedge pressure; PVR—pulmonary vascular resistance.

The clinical classification or hemodynamic types of PH described herein and the associated diagnostic parameters may be updated or varied based on the availability of new or existing sources of data or when additional clinical entities are considered.

### Characteristics of PH

The diagnosis of PH, including WHO PH class and functional group, can be determined based on symptoms and physical examination using a review of a comprehensive set of parameters to determine if the hemodynamic and other criteria are met. Some of the criteria which may considered include the patient's clinical presentation (e.g., shortness of breath, fatigue, weakness, angina, syncope, dry-couch, exercise-induced nausea and vomiting), electrocardiogram (ECG) results, chest radiograph results, pulmonary function tests, arterial blood gases, echocardiography results, ventilation/perfusion lung scan results, high-resolution computed tomography results, contrast-enhanced computed tomography results, pulmonary angiography results, cardiac magnetic resonance imaging, blood tests (e.g., biomarkers such as BNP or NT-proBNP), immunology, abdominal ultrasound scan, right heart catherization (RHC), vasoreactivity, and genetic testing. See, e.g., Galie N., et al Euro Heart J. (2016) 37, 67-119.

In some embodiments, a biomarker may be used to determine the diagnosis of PH. For instance, in some embodiments, the biomarker is a marker of vascular dysfunction (e.g., asymmetric dimethylarginine (ADMA), endothelin-1, angiopoietins, or von Willebrand factor). In some embodiments, the biomarker is a marker of inflammation (C-reactive protein, interleukin 6, chemokines). In some embodiments, the biomarker is a marker of myocardial stress (e.g., (atrial natriuretic peptide, brain natriuretic peptide (BNP)/NT-proBNP, or troponins). In some embodiments, the biomarker is a marker of low CO and/or tissue hypoxia (e.g., pCO2, uric acid, growth differentiation factor 15 (GDF15), or osteopontin). In some embodiments, the biomarker is a marker of secondary organ damage (e.g., creatinine or bilirubin). See, e.g., Galie N., et al Euro Heart J. (2016) 37, 67-119.

### Group 1 PH

Pulmonary arterial hypertension (WHO Group 1 PH) is a serious, progressive and life-threatening disease of the pulmonary vasculature, characterized by profound vasoconstriction and an abnormal proliferation of smooth muscle cells in the walls of the pulmonary arteries. Severe constriction of the blood vessels in the lungs leads to very high pulmonary arterial pressures. These high pressures make it difficult for the heart to pump blood through the lungs to be oxygenated. Patients with PAH suffer from extreme shortness of breath as the heart struggles to pump against these high pressures. Patients with PAH typically develop significant increases in PVR and sustained elevations in mPAP, which ultimately lead to right ventricular failure and death. Patients diagnosed with PAH have a poor prognosis and equally compromised quality of life, with a mean life expectancy of 2 to 5 years from the time of diagnosis if untreated.

A variety of factors contribute to the pathogenesis of pulmonary hypertension including proliferation of pulmonary cells which can contribute to vascular remodeling (i.e., hyperplasia). For example, pulmonary vascular remodeling occurs primarily by proliferation of arterial endothelial cells and smooth muscle cells of patients with pulmonary hypertension. Overexpression of various cytokines is believed to promote pulmonary hypertension. Further, it has been found that pulmonary hypertension may rise from the hyperproliferation of pulmonary arterial smooth cells and pulmonary endothelial cells. Still further, advanced PAH may be characterized by muscularization of distal pulmonary arterioles, concentric intimal thickening, and obstruction of the vascular lumen by proliferating endothelial cells. Pietra et al., J. Am. Coll. Cardiol., 43:255-325 (2004).

PAH can be diagnosed based on a mean pulmonary artery pressure of above 25 mmHg (or above 20 mmHg under updated guidelines) at rest, with a normal pulmonary artery capillary wedge pressure. PAH can lead to shortness of breath, dizziness, fainting, and other symptoms, all of which are exacerbated by exertion. PAH can be a severe disease with a markedly decreased exercise tolerance and heart failure. Two major types of PAH include idiopathic PAH (e.g., PAH in which no predisposing factor is identified) and heritable PAH (e.g., PAH associated with a mutation in BMPR2, ALK1, ENG, SMAD9, CAV1, KCNK3, or EIF2AK4). In 70% of familial PAH cases, mutations are located in the BMPR2 gene. Risk factors for the development of PAH include family history of PAH, drug and toxin use (e.g., methamphetamine or cocaine use), infection (e.g., HIV infection or schistosomiasis), cirrhosis of the liver, congenital heart abnormalities, portal hypertension, pulmonary veno-occlusive disease, pulmonary capillary hemangiomatosis, or connective tissue/autoimmune disorders (e.g., scleroderma or lupus). PAH may be associated with long term responders to calcium channel blockers, overt features of venous/capillaries (PVOD/PCH) involvement, and persistent PH of the newborn syndrome.

### Group 2 PH

Pulmonary hypertension due to left heart disease (PH-LHD) (WHO Group 2 PH) is a complex pathophenotype that, when present, may result in an increased susceptibility to adverse events and worse clinical outcome. PH-LHD is sometimes defined as patients having a pulmonary capillary wedge pressure (PCWP) >15 mmHg and a mean pulmonary arterial pressure (mPAP) ≥25 mmHg (or a mean pulmonary arterial pressure (mPAP) ≥20 mmHg under updated guidelines). PH-LHD occurs as a consequence of the backward transmission of high left sided filling pressures, mainly driven by LV diastolic function, directly to the post-capillary pulmonary vessels and, thereby, to the rest of the pulmonary circulation. PH-LHD may be associated with or caused by PH due to heart failure with preserved left ventricle ejection fraction (LVEF) [also known as HFpEF], PH due to heart failure with reduced LVEF (also known as HFrEF), valvular heart disease (VHD), or congenital/acquired cardiovascular conditions leading to post-capillary PH. Compared with PAH, patients with PH-LHD are often older, female, with a higher prevalence of cardiovascular co-morbidities and most, if not all, of the features of metabolic syndrome.

Valvular heart disease (VHD) associated with pulmonary hypertension may result from multiple mechanisms such as an increase in PVR, pulmonary blood flow, or pulmonary venous pressure. The chronic rise in PAP frequently leads to RV pressure overload and subsequent RV failure. Clinical signs and symptoms of left-sided VHD with PH are orthopnea and paroxysmal nocturnal dyspnea. In advanced stages of diseases, signs of RV failure including peripheral edema, ascites, and syncope are frequently observed. There are four valvular heart disease subtypes which include mitral valve stenosis, mitral valve regurgitation, aortic stenosis, and aortic regurgitation.

Mitral valve stenosis occurs when the heart's mitral valve is narrowed due to the valve becoming stiff or scarred, or the valve flaps partially joining together. This results in the valve not opening as widely as it should, which causes poor blood flow and may result in blood backing up into the lungs. Left untreated, mitral valve stenosis can lead to serious heart complications. Common causes of mitral valve stenosis include rheumatic heart disease, radiation, and mitral annulus calcification. Typical interventions for mitral stenosis include balloon valvuloplasty, commissurotomy, and surgical valve replacement.

Mitral valve regurgitation (also called mitral insufficiency) occurs when the flaps (leaflets) of the mitral valve do not close tightly, allowing blood to flow backward in the heart. As a result, blood can't move through the heart or to the rest of the body as efficiently, resulting in fatigue or shortness of breath. Additionally, the reduced flow increases pressure in the left atrium and lung vasculature. In moderate to severe cases, surgery may be recommended to either repair or replace the damaged valve. Left untreated, severe mitral valve regurgitation can cause heart failure or serious heart rhythm problems. Common causes of mitral valve regurgitation include degenerative mitral disease such as mitral valve prolapse and mitral annulus calcification. Typical interventions for mitral valve regurgitation include transcatheter mitral valve repair, surgical repair, or replacement.

In aortic stenosis, the aortic valve does not open fully. This decreases blood flow from the heart. As the aortic valve becomes more narrow, the pressure increases inside the left heart ventricle. This causes the left heart ventricle to become thicker, which decreases blood flow and can lead to chest pain. As the pressure continues to rise, blood may back up into the lungs causing dyspnea. Severe forms of aortic stenosis prevent enough blood from reaching the brain and rest of the body. Common causes of aortic stenosis include calcification of the aortic valve or the presence of a bicuspid aortic valve. Typical interventions include transcatheter aortic valve replacement (percutaneous valve replacement) and surgical valve replacement.

Aortic regurgitation (also known as aortic insufficiency) occurs when the aortic valve is unable to fully close. The valve leaks, resulting in reduced blood flow. As a result, the heart has to work harder to make up for the reduced blood flow, and over time it will weaken. Because of this, the amount of blood that flows from the heart to the rest of the body is reduced. Common causes of aortic regurgitation include aortic root dilatation and presence of a bicuspid aortic valve.

Among those patients with PH-LHD, two phenotypes have been described: 1) a group of isolated post-capillary (IpcPH) or “passive” PH in which elevated pulmonary pressures are reversible and in proportion to increases in left atrial pressure, and 2) a group with an added “pre-capillary” component [combined post-capillary and pre-capillary PH (CpcPH)]. This latter group, CpcPH, may have comorbid pulmonary vascular remodeling and therefore may demonstrate persistent PH after interventions to lower left sided filling pressures.

In some embodiments, a combination of mPAP, PAWP, PVR, or DPG may be used to define the different subtypes of PH-LHD, i.e., IpcPH and CpcPH (see, e.g., Table 9). In some embodiments, patients with CpcPH are characterized as having a TPG >12-15 mmHg and a PVR >2.5-3 Wood units (WU). In some embodiments, CpcPH is distinguished from IpcPH using the DPG. In some embodiments, a patient with CpcPH has a DPG ≥7 mmHg. In some embodiments, a patient with IpcPH has a DPG <7 mmHg.

In some embodiments, a combination of DPG and PVR may be used to define the different types of PH-LHD. For instance, in some embodiments, IpcPH patients have a DPG <7 mmHg and/or a PVR of ≤3 WU. In some embodiments, CpcPH patients have a DPG ≥7 mmHg and/or a PVR >3 WU.

The clinical classification or hematological classification described herein and the associated diagnostic parameters may be updated when new data are available or when additional clinical entities are considered. For instance, at the 5′ World Symposium on Pulmonary Hypertension (WSPH), a new terminology was adopted to distinguish IpcPH from CpcPH, based on the diastolic pressure difference/gradient (DPG) between the dPAP and PAWP. However, this definition was found to be too restrictive and exposed to interpretation, leading to controversies about whether the DPG would or would not predict outcome in patients with group 2 PH. Accordingly, at the 6′ WSPH, pulmonary vascular resistance (PVR) was subsequently reintroduced to better reflect the impact of the right ventricle on patient outcome. See, e.g., Vachiery J. L., et al. Eur Respir J 2019 Jan. 24; 53(1).

Therapies for treating PH-LHD primarily include treatment of the underlying condition (i.e., COPD, sleep apnea syndrome, CTEPH) prior to considering specific measures to treat the PH itself. Some therapies include repair of valvular heart disease (if indicated). Non-specific vasodilators such as nitrates and hydralazine may also be used. In some embodiments, an LV assist device (LVAD) may be used to lower pulmonary pressure. The lack of specific therapies is particularly problematic because PH-LHD is the most common cause of PH in western countries and its presence commonly results in adverse course of the disease. Specifically, the presence of PH-LHD can result in more severe symptoms in LHD, worse exercise tolerance, and a negative impact on outcome.

### Group 3 PH

Pulmonary hypertension due to lung disease and/or hypoxia (WHO Group 3 PH) refers to a form of pulmonary hypertension that is due to lung disease or chronic hypoxia. This form of PH is also known as “hypoxic PH” or “hypoxic pulmonary hypertension.” Hypoxic PH may be associated with or caused by chronic obstructive pulmonary disease (e.g., emphysema), interstitial lung disease, sleep-disordered breathing (e.g., sleep apnea), lung disease (e.g., pulmonary fibrosis), alveolar hypoventilation disorders, chronic exposure to high altitude, or developmental abnormalities.

### Group 4 PH

Pulmonary hypertension due to pulmonary artery obstructions (WHO Group 4 PH) is a form of pulmonary hypertension that is related to chronic arterial obstruction (e.g., blood clots). There may be multiple pathophysiological mechanisms driving development of PH in Group 4 including chronic thromboembolic PH, sarcoma (high or intermediate grade) or angiosarcoma, other malignant tumors (e.g., renal carcinoma, uterine carcinoma, germ cell tumors of the testis, or other tumors), non-malignant tumors (e.g., uterine leiomyoma), arteritis without connective tissue disease, congenital pulmonary artery stenosis, or parasites (e.g., hydatidosis).

Various pulmonary hemodynamic parameters are associated with Group 4 PH. For instance, in patients with PH due to pulmonary artery obstructions, those with severe PH (>40 mmHg) often have a marked increase in PVR (around 10 WU); more often these patients may have a mild PH (mPAP 20-30 mmHg), associated with lower PVR but remaining generally >3 WU. See, e.g., Simonneau (2019) Eur Respir J: 53:1801913. In these different chronic lung diseases, even a modest elevation in mPAP (20-29 mmHg) can be associated with a poor prognosis. Furthermore, in chronic thromboembolism, patients may have severe pre-capillary PH with a mPAP of about 47 mmHg and a mean PVR of about 8.9 WU. Id. In this setting, even in patients with mild elevation of mPAP (20-24 mmHg), PVR is generally >3 WU.

### Group 5 PH

Pulmonary hypertension with unclear and/or multifactorial mechanisms (WHO Group 5 PH) is a group which contains less-studied forms of PH in comparison with the other groups. However, many of the PH forms currently in group 5 represent a significant part of the PH burden. The diseases within Group 5 PH are characterized by having no identified predominant mechanism driving the development of PH. There may be multiple pathophysiological mechanisms driving development of PH, including hematological disorders (e.g., chronic hemolytic anemia or myeloproliferative disorders), systemic and metabolic disorders (e.g., Pulmonary Langerhans cell histiocytosis, Gaucher disease, glycogen storage disease, neurofibromatosis, or sarcoidosis), others (e.g., chronic renal failure with or without hemodialysis or fibrosing mediastinitis), or complex congenital heart disease.

### Measurements of PH

In certain aspects, the disclosure relates to methods of treating, preventing, or reducing the progression rate and/or severity of PcPH (e.g., treating, preventing, or reducing the progression rate and/or severity of one or more complications of post-capillary pulmonary hypertension in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to treating PcPH patients that have IpcPH. In some embodiments, the method relates to treating PcPH patients that have CpcPH. In some embodiments, the method relates to treating PcPH patients that have pulmonary hypertension due to left heart disease (PH-LHD). In some embodiments, the method relates to treating PcPH patients that have Group 2 PH as classified by the WHO. In some embodiments, the method relates to treating PcPH patients that have pulmonary hypertension due to heart failure with preserved LVEF (HFpEF). In some embodiments, the method relates to treating PcPH patients that have pulmonary hypertension due to heart failure with reduced LVEF (HFrEF). In some embodiments, the method relates to treating PcPH patients that have valvular heart disease. In some embodiments, the valvular heart disease is aortic regurgitation. In some embodiments, the valvular heart disease is aortic stenosis. In some embodiments, the valvular heart disease is mitral valve disease. In some embodiments, the valvular heart disease is mitral valve regurgitation. In some embodiments, the valvular heart disease is mitral valve stenosis. In some embodiments, the method relates to treating CpcPH patients who have PH due to valvular heart disease. In some embodiments, the method relates to treating IpcPH patients who have PH due to valvular heart disease. In some embodiments, the method relates to treating PcPH patients that have congenital/acquired cardiovascular conditions leading to post-capillary PH. In some embodiments, the method relates to treating PcPH patients that have pulmonary hypertension with unclear and/or multifactorial mechanisms. In some embodiments, the method relates to treating PcPH patients that have Group 5 PH as classified by the WHO.

In some embodiments, the disclosure relates to methods of treating, preventing, or reducing the progression rate and/or severity of pulmonary hypertension in combinations of certain patient populations. Each of the patient populations described herein can be combined and reorganized accordingly. For instance, in some embodiments, the method relates to treating CpcPH patients who have PH due to heart failure with preserved LVEF (HFpEF). In some embodiments, the method relates to treating CpcPH patients who have PH due to heart failure with reduced LVEF (HFrEF). In some embodiments, the method relates to treating CpcPH patients who have PH due to valvular heart disease. In some embodiments, the method relates to treating IpcPH patients who have PH due to heart failure with preserved LVEF (HFpEF). In some embodiments, the method relates to treating IpcPH patients who have PH due to heart failure with reduced LVEF (HFrEF). In some embodiments, the method relates to treating IpcPH patients who have PH due to valvular heart disease.

In some embodiments, the method relates to pulmonary hypertension patients that have pulmonary hypertension with unclear and/or multifactorial mechanisms. In some embodiments, the method relates to patients that have a hematological disorder (e.g., chronic hemolytic anemia and myeloproliferative disorders). In some embodiments, the method relates to patients that have a systemic and/or metabolic disorder (e.g., pulmonary langerhans cell histiocytosis, Gaucher disease, glycogen storage disease, neurofibromatosis, and sarcoidosis). In some embodiments, the method relates to pulmonary hypertension patients that have other disorders with unclear and/or multifactorial mechanisms (e.g., chronic renal failure with or without hemodialysis or fibrosing mediastinitis). In some embodiments, the method relates to patients that have complex congenital heart disease.

mPAP

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has resting mean pulmonary arterial pressure (mPAP) of at least 20 mmHg (e.g., at least 20, 25, 30, 35, 40, 45, or 50 mmHg). In some embodiments, the method relates to patients having a resting mPAP of at least 20 mmHg. In some embodiments, the method relates to patients having a resting mPAP of at least 25 mmHg. In some embodiments, the method relates to patients having a resting mPAP of at least 30 mmHg. In some embodiments, the method relates to patients having a resting mPAP of at least 35 mmHg. In some embodiments, the method relates to patients having a resting mPAP of at least 40 mmHg. In some embodiments, the method relates to patients having a resting mPAP of at least 45 mmHg. In some embodiments, the method relates to patients having a resting mPAP of at least 50 mmHg. As used herein, the terms “mean pulmonary arterial pressure” and “mean pulmonary artery pressure” are used interchangeably.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to improving the pulmonary arterial pressure in the patient. In some embodiments, the improvement in pulmonary arterial pressure is a reduction in the mean pulmonary arterial pressure (mPAP). In some embodiments, the method relates to reducing mPAP. In some embodiments, the method relates to reducing the patient's mPAP by at least 1 mmHg. In some embodiments, the method relates to reducing the patient's mPAP by at least 2 mmHg. In some embodiments, the method relates to reducing the patient's mPAP by at least 3 mmHg. In certain embodiments, the method relates to reducing the patient's mPAP by at least 5 mmHg. In certain embodiments, the method relates to reducing the patient's mPAP by at least 7 mmHg. In certain embodiments, the method relates to reducing the patient's mPAP by at least 10 mmHg. In certain embodiments, the method relates to reducing the patient's mPAP by at least 12 mmHg. In certain embodiments, the method relates to reducing the patient's mPAP by at least 15 mmHg. In certain embodiments, the method relates to reducing the patient's mPAP by at least 20 mmHg. In certain embodiments, the method relates to reducing the patient's mPAP by at least 25 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to decreasing the patient's mPAP by least 10% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's mPAP by at least 10%. In some embodiments, the method relates to decreasing the patient's mPAP by at least 5%. In some embodiments, the method relates to decreasing the patient's mPAP by at least 10%. In some embodiments, the method relates to decreasing the patient's mPAP by at least 15%. In some embodiments, the method relates to decreasing the patient's mPAP by at least 20%. In some embodiments, the method relates to decreasing the patient's mPAP by at least 25%. In some embodiments, the method relates to decreasing the patient's mPAP by at least 30%. In some embodiments, the method relates to decreasing the patient's mPAP by at least 35%. In some embodiments, the method relates to decreasing the patient's mPAP by at least 40%. In some embodiments, the method relates to decreasing the patient's mPAP by at least 45%. In some embodiments, the method relates to decreasing the patient's mPAP by at least 50%. In some embodiments, the method relates to decreasing the patient's mPAP by at least 55%. In some embodiments, the method relates to decreasing the patient's mPAP by at least 60%. In some embodiments, the method relates to decreasing the patient's mPAP by at least 65%. In some embodiments, the method relates to decreasing the patient's mPAP by at least 70%. In some embodiments, the method relates to decreasing the patient's mPAP by at least 75%. In some embodiments, the method relates to decreasing the patient's mPAP by at least 80%. In some embodiments, the method relates to decreasing the patient's mPAP by at least 85%. In some embodiments, the method relates to decreasing the patient's mPAP by at least 90%. In some embodiments, the method relates to decreasing the patient's mPAP by at least 95%. In some embodiments, the method relates to decreasing the patient's mPAP by at least 100%.

mRAP

In some patients, increased pulmonary vascular resistance to blood flow leads to increased right atrial pressure (RAP) and right heart failure. Patients with right heart failure typically have an increased ratio of RAP and pulmonary artery wedge pressure (PAWP). In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has resting mean right atrial pressure (mRAP) of at least 5 mmHg (e.g., at least 5, 6, 7, 8, 9, 10, 11, 12, 13, 14, 15, 16, 17, 18, 18, 20, 21, 22, 23, 24, or 25 mmHg). In some embodiments, the method relates to a patient having a resting mRAP of at least 5 mmHg. In some embodiments, the method relates to a patient having a resting mRAP of at least 6 mmHg. In some embodiments, the method relates to a patient having a resting mRAP of at least 7 mmHg. In some embodiments, the method relates to a patient having a resting mRAP of at least 8 mmHg. In some embodiments, the method relates to a patient having a resting mRAP of at least 9 mmHg. In some embodiments, the method relates to a patient having a resting mRAP of at least 10 mmHg. In some embodiments, the method relates to a patient having a resting mRAP of at least 11 mmHg. In some embodiments, the method relates to a patient having a resting mRAP of at least 12 mmHg. In some embodiments, the method relates to a patient having a resting mRAP of at least 13 mmHg. In some embodiments, the method relates to a patient having a resting mRAP of at least 14 mmHg. In some embodiments, the method relates to a patient having a resting mRAP of at least 15 mmHg. In some embodiments, the method relates to a patient having a resting mRAP of at least 16 mmHg. In some embodiments, the method relates to a patient having a resting mRAP of at least 17 mmHg. In some embodiments, the method relates to a patient having a resting mRAP of at least 18 mmHg. In some embodiments, the method relates to a patient having a resting mRAP of at least 19 mmHg. In some embodiments, the method relates to a patient having a resting mRAP of at least 20 mmHg. In some embodiments, the method relates to a patient having a resting mRAP of at least 21 mmHg. In some embodiments, the method relates to a patient having a resting mRAP of at least 22 mmHg. In some embodiments, the method relates to a patient having a resting mRAP of at least 23 mmHg. In some embodiments, the method relates to a patient having a resting mRAP of at least 24 mmHg. In some embodiments, the method relates to a patient having a resting mRAP of at least 25 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to improving the mean right atrial pressure in the patient. In some embodiments, the improvement in the mean right atrial pressure (mRAP) is a reduction in the mRAP. In some embodiments, the method relates to reducing mRAP. In some embodiments, the method relates to reducing the patient's mRAP by at least 1 mmHg. In some embodiments, the method relates to reducing the patient's mRAP by at least 2 mmHg. In some embodiments, the method relates to reducing the patient's mRAP by at least 3 mmHg. In some embodiments, the method relates to reducing the patient's mRAP by at least 4 mmHg. In certain embodiments, the method relates to reducing the patient's mRAP by at least 5 mmHg. In some embodiments, the method relates to reducing the patient's mRAP by at least 6 mmHg. In certain embodiments, the method relates to reducing the patient's mRAP by at least 7 mmHg. In some embodiments, the method relates to reducing the patient's mRAP by at least 8 mmHg. In some embodiments, the method relates to reducing the patient's mRAP by at least 9 mmHg. In certain embodiments, the method relates to reducing the patient's mRAP by at least 10 mmHg. In some embodiments, the method relates to reducing the patient's mRAP by at least 11 mmHg. In certain embodiments, the method relates to reducing the patient's mRAP by at least 12 mmHg. In some embodiments, the method relates to reducing the patient's mRAP by at least 13 mmHg. In some embodiments, the method relates to reducing the patient's mRAP by at least 14 mmHg. In certain embodiments, the method relates to reducing the patient's mRAP by at least 15 mmHg. In some embodiments, the method relates to reducing the patient's mRAP by at least 16 mmHg. In some embodiments, the method relates to reducing the patient's mRAP by at least 17 mmHg. In some embodiments, the method relates to reducing the patient's mRAP by at least 18 mmHg. In some embodiments, the method relates to reducing the patient's mRAP by at least 19 mmHg. In certain embodiments, the method relates to reducing the patient's mRAP by at least 20 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to decreasing the patient's mRAP by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's mRAP by at least 10%. In some embodiments, the method relates to decreasing the patient's mRAP by at least 5%. In some embodiments, the method relates to decreasing the patient's mRAP by at least 10%. In some embodiments, the method relates to decreasing the patient's mRAP by at least 15%. In some embodiments, the method relates to decreasing the patient's mRAP by at least 20%. In some embodiments, the method relates to decreasing the patient's mRAP by at least 25%. In some embodiments, the method relates to decreasing the patient's mRAP by at least 30%. In some embodiments, the method relates to decreasing the patient's mRAP by at least 35%. In some embodiments, the method relates to decreasing the patient's mRAP by at least 40%. In some embodiments, the method relates to decreasing the patient's mRAP by at least 45%. In some embodiments, the method relates to decreasing the patient's mRAP by at least 50%. In some embodiments, the method relates to decreasing the patient's mRAP by at least 55%. In some embodiments, the method relates to decreasing the patient's mRAP by at least 60%. In some embodiments, the method relates to decreasing the patient's mRAP by at least 65%. In some embodiments, the method relates to decreasing the patient's mRAP by at least 70%. In some embodiments, the method relates to decreasing the patient's mRAP by at least 75%. In some embodiments, the method relates to decreasing the patient's mRAP by at least 80%. In some embodiments, the method relates to decreasing the patient's mRAP by at least 85%. In some embodiments, the method relates to decreasing the patient's mRAP by at least 90%. In some embodiments, the method relates to decreasing the patient's mRAP by at least 95%. In some embodiments, the method relates to decreasing the patient's mRAP by at least 100%.

### PVR

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has a pulmonary vascular resistance (PVR) of at least 2.5 Woods Units (e.g., at least 2.5, 3, 4, 6, 8, 10, 12, 14, 16, 18, or 20 Woods Units). In some embodiments, the method relates to patients having a PVR of at least 2.5 Woods Units. In some embodiments, the method relates to patients having a PVR of at least 3 Woods Units. In some embodiments, the method relates to patients having a PVR of at least 4 Woods Units. In some embodiments, the method relates to patients having a PVR of at least 5 Woods Units. In some embodiments, the method relates to patients having a PVR of at least 6 Woods Units. In some embodiments, the method relates to patients having a PVR of at least 7 Woods Units. In some embodiments, the method relates to patients having a PVR of at least 8 Woods Units. In some embodiments, the method relates to patients having a PVR of at least 9 Woods Units. In some embodiments, the method relates to patients having a PVR of at least 10 Woods Units. In some embodiments, the method relates to patients having a PVR of at least 12 Woods Units. In some embodiments, the method relates to patients having a PVR of at least 14 Woods Units. In some embodiments, the method relates to patients having a PVR of at least 16 Woods Units. In some embodiments, the method relates to patients having a PVR of at least 18 Woods Units. In some embodiments, the method relates to patients having a PVR of at least 20 Woods Units.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to reducing the patient's PVR. In some embodiments, the reduction in the patient's PVR is a result of a decrease in the patient's mean pulmonary arterial pressure (mPAP). In some embodiments, the method relates to reducing the patient's PVR by at least 0.5 Wood Units. In some embodiments, the method relates to reducing the patient's PVR by at least 1 Wood Units. In some embodiments, the method relates to reducing the patient's PVR by at least 2 Wood Units. In some embodiments, the method relates to reducing the patient's PVR by at least 4 Wood Units. In some embodiments, the method relates to reducing the patient's PVR by at least 6 Wood Units. In some embodiments, the method relates to reducing the patient's PVR by at least 8 Wood Units. In some embodiments, the method relates to reducing the patient's PVR by at least 10 Wood Units.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to decreasing the patient's PVR. In some embodiments, the reduction in the patient's PVR is a result a decrease in the patient's mean pulmonary arterial pressure (mPAP). In some embodiments, the method relates to decreasing the patient's PVR by least 10% (e.g., at least 1%, 5, 10, 15, 20, 25, 30, 35, 40, 45, 50, 55, 60, 65, 70, 75, 80, 85, 90, 95, or 100%). In some embodiments, the method relates to decreasing the patient's PVR by at least 10%. In some embodiments, the method relates to decreasing the patient's PVR by at least 5%. In some embodiments, the method relates to decreasing the patient's PVR by at least 10%. In some embodiments, the method relates to decreasing the patient's PVR by at least 15%. In some embodiments, the method relates to decreasing the patient's PVR by at least 20%. In some embodiments, the method relates to decreasing the patient's PVR by at least 25%. In some embodiments, the method relates to decreasing the patient's PVR by at least 30%. In some embodiments, the method relates to decreasing the patient's PVR by at least 35%. In some embodiments, the method relates to decreasing the patient's PVR by at least 40%. In some embodiments, the method relates to decreasing the patient's PVR by at least 45%. In some embodiments, the method relates to decreasing the patient's PVR by at least 50%. In some embodiments, the method relates to decreasing the patient's PVR by at least 55%. In some embodiments, the method relates to decreasing the patient's PVR by at least 60%. In some embodiments, the method relates to decreasing the patient's PVR by at least 65%. In some embodiments, the method relates to decreasing the patient's PVR by at least 70%. In some embodiments, the method relates to decreasing the patient's PVR by at least 75%. In some embodiments, the method relates to decreasing the patient's PVR by at least 80%. In some embodiments, the method relates to decreasing the patient's PVR by at least 85%. In some embodiments, the method relates to decreasing the patient's PVR by at least 90%. In some embodiments, the method relates to decreasing the patient's PVR by at least 95%. In some embodiments, the method relates to decreasing the patient's PVR by at least 100%.

### PAWP

In certain aspects, the disclosure relates to methods of treating, preventing, or reducing the progression rate and/or severity of PcPH (e.g., treating, preventing, or reducing the progression rate and/or severity of one or more complications of PcPH WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has pulmonary arterial wedge pressure (PAWP) of at least 12 mmHg (e.g., at least 12, 15, 20, 25, 30, 35, 40, 45, or 50 mmHg). In some embodiments, the method relates to patients having a PAWP of at least 15 mmHg. In some embodiments, the method relates to patients having a PAWP of at least 20 mmHg. In some embodiments, the method relates to patients having a PAWP of at least 25 mmHg. In some embodiments, the method relates to patients having a PAWP of at least 30 mmHg. In some embodiments, the method relates to patients having a PAWP of at least 35 mmHg. In some embodiments, the method relates to patients having a PAWP of at least 40 mmHg. In some embodiments, the method relates to patients having a PAWP of at least 45 mmHg. In some embodiments, the method relates to patients having a PAWP of at least 50 mmHg. In some embodiments, the method relates to patients having a PCWP between 15 to 30 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to reducing the patient's PAWP by at least 1 mmHg. In some embodiments, the method relates to reducing the patient's PAWP by at least 2 mmHg. In some embodiments, the method relates to reducing the patient's PAWP by at least 4 mmHg. In some embodiments, the method relates to reducing the patient's PAWP by at least 6 mmHg. In some embodiments, the method relates to reducing the patient's PAWP by at least 10 mmHg. In some embodiments, the method relates to reducing the patient's PAWP by at least 15 mmHg. In some embodiments, the method relates to reducing the patient's PAWP by at least 20 mmHg. In some embodiments, the method relates to reducing the patient's PAWP by at least 25 mmHg. In some embodiments, the method relates to reducing the patient's PAWP by at least 30 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to decreasing the patient's PAWP by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's PAWP by at least 10%. In some embodiments, the method relates to decreasing the patient's PAWP by at least 5%. In some embodiments, the method relates to decreasing the patient's PAWP by at least 10%. In some embodiments, the method relates to decreasing the patient's PAWP by at least 15%. In some embodiments, the method relates to decreasing the patient's PAWP by at least 20%. In some embodiments, the method relates to decreasing the patient's PAWP by at least 25%. In some embodiments, the method relates to decreasing the patient's PAWP by at least 30%. In some embodiments, the method relates to decreasing the patient's PAWP by at least 35%. In some embodiments, the method relates to decreasing the patient's PAWP by at least 40%. In some embodiments, the method relates to decreasing the patient's PAWP by at least 45%. In some embodiments, the method relates to decreasing the patient's PAWP by at least 50%. In some embodiments, the method relates to decreasing the patient's PAWP by at least 55%. In some embodiments, the method relates to decreasing the patient's PAWP by at least 60%. In some embodiments, the method relates to decreasing the patient's PAWP by at least 65%. In some embodiments, the method relates to decreasing the patient's PAWP by at least 70%. In some embodiments, the method relates to decreasing the patient's PAWP by at least 75%. In some embodiments, the method relates to decreasing the patient's PAWP by at least 80%. In some embodiments, the method relates to decreasing the patient's PAWP by at least 85%. In some embodiments, the method relates to decreasing the patient's PAWP by at least 90%. In some embodiments, the method relates to decreasing the patient's PAWP by at least 95%. In some embodiments, the method relates to decreasing the patient's PAWP by at least 100%.

### LVEDP

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has left ventricular end diastolic pressure (LVEDP) of at least 12 mmHg (e.g., at least 12, 15, 20, 25, 30, 35, 40, 45, or 50 mmHg). In some embodiments, the method relates to patients having a LVEDP of at least 15 mmHg. In some embodiments, the method relates to patients having a LVEDP of at least 20 mmHg. In some embodiments, the method relates to patients having a LVEDP of at least 25 mmHg. In some embodiments, the method relates to patients having a LVEDP of at least 30 mmHg. In some embodiments, the method relates to patients having a LVEDP of at least 35 mmHg. In some embodiments, the method relates to patients having a LVEDP of at least 40 mmHg. In some embodiments, the method relates to patients having a LVEDP of at least 45 mmHg. In some embodiments, the method relates to patients having a LVEDP of at least 50 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to reducing the patient's LVEDP by at least 1 mmHg. In some embodiments, the method relates to reducing the patient's LVEDP by at least 2 mmHg. In some embodiments, the method relates to reducing the patient's LVEDP by at least 4 mmHg. In some embodiments, the method relates to reducing the patient's LVEDP by at least 6 mmHg. In some embodiments, the method relates to reducing the patient's LVEDP by at least 10 mmHg. In some embodiments, the method relates to reducing the patient's LVEDP by at least 15 mmHg. In some embodiments, the method relates to reducing the patient's LVEDP by at least 20 mmHg. In some embodiments, the method relates to reducing the patient's LVEDP by at least 25 mmHg. In some embodiments, the method relates to reducing the patient's LVEDP by at least 30 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to decreasing the patient's LVEDP by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's LVEDP by at least 10%. In some embodiments, the method relates to decreasing the patient's LVEDP by at least 5%. In some embodiments, the method relates to decreasing the patient's LVEDP by at least 10%. In some embodiments, the method relates to decreasing the patient's LVEDP by at least 15%. In some embodiments, the method relates to decreasing the patient's LVEDP by at least 20%. In some embodiments, the method relates to decreasing the patient's LVEDP by at least 25%. In some embodiments, the method relates to decreasing the patient's LVEDP by at least 30%. In some embodiments, the method relates to decreasing the patient's LVEDP by at least 35%. In some embodiments, the method relates to decreasing the patient's LVEDP by at least 40%. In some embodiments, the method relates to decreasing the patient's LVEDP by at least 45%. In some embodiments, the method relates to decreasing the patient's LVEDP by at least 50%. In some embodiments, the method relates to decreasing the patient's LVEDP by at least 55%. In some embodiments, the method relates to decreasing the patient's LVEDP by at least 60%. In some embodiments, the method relates to decreasing the patient's LVEDP by at least 65%. In some embodiments, the method relates to decreasing the patient's LVEDP by at least 70%. In some embodiments, the method relates to decreasing the patient's LVEDP by at least 75%. In some embodiments, the method relates to decreasing the patient's LVEDP by at least 80%. In some embodiments, the method relates to decreasing the patient's LVEDP by at least 85%. In some embodiments, the method relates to decreasing the patient's LVEDP by at least 90%. In some embodiments, the method relates to decreasing the patient's LVEDP by at least 95%. In some embodiments, the method relates to decreasing the patient's LVEDP by at least 100%.

### DPG

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has resting diastolic pressure gradient (DPG) of at least 5 mmHg (e.g., at least 5, 6, 7, 8, 9, 10, 15, 20, 25, 30, 35, 40, or 45 mmHg). In some embodiments, the method relates to patients having a DPG of at least 5 mmHg. In some embodiments, the method relates to patients having a DPG of at least 6 mmHg. In some embodiments, the method relates to patients having a DPG of at least 7 mmHg. In some embodiments, the method relates to patients having a DPG of at least 8 mmHg. In some embodiments, the method relates to patients having a DPG of at least 9 mmHg. In some embodiments, the method relates to patients having a DPG of at least 10 mmHg. In some embodiments, the method relates to patients having a DPG of at least 15 mmHg. In some embodiments, the method relates to patients having a DPG of at least 20 mmHg. In some embodiments, the method relates to patients having a DPG of at least 25 mmHg. In some embodiments, the method relates to patients having a DPG of at least 30 mmHg. In some embodiments, the method relates to patients having a DPG of at least 35 mmHg. In some embodiments, the method relates to patients having a DPG of at least 40 mmHg. In some embodiments, the method relates to patients having a DPG of at least 45 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to reducing the patient's DPG by at least 1 mmHg. In some embodiments, the method relates to reducing the patient's DPG by at least 2 mmHg. In some embodiments, the method relates to reducing the patient's DPG by at least 4 mmHg. In some embodiments, the method relates to reducing the patient's DPG by at least 6 mmHg. In some embodiments, the method relates to reducing the patient's DPG by at least 10 mmHg. In some embodiments, the method relates to reducing the patient's DPG by at least 15 mmHg. In some embodiments, the method relates to reducing the patient's DPG by at least 20 mmHg. In some embodiments, the method relates to reducing the patient's DPG by at least 25 mmHg. In some embodiments, the method relates to reducing the patient's DPG by at least 30 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to decreasing the patient's DPG by least 10% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's DPG by at least 1%. In some embodiments, the method relates to decreasing the patient's DPG by at least 5%. In some embodiments, the method relates to decreasing the patient's DPG by at least 10%. In some embodiments, the method relates to decreasing the patient's DPG by at least 15%. In some embodiments, the method relates to decreasing the patient's DPG by at least 20%. In some embodiments, the method relates to decreasing the patient's DPG by at least 25%. In some embodiments, the method relates to decreasing the patient's DPG by at least 30%. In some embodiments, the method relates to decreasing the patient's DPG by at least 35%. In some embodiments, the method relates to decreasing the patient's DPG by at least 40%. In some embodiments, the method relates to decreasing the patient's DPG by at least 45%. In some embodiments, the method relates to decreasing the patient's DPG by at least 50%. In some embodiments, the method relates to decreasing the patient's DPG by at least 55%. In some embodiments, the method relates to decreasing the patient's DPG by at least 60%. In some embodiments, the method relates to decreasing the patient's DPG by at least 65%. In some embodiments, the method relates to decreasing the patient's DPG by at least 70%. In some embodiments, the method relates to decreasing the patient's DPG by at least 75%. In some embodiments, the method relates to decreasing the patient's DPG by at least 80%. In some embodiments, the method relates to decreasing the patient's DPG by at least 85%. In some embodiments, the method relates to decreasing the patient's DPG by at least 90%. In some embodiments, the method relates to decreasing the patient's DPG by at least 95%. In some embodiments, the method relates to decreasing the patient's DPG by at least 100%.

### TPG

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has a transpulmonary gradient (TPG) of at least 10 mmHg (e.g., at least 10, 11, 12, 13, 14, 15, 20, 25, 30, 35, 40, 45, or 50 mmHg). In some embodiments, the method relates to patients having a TPG of at least 10 mmHg. In some embodiments, the method relates to patients having a TPG of at least 11 mmHg. In some embodiments, the method relates to patients having a TPG of at least 12 mmHg. In some embodiments, the method relates to patients having a TPG of at least 13 mmHg. In some embodiments, the method relates to patients having a TPG of at least 14 mmHg. In some embodiments, the method relates to patients having a TPG of at least 15 mmHg. In some embodiments, the method relates to patients having a TPG of at least 20 mmHg. In some embodiments, the method relates to patients having a TPG of at least 25 mmHg. In some embodiments, the method relates to patients having a TPG of at least 30 mmHg. In some embodiments, the method relates to patients having a TPG of at least 35 mmHg. In some embodiments, the method relates to patients having a TPG of at least 40 mmHg. In some embodiments, the method relates to patients having a TPG of at least 45 mmHg. In some embodiments, the method relates to patients having a TPG of at least 50 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to reducing the patient's TPG by at least 1 mmHg. In some embodiments, the method relates to reducing the patient's TPG by at least 2 mmHg. In some embodiments, the method relates to reducing the patient's TPG by at least 4 mmHg. In some embodiments, the method relates to reducing the patient's TPG by at least 6 mmHg. In some embodiments, the method relates to reducing the patient's TPG by at least 10 mmHg. In some embodiments, the method relates to reducing the patient's TPG by at least 15 mmHg. In some embodiments, the method relates to reducing the patient's TPG by at least 20 mmHg. In some embodiments, the method relates to reducing the patient's TPG by at least 25 mmHg. In some embodiments, the method relates to reducing the patient's TPG by at least 30 mmHg. In some embodiments, the method relates to reducing the patient's TPG by at least 40 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to decreasing the patient's TPG by least 10% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's TPG by at least 10%. In some embodiments, the method relates to decreasing the patient's TPG by at least 5%. In some embodiments, the method relates to decreasing the patient's TPG by at least 10%. In some embodiments, the method relates to decreasing the patient's TPG by at least 15%. In some embodiments, the method relates to decreasing the patient's TPG by at least 20%. In some embodiments, the method relates to decreasing the patient's TPG by at least 25%. In some embodiments, the method relates to decreasing the patient's TPG by at least 30%. In some embodiments, the method relates to decreasing the patient's TPG by at least 35%. In some embodiments, the method relates to decreasing the patient's TPG by at least 40%. In some embodiments, the method relates to decreasing the patient's TPG by at least 45%. In some embodiments, the method relates to decreasing the patient's TPG by at least 50%. In some embodiments, the method relates to decreasing the patient's TPG by at least 55%. In some embodiments, the method relates to decreasing the patient's TPG by at least 60%. In some embodiments, the method relates to decreasing the patient's TPG by at least 65%. In some embodiments, the method relates to decreasing the patient's TPG by at least 70%. In some embodiments, the method relates to decreasing the patient's TPG by at least 75%. In some embodiments, the method relates to decreasing the patient's TPG by at least 80%. In some embodiments, the method relates to decreasing the patient's TPG by at least 85%. In some embodiments, the method relates to decreasing the patient's TPG by at least 90%. In some embodiments, the method relates to decreasing the patient's TPG by at least 95%. In some embodiments, the method relates to decreasing the patient's TPG by at least 100%.

### BNP

Both BNP and NT-proBNP are markers of atrial and ventricular distension due to increased intracardiac pressure. The New York Heart Association (NYHA) developed a 4-stage functional classification system for congestive heart failure (CHF) based on the severity of symptoms. Studies have demonstrated that the measured concentrations of circulating BNP and NT-proBNP increase with the severity of CHF based on the NYHA classification. In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has a brain natriuretic peptide (BNP) level of at least 100 pg/mL (e.g., at least 100, 150, 200, 300, 400, 500, 600, 700, 800, 900, 1000, 3000, 5000, 10,000, 15,000, or 20,000 pg/mL). In some embodiments, the method relates to patient's having a BNP level of at least 100 pg/mL. In some embodiments, the method relates to patient's having a BNP level of at least 150 pg/mL. In some embodiments, the method relates to patient's having a BNP level of at least 200 pg/mL. In some embodiments, the method relates to patient's having a BNP level of at least 300 pg/mL. In some embodiments, the method relates to patient's having a BNP level of at least 400 pg/mL. In some embodiments, the method relates to patient's having a BNP level of at least 500 pg/mL. In some embodiments, the method relates to patient's having a BNP level of at least 600 pg/mL. In some embodiments, the method relates to patient's having a BNP level of at least 700 pg/mL. In some embodiments, the method relates to patient's having a BNP level of at least 800 pg/mL. In some embodiments, the method relates to patient's having a BNP level of at least 900 pg/mL. In some embodiments, the method relates to patient's having a BNP level of at least 1000 pg/mL. In some embodiments, the method relates to patient's having a BNP level of at least 5000 pg/mL. In some embodiments, the method relates to patient's having a BNP level of at least 10,000 pg/mL. In some embodiments, the method relates to patient's having a BNP level of at least 15,000 pg/mL. In some embodiments, the method relates to patient's having a BNP level of at least 20,000 pg/mL. In some embodiments, the method relates to treatment of a patient who has elevated BNP levels as compared to a healthy patient.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to reducing the patient's BNP levels by at least 10 pg/mL. In some embodiments, the method relates to reducing the patient's BNP levels by at least 50 pg/mL. In some embodiments, the method relates to reducing the patient's BNP levels by at least 100 pg/mL. In some embodiments, the method relates to reducing the patient's BNP levels by at least 200 pg/mL. In some embodiments, the method relates to reducing the patient's BNP levels by at least 300 pg/mL. In some embodiments, the method relates to reducing the patient's BNP levels by at least 400 pg/mL. In some embodiments, the method relates to reducing the patient's BNP levels by at least 500 pg/mL. In some embodiments, the method relates to reducing the patient's BNP levels by at least 600 pg/mL. In some embodiments, the method relates to reducing the patient's BNP levels by at least 700 pg/mL. In some embodiments, the method relates to reducing the patient's BNP levels by at least 800 pg/mL. In some embodiments, the method relates to reducing the patient's BNP levels by at least 900 pg/mL. In some embodiments, the method relates to reducing the patient's BNP levels by at least 1000 pg/mL. In some embodiments, the method relates to reducing the patient's BNP levels by at least 5000 pg/mL. In some embodiments, the method relates to reducing the patient's BNP levels to normal levels. In some embodiments, normal levels correspond to levels of <100 pg/mL.

In some embodiments, the method relates to reducing the patient's BNP by at least 5% (e.g., at least 5, 10, 15, 20, 25, 30, 35, 40, 45, 50, 55, 60, 65, 70, 75, 80, 85, 90, 95, or 100%). In some embodiments, the method relates to reducing the patient's BNP by at least 5%. In some embodiments, the method relates to reducing the patient's BNP by at least 10%. In some embodiments, the method relates to reducing the patient's BNP by at least 15%. In some embodiments, the method relates to reducing the patient's BNP by at least 20%. In some embodiments, the method relates to reducing the patient's BNP by at least 25%. In some embodiments, the method relates to reducing the patient's BNP by at least 30%. In some embodiments, the method relates to reducing the patient's BNP by at least 35%. In some embodiments, the method relates to reducing the patient's BNP by at least 40%. In some embodiments, the method relates to reducing the patient's BNP by at least 45%. In some embodiments, the method relates to reducing the patient's BNP by at least 50%. In some embodiments, the method relates to reducing the patient's BNP by at least 55%. In some embodiments, the method relates to reducing the patient's BNP by at least 60%. In some embodiments, the method relates to reducing the patient's BNP by at least 65%. In some embodiments, the method relates to reducing the patient's BNP by at least 70%. In some embodiments, the method relates to reducing the patient's BNP by at least 75%. In some embodiments, the method relates to reducing the patient's BNP by at least 80%. In some embodiments, the method relates to reducing the patient's BNP by at least 85%. In some embodiments, the method relates to reducing the patient's BNP by at least 90%. In some embodiments, the method relates to reducing the patient's BNP by at least 95%. In some embodiments, the method relates to reducing the patient's BNP by at least 100%.

### NT-proBNP

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has a NT-proBNP level of at least 100 pg/mL (e.g., at least 100, 150, 200, 300, 400, 500, 600, 700, 800, 900, 1000, 3000, 5000, 10,000, 15,000, 20,000, 25,000, or 30,000 pg/mL). In some embodiments, the method relates to patient's having a NT-proBNP level of at least 100 pg/mL. In some embodiments, the method relates to patient's having a NT-proBNP level of at least 150 pg/mL. In some embodiments, the method relates to patient's having a NT-proBNP level of at least 200 pg/mL. In some embodiments, the method relates to patient's having a NT-proBNP level of at least 300 pg/mL. In some embodiments, the method relates to patient's having a NT-proBNP level of at least 400 pg/mL. In some embodiments, the method relates to patient's having a NT-proBNP level of at least 500 pg/mL. In some embodiments, the method relates to patient's having a NT-proBNP level of at least 600 pg/mL. In some embodiments, the method relates to patient's having a NT-proBNP level of at least 700 pg/mL. In some embodiments, the method relates to patient's having a NT-proBNP level of at least 800 pg/mL. In some embodiments, the method relates to patient's having a NT-proBNP level of at least 900 pg/mL. In some embodiments, the method relates to patient's having a NT-proBNP level of at least 1000 pg/mL. In some embodiments, the method relates to patient's having a NT-proBNP level of at least 5000 pg/mL. In some embodiments, the method relates to patient's having a NT-proBNP level of at least 10,000 pg/mL. In some embodiments, the method relates to patient's having a NT-proBNP level of at least 15,000 pg/mL. In some embodiments, the method relates to patient's having a NT-proBNP level of at least 20,000 pg/mL. In some embodiments, the method relates to patient's having a NT-proBNP level of at least 25,000 pg/mL. In some embodiments, the method relates to patient's having a NT-proBNP level of at least 30,000 pg/mL. In some embodiments, the method relates to treatment of a patient who has elevated NT-proBNP levels as compared to a healthy patient.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to reducing the patient's NT-proBNP levels. In some embodiments, the method relates to reducing the patient's NT-proBNP by at least 10 pg/mL. In some embodiments, the method relates to reducing the patient's NT-proBNP by at least 50 pg/mL. In some embodiments, the method relates to reducing the patient's NT-proBNP by at least 100 pg/mL. In some embodiments, the method relates to reducing the patient's NT-proBNP by at least 200 pg/mL. In some embodiments, the method relates to reducing the patient's NT-proBNP by at least 300 pg/mL. In some embodiments, the method relates to reducing the patient's NT-proBNP by at least 400 pg/mL. In some embodiments, the method relates to reducing the patient's NT-proBNP by at least 500 pg/mL. In some embodiments, the method relates to reducing the patient's NT-proBNP by at least 600 pg/mL. In some embodiments, the method relates to reducing the patient's NT-proBNP by at least 700 pg/mL. In some embodiments, the method relates to reducing the patient's NT-proBNP by at least 800 pg/mL. In some embodiments, the method relates to reducing the patient's NT-proBNP by at least 900 pg/mL. In some embodiments, the method relates to reducing the patient's NT-proBNP by at least 1000 pg/mL. In some embodiments, the method relates to reducing the patient's NT-proBNP by at least 5000 pg/mL. In some embodiments, the method relates to reducing the patient's NT-proBNP by at least 10,000 pg/mL. In some embodiments, the method relates to reducing the patient's NT-proBNP by at least 15,000 pg/mL. In some embodiments, the method relates to reducing the patient's NT-proBNP by at least 20,000 pg/mL. In some embodiments, the method relates to reducing the patient's NT-proBNP by at least 25,000 pg/mL.

In some embodiments, the method relates to decreasing the patient's NT-proBNP levels to a normal level and maintaining their normal NT-proBNP levels. In some embodiments, the disclosure relates to methods of maintaining one or more hemodynamic parameters in the PcPH patient at a normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to maintaining the patient's NT-proBNP levels at a normal level. In some embodiments, the method relates to maintaining the patient's NT-proBNP level at less than 100 pg/mL. In some embodiments, the method relates to maintaining the patient's NT-proBNP level at less than 200 pg/mL. In some embodiments, the method relates to maintaining the patient's NT-proBNP level at less than 300 pg/mL. In some embodiments, the method relates to maintaining the patient's NT-proBNP level at less than 400 pg/mL.

In some embodiments, the method relates to reducing the patient's NT-proBNP by at least 5% (e.g., at least 5, 10, 15, 20, 25, 30, 35, 40, 45, 50, 55, 60, 65, 70, 75, 80, 85, 90, 95, or 100%). In some embodiments, the method relates to reducing the patient's NT-proBNP by at least 5%. In some embodiments, the method relates to reducing the patient's NT-proBNP by at least 10%. In some embodiments, the method relates to reducing the patient's NT-proBNP by at least 15%. In some embodiments, the method relates to reducing the patient's NT-proBNP by at least 20%. In some embodiments, the method relates to reducing the patient's NT-proBNP by at least 25%. In some embodiments, the method relates to reducing the patient's NT-proBNP by at least 30%. In some embodiments, the method relates to reducing the patient's NT-proBNP by at least 35%. In some embodiments, the method relates to reducing the patient's NT-proBNP by at least 40%. In some embodiments, the method relates to reducing the patient's NT-proBNP by at least 45%. In some embodiments, the method relates to reducing the patient's NT-proBNP by at least 50%. In some embodiments, the method relates to reducing the patient's NT-proBNP by at least 55%. In some embodiments, the method relates to reducing the patient's NT-proBNP by at least 60%. In some embodiments, the method relates to reducing the patient's NT-proBNP by at least 65%. In some embodiments, the method relates to reducing the patient's NT-proBNP by at least 70%. In some embodiments, the method relates to reducing the patient's NT-proBNP by at least 75%. In some embodiments, the method relates to reducing the patient's NT-proBNP by at least 80%. In some embodiments, the method relates to reducing the patient's NT-proBNP by at least 85%. In some embodiments, the method relates to reducing the patient's NT-proBNP by at least 90%. In some embodiments, the method relates to reducing the patient's NT-proBNP by at least 95%. In some embodiments, the method relates to reducing the patient's NT-proBNP by at least 100%.

### Smooth Muscle Hypertrophy

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has smooth muscle hypertrophy. In some embodiments, the disclosure relates to methods of adjusting one or more parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to decreasing smooth muscle hypertrophy in the patient. In some embodiments, the method relates to decreasing the patient's smooth muscle hypertrophy by least 10% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's smooth muscle hypertrophy by at least 10%. In some embodiments, the method relates to decreasing the patient's smooth muscle hypertrophy by at least 5%. In some embodiments, the method relates to decreasing the patient's smooth muscle hypertrophy by at least 10%. In some embodiments, the method relates to decreasing the patient's smooth muscle hypertrophy by at least 15%. In some embodiments, the method relates to decreasing the patient's smooth muscle hypertrophy by at least 20%. In some embodiments, the method relates to decreasing the patient's smooth muscle hypertrophy by at least 25%. In some embodiments, the method relates to decreasing the patient's smooth muscle hypertrophy by at least 30%. In some embodiments, the method relates to decreasing the patient's smooth muscle hypertrophy by at least 35%. In some embodiments, the method relates to decreasing the patient's smooth muscle hypertrophy by at least 40%. In some embodiments, the method relates to decreasing the patient's smooth muscle hypertrophy by at least 45%. In some embodiments, the method relates to decreasing the patient's smooth muscle hypertrophy by at least 50%. In some embodiments, the method relates to decreasing the patient's smooth muscle hypertrophy by at least 55%. In some embodiments, the method relates to decreasing the patient's smooth muscle hypertrophy by at least 60%. In some embodiments, the method relates to decreasing the patient's smooth muscle hypertrophy by at least 65%. In some embodiments, the method relates to decreasing the patient's smooth muscle hypertrophy by at least 70%. In some embodiments, the method relates to decreasing the patient's smooth muscle hypertrophy by at least 75%. In some embodiments, the method relates to decreasing the patient's smooth muscle hypertrophy by at least 80%. In some embodiments, the method relates to decreasing the patient's smooth muscle hypertrophy by at least 85%. In some embodiments, the method relates to decreasing the patient's smooth muscle hypertrophy by at least 90%. In some embodiments, the method relates to decreasing the patient's smooth muscle hypertrophy by at least 95%. In some embodiments, the method relates to decreasing the patient's smooth muscle hypertrophy by at least 100%.

### Pulmonary Arteriole Muscularity

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has increased pulmonary arteriole muscularity. In some embodiments, the disclosure relates to methods of adjusting one or more parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to decreasing the patient's pulmonary arteriole muscularity in the patient. In some embodiments, the method relates to decreasing the patient's pulmonary arteriole muscularity by least 10% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's pulmonary arteriole muscularity by at least 1%. In some embodiments, the method relates to decreasing the patient's pulmonary arteriole muscularity by at least 5%. In some embodiments, the method relates to decreasing the patient's pulmonary arteriole muscularity by at least 10%. In some embodiments, the method relates to decreasing the patient's pulmonary arteriole muscularity by at least 15%. In some embodiments, the method relates to decreasing the patient's pulmonary arteriole muscularity by at least 20%. In some embodiments, the method relates to decreasing the patient's pulmonary arteriole muscularity by at least 25%. In some embodiments, the method relates to decreasing the patient's pulmonary arteriole muscularity by at least 30%. In some embodiments, the method relates to decreasing the patient's pulmonary arteriole muscularity by at least 35%. In some embodiments, the method relates to decreasing the patient's pulmonary arteriole muscularity by at least 40%. In some embodiments, the method relates to decreasing the patient's pulmonary arteriole muscularity by at least 45%. In some embodiments, the method relates to decreasing the patient's pulmonary arteriole muscularity by at least 50%. In some embodiments, the method relates to decreasing the patient's pulmonary arteriole muscularity by at least 55%. In some embodiments, the method relates to decreasing the patient's pulmonary arteriole muscularity by at least 60%. In some embodiments, the method relates to decreasing the patient's pulmonary arteriole muscularity by at least 65%. In some embodiments, the method relates to decreasing the patient's pulmonary arteriole muscularity by at least 70%. In some embodiments, the method relates to decreasing the patient's pulmonary arteriole muscularity by at least 75%. In some embodiments, the method relates to decreasing the patient's pulmonary arteriole muscularity by at least 80%. In some embodiments, the method relates to decreasing the patient's pulmonary arteriole muscularity by at least 85%. In some embodiments, the method relates to decreasing the patient's pulmonary arteriole muscularity by at least 90%. In some embodiments, the method relates to decreasing the patient's pulmonary arteriole muscularity by at least 95%. In some embodiments, the method relates to decreasing the patient's pulmonary arteriole muscularity by at least 100%.

### Rate of Hospitalization

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the method reduces the patient's hospitalization rate by at least 10% (e.g., at least 1%, 2%, 3%, 4%, 5%, 10%, 15%, 20%, 25%, 30%, 3%, 40%, 45%, 50%, 55%, 60%, 65%, 70%, 75%, 80%, 85%, 90%, 95%, or 100%. In some embodiments, the method relates to reducing the patient's hospitalization rate by at least 1%. In some embodiments, the method relates to reducing the patient's hospitalization rate by at least 2%. In some embodiments, the method relates to reducing the patient's hospitalization rate by at least 3%. In some embodiments, the method relates to reducing the patient's hospitalization rate by at least 4%. In some embodiments, the method relates to reducing the patient's hospitalization rate by at least 5%. In some embodiments, the method relates to reducing the patient's hospitalization rate by at least 10%. In some embodiments, the method relates to reducing the patient's hospitalization rate by at least 15%. In some embodiments, the method relates to reducing the patient's hospitalization rate by at least 20%. In some embodiments, the method relates to reducing the patient's hospitalization rate by at least 25%. In some embodiments, the method relates to reducing the patient's hospitalization rate by at least 30%. In some embodiments, the method relates to reducing the patient's hospitalization rate by at least 35%. In some embodiments, the method relates to reducing the patient's hospitalization rate by at least 40%. In some embodiments, the method relates to reducing the patient's hospitalization rate by at least 45%. In some embodiments, the method relates to reducing the patient's hospitalization rate by at least 50%. In some embodiments, the method relates to reducing the patient's hospitalization rate by at least 55%. In some embodiments, the method relates to reducing the patient's hospitalization rate by at least 60%. In some embodiments, the method relates to reducing the patient's hospitalization rate by at least 65%. In some embodiments, the method relates to reducing the patient's hospitalization rate by at least 70%. In some embodiments, the method relates to reducing the patient's hospitalization rate by at least 75%. In some embodiments, the method relates to reducing the patient's hospitalization rate by at least 80%. In some embodiments, the method relates to reducing the patient's hospitalization rate by at least 85%. In some embodiments, the method relates to reducing the patient's hospitalization rate by at least 90%. In some embodiments, the method relates to reducing the patient's hospitalization rate by at least 95%. In some embodiments, the method relates to reducing the patient's hospitalization rate by at least 100%. In some embodiments, the method reduces the risk of hospitalization for one or more complications associated with PcPH.

### Quality of Life

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the method increases the patient's quality of life by at least 10% (e.g., at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%). In some embodiments, the method relates to increasing the patient's quality of life by at least 10%. In some embodiments, the method relates to increasing the patient's quality of life by at least 2%. In some embodiments, the method relates to increasing the patient's quality of life by at least 3%. In some embodiments, the method relates to increasing the patient's quality of life by at least 4%. In some embodiments, the method relates to increasing the patient's quality of life by at least 5%. In some embodiments, the method relates to increasing the patient's quality of life by at least 10%. In some embodiments, the method relates to increasing the patient's quality of life by at least 15%. In some embodiments, the method relates to increasing the patient's quality of life by at least 20%. In some embodiments, the method relates to increasing the patient's quality of life by at least 25%. In some embodiments, the method relates to increasing the patient's quality of life by at least 30%. In some embodiments, the method relates to increasing the patient's quality of life by at least 35%. In some embodiments, the method relates to increasing the patient's quality of life by at least 40%. In some embodiments, the method relates to increasing the patient's quality of life by at least 45%. In some embodiments, the method relates to increasing the patient's quality of life by at least 50%. In some embodiments, the method relates to increasing the patient's quality of life by at least 55%. In some embodiments, the method relates to increasing the patient's quality of life by at least 60%. In some embodiments, the method relates to increasing the patient's quality of life by at least 65%. In some embodiments, the method relates to increasing the patient's quality of life by at least 70%. In some embodiments, the method relates to increasing the patient's quality of life by at least 75%. In some embodiments, the method relates to increasing the patient's quality of life by at least 80%. In some embodiments, the method relates to increasing the patient's quality of life by at least 85%. In some embodiments, the method relates to increasing the patient's quality of life by at least 90%. In some embodiments, the method relates to increasing the patient's quality of life by at least 95%. In some embodiments, the method relates to increasing the patient's quality of life by at least 100%.

In some embodiments, the patient's quality of life is measured using the Cambridge Pulmonary Hypertension Outcome Review (CAMPHOR). In some embodiments, the patient's quality of life is measured using PAH-SYMPACT®. In some embodiments, the patient's quality of life is measured using the Medical Outcomes Survey Short Form-36 (SF-36). In some embodiments, the patient's quality of life is measured using the Euro Quality of Life (EuroQol). In some embodiments, the patient's quality of life is measured using the Euro Quality of Life-5 dimensions (EQ-5D). In some embodiments, the patient's quality of life is measured using the Euro Quality of Life-5 dimensions 5-levels (EQ-5D-5L). In some embodiments, the patient's quality of life is measured using the Kansas City Cardiomyopathy Questionnaire (KCCQ).

### Diastolic Function

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the method increases the patient's LV diastolic function by at least 5% (e.g., at least 5, 10, 15, 20, 25, 30, 35, 40, 45, 50, 55, 60, 65, 70, 75, 80, 85, 90, 95, or 100%). In some embodiments, the method relates to increasing the patient's LV diastolic function by at least 5%. In some embodiments, the method relates to increasing the patient's LV diastolic function by at least 10%. In some embodiments, the method relates to increasing the patient's LV diastolic function by at least 15%. In some embodiments, the method relates to increasing the patient's LV diastolic function by at least 20%. In some embodiments, the method relates to increasing the patient's LV diastolic function by at least 25%. In some embodiments, the method relates to increasing the patient's LV diastolic function by at least 30%. In some embodiments, the method relates to increasing the patient's LV diastolic function by at least 35%. In some embodiments, the method relates to increasing the patient's LV diastolic function by at least 40%. In some embodiments, the method relates to increasing the patient's LV diastolic function by at least 45%. In some embodiments, the method relates to increasing the patient's LV diastolic function by at least 50%. In some embodiments, the method relates to increasing the patient's LV diastolic function by at least 55%. In some embodiments, the method relates to increasing the patient's LV diastolic function by at least 60%. In some embodiments, the method relates to increasing the patient's LV diastolic function by at least 65%. In some embodiments, the method relates to increasing the patient's LV diastolic function by at least 70%. In some embodiments, the method relates to increasing the patient's LV diastolic function by at least 75%. In some embodiments, the method relates to increasing the patient's LV diastolic function by at least 80%. In some embodiments, the method relates to increasing the patient's LV diastolic function by at least 85%. In some embodiments, the method relates to increasing the patient's LV diastolic function by at least 90%. In some embodiments, the method relates to increasing the patient's LV diastolic function by at least 95%. In some embodiments, the method relates to increasing the patient's LV diastolic function by at least 100%.

### Ejection Fraction

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has an ejection fraction of less than 10% (e.g., less than 10, 15, 20, 25, 30, 35, 40, or 45%). In some embodiments, the method relates to patient's having an ejection fraction of less than 10%. In some embodiments, the method relates to patient's having an ejection fraction of less than 15%. In some embodiments, the method relates to patient's having an ejection fraction of less than 20%. In some embodiments, the method relates to patient's having an ejection fraction of less than 25%. In some embodiments, the method relates to patient's having an ejection fraction of less than 30%. In some embodiments, the method relates to patient's having an ejection fraction of less than 35%. In some embodiments, the method relates to patient's having an ejection fraction of less than 40%. In some embodiments, the method relates to patient's having an ejection fraction of less than 45%. In some embodiments, the method relates to patient's having an ejection fraction of less than 50%. In some embodiments, the method relates to patient's having an ejection fraction of less than 55%.

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has an ejection fraction of at least 35% (e.g., at least 35, 40, 45, 50, or 55%). In some embodiments, the method relates to patient's having an ejection fraction of at least 35%. In some embodiments, the method relates to patient's having an ejection fraction of at least 40%. In some embodiments, the method relates to patient's having an ejection fraction of at least 45%. In some embodiments, the method relates to patient's having an ejection fraction of at least 50%. In some embodiments, the method relates to patient's having an ejection fraction of at least 55%. In some embodiments, the ejection fraction is the right ventricular ejection fraction. In some embodiments, the ejection fraction is the left ventricular ejection fraction (LVEF). In some embodiments, the ejection fraction is measured using an echocardiogram. In some embodiments, the patient has a preserved left ventricular ejection fraction. In some embodiments, the disclosure relates to methods of adjusting one or more hemodynamic parameters in the PcPH patient toward a more normal level (e.g., >50% ejection fraction), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to reducing to increasing the patient's ejection fraction by least 1%. In some embodiments, the method relates to increasing the patient's ejection fraction by at least 5%. In some embodiments, the method relates to increasing the patient's ejection fraction by at least 10%. In some embodiments, the method relates to increasing the patient's ejection fraction by at least 15%. In some embodiments, the method relates to increasing the patient's ejection fraction by at least 20%. In some embodiments, the method relates to increasing the patient's ejection fraction by at least 25%. In some embodiments, the method relates to increasing the patient's ejection fraction by at least 30%. In some embodiments, the method relates to increasing the patient's ejection fraction by at least 35%. In some embodiments, the method relates to increasing the patient's ejection fraction by at least 40%. In some embodiments, the method relates to increasing the patient's ejection fraction by at least 45%. In some embodiments, the method relates to increasing the patient's ejection fraction by at least 50%. In some embodiments, the method relates to increasing the patient's ejection fraction by at least 55%. In some embodiments, the method relates to increasing the patient's ejection fraction by at least 60%. In some embodiments, the method relates to increasing the patient's ejection fraction by at least 65%. In some embodiments, the method relates to increasing the patient's ejection fraction by at least 70%. In some embodiments, the method relates to increasing the patient's ejection fraction by at least 75%. In some embodiments, the method relates to increasing the patient's ejection fraction by at least 80%. In some embodiments, the method relates to increasing the patient's ejection fraction by at least 85%. In some embodiments, the method relates to increasing the patient's ejection fraction by at least 90%. In some embodiments, the method relates to increasing the patient's ejection fraction by at least 95%. In some embodiments, the method relates to increasing the patient's ejection fraction by at least 100%.

### Ventricular Function

In certain aspects, the disclosure relates to methods of improving or maintaining ventricular function (e.g., left ventricular function or right ventricular function) in PcPH comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). Echocardiography is a useful noninvasive screening tool for determining the severity of pulmonary hypertension in a patient. Improvement or maintenance of ventricular function (e.g., left ventricular function or right ventricular function) can be assessed by many echocardiographic measurements. One such quantitative approach to assess ventricular function is the measurement of the tricuspid annular plane systolic excursion (TAPSE). The TAPSE estimates RV systolic function by measuring the level of systolic excursion of the lateral tricuspid valve annulus towards the apex. Other echocardiographic measurements that may be used to assess maintenance and/or improvements in ventricular function include, but are not limited to, right ventricular fractional area change (RVFAC), right ventricular end-diastolic area (RVEDA), right ventricular end-systolic area (RVESA), right ventricular free wall thickness (RVFWT), right ventricular ejection fraction (RVEF), right ventricular-pulmonary artery (RV-PA) coupling, pulmonary arterial systolic pressure (PASP), right ventricular systolic pressure (RVSP), pulmonary artery acceleration time (PAAT), tricuspid regurgitation velocity (TRV), left ventricular hypertrophy, and right ventricular hypertrophy.

### TAPSE

The tricuspid annular plane systolic excursion (TAPSE) can be obtained using echocardiography and represents a measure of RV longitudinal function. The TAPSE has previously been shown to have good correlations with parameters estimating RV global systolic function. A TAPSE <17 mm is highly suggestive of RV systolic dysfunction. In some embodiments, an improvement or maintenance of right ventricular function in a PcPH patient is measured as an increase in TAPSE. In some embodiments, a PcPH patient with an improvement or maintenance of right ventricular function has a TAPSE between 20 mm-28 mm. In some embodiments, a PcPH patient with an improvement or maintenance of right ventricular function has a TAPSE of at least 20 mm. In some embodiments, a PcPH patient with an improvement or maintenance of right ventricular function has a TAPSE of at least 22 mm. In some embodiments, a PcPH patient with an improvement or maintenance of right ventricular function has a TAPSE of at least 24 mm. In some embodiments, a PcPH patient with an improvement or maintenance of right ventricular function has a TAPSE of at least 26 mm. In some embodiments, a PcPH patient with an improvement or maintenance of right ventricular function has a TAPSE of at least 28 mm. In some embodiments, the TAPSE is measured using echocardiography.

In some embodiments, a PcPH patient with an improvement or maintenance of right ventricular function has a TAPSE between 16 mm-30 mm. In some embodiments, a PcPH patient with an improvement or maintenance of right ventricular function has a TAPSE between 18 mm-28 mm. In some embodiments, a PcPH patient with an improvement or maintenance of right ventricular function has a TAPSE of at least 18 mm. In some embodiments, the TAPSE is measured using echocardiography.

### PASP and RVSP

In certain aspects, the disclosure relates to methods of treating, preventing, or reducing the progression rate and/or severity of PcPH comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has a pulmonary arterial systolic pressure (PASP) of at least 30 mmHg (e.g., at least 30, 35, 40, 45, 50, 55, 60, 65, 70, 75, or 80 mmHg). In some embodiments, the method relates to patients having a PASP of at least 30 mmHg. In some embodiments, the method relates to patients having a PASP of at least 35 mmHg. In some embodiments, the method relates to patients having a PASP of at least 40 mmHg. In some embodiments, the method relates to patients having a PASP of at least 45 mmHg. In some embodiments, the method relates to patients having a PASP of at least 50 mmHg. In some embodiments, the method relates to patients having a PASP of at least 55 mmHg. In some embodiments, the method relates to patients having a PASP of at least 60 mmHg. In some embodiments, the method relates to patients having a PASP of at least 65 mmHg. In some embodiments, the method relates to patients having a PASP of at least 70 mmHg. In some embodiments, the method relates to patients having a PASP of at least 75 mmHg. In some embodiments, the method relates to patients having a PASP of at least 80 mmHg. In some embodiments, the PASP is a resting PASP. In some embodiments, the PASP is determined using the tricuspid regurgitation velocity (TRV) and right arterial (RA) pressure. In some embodiments, the PASP is determined using the following formula:

$$P A S P = {TRV}^{2} \times 4 + RA pressure$$

TRV has been shown to correlate with PASP at rest and with exercise. The pressure gradient between the right ventricle and the right atrium can be calculated using the modified Bernoulli equation (Δp=4V2).

In some embodiments, the right ventricular systolic pressure (RVSP) is equal to PASP. In some embodiments, the RVSP is measured in the absence of right ventricular outflow tract obstruction. In some embodiments, the RVSP is determined using the following formula:

$$RVSP = 4 {V}^{2} + R A P$$

In the above formula, V represents the peak tricuspid regurgitant jet velocity and RAP is the mean right atrial pressure. RVSP is frequently used for estimating PASP.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to improving the pulmonary arterial systolic pressure (PASP) in the patient. In some embodiments, the method relates to reducing PASP. In some embodiments, the method relates to reducing the patient's PASP by at least 1 mmHg (e.g., at least 1, 2, 3, 5, 7, 10, 12, 15, 20, 25, 30, or 35 mmHg). In some embodiments, the method relates to reducing the patient's PASP by at least 2 mmHg. In some embodiments, the method relates to reducing the patient's PASP by at least 3 mmHg. In certain embodiments, the method relates to reducing the patient's PASP by at least 5 mmHg. In certain embodiments, the method relates to reducing the patient's PASP by at least 7 mmHg. In certain embodiments, the method relates to reducing the patient's PASP by at least 10 mmHg. In certain embodiments, the method relates to reducing the patient's PASP by at least 12 mmHg. In certain embodiments, the method relates to reducing the patient's PASP by at least 15 mmHg. In certain embodiments, the method relates to reducing the patient's PASP by at least 20 mmHg. In certain embodiments, the method relates to reducing the patient's PASP by at least 25 mmHg. In certain embodiments, the method relates to reducing the patient's PASP by at least 30 mmHg. In certain embodiments, the method relates to reducing the patient's PASP by at least 35 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to reducing the patient's PASP by least 10% (e.g., at least 1, 5, 10, 15, 20, 25, 30, 35, 40, 45, 50, 55, 60, 65, 70, 75, 80, 85, 90, 95, or 100%). In some embodiments, the method relates to reducing the patient's PASP by at least 10%. In some embodiments, the method relates to reducing the patient's PASP by at least 5%. In some embodiments, the method relates to reducing the patient's PASP by at least 10%. In some embodiments, the method relates to reducing the patient's PASP by at least 15%. In some embodiments, the method relates to reducing the patient's PASP by at least 20%. In some embodiments, the method relates to reducing the patient's PASP by at least 25%. In some embodiments, the method relates to reducing the patient's PASP by at least 30%. In some embodiments, the method relates to reducing the patient's PASP by at least 35%. In some embodiments, the method relates to reducing the patient's PASP by at least 40%. In some embodiments, the method relates to reducing the patient's PASP by at least 45%. In some embodiments, the method relates to reducing the patient's PASP by at least 50%. In some embodiments, the method relates to reducing the patient's PASP by at least 55%. In some embodiments, the method relates to reducing the patient's PASP by at least 60%. In some embodiments, the method relates to reducing the patient's PASP by at least 65%. In some embodiments, the method relates to reducing the patient's PASP by at least 70%. In some embodiments, the method relates to reducing the patient's PASP by at least 75%. In some embodiments, the method relates to reducing the patient's PASP by at least 80%. In some embodiments, the method relates to reducing the patient's PASP by at least 85%. In some embodiments, the method relates to reducing the patient's PASP by at least 90%. In some embodiments, the method relates to reducing the patient's PASP by at least 95%. In some embodiments, the method relates to reducing the patient's PASP by at least 100%.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to improving the right ventricular systolic pressure (RVSP) in the patient. In some embodiments, the method relates to reducing RVSP. In some embodiments, the method relates to reducing the patient's RVSP by at least 1 mmHg (e.g., at least 1, 2, 3, 5, 7, 10, 12, 15, 20, 25, 30, or 35 mmHg). In some embodiments, the method relates to reducing the patient's RVSP by at least 2 mmHg. In some embodiments, the method relates to reducing the patient's RVSP by at least 3 mmHg. In certain embodiments, the method relates to reducing the patient's RVSP by at least 5 mmHg. In certain embodiments, the method relates to reducing the patient's RVSP by at least 7 mmHg. In certain embodiments, the method relates to reducing the patient's RVSP by at least 10 mmHg. In certain embodiments, the method relates to reducing the patient's RVSP by at least 12 mmHg. In certain embodiments, the method relates to reducing the patient's RVSP by at least 15 mmHg. In certain embodiments, the method relates to reducing the patient's RVSP by at least 20 mmHg. In certain embodiments, the method relates to reducing the patient's RVSP by at least 25 mmHg. In certain embodiments, the method relates to reducing the patient's RVSP by at least 30 mmHg. In certain embodiments, the method relates to reducing the patient's RVSP by at least 35 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to reducing the patient's RVSP by least 1% (e.g., at least 1, 5, 10, 15, 20, 25, 30, 35, 40, 45, 50, 55, 60, 65, 70, 75, 80, 85, 90, 95, or 100%). In some embodiments, the method relates to reducing the patient's RVSP by at least 5%. In some embodiments, the method relates to reducing the patient's RVSP by at least 10%. In some embodiments, the method relates to reducing the patient's RVSP by at least 15%. In some embodiments, the method relates to reducing the patient's RVSP by at least 20%. In some embodiments, the method relates to reducing the patient's RVSP by at least 25%. In some embodiments, the method relates to reducing the patient's RVSP by at least 30%. In some embodiments, the method relates to reducing the patient's RVSP by at least 35%. In some embodiments, the method relates to reducing the patient's RVSP by at least 40%. In some embodiments, the method relates to reducing the patient's RVSP by at least 45%. In some embodiments, the method relates to reducing the patient's RVSP by at least 50%. In some embodiments, the method relates to reducing the patient's RVSP by at least 55%. In some embodiments, the method relates to reducing the patient's RVSP by at least 60%. In some embodiments, the method relates to reducing the patient's RVSP by at least 65%. In some embodiments, the method relates to reducing the patient's RVSP by at least 70%. In some embodiments, the method relates to reducing the patient's RVSP by at least 75%. In some embodiments, the method relates to reducing the patient's RVSP by at least 80%. In some embodiments, the method relates to reducing the patient's RVSP by at least 85%. In some embodiments, the method relates to reducing the patient's RVSP by at least 90%. In some embodiments, the method relates to reducing the patient's RVSP by at least 95%. In some embodiments, the method relates to reducing the patient's RVSP by at least 100%.

### RV-PA Coupling

Right ventricular dysfunction can occur in PcPH and is a factor affecting prognosis. Energy transfer between ventricle contractility and arterial afterload is termed coupling. Energy transfer specifically between the right ventricle (RV) and pulmonary artery is termed right ventricle-pulmonary artery (RV-PA) coupling. In some embodiments, right ventricular dysfunction is due to a decrease in RV-PA coupling. RV-PA coupling can be estimated non-invasively as a ratio of TAPSE/PASP values. In some embodiments, a TAPSE/PASP ratio of ≥0.31 mm/mm Hg may be associated with a better prognosis and reduced risk of clinical worsening. In some embodiments, the improvement in RV-PA coupling is due to an improvement in PASP. In some embodiments, the calculation of RV-PA coupling is dependent upon paired results for three parameters (e.g., TRV, RAP, and TAPSE).

In certain aspects, the disclosure relates to methods of treating, preventing, or reducing the progression rate and/or severity of PcPH comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has a TAPSE/PASP ratio less than 0.31 mm/mmHg (e.g., less than 0.3, 0.25, 0.2, 0.15, or 0.1 mm/mmHg). In some embodiments, the method relates to patients having a TAPSE/PASP ratio less than 0.31 mm/mmHg. In some embodiments, the method relates to patients having a TAPSE/PASP ratio less than 0.3 mm/mmHg. In some embodiments, the method relates to patients having a TAPSE/PASP ratio less than 0.25 mm/mmHg. In some embodiments, the method relates to patients having a TAPSE/PASP ratio less than 0.2 mm/mmHg. In some embodiments, the method relates to patients having a TAPSE/PASP ratio less than 0.15 mm/mmHg. In some embodiments, the method relates to patients having a TAPSE/PASP ratio less than 0.1 mm/mmHg. In some embodiments, the method relates to patients having a decreased TAPSE/PASP ratio as compared to a normal TAPSE/PASP ratio.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to improving or maintaining the right ventricular function in the patient. In some embodiments, a PcPH patient with an improvement or maintenance of right ventricular function has a TAPSE/PASP ratio greater than 0.3 mm/mmHg (e.g., greater than 0.31, 0.32, 0.33, 0.34, or 0.35 mm/mmHg). In some embodiments, a PcPH patient with an improvement or maintenance of right ventricular function has a TAPSE/PASP ratio greater than 0.31 mm/mmHg. In some embodiments, a PcPH patient with an improvement or maintenance of right ventricular function has a TAPSE/PASP ratio greater than 0.32 mm/mmHg. In some embodiments, a PcPH patient with an improvement or maintenance of right ventricular function has a TAPSE/PASP ratio greater than 0.33 mm/mmHg. In some embodiments, a PcPH patient with an improvement or maintenance of right ventricular function has a TAPSE/PASP ratio greater than 0.34 mm/mmHg. In some embodiments, a PcPH patient with an improvement or maintenance of right ventricular function has a TAPSE/PASP ratio greater than 0.35 mm/mmHg. In some embodiments, the improvement in right ventricular function is an increase in TAPSE/PASP ratio. In some embodiments, the method relates to increasing the TAPSE/PASP ratio. In some embodiments, the method relates to increasing the patient's TAPSE/PASP ratio by at least 0.05 mm/mmHg. In some embodiments, the method relates to increasing the patient's TAPSE/PASP ratio by at least 0.07 mm/mmHg. In some embodiments, the method relates to increasing the patient's TAPSE/PASP ratio by at least 0.10 mm/mmHg. In some embodiments, the method relates to increasing the patient's TAPSE/PASP ratio by at least 0.12 mm/mmHg. In some embodiments, the method relates to increasing the patient's TAPSE/PASP ratio by at least 0.15 mm/mmHg. In some embodiments, the method relates to increasing the patient's TAPSE/PASP ratio by at least 0.18 mm/mmHg. In some embodiments, the method relates to increasing the patient's TAPSE/PASP ratio by at least 0.20 mm/mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to increasing the patient's TAPSE/PASP ratio by least 10% (e.g., at least 1, 5, 10, 15, 20, 25, 30, 35, 40, 45, 50, 55, 60, 65, 70, 75, 80, 85, 90, 95, or 100%). In some embodiments, the method relates to increasing the patient's TAPSE/PASP ratio by at least 5%. In some embodiments, the method relates to increasing the patient's TAPSE/PASP ratio by at least 10%. In some embodiments, the method relates to increasing the patient's TAPSE/PASP ratio by at least 15%. In some embodiments, the method relates to increasing the patient's TAPSE/PASP ratio by at least 20%. In some embodiments, the method relates to increasing the patient's TAPSE/PASP ratio by at least 25%. In some embodiments, the method relates to increasing the patient's TAPSE/PASP ratio by at least 30%. In some embodiments, the method relates to increasing the patient's TAPSE/PASP ratio by at least 35%. In some embodiments, the method relates to increasing the patient's TAPSE/PASP ratio by at least 40%. In some embodiments, the method relates to increasing the patient's TAPSE/PASP ratio by at least 45%. In some embodiments, the method relates to increasing the patient's TAPSE/PASP ratio by at least 50%. In some embodiments, the method relates to increasing the patient's TAPSE/PASP ratio by at least 55%. In some embodiments, the method relates to increasing the patient's TAPSE/PASP ratio by at least 60%. In some embodiments, the method relates to increasing the patient's TAPSE/PASP ratio by at least 65%. In some embodiments, the method relates to increasing the patient's TAPSE/PASP ratio by at least 70%. In some embodiments, the method relates to increasing the patient's TAPSE/PASP ratio by at least 75%. In some embodiments, the method relates to increasing the patient's TAPSE/PASP ratio by at least 80%. In some embodiments, the method relates to increasing the patient's TAPSE/PASP ratio by at least 85%. In some embodiments, the method relates to increasing the patient's TAPSE/PASP ratio by at least 90%. In some embodiments, the method relates to increasing the patient's TAPSE/PASP ratio by at least 100%.

### RVFAC, RVEDA, and RVESA

Right ventricular fractional area change (RVFAC) is a non-invasive quantitative measure of right ventricular function. RVFAC can be calculated using the formula [(RVEDA-RVESA)/RVEDA]*100. In some embodiments, the RVFAC is measured using echocardiography. In some embodiments, normal RVFAC is approximately 47.5±8.6% in men and approximately 50.9±8.0% in women. See, e.g., Kou S, et al. European Heart Journal—Cardiovascular Imaging. 2014 Jun. 1; 15(6):680-90. In some embodiments, PcPH patients have a decrease in RVFAC.

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has a RVFAC of less than 20% (e.g., less than 20, 25, 30, 35, or 40%). In some embodiments, the method relates to patients having a RVFAC of less than 25%. In some embodiments, the method relates to patients having a RVFAC of less than 30%. In some embodiments, the method relates to patients having a RVFAC of less than 35%. In some embodiments, the method relates to patients having a RVFAC of less than 40%.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to improving or maintaining the right ventricular function in the patient. In some embodiments, the improvement or maintenance of right ventricular function is due to an increase in right ventricular fractional area change (RVFAC). In some embodiments, a PcPH patient with an improvement or maintenance of right ventricular function has a RVFAC between 32-56%.

In some embodiments, a PcPH patient with an improvement or maintenance of right ventricular function has a RVFAC of at least 32%. In some embodiments, a PcPH patient with an improvement or maintenance of right ventricular function has a RVFAC of at least 34%. In some embodiments, a PcPH patient with an improvement or maintenance of right ventricular function has a RVFAC of at least 35%. In some embodiments, a PcPH patient with an improvement or maintenance of right ventricular function has a RVFAC of at least 36%. In some embodiments, a PcPH patient with an improvement or maintenance of right ventricular function has a RVFAC of at least 38%. In some embodiments, a PcPH patient with an improvement or maintenance of right ventricular function has a RVFAC of at least 40%. In some embodiments, a PcPH patient with an improvement or maintenance of right ventricular function has a RVFAC of at least 42%. In some embodiments, a PcPH patient with an improvement or maintenance of right ventricular function has a RVFAC of at least 44%. In some embodiments, a PcPH patient with an improvement or maintenance of right ventricular function has a RVFAC of at least 46%. In some embodiments, a PcPH patient with an improvement or maintenance of right ventricular function has a RVFAC of at least 48%. In some embodiments, a PcPH patient with an improvement or maintenance of right ventricular function has a RVFAC of at least 50%. In some embodiments, a PcPH patient with an improvement or maintenance of right ventricular function has a RVFAC of at least 52%. In some embodiments, a PcPH patient with an improvement or maintenance of right ventricular function has a RVFAC of at least 54%. In some embodiments, a PcPH patient with an improvement or maintenance of right ventricular function has a RVFAC of at least 56%.

In some embodiments, the disclosure relates to methods of adjusting one or more echocardiogram parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to decreasing the patient's RVEDA by least 1% (e.g., at least 1, 2, 3, 4, 5, 6, 7, 8, 9, 10, 12, 14, 16, 18, or 20%). In some embodiments, the method relates to increasing the patient's RVFAC by least 2%. In some embodiments, the method relates to increasing the patient's RVFAC by least 3%. In some embodiments, the method relates to increasing the patient's RVFAC by least 4%. In some embodiments, the method relates to increasing the patient's RVFAC by least 5%. In some embodiments, the method relates to increasing the patient's RVFAC by least 6%. In some embodiments, the method relates to increasing the patient's RVFAC by least 7%. In some embodiments, the method relates to increasing the patient's RVFAC by least 8%. In some embodiments, the method relates to increasing the patient's RVFAC by least 9%. In some embodiments, the method relates to increasing the patient's RVFAC by least 10%. In some embodiments, the method relates to increasing the patient's RVFAC by least 12%. In some embodiments, the method relates to increasing the patient's RVFAC by least 14%. In some embodiments, the method relates to increasing the patient's RVFAC by least 16%. In some embodiments, the method relates to increasing the patient's RVFAC by least 18%. In some embodiments, the method relates to increasing the patient's RVFAC by least 20%.

In some embodiments, the improvement in right ventricular function is due to an increase in ejection fraction. In some embodiments, the improvement in right ventricular function is due to an increase in ejection fraction and an increase in the patient's RVFAC.

The right ventricular end-diastolic area (RVEDA) can be measured using echocardiography. In some embodiments, normal RVEDA is approximately 18.2±4.3 cm² in men and approximately 14.8±3.5 cm² in women. See, e.g., Kou S, et al. European Heart Journal—Cardiovascular Imaging. 2014 Jun. 1; 15(6):680-90.

In certain aspects, the disclosure relates to methods of treating, preventing, or reducing the progression rate and/or severity of PcPH comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has a RVEDA of at least 22 cm² (e.g., at least 22, 24, 26, 28, 30, 32, or 34 cm²). In some embodiments, the method relates to patients having a RVEDA of at least 24 cm². In some embodiments, the method relates to patients having a RVEDA of at least 26 cm². In some embodiments, the method relates to patients having a RVEDA of at least 28 cm². In some embodiments, the method relates to patients having a RVEDA of at least 30 cm². In some embodiments, the method relates to patients having a RVEDA of at least 32 cm². In some embodiments, the method relates to patients having a RVEDA of at least 34 cm². In some embodiments, the method relates to patients having increased RVEDA as compared to normal RVEDA.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to improving or maintaining the right ventricular function in the patient. In some embodiments, a patient with an improvement or maintenance of right ventricular function has a RVEDA of 14-22 cm². In some embodiments, the improvement in right ventricular function is a reduction in RVEDA. In some embodiments, the method relates to reducing the RVEDA. In some embodiments, the method relates to reducing the patients RVEDA by at least 1 cm². In some embodiments, the method relates to reducing the patients RVEDA by at least 2 cm². In some embodiments, the method relates to reducing the patients RVEDA by at least 3 cm². In some embodiments, the method relates to reducing the patients RVEDA by at least 4 cm². In some embodiments, the method relates to reducing the patients RVEDA by at least 5 cm². In some embodiments, the method relates to reducing the patients RVEDA by at least 6 cm². In some embodiments, the method relates to reducing the patients RVEDA by at least 7 cm². In some embodiments, the method relates to reducing the patients RVEDA by at least 8 cm². In some embodiments, the method relates to reducing the patients RVEDA by at least 9 cm². In some embodiments, the method relates to reducing the patients RVEDA by at least 10 cm².

In some embodiments, the disclosure relates to methods of adjusting one or more echocardiogram parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to decreasing the patient's RVEDA by least 10% (e.g., at least 1, 5, 10, 15, 20, 25, 30, 35, or 40%). In some embodiments, the method relates to decreasing the patient's RVEDA by at least 5%. In some embodiments, the method relates to decreasing the patient's RVEDA by at least 10%. In some embodiments, the method relates to decreasing the patient's RVEDA by at least 15%. In some embodiments, the method relates to decreasing the patient's RVEDA by at least 20%. In some embodiments, the method relates to decreasing the patient's RVEDA by at least 25%. In some embodiments, the method relates to decreasing the patient's RVEDA by at least 30%. In some embodiments, the method relates to decreasing the patient's RVEDA by at least 35%. In some embodiments, the method relates to decreasing the patient's RVEDA by at least 40%.

The right ventricular end-systolic area (RVESA) can be measured using echocardiography. In some embodiments, normal RVESA is approximately 9.6±2.8 cm² in men and approximately 7.3±2.3 cm² in women. See, e.g., Kou S, et al. European Heart Journal—Cardiovascular Imaging. 2014 Jun. 1; 15(6):680-90.

In certain aspects, the disclosure relates to methods of treating, preventing, or reducing the progression rate and/or severity of PcPH comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has a RVESA of at least 12 cm² (e.g., at least 12, 14, 16, 18, 20, 22, 24, 26, 28, 30, or 32 cm²). In some embodiments, the method relates to patients having a RVESA of at least 14 cm². In some embodiments, the method relates to patients having a RVESA of at least 16 cm². In some embodiments, the method relates to patients having a RVESA of at least 18 cm². In some embodiments, the method relates to patients having a RVESA of at least 20 cm². In some embodiments, the method relates to patients having a RVESA of at least 22 cm². In some embodiments, the method relates to patients having a RVESA of at least 24 cm². In some embodiments, the method relates to patients having a RVESA of at least 26 cm². In some embodiments, the method relates to patients having a RVESA of at least 28 cm². In some embodiments, the method relates to patients having a RVESA of at least 30 cm². In some embodiments, the method relates to patients having a RVESA of at least 32 cm². In some embodiments, the method relates to patients having increased RVESA as compared to normal RVESA.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to improving or maintaining the right ventricular function in the patient. In some embodiments, a patient with an improvement or maintenance of right ventricular function has a RVESA of 7-20 cm². In some embodiments, the improvement in right ventricular function is a reduction in RVESA. In some embodiments, the method relates to reducing the RVESA. In some embodiments, the method relates to reducing the patient's RVESA by at least 1 cm². In some embodiments, the method relates to reducing the patient's RVESA by at least 2 cm². In some embodiments, the method relates to reducing the patient's RVESA by at least 3 cm². In some embodiments, the method relates to reducing the patient's RVESA by at least 4 cm². In some embodiments, the method relates to reducing the patient's RVESA by at least 5 cm². In some embodiments, the method relates to reducing the patient's RVESA by at least 6 cm². In some embodiments, the method relates to reducing the patient's RVESA by at least 7 cm². In some embodiments, the method relates to reducing the patient's RVESA by at least 8 cm². In some embodiments, the method relates to reducing the patient's RVESA by at least 9 cm². In some embodiments, the method relates to reducing the patient's RVESA by at least 10 cm².

In some embodiments, the disclosure relates to methods of adjusting one or more echocardiogram parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to decreasing the patient's RVESA by least 10% (e.g., at least 1, 2, 3, 4, 5, 10, 15, 20, 25, 30, 35, or 40%). In some embodiments, the method relates to decreasing the patient's RVESA by at least 2%. In some embodiments, the method relates to decreasing the patient's RVESA by at least 3%. In some embodiments, the method relates to decreasing the patient's RVESA by at least 4%. In some embodiments, the method relates to decreasing the patient's RVESA by at least 5%. In some embodiments, the method relates to decreasing the patient's RVESA by at least 10%. In some embodiments, the method relates to decreasing the patient's RVESA by at least 15%. In some embodiments, the method relates to decreasing the patient's RVESA by at least 20%. In some embodiments, the method relates to decreasing the patient's RVESA by at least 25%. In some embodiments, the method relates to decreasing the patient's RVESA by at least 30%. In some embodiments, the method relates to decreasing the patient's RVESA by at least 35%. In some embodiments, the method relates to decreasing the patient's RVESA by at least 40%.

### RVFWT

In patients with pulmonary hypertension, the right ventricle dilates in response to increased PAP and right ventricular remodeling. As the disease progresses right ventricular hypertrophy develops, resulting in increased right ventricle free wall thickness. In some embodiments, the right ventricular free wall thickness (RVFWT) can be measured using echocardiography. In some embodiments, normal RVFWT is approximately 0.22-0.42 cm in women and approximately 0.24-0.42 cm in men. See, e.g., Lang R M, J Am Soc Echocardiogr. 2015; 28(1):1-39.e14.

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has a RVFWT of at least 0.42 cm (e.g., at least 0.42, 0.44, 0.46, 0.48, 0.50, 0.52, 0.54, 0.56, 0.58, or 0.60 cm). In some embodiments, the method relates to patients having a RVFWT of at least 0.44 cm. In some embodiments, the method relates to patients having a RVFWT of at least 0.46 cm. In some embodiments, the method relates to patients having a RVFWT of at least 0.48 cm. In some embodiments, the method relates to patients having a RVFWT of at least 0.50 cm. In some embodiments, the method relates to patients having a RVFWT of at least 0.52 cm. In some embodiments, the method relates to patients having a RVFWT of at least 0.54 cm. In some embodiments, the method relates to patients having a RVFWT of at least 0.56 cm. In some embodiments, the method relates to patients having a RVFWT of at least 0.58 cm. In some embodiments, the method relates to patients having a RVFWT of at least 0.60 cm. In some embodiments, the method relates to patients having increased RVFWT as compared to normal RVFWT.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to improving or maintaining the right ventricular function in the patient. In some embodiments, a patient with an improvement or maintenance of right ventricular function has a RVFWT of between 0.22-0.42 cm. In some embodiments, the improvement in right ventricular function is a reduction in RVFWT. In some embodiments, the method relates to reducing the RVFWT. In some embodiments, the method relates to reducing the patients RVFWT by at least 0.05 cm (e.g., at least 0.05, 0.1, 0.15, 0.2, 0.25, 0.3, 0.35, or 0.4 cm). In some embodiments, the method relates to reducing the patients RVFWT by at least 0.1 cm. In some embodiments, the method relates to reducing the patients RVFWT by at least 0.15 cm. In some embodiments, the method relates to reducing the patients RVFWT by at least 0.2 cm. In some embodiments, the method relates to reducing the patients RVFWT by at least 0.25 cm. In some embodiments, the method relates to reducing the patients RVFWT by at least 0.3 cm. In some embodiments, the method relates to reducing the patients RVFWT by at least 0.35 cm. In some embodiments, the method relates to reducing the patients RVFWT by at least 0.4 cm.

In some embodiments, the disclosure relates to methods of adjusting the RVFWT in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to decreasing the patient's RVFWT by least 1% (e.g., at least 1, 5, 10, 15, 20, 25, 30, 35, 40, 45, 50, 55, 60, 65, 70, or 75%). In some embodiments, the method relates to decreasing the patient's RVFWT by at least 5%. In some embodiments, the method relates to decreasing the patient's RVFWT by at least 10%. In some embodiments, the method relates to decreasing the patient's RVFWT by at least 15%. In some embodiments, the method relates to decreasing the patient's RVFWT by at least 20%. In some embodiments, the method relates to decreasing the patient's RVFWT by at least 25%. In some embodiments, the method relates to decreasing the patient's RVFWT by at least 30%. In some embodiments, the method relates to decreasing the patient's RVFWT by at least 35%. In some embodiments, the method relates to decreasing the patient's RVFWT by at least 40%. In some embodiments, the method relates to decreasing the patient's RVFWT by at least 45%. In some embodiments, the method relates to decreasing the patient's RVFWT by at least 50%. In some embodiments, the method relates to decreasing the patient's RVFWT by at least 55%. In some embodiments, the method relates to decreasing the patient's RVFWT by at least 60%. In some embodiments, the method relates to decreasing the patient's RVFWT by at least 65%. In some embodiments, the method relates to decreasing the patient's RVFWT by at least 70%. In some embodiments, the method relates to decreasing the patient's RVFWT by at least 75%.

### RVEF

Right ventricular ejection fraction is a global measure of RV systolic performance. RVEF can be calculated using the RV end-diastolic volume (RVEDV) and RV end systolic volume (RVESV). Specifically, RVEF can be calculated using the following formula: RVEF (%)=((RVEDV−RVESV)/RVEDV)*100. Normal RVEF is approximately 56-65% in men and 60-710% in women. See, e.g., Lang R M, J Am Soc Echocardiogr. 2015; 28(1):1-39.e14. In some embodiments, the RVEF is measured using echocardiography. In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to improving or maintaining the right ventricular function in the patient. In some embodiments, a patient with an improvement or maintenance of right ventricular function has a RVEF of 45-710%. In some embodiments, a patient with an improvement or maintenance of right ventricular function has a RVEF of at least 45%. In some embodiments, a patient with an improvement or maintenance of right ventricular function has a RVEF of at least 50%. In some embodiments, a patient with an improvement or maintenance of right ventricular function has a RVEF of at least 55%. In some embodiments, a patient with an improvement or maintenance of right ventricular function has a RVEF of at least 60%. In some embodiments, a patient with an improvement or maintenance of right ventricular function has a RVEF of at least 65%. In some embodiments, a patient with an improvement or maintenance of right ventricular function has a RVEF of at least 70%.

In some embodiments, the disclosure relates to methods of adjusting one or more echocardiogram parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to increasing the patient's RVEF by least 2%. In some embodiments, the method relates to increasing the patient's RVEF by least 3%. In some embodiments, the method relates to increasing the patient's RVEF by least 4%. In some embodiments, the method relates to increasing the patient's RVEF by least 5%. In some embodiments, the method relates to increasing the patient's RVEF by least 6%. In some embodiments, the method relates to increasing the patient's RVEF by least 7%. In some embodiments, the method relates to increasing the patient's RVEF by least 8%. In some embodiments, the method relates to increasing the patient's RVEF by least 9%. In some embodiments, the method relates to increasing the patient's RVEF by least 10%. In some embodiments, the method relates to increasing the patient's RVEF by least 110%. In some embodiments, the method relates to increasing the patient's RVEF by least 12%. In some embodiments, the method relates to increasing the patient's RVEF by least 13%. In some embodiments, the method relates to increasing the patient's RVEF by least 14%. In some embodiments, the method relates to increasing the patient's RVEF by least 15%.

### Right Ventricular Hypertrophy

In certain aspects, the improvement in right ventricular function is measured as a decrease in right ventricular hypertrophy. In some embodiment, the right ventricular hypertrophy is measured using the Fulton Index (RV/(LV+S)).

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has right ventricular hypertrophy. In some embodiments, the disclosure relates to methods of adjusting one or more parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the right ventricular hypertrophy is measured using the Fulton index (RV/(LV+S)). In some embodiments, the method relates to decreasing the patient's right ventricular hypertrophy by least 10% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's right ventricular hypertrophy by at least 10%. In some embodiments, the method relates to decreasing the patient's right ventricular hypertrophy by at least 5%. In some embodiments, the method relates to decreasing the patient's right ventricular hypertrophy by at least 10%. In some embodiments, the method relates to decreasing the patient's right ventricular hypertrophy by at least 15%. In some embodiments, the method relates to decreasing the patient's right ventricular hypertrophy by at least 20%. In some embodiments, the method relates to decreasing the patient's right ventricular hypertrophy by at least 25%. In some embodiments, the method relates to decreasing the patient's right ventricular hypertrophy by at least 30%. In some embodiments, the method relates to decreasing the patient's right ventricular hypertrophy by at least 35%. In some embodiments, the method relates to decreasing the patient's right ventricular hypertrophy by at least 40%. In some embodiments, the method relates to decreasing the patient's right ventricular hypertrophy by at least 45%. In some embodiments, the method relates to decreasing the patient's right ventricular hypertrophy by at least 50%. In some embodiments, the method relates to decreasing the patient's right ventricular hypertrophy by at least 55%. In some embodiments, the method relates to decreasing the patient's right ventricular hypertrophy by at least 60%. In some embodiments, the method relates to decreasing the patient's right ventricular hypertrophy by at least 65%. In some embodiments, the method relates to decreasing the patient's right ventricular hypertrophy by at least 70%. In some embodiments, the method relates to decreasing the patient's right ventricular hypertrophy by at least 75%. In some embodiments, the method relates to decreasing the patient's right ventricular hypertrophy by at least 80%. In some embodiments, the method relates to decreasing the patient's right ventricular hypertrophy by at least 85%. In some embodiments, the method relates to decreasing the patient's right ventricular hypertrophy by at least 90%. In some embodiments, the method relates to decreasing the patient's right ventricular hypertrophy by at least 95%. In some embodiments, the method relates to decreasing the patient's right ventricular hypertrophy by at least 100%.

### Left Ventricular Hypertrophy

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has left ventricular hypertrophy. In some embodiments, the disclosure relates to methods of adjusting one or more parameters in the PcPH patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to decreasing the patient's left ventricular hypertrophy by least 10% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's left ventricular hypertrophy by at least 10%. In some embodiments, the method relates to decreasing the patient's left ventricular hypertrophy by at least 5%. In some embodiments, the method relates to decreasing the patient's left ventricular hypertrophy by at least 10%. In some embodiments, the method relates to decreasing the patient's left ventricular hypertrophy by at least 15%. In some embodiments, the method relates to decreasing the patient's left ventricular hypertrophy by at least 20%. In some embodiments, the method relates to decreasing the patient's left ventricular hypertrophy by at least 25%. In some embodiments, the method relates to decreasing the patient's left ventricular hypertrophy by at least 30%. In some embodiments, the method relates to decreasing the patient's left ventricular hypertrophy by at least 35%. In some embodiments, the method relates to decreasing the patient's left ventricular hypertrophy by at least 40%. In some embodiments, the method relates to decreasing the patient's left ventricular hypertrophy by at least 45%. In some embodiments, the method relates to decreasing the patient's left ventricular hypertrophy by at least 50%. In some embodiments, the method relates to decreasing the patient's left ventricular hypertrophy by at least 55%. In some embodiments, the method relates to decreasing the patient's left ventricular hypertrophy by at least 60%. In some embodiments, the method relates to decreasing the patient's left ventricular hypertrophy by at least 65%. In some embodiments, the method relates to decreasing the patient's left ventricular hypertrophy by at least 70%. In some embodiments, the method relates to decreasing the patient's left ventricular hypertrophy by at least 75%. In some embodiments, the method relates to decreasing the patient's left ventricular hypertrophy by at least 80%. In some embodiments, the method relates to decreasing the patient's left ventricular hypertrophy by at least 85%. In some embodiments, the method relates to decreasing the patient's left ventricular hypertrophy by at least 90%. In some embodiments, the method relates to decreasing the patient's left ventricular hypertrophy by at least 95%. In some embodiments, the method relates to decreasing the patient's left ventricular hypertrophy by at least 100%.

### Cardiac Output

Cardiac output is the volume of blood the heart pumps per minute. Cardiac output is calculated by multiplying the stroke volume by the heart rate. In general, normal cardiac output at rest is about 4 to 8 L/min. The cardiac index is an assessment of the cardiac output value based on the patient's size. To find the cardiac index, the cardiac output is divided by the person's body surface area (BSA). The normal range for CI is 2.5 to 4 L/min/m². Cardiac output can decline by almost 40% without deviating from the normal limits. A low cardiac index of less than about 2.5 L/min/m² usually indicates a disturbance in cardiovascular performance. The cardiac output can be utilized to calculate the cardiac index (e.g., cardiac index=cardiac output/body surface area). The cardiac output can also be utilized to calculate the stroke volume (e.g., stroke volume=CO/heart rate). In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the method increases the patient's cardiac output by at least 5% (e.g., at least 5, 10, 15, 20, 25, 30, 35, 40, 45, 50, 55, 60, 65, 70, 75, 80, 85, 90, 95, or 100%). In some embodiments, the method relates to increasing the patient's cardiac output by at least 5%. In some embodiments, the method relates to increasing the patient's cardiac output by at least 10%. In some embodiments, the method relates to increasing the patient's cardiac output by at least 15%. In some embodiments, the method relates to increasing the patient's cardiac output by at least 20%. In some embodiments, the method relates to increasing the patient's cardiac output by at least 25%. In some embodiments, the method relates to increasing the patient's cardiac output by at least 30%. In some embodiments, the method relates to increasing the patient's cardiac output by at least 35%. In some embodiments, the method relates to increasing the patient's cardiac output by at least 40%. In some embodiments, the method relates to increasing the patient's cardiac output by at least 45%. In some embodiments, the method relates to increasing the patient's cardiac output by at least 50%. In some embodiments, the method relates to increasing the patient's cardiac output by at least 55%. In some embodiments, the method relates to increasing the patient's cardiac output by at least 60%. In some embodiments, the method relates to increasing the patient's cardiac output by at least 65%. In some embodiments, the method relates to increasing the patient's cardiac output by at least 70%. In some embodiments, the method relates to increasing the patient's cardiac output by at least 75%. In some embodiments, the method relates to increasing the patient's cardiac output by at least 80%. In some embodiments, the method relates to increasing the patient's cardiac output by at least 85%. In some embodiments, the method relates to increasing the patient's cardiac output by at least 90%. In some embodiments, the method relates to increasing the patient's cardiac output by at least 95%. In some embodiments, the method relates to increasing the patient's cardiac output by at least 100%. In some embodiments, the method relates to increasing the patient's cardiac index to at least 4.2 L/min/m². In some embodiments, the cardiac index is measured at rest. In some embodiments, the method relates to increasing the patient's cardiac output to at least 4 L/min. In some embodiments, the cardiac output is measured at rest. In some embodiments, the cardiac output is using a right heart catheter. In some embodiments, cardiac output is measured by thermodilution. In some embodiments, cardiac output is measured using the Fick method.

### Progression of IpcPH to CpcPH

The predominant mechanism underlying PcPH (e.g., WHO Group 2 and/or Group 5 PH) is elevated left-side filling pressure (i.e., left atrial pressure). Sustained elevations in left atrial pressure may cause passive pulmonary venous congestion with elevation of pulmonary pressures. In some patients, transmission of venous congestion to the pulmonary capillaries results in leakage and damage, ultimately leading to the creation of an obstructive vasculopathy such that higher pulmonary pressures are needed to sustain forward flow. This is sometimes referred to as the development of a “pre-capillary” component of PH. In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the method reduces the development of a pre-capillary component of PH by at least 1% (e.g., at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, the method relates to reducing the development of a pre-capillary component of PH in a patient by at least 1%. In some embodiments, the method relates to reducing the development of a pre-capillary component of PH in a patient by at least 2%. In some embodiments, the method relates to reducing the development of a pre-capillary component of PH in a patient by at least 3%. In some embodiments, the method relates to reducing the development of a pre-capillary component of PH in a patient by at least 4%. In some embodiments, the method relates to reducing the development of a pre-capillary component of PH in a patient by at least 5%. In some embodiments, the method relates to reducing the development of a pre-capillary component of PH in a patient by at least 10%. In some embodiments, the method relates to reducing the development of a pre-capillary component of PH in a patient by at least 15%. In some embodiments, the method relates to reducing the development of a pre-capillary component of PH in a patient by at least 20%. In some embodiments, the method relates to reducing the development of a pre-capillary component of PH in a patient by at least 25%. In some embodiments, the method relates to reducing the development of a pre-capillary component of PH in a patient by at least 30%. In some embodiments, the method relates to reducing the development of a pre-capillary component of PH in a patient by at least 35%. In some embodiments, the method relates to reducing the development of a pre-capillary component of PH in a patient by at least 40%. In some embodiments, the method relates to reducing the development of a pre-capillary component of PH in a patient by at least 45%. In some embodiments, the method relates to reducing the development of a pre-capillary component of PH in a patient by at least 50%. In some embodiments, the method relates to reducing the development of a pre-capillary component of PH in a patient by at least 55%. In some embodiments, the method relates to reducing the development of a pre-capillary component of PH in a patient by at least 60%. In some embodiments, the method relates to reducing the development of a pre-capillary component of PH in a patient by at least 65%. In some embodiments, the method relates to reducing the development of a pre-capillary component of PH in a patient by at least 70%. In some embodiments, the method relates to reducing the development of a pre-capillary component of PH in a patient by at least 75%. In some embodiments, the method relates to reducing the development of a pre-capillary component of PH in a patient by at least 80%. In some embodiments, the method relates to reducing the development of a pre-capillary component of PH in a patient by at least 85%. In some embodiments, the method relates to reducing the development of a pre-capillary component of PH in a patient by at least 90%. In some embodiments, the method relates to reducing the development of a pre-capillary component of PH in a patient by at least 95%. In some embodiments, the method relates to reducing the development of a pre-capillary component of PH in a patient by at least 100%.

In some embodiments, sustained left atrial pressure in IpcPH has been shown to lead to the development of CpcPH. In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the method reduces the progression of IpcPH to CpcPH in a patient by at least 1% (e.g., at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, the method relates to reducing the progression of IpcPH to CpcPH in a patient by at least 1%. In some embodiments, the method relates to reducing the progression of IpcPH to CpcPH in a patient by at least 2%. In some embodiments, the method relates to reducing the progression of IpcPH to CpcPH in a patient by at least 3%. In some embodiments, the method relates to reducing the progression of IpcPH to CpcPH in a patient by at least 4%. In some embodiments, the method relates to reducing the progression of IpcPH to CpcPH in a patient by at least 5%. In some embodiments, the method relates to reducing the progression of IpcPH to CpcPH in a patient by at least 10%. In some embodiments, the method relates to reducing the progression of IpcPH to CpcPH in a patient by at least 15%. In some embodiments, the method relates to reducing the progression of IpcPH to CpcPH in a patient by at least 20%. In some embodiments, the method relates to reducing the progression of IpcPH to CpcPH in a patient by at least 25%. In some embodiments, the method relates to reducing the progression of IpcPH to CpcPH in a patient by at least 30%. In some embodiments, the method relates to reducing the progression of IpcPH to CpcPH in a patient by at least 35%. In some embodiments, the method relates to reducing the progression of IpcPH to CpcPH in a patient by at least 40%. In some embodiments, the method relates to reducing the progression of IpcPH to CpcPH in a patient by at least 45%. In some embodiments, the method relates to reducing the progression of IpcPH to CpcPH in a patient by at least 50%. In some embodiments, the method relates to reducing the progression of IpcPH to CpcPH in a patient by at least 55%. In some embodiments, the method relates to reducing the progression of IpcPH to CpcPH in a patient by at least 60%. In some embodiments, the method relates to reducing the progression of IpcPH to CpcPH in a patient by at least 65%. In some embodiments, the method relates to reducing the progression of IpcPH to CpcPH in a patient by at least 70%. In some embodiments, the method relates to reducing the progression of IpcPH to CpcPH in a patient by at least 75%. In some embodiments, the method relates to reducing the progression of IpcPH to CpcPH in a patient by at least 80%. In some embodiments, the method relates to reducing the progression of IpcPH to CpcPH in a patient by at least 85%. In some embodiments, the method relates to reducing the progression of IpcPH to CpcPH in a patient by at least 90%. In some embodiments, the method relates to reducing the progression of IpcPH to CpcPH in a patient by at least 95%. In some embodiments, the method relates to reducing the progression of IpcPH to CpcPH in a patient by at least 100%.

### Exercise Capacity (6MWD and BDI)

In certain aspects, the disclosure relates to methods of increasing exercise capacity in a patient having PcPH (e.g., WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). Any suitable measure of exercise capacity can be used. For example, exercise capacity in a 6-minute walk test (6MWT), which measures how far the subject can walk in 6 minutes, i.e., the 6-minute walk distance (6MWD), is frequently used to assess pulmonary hypertension severity and disease progression. In certain aspects, the Borg dyspnea index (BDI) may be used to measure exercise capacity. The BDI is a numerical scale for assessing perceived dyspnea (breathing discomfort). It measures the degree of breathlessness, for example, after completion of the 6MWT, where a BDI of 0 indicates no breathlessness and 10 indicates maximum breathlessness. In some embodiments, the BDI is measured using BORG CR10 scale.

In certain aspects, the disclosure relates to methods of treating, preventing, or reducing the progression rate and/or severity of PcPH (e.g., WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has a 6MWD of less than 550 meters (e.g., a 6MWD of less than 550, 500, 450, 440, 400, 380, 350, 300, 250, 200, or 150 meters). In some embodiments, the method relates to patient's having a 6MWD of between 150 to 550 meters. In some embodiments, the method relates to patient's having a 6MWD of between 100 to 500 meters. In some embodiments, the method relates to patient's having a 6MWD of between 150 to 500 meters. In some embodiments, the method relates to patient's having a 6MWD of at least 100 meters. In some embodiments, the method relates to patient's having a 6MWD of greater than 150 meters. In some embodiments, the method relates to patient's having a 6MWD of less than 550 meters. In some embodiments, the method relates to patient's having a 6MWD of less than 500 meters. In some embodiments, the method relates to patient's having a 6MWD of less than 450 meters. In some embodiments, the method relates to patient's having a 6MWD of less than 440 meters. In some embodiments, the method relates to patient's having a 6MWD of less than 400 meters. In some embodiments, the method relates to patient's having a 6MWD of less than 380 meters. In some embodiments, the method relates to patient's having a 6MWD of less than 350 meters. In some embodiments, the method relates to patient's having a 6MWD of less than 300 meters. In some embodiments, the method relates to patient's having a 6MWD of less than 250 meters. In some embodiments, the method relates to patient's having a 6MWD of less than 200 meters. In some embodiments, the method relates to patient's having a 6MWD of less than 150 meters. In some embodiments, the method relates to increasing the patient's 6MWD to >380 meters. In some embodiments, the method relates to increasing the patient's 6MWD to >440 meters. In some embodiments, the method relates to increasing the patient's 6MWD to >500 meters. See, e.g., Galie N., et al Euro Heart J. (2016) 37, 67-119. e.g. e.g.

In some embodiments, the disclosure relates to methods of adjusting one or more measurements of exercise capacity in the PcPH (e.g., WHO Group 2 and/or Group 5 PH) patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to increasing the patient's 6MWD by at least 10 meters. In some embodiments, the method relates to increasing the patient's 6MWD by at least 20 meters. In some embodiments, the method relates to increasing the patient's 6MWD by at least 25 meters. In some embodiments, the method relates to increasing the patient's 6MWD by at least 30 meters. In some embodiments, the method relates to increasing the patient's 6MWD by at least 40 meters. In some embodiments, the method relates to increasing the patient's 6MWD by at least 50 meters. In some embodiments, the method relates to increasing the patient's 6MWD by at least 60 meters. In some embodiments, the method relates to increasing the patient's 6MWD by at least 70 meters. In some embodiments, the method relates to increasing the patient's 6MWD by at least 80 meters. In some embodiments, the method relates to increasing the patient's 6MWD by at least 90 meters. In some embodiments, the method relates to increasing the patient's 6MWD by at least 100 meters. In some embodiments, the method relates to increasing the patient's 6MWD by at least 125 meters. In some embodiments, the method relates to increasing the patient's 6MWD by at least 150 meters. In some embodiments, the method relates to increasing the patient's 6MWD by at least 175 meters. In some embodiments, the method relates to increasing the patient's 6MWD by at least 200 meters. In some embodiments, the method relates to increasing the patient's 6MWD by at least 250 meters. In some embodiments, the method relates to increasing the patient's 6MWD by at least 300 meters. In some embodiments, the method relates to increasing the patient's 6MWD by at least 400 meters. In some embodiments, the 6MWD is tested after the patient has received 4 weeks of treatment utilizing an ActRII antagonist disclosed herein. In some embodiments, the 6MWD is tested after the patient has received 8 weeks of treatment utilizing an ActRII polypeptide disclosed herein. In some embodiments, the 6MWD is tested after the patient has received 12 weeks of treatment utilizing an ActRII antagonist disclosed herein. In some embodiments, the 6MWD is tested after the patient has received 16 weeks of treatment utilizing an ActRII antagonist disclosed herein. In some embodiments, the 6MWD is tested after the patient has received 20 weeks of treatment utilizing an ActRII antagonist disclosed herein. In some embodiments, the 6MWD is tested after the patient has received 22 weeks of treatment utilizing an ActRII antagonist disclosed herein. In some embodiments, the 6MWD is tested after the patient has received 24 weeks of treatment utilizing an ActRII antagonist disclosed herein. In some embodiments, the 6MWD is tested after the patient has received 26 weeks of treatment utilizing an ActRII antagonist disclosed herein. In some embodiments, the 6MWD is tested after the patient has received 28 weeks of treatment utilizing an ActRII antagonist disclosed herein. In some embodiments, the 6MWD is tested after the patient has received 48 weeks of treatment utilizing an ActRII polypeptide disclosed herein.

In some embodiments, the disclosure relates to methods of adjusting one or more measurements of exercise capacity (e.g., BDI) in the PcPH (e.g., WHO Group 2 and/or Group 5 PH) patient toward a more normal level (e.g., normal as compared to healthy people of similar age and sex), comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to reducing the patient's BDI. In some embodiments, the method relates to lowering the patient's BDI by at least 0.5 index points. In some embodiments, the method relates to lowering the patient's BDI by at least 1 index points. In some embodiments, the method relates to lowering the patient's BDI by at least 1.5 index points. In some embodiments, the method relates to lowering the patient's BDI by at least 2 index points. In some embodiments, the method relates to lowering the patient's BDI by at least 2.5 index points. In some embodiments, the method relates to lowering the patient's BDI by at least 3 index points. In some embodiments, the method relates to lowering the patient's BDI by at least 3.5 index points. In some embodiments, the method relates to lowering the patient's BDI by at least 4 index points. In some embodiments, the method relates to lowering the patient's BDI by at least 4.5 index points. In some embodiments, the method relates to lowering the patient's BDI by at least 5 index points. In some embodiments, the method relates to lowering the patient's BDI by at least 5.5 index points. In some embodiments, the method relates to lowering the patient's BDI by at least 6 index points. In some embodiments, the method relates to lowering the patient's BDI by at least 6.5 index points. In some embodiments, the method relates to lowering the patient's BDI by at least 7 index points. In some embodiments, the method relates to lowering the patient's BDI by at least 7.5 index points. In some embodiments, the method relates to lowering the patient's BDI by at least 8 index points. In some embodiments, the method relates to lowering the patient's BDI by at least 8.5 index points. In some embodiments, the method relates to lowering the patient's BDI by at least 9 index points. In some embodiments, the method relates to lowering the patient's BDI by at least 9.5 index points. In some embodiments, the method relates to lowering the patient's BDI by at least 3 index points. In some embodiments, the method relates to lowering the patient's BDI by at least 10 index points.

### Echocardiogram

There are numerous clinical presentation factors, echocardiography features, and other features that could be indicative of PcPH (e.g., WHO Group 2 and/or Group 5 PH). For instance, patients who are ≥65 years old are at higher risk for WHO Group 2 (also known as PH-LHD). In patients suspected of having PcPH (e.g., WHO Group 2 and/or Group 5 PH), an echocardiogram may be used to image the effects of PH on the heart and estimate the mPAP from continuous wave Doppler measurements. In some embodiments, an echocardiogram performed on a patient shows structural left heart abnormalities. In some embodiments, the structural left heart abnormality is a disease of the left heart valves. In some embodiments, the structural left heart abnormality is left atrium enlargement (e.g., >4.2 cm). In some embodiments, an electrocardiogram performed on a patient shows left ventricular hypertrophy (LVH) and/or left atrial hypertrophy/dilation (LAH). In some embodiments, an electrocardiogram performed on a patient shows atrial flutter/atrial fibrillation (AF/Afib). In some embodiments, an electrocardiogram performed on a patient shows left bundle branch block (LBBB). In some embodiments, an electrocardiogram performed on a patient shows the presence of Q waves. See, e.g., Galie N., et al Euro Heart J. (2016) 37, 67-119.

In a patient that has symptoms of left heart failure, an echocardiogram may be performed to evaluate various parameters. For instance, in some embodiments, an echocardiogram using Doppler performed on a patient may show indices of increased filling pressures and/or diastolic dysfunction (e.g., increased E/E′ or >Type 2-3 mitral flow abnormality). In some embodiments, imaging (e.g. echocardiogram, CT scan, chest X-ray, or MRI) performed on a patient shows Kerley B lines. In some embodiments, imaging (e.g. echocardiogram, CT scan, chest X-ray, or MRI) performed on a patient shows pleural effusion. In some embodiments, imaging (e.g. echocardiogram, CT scan, chest X-ray, or MRI) performed on a patient shows pulmonary edema. In some embodiments, imaging (e.g., echocardiogram, CT scan, chest X-ray, or MRI) performed on a patient shows left atrium enlargement. Id.

Furthermore, in a patient that has features of metabolic syndrome, imaging (e.g. an echocardiogram) may be performed to evaluate various parameters. For instance, in some embodiments, an echocardiogram performed on a patient shows the absence of right ventricle dysfunction (e.g., IpcPH). In some embodiments, an echocardiogram performed on a patient shows the presence of right ventricle dysfunction (e.g., CpcPH). In some embodiments, an echocardiogram performed on a patient shows the absence of mid systolic notching of the pulmonary artery flow. In some embodiments, an echocardiogram performed on a patient shows the absence of pericardial effusion. In some embodiments, the patient has a history of heart disease (past or current). In some embodiments, the patient has persistent atrial fibrillation. Id. In some embodiments, an Echo Score or the TAPSE/systolic pulmonary arterial pressure ratio are used to differentiate Cpc-PH from Ipc-PH. In some embodiments, an integrative score of five echocardiographic parameters (RV/LV ratio, left ventricular eccentricity index (LVEI), E/E′, RV forming apex, width and inspiratory collapse of IVC) as well as “notching” of the RV outflow tract Doppler envelope may be used to distinguish between precapillary PH (e.g., PAH) from post-capillary PH (e.g., WHO Group 2 and/or Group 5 PH).

In some embodiments, a patient has diastolic dysfunction. In some embodiments, the method improves diastolic dysfunction in the patient. In some embodiments, the improvement in diastolic dysfunction is an improvement in the E/E′ ratio (a ratio of mitral inflow velocity (E) to mitral annular velocity (E′). In some embodiments, the improvement in diastolic dysfunction is an improvement in the isovolumic relaxation time (IVRT). In some embodiments, the improvement in the diastolic dysfunction is a lower RVSP. In some embodiments, the diastolic dysfunction results from one or more conditions selected from the group consisting of hypertension, diabetes, and advanced age.

Complications of PH In certain aspects, the disclosure relates to methods of treating, preventing, or reducing the progression rate and/or severity of one or more complications of PcPH (e.g., WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method relates to treating, preventing, or reducing the progression rate and/or severity of cell proliferation in the pulmonary artery of a PcPH patient. In some embodiments, the method relates to treating, preventing, or reducing the progression rate and/or severity of smooth muscle and/or endothelial cells proliferation in the pulmonary artery of a PcPH patient. In some embodiments, the method relates to treating, preventing, or reducing the progression rate and/or severity of angiogenesis in the pulmonary artery of a PcPH patient. In some embodiments, the method relates to increasing physical activity of a patient having PcPH. In some embodiments, the method relates to treating, preventing, or reducing the progression rate and/or severity of dyspnea in a PcPH patient. In some embodiments, the method relates to treating, preventing, or reducing the progression rate and/or severity of chest pain in a PcPH patient. In some embodiments, the method relates to treating, preventing, or reducing the progression rate and/or severity of fatigue in a PcPH patient. In some embodiments, the method relates to treating, preventing, or reducing the progression rate and/or severity of fibrosis in a PcPH patient. In some embodiments, the fibrosis is selected from the group consisting of left ventricular fibrosis, right ventricular fibrosis, and pulmonary fibrosis. In some embodiments, the method relates to treating, preventing, or reducing the progression rate and/or severity of left ventricular fibrosis in a PcPH patient. In some embodiments, the method relates to treating, preventing, or reducing the progression rate and/or severity of right ventricular fibrosis in a PcPH patient. In some embodiments, the method relates to treating, preventing, or reducing the progression rate and/or severity of pulmonary fibrosis in a PcPH patient. In some embodiments, the method relates to decreasing the patient's fibrosis by least 1% (e.g., at least 1%, 5, 10, 15, 20, 25, 30, 35, 40, 45, 50, 55, 60, 65, 70, 75, 80, 85, 90, 95, or 100%). In some embodiments, the method relates to decreasing the patient's fibrosis by at least 10%. In some embodiments, the method relates to decreasing the patient's fibrosis by at least 5%. In some embodiments, the method relates to decreasing the patient's fibrosis by at least 10%. In some embodiments, the method relates to decreasing the patient's fibrosis by at least 15%. In some embodiments, the method relates to decreasing the patient's fibrosis by at least 20%. In some embodiments, the method relates to decreasing the patient's fibrosis by at least 25%. In some embodiments, the method relates to decreasing the patient's fibrosis by at least 30%. In some embodiments, the method relates to decreasing the patient's fibrosis by at least 35%. In some embodiments, the method relates to decreasing the patient's fibrosis by at least 40%. In some embodiments, the method relates to decreasing the patient's fibrosis by at least 45%. In some embodiments, the method relates to decreasing the patient's fibrosis by at least 50%. In some embodiments, the method relates to decreasing the patient's fibrosis by at least 55%. In some embodiments, the method relates to decreasing the patient's fibrosis by at least 60%. In some embodiments, the method relates to decreasing the patient's fibrosis by at least 65%. In some embodiments, the method relates to decreasing the patient's fibrosis by at least 70%. In some embodiments, the method relates to decreasing the patient's fibrosis by at least 75%. In some embodiments, the method relates to decreasing the patient's fibrosis by at least 80%. In some embodiments, the method relates to decreasing the patient's fibrosis by at least 85%. In some embodiments, the method relates to decreasing the patient's fibrosis by at least 90%. In some embodiments, the method relates to decreasing the patient's fibrosis by at least 95%. In some embodiments, the method relates to decreasing the patient's fibrosis by at least 100%.

In some embodiments, the method relates to treating, preventing, or reducing the progression rate and/or severity of pulmonary vascular remodeling in a PcPH patient. In some embodiments, the method relates to treating, preventing, or reducing the progression rate and/or severity of cardiac remodeling in a PcPH patient. In some embodiments, the method relates to treating, preventing, or reducing the progression rate and/or severity of left cardiac remodeling in a PcPH patient. In some embodiments, the method relates to treating, preventing, or reducing the progression rate and/or severity of right cardiac remodeling in a PcPH patient. In some embodiments, the method relates to treating, preventing, or reducing the progression rate and/or severity of right ventricular hypertrophy in a PcPH patient. In some embodiments, the method relates to treating, preventing, or reducing the progression rate and/or severity of left ventricular hypertrophy in a PcPH patient. In some embodiments, the method relates to treating, preventing, or reducing the progression rate and/or severity of metabolic syndrome in a PcPH patient. In some embodiments, the method relates to treating, preventing, or reducing the progression rate and/or severity of left atrium dilation in a PcPH patient. In some embodiments, the method relates to treating, preventing, or reducing the progression rate and/or severity of an underlying condition (e.g., COPD, sleep apnea syndrome, CTEPH) in a PcPH patient.

### Complications or Comorbidities and Combination Therapies

In some embodiments, the disclosure contemplates methods of treating one or more complications of PcPH (e.g., smooth muscle and/or endothelial cell proliferation in the pulmonary artery, angiogenesis in the pulmonary artery, dyspnea, chest pain, pulmonary vascular remodeling, cardiac remodeling, right ventricular hypertrophy, left ventricular hypertrophy, left atrium dilation, pulmonary fibrosis, need for lung and/or heart transplant, and need for atrial septostomy) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the disclosure contemplates methods of preventing one or more complications of PcPH comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the disclosure contemplates methods of reducing the progression rate of one or more complications of PcPH comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the disclosure contemplates methods of reducing the severity of one or more complications of PcPH comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins).

In some embodiments, the disclosure contemplates methods of treating one or more comorbidities of PcPH (e.g., systemic hypertension, decreased renal function, diabetes mellitus, obesity, coronary artery disease (CAD), heart failure, and anemia) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the method results in the improvement of one or more comorbidities of PcPH (e.g., systemic hypertension, decreased renal function, diabetes mellitus, obesity, coronary artery disease (CAD), heart failure, and anemia). In some embodiments, the one or more comorbidities of PcPH are improved indirectly (e.g., due to an improvement in the patient's PH).

In some embodiments, the disclosure contemplates methods of reducing the progression rate of PcPH (e.g., WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the disclosure contemplates methods of reducing the progression rate of one or more complications of PcPH (e.g., WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the disclosure contemplates methods of reducing the severity of PcPH (e.g., WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the disclosure contemplates methods of reducing the severity of one or more complications of PcPH (e.g., WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the disclosure contemplates method of reducing the need to initiate treatment with a known treatment for PcPH comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the disclosure contemplates a method of reducing the need to increase the dose of prostacyclin in a patient (e.g., increasing the dose by at least 10%) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the disclosure contemplates a method of reducing the need for PcPH-specific hospitalization comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, PcPH-specific hospitalization is hospitalization of patient for at least 24 hours. In some embodiments, the disclosure contemplates a method of reducing the deterioration of PcPH comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, deterioration of PcPH comprises worsening in WHO functional class and/or a decrease of at least 15% in the 6MWD of the patient.

Optionally, methods disclosed herein for treating, preventing, or reducing the progression rate and/or severity of PcPH (e.g., WHO Group 2 and/or Group 5 PH), particularly treating, preventing, or reducing the progression rate and/or severity of one or more complications of PcPH, may further comprise administering to the patient one or more supportive therapies or additional active agents for treating PcPH. For example, the patient also may be administered one or more supportive therapies or active agents selected from the group consisting of: nitrates, hydralazine, prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, darusentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat, vericiguat, and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1-->3)-beta-D-glucopyranosyl] oleanolic acid; 3-O-[beta-D-glucopyranosyl-(1-->2)-beta-D-glucopyranosyl] oleanolic acid; 3-O-[beta-D-glucopyranosyl-(1-->3)-beta-D-glucopyranosyl] oleanolic acid 28-O-beta-D-glucopyranosyl ester; 3-O-[beta-D-glucopyranosyl-(1-->2)-beta-D-glucopyranosyl] oleanolic acid 28-O-beta-D-glucopyranosyl ester; 3-O-[a-L-rhamnopyranosyl-(1-->3)-beta-D-glucuronopyranosyl]oleanolic acid; 3-O-[alpha-L-rhamnopyranosyl-(1-->3)-beta-D-glucuronopyranosyl]oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate), an LV assist device (LVAD), implantable cardioverter-defibrillator (ICD), valve replacement, valve repair, lung and/or heart transplantation. In some embodiments, the methods described herein may further comprise administering to the patient parental prostacyclin. In some embodiments, the methods described herein may further comprise administering to the patient one additional supportive therapy or additional active agent (i.e., double therapy) for treating PcPH. In some embodiments, the methods described herein may further comprise administering to the patient two additional supportive therapies or additional active agents (i.e., triple therapy) for treating PcPH. In some embodiments, the methods described herein may further comprise administering to the patient three additional supportive therapies or additional active agents (i.e., quadruple therapy) for treating PcPH.

In some embodiments, the methods described herein may further comprise administering to the patient an angiotensin antagonist (e.g., angiotensin receptor blocker, ARB). In some embodiments, a patient is further administered one or more ARBs selected from the group consisting of losartan, irbesartan, olmesartan, candesartan, valsartan, fimasartan, azilsartan, salprisartan, and telmisartan. In some embodiments, a patient is administered losartan. In some embodiments, a patient is administered irbesartan. In some embodiments, a patient is administered olmesartan. In some embodiments, a patient is administered candesartan. In some embodiments, a patient is administered valsartan. In some embodiments, a patient is administered fimasartan. In some embodiments, a patient is administered azilsartan. In some embodiments, a patient is administered salprisartan. In some embodiments, a patient is administered telmisartan. In some embodiments, the methods described herein may further comprise administering to the patient a neprilysin inhibitor and an angiotensin antagonist (e.g., sacubitril/valsartan; also known as LCZ696).

In some embodiments, the methods described herein may further comprise administering to the patient one or more ACE inhibitors. In some embodiments, the one or more ACE inhibitors are selected from the group consisting of benazepril, captopril, enalapril, lisinopril, perindopril, ramipril (e.g., ramipen), trandolapril, and zofenopril. In some embodiments, a patient is administered benazepril. In some embodiments, a patient is administered captopril. In some embodiments, a patient is administered enalapril. In some embodiments, a patient is administered lisinopril. In some embodiments, a patient is administered perindopril. In some embodiments, a patient is administered ramipril. In some embodiments, a patient is administered trandolapril. In some embodiments, a patient is administered zofenopril. In some embodiments, the methods described herein may further comprise administering to the patient an ARB and an ACE inhibitor. In some embodiments, an alternative approach to angiotensin antagonism is to combine an ACE inhibitor and/or ARB with an aldosterone antagonist.

In some embodiments, the methods described herein may further comprise administering to the patient one or more neprilysin inhibitors. In some embodiments, the one or more neprilysin inhibitors are selected from the group consisting of thiorphan, phosphoramidon, candoxatrilat, candoxatril, ecadotril, omapatrilat, LBQ657, and sacubitril. In some embodiments, a patient is administered thiorphan. In some embodiments, a patient is administered phosphoramidon. In some embodiments, a patient is administered candoxatrilat. In some embodiments, a patient is administered candoxatril. In some embodiments, a patient is administered ecadotril. In some embodiments, a patient is administered omapatrilat. In some embodiments, a patient is administered LBQ657. In some embodiments, a patient is administered sacubitril. In some embodiments, the methods described herein may further comprise administering to the patient a neprilysin inhibitor and an ARB (e.g., sacubitril/valsartan; also known as LCZ696).

In some embodiments, the methods described herein may further comprise administering to the patient an angiotensin receptor-neprilysin inhibitor (ARNI). In some embodiments, the ARNI is sacubitril/valsartan (Entresto®). In some embodiments, a patient is administered sacubitril/valsartan (Entresto®).

In some embodiments, the methods described herein may further comprise administering to the patient one or more beta-blockers. In some embodiments, the one or more beta-blockers are selected from the group consisting of bisoprolol, carvedilol, metoprolol succinate (CR/XL), and nebivolol. In some embodiments, a patient is administered bisoprolol. In some embodiments, a patient is administered carvedilol. In some embodiments, a patient is administered metoprolol succinate (CR/XL). In some embodiments, a patient is administered nebivolol.

In some embodiments, the methods described herein may further comprise administering to the patient one or more mineralocorticoid receptor antagonists (MRA). In some embodiments, the one or more MRA are selected from the group consisting of eplerenone and spironolactone. In some embodiments, a patient is administered eplerenone. In some embodiments, a patient is administered spironolactone.

In some embodiments, the methods described herein may further comprise administering to the patient one or more hyperpolarization-activated cyclic nucleotide-gated (HCN) channel blockers. In some embodiments, the one or more HCN are selected from the group consisting of ivabradine, ZD7288, cilobradine, zatebradine, alinidine, clonidine, falipamil, TH92:20, and YM758. In some embodiments, a patient is administered ivabradine. In some embodiments, a patient is administered ZD7288. In some embodiments, a patient is administered cilobradine. In some embodiments, a patient is administered zatebradine. In some embodiments, a patient is administered alinidine. In some embodiments, a patient is administered clonidine. In some embodiments, a patient is administered falipamil. In some embodiments, a patient is administered TH92:20. In some embodiments, a patient is administered YM758.

In some embodiments, the one or more supportive therapies or additional active agents for treating PcPH are administered prior to administration of the ActRII antagonist. In some embodiments, the one or more supportive therapies or additional active agents for treating PcPH are administered in combination with the ActRII antagonist. In some embodiments, the one or more supportive therapies or additional active agents for treating PcPH are administered after the administration of the ActRII antagonist. As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

### Transplant Free Survival

Lung and/or heart transplantation is a surgical treatment option for patients with PcPH, and is often recommended for patients who don't respond to less invasive therapies (e.g., vasodilator therapy). Generally, PcPH patients who receive lung and/or heart transplantation have Functional Class III or Class IV pulmonary hypertension in accordance with the World Health Organization's functional classification system for pulmonary hypertension.

In certain aspects, the disclosure relates to methods of treating, preventing, or reducing the progression rate and/or severity of PcPH comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the method increases the patient's transplant free survival by at least 10% (e.g., at least 10%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, the method relates to increasing the patient's transplant free survival by at least 10%. In some embodiments, the method relates to increasing the patient's transplant free survival by at least 2%. In some embodiments, the method relates to increasing the patient's transplant free survival by at least 3%. In some embodiments, the method relates to increasing the patient's transplant free survival by at least 4%. In some embodiments, the method relates to increasing the patient's transplant free survival by at least 5%. In some embodiments, the method relates to increasing the patient's transplant free survival by at least 10%. In some embodiments, the method relates to increasing the patient's transplant free survival by at least 15%. In some embodiments, the method relates to increasing the patient's transplant free survival by at least 20%. In some embodiments, the method relates to increasing the patient's transplant free survival by at least 25%. In some embodiments, the method relates to increasing the patient's transplant free survival by at least 30%. In some embodiments, the method relates to increasing the patient's transplant free survival by at least 35%. In some embodiments, the method relates to increasing the patient's transplant free survival by at least 40%. In some embodiments, the method relates to increasing the patient's transplant free survival by at least 45%. In some embodiments, the method relates to increasing the patient's transplant free survival by at least 50%. In some embodiments, the method relates to increasing the patient's transplant free survival by at least 55%. In some embodiments, the method relates to increasing the patient's transplant free survival by at least 60%. In some embodiments, the method relates to increasing the patient's transplant free survival by at least 65%. In some embodiments, the method relates to increasing the patient's transplant free survival by at least 70%. In some embodiments, the method relates to increasing the patient's transplant free survival by at least 75%. In some embodiments, the method relates to increasing the patient's transplant free survival by at least 80%. In some embodiments, the method relates to increasing the patient's transplant free survival by at least 85%. In some embodiments, the method relates to increasing the patient's transplant free survival by at least 90%. In some embodiments, the method relates to increasing the patient's transplant free survival by at least 95%. In some embodiments, the method relates to increasing the patient's transplant free survival by at least 100%. In some embodiments, the method relates to increasing the patient's transplant free survival as compared to controls over 1 year. In some embodiments, the method relates to increasing the patient's transplant free survival as compared to controls over 2 years. In some embodiments, the method relates to increasing the patient's transplant free survival as compared to controls over 3 years. In some embodiments, the method relates to increasing the patient's transplant free survival as compared to controls over 4 years. In some embodiments, the method relates to increasing the patient's transplant free survival as compared to controls over 5 years. In some embodiments, the method relates to increasing the patient's transplant free survival as compared to controls over 6 years. In some embodiments, the method relates to increasing the patient's transplant free survival as compared to controls over 7 years.

### Death

In certain aspects, the disclosure relates to methods of reducing the risk of death in patients with PcPH comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the method reduces the patient's risk of death by at least 10% (e.g., at least 1%, 2%, 3%, 4%, 5%, 1, 1%, 20%, 2%, 30%, 3%, 40%, 45%, 0%, 5%, 60%, 65%, 70%, 75%, 80%, 85%, 90%, 95%, or 100%). In some embodiments, the method relates to reducing the patient's risk of death by at least 10%. In some embodiments, the method relates to reducing the patient's risk of death by at least 2%. In some embodiments, the method relates to reducing the patient's risk of death by at least 3%. In some embodiments, the method relates to reducing the patient's risk of death by at least 4%. In some embodiments, the method relates to reducing the patient's risk of death by at least 5%. In some embodiments, the method relates to reducing the patient's risk of death by at least 10%. In some embodiments, the method relates to reducing the patient's risk of death by at least 15%. In some embodiments, the method relates to reducing the patient's risk of death by at least 20%. In some embodiments, the method relates to reducing the patient's risk of death by at least 25%. In some embodiments, the method relates to reducing the patient's risk of death by at least 30%. In some embodiments, the method relates to reducing the patient's risk of death by at least 35%. In some embodiments, the method relates to reducing the patient's risk of death by at least 40%. In some embodiments, the method relates to reducing the patient's risk of death by at least 45%. In some embodiments, the method relates to reducing the patient's risk of death by at least 50%. In some embodiments, the method relates to reducing the patient's risk of death by at least 55%. In some embodiments, the method relates to reducing the patient's risk of death by at least 60%. In some embodiments, the method relates to reducing the patient's risk of death by at least 65%. In some embodiments, the method relates to reducing the patient's risk of death by at least 70%. In some embodiments, the method relates to reducing the patient's risk of death by at least 75%. In some embodiments, the method relates to reducing the patient's risk of death by at least 80%. In some embodiments, the method relates to reducing the patient's risk of death by at least 85%. In some embodiments, the method relates to reducing the patient's risk of death by at least 90%. In some embodiments, the method relates to reducing the patient's risk of death by at least 95%. In some embodiments, the method relates to reducing the patient's risk of death by at least 100%. In some embodiments, the method reduces the risk of hospitalization for one or more complications associated with PcPH.

### Composite Clinical Endpoint

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the method reduces the patient's composite clinical endpoint by at least 1% (e.g., at least 1%, 2%, 3%, 4%, 5%, 10%, 15%, 20%, 25%, 30%, 35%, 40%, 45%, 50%5, 5%, 60%, 65%, 70%, 75%, 80%, 85%, 90%, 95%, or 100%). In some embodiments, the method relates to reducing the patient's composite clinical endpoint by at least 10%. In some embodiments, the method relates to reducing the patient's composite clinical endpoint by at least 2%. In some embodiments, the method relates to reducing the patient's composite clinical endpoint by at least 3%. In some embodiments, the method relates to reducing the patient's composite clinical endpoint by at least 4%. In some embodiments, the method relates to reducing the patient's composite clinical endpoint by at least 5%. In some embodiments, the method relates to reducing the patient's composite clinical endpoint by at least 10%. In some embodiments, the method relates to reducing the patient's composite clinical endpoint by at least 15%. In some embodiments, the method relates to reducing the patient's composite clinical endpoint by at least 20%. In some embodiments, the method relates to reducing the patient's composite clinical endpoint by at least 25%. In some embodiments, the method relates to reducing the patient's composite clinical endpoint by at least 30%. In some embodiments, the method relates to reducing the patient's composite clinical endpoint by at least 35%. In some embodiments, the method relates to reducing the patient's composite clinical endpoint by at least 40%. In some embodiments, the method relates to reducing the patient's composite clinical endpoint by at least 45%. In some embodiments, the method relates to reducing the patient's composite clinical endpoint by at least 50%. In some embodiments, the method relates to reducing the patient's composite clinical endpoint by at least 55%. In some embodiments, the method relates to reducing the patient's composite clinical endpoint by at least 60%. In some embodiments, the method relates to reducing the patient's composite clinical endpoint by at least 65%. In some embodiments, the method relates to reducing the patient's composite clinical endpoint by at least 70%. In some embodiments, the method relates to reducing the patient's composite clinical endpoint by at least 75%. In some embodiments, the method relates to reducing the patient's composite clinical endpoint by at least 80%. In some embodiments, the method relates to reducing the patient's composite clinical endpoint by at least 85%. In some embodiments, the method relates to reducing the patient's composite clinical endpoint by at least 90%. In some embodiments, the method relates to reducing the patient's composite clinical endpoint by at least 95%. In some embodiments, the method relates to reducing the patient's composite clinical endpoint by at least 100%. In some embodiments, the method reduces the risk of hospitalization for one or more complications associated with PcPH. In some embodiments, the composite clinical endpoint comprises one or more endpoints selected from the group consisting of clinical worsening, hospitalization, and death.

### Functional Classes

PcPH (e.g., WHO Group 2 and Group 5 PH) at baseline can be mild, moderate or severe, as measured for example by World Health Organization (WHO) functional class, which is a measure of disease severity in patients with pulmonary hypertension. The WHO functional classification is an adaptation of the New York Heart Association (NYHA) system and is routinely used to qualitatively assess activity tolerance, for example in monitoring disease progression and response to treatment (Rubin (2004) Chest 126:7-10). Four functional classes are recognized in the WHO system: Class I: pulmonary hypertension without resulting limitation of physical activity; ordinary physical activity does not cause undue dyspnea or fatigue, chest pain or near syncope; Class II: pulmonary hypertension resulting in slight limitation of physical activity; patient comfortable at rest; ordinary physical activity causes undue dyspnea or fatigue, chest pain or near syncope; Class III: pulmonary hypertension resulting in marked limitation of physical activity; patient comfortable at rest; less than ordinary activity causes undue dyspnea or fatigue, chest pain or near syncope; Class IV: pulmonary hypertension resulting in inability to carry out any physical activity without symptoms; patient manifests signs of right-heart failure; dyspnea and/or fatigue may be present even at rest; discomfort is increased by any physical activity.

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has Functional Class I, Functional Class II, Functional Class III, or Functional Class IV pulmonary hypertension as recognized by the WHO. In some embodiments, the method relates to a patient that has Functional Class I pulmonary hypertension as recognized by the WHO. In some embodiments, the method relates to a patient that has Functional Class II pulmonary hypertension as recognized by the WHO. In some embodiments, the method relates to a patient that has Functional Class III pulmonary hypertension as recognized by the WHO. In some embodiments, the method relates to a patient that has Functional Class IV pulmonary hypertension as recognized by the WHO. In some embodiments, the method relates to patients having Functional Class II or Class III pulmonary hypertension as recognized by the WHO. In some embodiments, the method relates to patients having Functional Class II, Class III, or Class IV pulmonary hypertension as recognized by the WHO. In some embodiments, the method relates to patients having Functional Class I, Class II, Class III, or Class IV pulmonary hypertension as recognized by the WHO. In some embodiments, the method delays clinical worsening of PcPH. In some embodiments, the method delays clinical worsening of PcPH in accordance with the WHO's functional classification system for pulmonary hypertension.

In some embodiments, the disclosure relates to methods of preventing or reducing pulmonary hypertension Functional Class progression comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the reduction in Functional Class progression is a delay in Functional Class progression. In some embodiments, the method relates to preventing or decreasing pulmonary hypertension functional class progression as recognized by the WHO. In some embodiments, the disclosure relates to methods of promoting or increasing pulmonary hypertension Functional Class regression in a PcPH patient comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has Functional Class I, Functional Class II, Functional Class III, or Functional Class IV pulmonary hypertension as recognized by the WHO. In some embodiments, the method relates to preventing or delaying patient progression from Functional Class I pulmonary hypertension to Functional Class II pulmonary hypertension as recognized by the WHO. In some embodiments, the method relates to promoting patient regression from Functional Class II pulmonary hypertension to Functional Class I pulmonary hypertension as recognized by the WHO. In some embodiments, the method relates to preventing or delaying patient progression from Functional Class II pulmonary hypertension to Functional Class III pulmonary hypertension as recognized by the WHO. In some embodiments, the method relates to promoting patient regression from Functional Class III pulmonary hypertension to Functional Class II pulmonary hypertension as recognized by the WHO. In some embodiments, the method relates to promoting patient regression from Functional Class III pulmonary hypertension to Functional Class I pulmonary hypertension as recognized by the WHO. In some embodiments, the method relates to preventing or delaying patient progression from Functional Class III pulmonary hypertension to Functional Class IV pulmonary hypertension as recognized by the WHO. In some embodiments, the method relates to promoting patient regression from Functional Class IV pulmonary hypertension to Functional Class III pulmonary hypertension as recognized by the WHO. In some embodiments, the method relates to promoting patient regression from Functional Class IV pulmonary hypertension to Functional Class II pulmonary hypertension as recognized by the WHO. In some embodiments, the method relates to promoting patient regression from Functional Class IV pulmonary hypertension to Functional Class I pulmonary hypertension as recognized by the WHO.

The New York Heart Association (NYHA) functional classification (Table 10) has been used to describe the severity of symptoms and exercise intolerance in patients with pulmonary hypertension. The NYHA functional classification system provides a rapid assessment of patients' functional status in everyday clinical practice and is a well-established means of predicting prognosis. The four functional classes recognized by the NYHA functional classification system are shown in Table 10.

TABLE 10
New York Heart Association (NYHA) functional classification
of pulmonary hypertension based on severity of
symptoms and physical activity

| | |
|---|---|
| Class I | No limitation of physical activity. Ordinary |
| | physical activity does not cause undue |
| | breathlessness, fatigue, or palpitations. |
| Class II | Slight limitation of physical activity. |
| | Comfortable at rest, but ordinary physical |
| | activity results in undue breathlessness, fatigue, |
| | or palpitations. |
| Class III | Marked limitation of physical activity. |
| | Comfortable at rest, but less than ordinary |
| | physical activity results in undue breathlessness, |
| | fatigue, or palpitations. |
| Class IV | Unable to carry on any physical activity without |
| | discomfort. Symptoms at rest can be present |
| | If any physical activity is undertaken, |
| | discomfort is increased. |

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has Functional Class I, Functional Class II, Functional Class III, or Functional Class IV pulmonary hypertension as recognized by the NYHA.

In some embodiments, the method relates to a patient that has Functional Class I pulmonary hypertension as recognized by the NYHA. In some embodiments, a patient with Functional Class I pulmonary hypertension as recognized by the NYHA has no limitation of physical activity. In some embodiments, a patient with Functional Class I pulmonary hypertension as recognized by the NYHA experiences physical activity that does not cause undue breathlessness, fatigue, and/or palpitations. In some embodiments, the method relates to a patient that has Functional Class II pulmonary hypertension as recognized by the NYHA. In some embodiments, a patient with Functional Class II pulmonary hypertension as recognized by the NYHA has slight limitation of physical activity. In some embodiments, a patient with Functional Class II pulmonary hypertension as recognized by the NYHA experiences ordinary physical activity resulting in undue breathlessness, fatigue, or palpitations. In some embodiments, the method relates to a patient that has Functional Class III pulmonary hypertension as recognized by the NYHA. In some embodiments, a patient with Functional Class III pulmonary hypertension as recognized by the NYHA has marked limitation of physical activity. In some embodiments, a patient with Functional Class III pulmonary hypertension as recognized by the NYHA experiences less than ordinary physical activity resulting in undue breathlessness, fatigue, or palpitations. In some embodiments, the method relates to a patient that has Functional Class IV pulmonary hypertension as recognized by the NYHA. In some embodiments, a patient with Functional Class IV pulmonary hypertension as recognized by the NYHA is unable to carry on any physical activity without discomfort. In some embodiments, a patient with Functional Class IV pulmonary hypertension as recognized by the NYHA experiences symptoms at rest, as well as when any physical activity is undertaken, discomfort is increased. In some embodiments, the method relates to patients having Functional Class II or Class III pulmonary hypertension as recognized by the NYHA. In some embodiments, the method relates to patients having Functional Class II, Class III, or Class IV pulmonary hypertension as recognized by the NYHA. In some embodiments, the method relates to patients having Functional Class I, Class II, Class III, or Class IV pulmonary hypertension as recognized by the NYHA. In some embodiments, the method delays clinical worsening of PcPH. In some embodiments, the method delays clinical worsening of PcPH in accordance with the NYHA's functional classification system for pulmonary hypertension.

In some embodiments, the disclosure relates to methods of preventing or reducing pulmonary hypertension Functional Class progression comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the reduction in Functional Class progression is a delay in Functional Class progression. In some embodiments, the method relates to preventing or decreasing pulmonary hypertension functional class progression as recognized by the NYHA. In some embodiments, the disclosure relates to methods of promoting or increasing pulmonary hypertension Functional Class regression in a PcPH patient comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein the patient has Functional Class I, Functional Class II, Functional Class III, or Functional Class IV pulmonary hypertension as recognized by the NYHA. In some embodiments, the method relates to preventing or delaying patient progression from Functional Class I pulmonary hypertension to Functional Class II pulmonary hypertension as recognized by the NYHA. In some embodiments, the method relates to promoting patient regression from Functional Class II pulmonary hypertension to Functional Class I pulmonary hypertension as recognized by the NYHA. In some embodiments, the method relates to preventing or delaying patient progression from Functional Class II pulmonary hypertension to Functional Class III pulmonary hypertension as recognized by the NYHA. In some embodiments, the method relates to promoting patient regression from Functional Class III pulmonary hypertension to Functional Class II pulmonary hypertension as recognized by the NYHA. In some embodiments, the method relates to promoting patient regression from Functional Class III pulmonary hypertension to Functional Class I pulmonary hypertension as recognized by the NYHA. In some embodiments, the method relates to preventing or delaying patient progression from Functional Class III pulmonary hypertension to Functional Class IV pulmonary hypertension as recognized by the NYHA. In some embodiments, the method relates to promoting patient regression from Functional Class IV pulmonary hypertension to Functional Class III pulmonary hypertension as recognized by the NYHA. In some embodiments, the method relates to promoting patient regression from Functional Class IV pulmonary hypertension to Functional Class II pulmonary hypertension as recognized by the NYHA. In some embodiments, the method relates to promoting patient regression from Functional Class IV pulmonary hypertension to Functional Class I pulmonary hypertension as recognized by the NYHA.

In some embodiments, functional class regression is tested after the patient has received 4 weeks of treatment utilizing an ActRII polypeptide disclosed herein. In some embodiments, functional class regression is tested after the patient has received 8 weeks of treatment utilizing an ActRII polypeptide disclosed herein. In some embodiments, functional class regression is tested after the patient has received 12 weeks of treatment utilizing an ActRII polypeptide disclosed herein. In some embodiments, functional class regression is tested after the patient has received 16 weeks of treatment utilizing an ActRII polypeptide disclosed herein. In some embodiments, functional class regression is tested after the patient has received 20 weeks of treatment utilizing an ActRII polypeptide disclosed herein. In some embodiments, functional class regression is tested after the patient has received 22 weeks of treatment utilizing an ActRII polypeptide disclosed herein. In some embodiments, functional class regression is tested after the patient has received 24 weeks of treatment utilizing an ActRII polypeptide disclosed herein. In some embodiments, functional class regression is tested after the patient has received 26 weeks of treatment utilizing an ActRII polypeptide disclosed herein. In some embodiments, functional class regression is tested after the patient has received 28 weeks of treatment utilizing an ActRII polypeptide disclosed herein. In some embodiments, functional class regression is tested after the patient has received 48 weeks of treatment utilizing an ActRII polypeptide disclosed herein.

### Sustained Therapeutic Effect

In certain aspects, the disclosure relates to methods of treating, preventing, or reducing the progression rate and/or severity of PcPH in a sustained manner comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins). In some embodiments, the sustained manner comprises a persistent therapeutic effect following the reduction in administration of an ActRII polypeptide described herein. In some embodiments, the sustained manner comprises a persistent therapeutic effect following the withdrawal of administration of an ActRII polypeptide described herein. In some embodiments, the persistent therapeutic effect relates to maintaining functional or hematologic measurements over time. In some embodiments, the persistent therapeutic effect is measured as a sustained reduction in PVR. In some embodiments, the patient's PVR level does not increase for at least 1 week to at least 12 weeks following withdrawal of an ActRII polypeptide treatment described herein. In some embodiments, the patient's PVR level does not increase for at least 1 week following withdrawal of an ActRII polypeptide treatment described herein. In some embodiments, the patient's PVR level does not increase for at least 2 weeks following withdrawal of an ActRII polypeptide treatment described herein. In some embodiments, the patient's PVR level does not increase for at least 3 weeks following withdrawal of an ActRII polypeptide treatment described herein. In some embodiments, the patient's PVR level does not increase for at least 4 weeks following withdrawal of an ActRII polypeptide treatment described herein. In some embodiments, the patient's PVR level does not increase for at least 5 weeks following withdrawal of an ActRII polypeptide treatment described herein. In some embodiments, the patient's PVR level does not increase for at least 6 weeks following withdrawal of an ActRII polypeptide treatment described herein. In some embodiments, the patient's PVR level does not increase for at least 1 month to at least 6 months following withdrawal of an ActRII polypeptide treatment described herein.

In certain aspects, the disclosure relates to methods of treating or preventing cardiopulmonary remodeling associated with PcPH in a patient, comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins), wherein said method slows down cardiac remodeling and/or reverses cardiac remodeling. In some embodiments, the reversal is a sustained reversal. In some embodiments, the cardiac remodeling is left cardiac remodeling. In some embodiments, the cardiac remodeling is right cardiac remodeling. In some embodiments, the cardiac remodeling is ventricle remodeling. In some embodiments, the ventricle remodeling is left ventricular remodeling. In some embodiments, the ventricle remodeling is right ventricular remodeling. In some embodiments, the cardiac remodeling is ventricular dilation. In some embodiments, the method decreases interventricular septal end diastole. In some embodiments, the method decreases posterior wall end diastole.

In some embodiments, echocardiographic measurements may be used to assess the persistent therapeutic effect. In some embodiments, the echocardiographic measurements include, but are not limited to, RV fractional area change (RVFAC), sPAP, tricuspid annular systolic velocity (TASV), and Tei index. In some embodiments, a patient treated with an ActRII polypeptide disclosed herein shows a persistent therapeutic effect. In some embodiments, the persistent therapeutic effect results in decreased intrusion of the ventral wall into the left ventricle. In some embodiments, the persistent therapeutic effect results in an increase in right ventricular fractional area change (RVFAC).

### Known Treatments for PcPH

There is no known cure for PcPH (e.g., WHO Group 2 and/or Group 5 PH); current methods of treatment focus on prolonging patient lifespan and enhancing patient quality of life. This is usually associated with good exercise capacity, good right ventricle function, and a low mortality risk (e.g., bringing the patient to and/or keeping the patient in WHO Functional Class I or Functional Class II). Current methods of treatment of PcPH may include administration of: vasodilators such as prostacyclin, epoprostenol, and sildenafil; endothelin receptor antagonists such as bosentan; calcium channel blockers such as amlodipine, diltiazem, and nifedipine; anticoagulants such as warfarin; and diuretics. Treatment of PcPH has also been carried out using oxygen therapy, atrial septostomy, pulmonary thromboendarterectomy, and lung and/or heart transplantation. Each of these methods, however, suffers from one or multiple drawbacks which may include lack of effectiveness, serious side effects, low patient compliance, and high cost. In certain aspects, the method relate to treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of an ActRII antagonist (e.g., an antagonist of one or more of activin A, activin B, activin AB, activin AC, BMP6, BMP7, BMP9, BMP10, GDF3, GDF8, GDF11, and one or more Smad proteins) in combination with one or more additional active agents and/or supportive therapies for treating PcPH (e.g., vasodilators such as prostacyclin, epoprostenol, and sildenafil; endothelin receptor antagonists such as bosentan; calcium channel blockers such as amlodipine, diltiazem, and nifedipine; anticoagulants such as warfarin; diuretics; oxygen therapy; atrial septostomy; pulmonary thromboendarterectomy: LVAD; implantable cardioverter-defibrillator (ICD); valve replacement; valve repair; and lung and/or heart transplantation); bardoxolone methyl or a derivative thereof; oleanolic acid or derivative thereof.

### Measuring Hematologic Parameters in a Patient

In certain embodiments, the present disclosure provides methods for managing a patient that has been treated with, or is a candidate to be treated with, one or more one or more ActRII antagonist (e.g., an antagonist of one or more of activin A, activin B, activin AB, activin AC, BMP6, BMP7, BMP9, BMP10, GDF3, GDF8, GDF11, and one or more Smad proteins) by measuring one or more hematologic parameters in the patient. The hematologic parameters may be used to evaluate appropriate dosing for a patient who is a candidate to be treated with one or more ActRII antagonists of the present disclosure, to monitor the hematologic parameters during treatment, to evaluate whether to adjust the dosage during treatment with one or more ActRII antagonists of the disclosure, and/or to evaluate an appropriate maintenance dose of one or more ActRII antagonists of the disclosure. If one or more of the hematologic parameters are outside the normal level, dosing with one or more ActRII antagonists may be reduced, delayed or terminated.

Hematologic parameters that may be measured in accordance with the methods provided herein include, for example, red blood cell levels, blood pressure, iron stores, and other agents found in bodily fluids that correlate with increased red blood cell levels, using art recognized methods. In other embodiments, hematologic parameters such as white blood cell levels, platelet levels, and neutrophil levels may be measured using art recognized methods. Such parameters may be determined using a blood sample from a patient. Increases in red blood cell levels, hemoglobin levels, and/or hematocrit levels may cause increases in blood pressure. Decreases in white blood cell levels, platelet levels, and/or neutrophil levels may indicate a need to decrease, delay, or discontinue treatment of the administration of one or more ActRII polypeptides of the disclosure.

In one embodiment, if one or more hematologic parameters are outside the normal range or on the high side of normal in a patient who is a candidate to be treated with one or more ActRII antagonists (e.g., ActRII polypeptides), then onset of administration of the one or more ActRII antagonists of the disclosure may be delayed until the hematologic parameters have returned to a normal or acceptable level either naturally or via therapeutic intervention. For example, if a candidate patient is hypertensive or pre-hypertensive, then the patient may be treated with a blood pressure lowering agent in order to reduce the patient's blood pressure. Any blood pressure lowering agent appropriate for the individual patient's condition may be used including, for example, diuretics, adrenergic inhibitors (including alpha blockers and beta blockers), vasodilators, calcium channel blockers, angiotensin-converting enzyme (ACE) inhibitors, or angiotensin II receptor blockers. Blood pressure may alternatively be treated using a diet and exercise regimen. Similarly, if a candidate patient has iron stores that are lower than normal, or on the low side of normal, then the patient may be treated with an appropriate regimen of diet and/or iron supplements until the patient's iron stores have returned to a normal or acceptable level. For patients having higher than normal red blood cell levels and/or hemoglobin levels (e.g., hemoglobin levels >16.0 g/dL or hemoglobin levels >18.0 g/dL), then administration of the one or more ActRII polypeptides of the disclosure may be delayed or reduced until the levels have returned to a normal or acceptable level. In some embodiments, a normal or acceptable level of hemoglobin includes patients with hemoglobin levels between 8-15 g/dl. In some embodiments, a normal or acceptable level of hemoglobin includes patients with hemoglobin levels of <18 g/dl. In some embodiments, a normal or acceptable level of hemoglobin increase over time includes patients whose hemoglobin levels increase less than 2 g/dL over the first period of time in treatment. In some embodiments, the first period of time is 3 weeks. For patients having lower than normal white blood cell counts (e.g., leukopenia; white blood cell count <3000/mm³ or <3.0×10⁹/L (Grade 2)), then administration of the one or more ActRII polypeptides of the disclosure may be delayed or reduced until the levels have returned to a normal or acceptable level. For patients having lower than normal white blood cell counts (e.g., leukopenia; white blood cell count <2000/mm³ or <2.0×10⁹/L (Grade 3)), then administration of the one or more ActRII polypeptides of the disclosure may be delayed or reduced until the levels have returned to a normal or acceptable level. For patients having lower than normal platelet counts (e.g., thrombocytopenia; platelet count <75,000/mm³ or <75.0×10⁹/L (Grade 2)), then administration of the one or more ActRII polypeptides of the disclosure may be delayed or reduced until the levels have returned to a normal or acceptable level. For patients having lower than normal platelet counts (e.g., thrombocytopenia; platelet count <50,000/mm³ or <50.0×10⁹/L (Grade 3)), then administration of the one or more ActRII polypeptides of the disclosure may be delayed or reduced until the levels have returned to a normal or acceptable level. For patients having lower than normal neutrophil counts (e.g., neutropenia; neutrophil count <1500/mm³ or <1.5×10⁹/L (Grade 2)), then administration of the one or more ActRII polypeptides of the disclosure may be delayed or reduced until the levels have returned to a normal or acceptable level. For patients having lower than normal neutrophil counts (e.g., neutropenia; neutrophil count <1000/mm³ or <1.0×10⁹/L (Grade 3)), then administration of the one or more ActRII polypeptides of the disclosure may be delayed or reduced until the levels have returned to a normal or acceptable level.

In certain embodiments, if one or more hematologic parameters are outside the normal range or on the high side of normal in a patient who is a candidate to be treated with one or more ActRII antagonists (e.g., ActRII polypeptides), then the onset of administration may not be delayed. However, the dosage amount or frequency of dosing of the one or more ActRII antagonists of the disclosure may be set at an amount that would reduce the risk of an unacceptable increase in the hematologic parameters arising upon administration of the one or more ActRII antagonists of the disclosure. Alternatively, a therapeutic regimen may be developed for the patient that combines one or more ActRII antagonists with a therapeutic agent that addresses the undesirable level of the hematologic parameter. For example, if the patient has elevated blood pressure, then a therapeutic regimen may be designed involving administration of one or more ActRII antagonists and a blood pressure lowering agent. For a patient having lower than desired iron stores, a therapeutic regimen may be developed involving one or more ActRII antagonists of the disclosure and iron supplementation.

In one embodiment, baseline parameter(s) for one or more hematologic parameters may be established for a patient who is a candidate to be treated with one or more ActRII antagonists of the disclosure and an appropriate dosing regimen established for that patient based on the baseline value(s). Alternatively, established baseline parameters based on a patient's medical history could be used to inform an appropriate ActRII antagonist dosing regimen for a patient. For example, if a healthy patient has an established baseline blood pressure reading that is above the defined normal range it may not be necessary to bring the patient's blood pressure into the range that is considered normal for the general population prior to treatment with the one or more ActRII antagonists of the disclosure. A patient's baseline values for one or more hematologic parameters prior to treatment with one or more ActRII antagonists of the disclosure may also be used as the relevant comparative values for monitoring any changes to the hematologic parameters during treatment with the one or more ActRII antagonists of the disclosure.

In certain embodiments, one or more hematologic parameters are measured in patients who are being treated with one or more ActRII antagonists. The hematologic parameters may be used to monitor the patient during treatment and permit adjustment or termination of the dosing with the one or more ActRII antagonists of the disclosure or additional dosing with another therapeutic agent. For example, if administration of one or more ActRII antagonists results in an increase in blood pressure, red blood cell level, or hemoglobin level, or a reduction in iron stores, white blood cell count, platelet count, or absolute neutrophil count, then the dose of the one or more ActRII antagonists of the disclosure may be reduced in amount or frequency in order to decrease the effects of the one or more ActRII antagonists of the disclosure on the one or more hematologic parameters. If administration of one or more ActRII antagonists results in a change in one or more hematologic parameters that is adverse to the patient, then the dosing of the one or more ActRII antagonists of the disclosure may be terminated either temporarily, until the hematologic parameter(s) return to an acceptable level, or permanently. Similarly, if one or more hematologic parameters are not brought within an acceptable range after reducing the dose or frequency of administration of the one or more ActRII antagonists of the disclosure, then the dosing may be terminated. As an alternative, or in addition to, reducing or terminating the dosing with the one or more ActRII antagonists of the disclosure, the patient may be dosed with an additional therapeutic agent that addresses the undesirable level in the hematologic parameter(s), such as, for example, a blood pressure lowering agent or an iron supplement. For example, if a patient being treated with one or more ActRII antagonists has elevated blood pressure, then dosing with the one or more ActRII antagonists of the disclosure may continue at the same level and a blood-pressure-lowering agent is added to the treatment regimen, dosing with the one or more antagonist of the disclosure may be reduced (e.g., in amount and/or frequency) and a blood-pressure-lowering agent is added to the treatment regimen, or dosing with the one or more antagonist of the disclosure may be terminated and the patient may be treated with a blood-pressure-lowering agent.

### Measuring Various Parameters Over Time

In certain embodiments, one or more of the measurements of pulmonary hypertension (e.g., PcPH) described herein can be measured over various periods of treatment time. In some embodiments, one or more of the measurements of pulmonary hypertension described herein is measured after the patient has received 4 weeks of treatment utilizing an ActRII antagonist disclosed herein. In some embodiments, one or more of the measurements of pulmonary hypertension described herein is measured after the patient has received 8 weeks of treatment utilizing an ActRII antagonist disclosed herein. In some embodiments, one or more of the measurements of pulmonary hypertension described herein is measured after the patient has received 12 weeks of treatment utilizing an ActRII antagonist disclosed herein. In some embodiments, one or more of the measurements of pulmonary hypertension described herein is measured after the patient has received 16 weeks of treatment utilizing an ActRII antagonist disclosed herein. In some embodiments, one or more of the measurements of pulmonary hypertension described herein is measured after the patient has received 20 weeks of treatment utilizing an ActRII antagonist disclosed herein. In some embodiments, one or more of the measurements of pulmonary hypertension described herein is measured after the patient has received 22 weeks of treatment utilizing an ActRII antagonist disclosed herein. In some embodiments, one or more of the measurements of pulmonary hypertension described herein is measured after the patient has received 24 weeks of treatment utilizing an ActRII antagonist disclosed herein. In some embodiments, one or more of the measurements of pulmonary hypertension described herein is measured after the patient has received 26 weeks of treatment utilizing an ActRII antagonist disclosed herein. In some embodiments, one or more of the measurements of pulmonary hypertension described herein is measured after the patient has received 28 weeks of treatment utilizing an ActRII antagonist disclosed herein. In some embodiments, one or more of the measurements of pulmonary hypertension described herein is measured after the patient has received 48 weeks of treatment utilizing an ActRII antagonist disclosed herein.

### 5. Pharmaceutical Compositions & Modes of Administration

In certain embodiments, the therapeutic methods of the disclosure include administering the composition systemically, or locally as an implant or device. When administered, the therapeutic composition for use in this disclosure is in a substantially pyrogen-free, or pyrogen-free, physiologically acceptable form. Therapeutically useful agents other than the ActRII antagonists which may also optionally be included in the composition as described above, may be administered simultaneously or sequentially with the subject compounds in the methods disclosed herein.

Typically, protein therapeutic agents disclosed herein will be administered parentally, and particularly intravenously or subcutaneously. Pharmaceutical compositions suitable for parenteral administration may comprise one or more ActRII antagonists in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind described herein.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Further, the composition may be encapsulated or injected in a form for delivery to a target tissue site. In certain embodiments, compositions of the present invention may include a matrix capable of delivering one or more therapeutic compounds (e.g., ActRII antagonists) to a target tissue site, providing a structure for the developing tissue and optimally capable of being resorbed into the body. For example, the matrix may provide slow release of the ActRII antagonist. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

In certain embodiments, methods of the invention can be administered for orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds of the disclosure (e.g., ActRII antagonists). The various factors include, but are not limited to, the patient's age, sex, and diet, the severity disease, time of administration, and other clinical factors. Optionally, the dosage may vary with the type of matrix used in the reconstitution and the types of compounds in the composition. The addition of other known growth factors to the final composition, may also affect the dosage. Progress can be monitored by periodic assessment of bone growth and/or repair, for example, X-rays (including DEXA), histomorphometric determinations, and tetracycline labeling. In some embodiments, ActRII antagonists of the disclosure are administered at 0.1-2.0 mg/kg. In some embodiments, ActRII antagonists of the disclosure are administered at 0.1 mg/kg. In some embodiments, ActRII antagonists of the disclosure are administered at 0.2 mg/kg. In some embodiments, ActRII antagonists of the disclosure are administered at 0.3 mg/kg. In some embodiments, ActRII antagonists of the disclosure are administered at 0.4 mg/kg. In some embodiments, ActRII antagonists of the disclosure are administered at 0.5 mg/kg. In some embodiments, ActRII antagonists of the disclosure are administered at 0.6 mg/kg. In some embodiments, ActRII antagonists of the disclosure are administered at 0.7 mg/kg. In some embodiments, ActRII antagonists of the disclosure are administered at 0.8 mg/kg. In some embodiments, ActRII antagonists of the disclosure are administered at 0.9 mg/kg. In some embodiments, ActRII antagonists of the disclosure are administered at 1.0 mg/kg. In some embodiments, ActRII antagonists of the disclosure are administered at 1.1 mg/kg. In some embodiments, ActRII antagonists of the disclosure are administered at 1.2 mg/kg. In some embodiments, ActRII antagonists of the disclosure are administered at 1.3 mg/kg. In some embodiments, ActRII antagonists of the disclosure are administered at 1.4 mg/kg. In some embodiments, ActRII antagonists of the disclosure are administered at 1.5 mg/kg. In some embodiments, ActRII antagonists of the disclosure are administered at 1.6 mg/kg. In some embodiments, ActRII antagonists of the disclosure are administered at 1.7 mg/kg. In some embodiments, ActRII antagonists of the disclosure are administered at 1.8 mg/kg. In some embodiments, ActRII antagonists of the disclosure are administered at 1.9 mg/kg. In some embodiments, ActRII antagonists of the disclosure are administered at 2.0 mg/kg. The various factors include, but are not limited to, the patient's age, sex, and diet, the severity disease, time of administration, and other clinical factors. Optionally, the dosage may vary with the type of matrix used in the reconstitution and the types of compounds in the composition.

In some embodiments, a patient's hematologic parameters can be monitored by periodic assessments in order to determine if they have higher than normal red blood cell levels and/or hemoglobin levels (e.g., hemoglobin levels >16.0 g/dL or hemoglobin levels >18.0 g/dL). In some embodiments, patient's having higher than normal red blood cell levels and/or hemoglobin levels may receive a delayed or reduced dose until the levels have returned to a normal or acceptable level.

The probability of a patient having hemoglobin levels greater than 18 g/dL or increases in hemoglobin of greater than 2 g/dL may be higher during initial treatment with an ActRII antagonist. In certain embodiments, a dosing regimen can be used to prevent, ameliorate, or decrease the adverse changes in hemoglobin levels. In some embodiments, ActRII antagonists of the disclosure are administered using a dosing regimen. In some embodiments, the method comprises administering a dosing regimen of a therapeutically effective amount of an ActRII antagonist as disclosed herein to a patient, comprising a first dose of between 0.1 mg/kg and 1.0 mg/kg of said antagonist for a first period of time, and a second dose of between 0.1 mg/kg and 1.0 mg/kg of said antagonist subsequently administered for a second period of time. In some embodiments, the method comprises administering a dosing regimen of therapeutically effective amount of an ActRII antagonist as disclosed herein to a patient, comprising a first dose of between 0.1 mg/kg and 1.0 mg/kg of said antagonist for a first period of time, a second dose of between 0.1 mg/kg and 1.0 mg/kg of said antagonist administered for a second period of time, and a third dose of between 0.1 mg/kg and 1.0 mg/kg of said antagonist subsequently administered for a third period of time. In some embodiments, the first dose of ActRII antagonist is administered to a patient in an amount from about 0.2 mg/kg to about 0.4 mg/kg. In some embodiments, the first dose of ActRII antagonist is administered to a patient at a dose of 0.3 mg/kg. In some embodiments, the second dose of ActRII antagonist is administered to a patient in an amount from about 0.5 mg/kg to about 0.8 mg/kg. In some embodiments, the second dose of ActRII antagonist is administered to a patient at a dose of 0.7 mg/kg. In some embodiments, the third dose of ActRII antagonist is administered to a patient in an amount from about 0.2 mg/kg to about 0.4 mg/kg. In some embodiments, the third dose of ActRII antagonist is administered to a patient at a dose of 0.3 mg/kg.

In some embodiments, the dosing regimen comprises administering a first dose of ActRII antagonist to a patient in an amount of 0.3 mg/kg followed by administration of a second dose of ActRII antagonist to the patient in an amount of 0.7 mg/kg. In some embodiments, the dosing regimen comprises administering a first dose of ActRII antagonist to a patient in an amount of 0.3 mg/kg, administering a second dose of ActRII antagonist to the patient in an amount of 0.7 mg/kg, and administering a third dose of ActRII antagonist to the patient in an amount of 0.3 mg/kg. In some embodiments, the second dose exceeds the first dose. In some embodiments, the first dose exceeds the second dose. In some embodiments, the third dose exceeds the second dose. In some embodiments, the second dose exceeds the third dose. In some embodiments, the first period of time is at least 3 weeks. In some embodiments, the second period of time is at least 3 weeks. In some embodiments, the third period of time is at least 3 weeks. In some embodiments, the second period of time is at least 21 weeks. In some embodiments, the second period of time is at least 45 weeks. In some embodiments, the second period of time exceeds the first period of time. In some embodiments, the third period of time exceeds the first period of time. In some embodiments, the third period of time exceeds the second period of time.

In some embodiments, the change in dosing between the first dose and the second dose is determined by the attending physician considering various factors (e.g., hemoglobin levels). In some embodiments, the change in dosing between the second dose and the third dose is determined by the attending physician considering various factors (e.g., hemoglobin levels). In some embodiments, the various factors include, but are not limited to, the patient's change in hematologic parameters over a period of time. In some embodiments, a patient's hematologic parameters are monitored in order to determine if they have higher than normal red blood cell levels and/or hemoglobin levels (e.g., hemoglobin levels >16.0 g/dL or hemoglobin levels >18.0 g/dL). In some embodiments, a patient's hematologic parameters are monitored in order to determine if they have a higher than normal increase in hemoglobin levels over a period of time (e.g., hemoglobin level increase of >2 g/dL in less than 3 weeks). In some embodiments, the patient's dose of an ActRII antagonist as disclosed herein will be decreased (e.g., decrease in dose from 0.7 mg/kg to 0.3 mg/kg) if one or more of the patient's hematologic parameters before or during treatment is abnormal. In some embodiments, the patient's dose of an ActRII antagonist as disclosed herein will be maintained (e.g., maintained at 0.3 mg/kg or 0.7 mg/kg) if one or more of the patient's hematologic parameters before or during treatment is abnormal.

In some embodiments, the dosing regimen prevents, ameliorates, or decreases adverse effects of the ActRII antagonist. In some embodiments, administration of an ActRII antagonist in accordance with the dosage regimen as provided herein results in decreased adverse side effects. In some embodiments, administration of an ActRII antagonist in accordance with the dosage regimen as provided herein decreases the probability of having hemoglobin levels greater than 18 g/dL during the first period of time. In some embodiments, administration of an ActRII antagonist in accordance with the dosage regimen as provided herein decreases the probability of having hemoglobin levels greater than 18 g/dL in the first 3 weeks of treatment. In some embodiments, administration of an ActRII antagonist in accordance with the dosage regimen as provided herein decreases the probability of increasing hemoglobin levels by greater than 2 g/dL during the first period of time. In some embodiments, administration of an ActRII antagonist in accordance with the dosage regimen as provided herein decreases the probability of increasing hemoglobin levels by greater than 2 g/dL in the first 3 weeks of treatment.

In certain embodiments, ActRII antagonists of the disclosure are administered once a day. In certain embodiments, ActRII antagonists of the disclosure are administered twice a day. In certain embodiments, ActRII antagonists of the disclosure are administered once a week. In certain embodiments, ActRII antagonists of the disclosure are administered twice a week. In certain embodiments, ActRII antagonists of the disclosure are administered three times a week. In certain embodiments, ActRII antagonists of the disclosure are administered every two weeks. In certain embodiments, ActRII antagonists of the disclosure are administered every three weeks. In certain embodiments, ActRII antagonists of the disclosure are administered every four weeks. In certain embodiments, ActRII antagonists of the disclosure are administered every month.

In certain embodiments, the present invention also provides gene therapy for the in vivo production of ActRII antagonists (e.g., ActRII polypeptides). Such therapy would achieve its therapeutic effect by introduction of the ActRII antagonist polynucleotide sequences into cells or tissues having the disorders as listed above. Delivery of ActRII antagonist polynucleotide sequences can be achieved using a recombinant expression vector such as a chimeric virus or a colloidal dispersion system. Preferred for therapeutic delivery of ActRII antagonist polynucleotide sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing the ActRII antagonist. In a preferred embodiment, the vector is targeted to bone or cartilage.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for ActRII antagonist polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

The disclosure provides formulations that may be varied to include acids and bases to adjust the pH; and buffering agents to keep the pH within a narrow range.

### 6. Kits

The present disclosure provides a kit comprising a lyophilized polypeptide and an injection device. In certain embodiments, the lyophilized polypeptide comprises an ActRII antagonist (e.g., an antagonist of one or more of activin A, activin B, activin AB, activin AC, BMP6, BMP7, BMP9, BMP10, GDF3, GDF8, GDF11, and one or more Smad proteins), or fragments, functional variants, or modified forms thereof. In certain embodiments, the lyophilized polypeptide binds to one or more ligands selected from the group consisting of activin A, activin B, and GDF11. In certain such embodiments, the lyophilized polypeptide further binds to one or more ligands selected from the group consisting of BMP10, GDF8, and BMP6. In certain embodiments, the lyophilized polypeptide binds to activin and/or GDF11.

In some embodiments, the lyophilized polypeptide comprises a polypeptide that comprises, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of any one of SEQ ID NOs: 9, 10, 11, 36, 39, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184,185, 186, 187, 188, 189, 190, 191, 192, 193, 194, 195, 196, 197, 198, 199, 200, 201, 202, 203, 204, 205, 206, 207, 208, 209, 210, 211, 283, 304, 408, and 409.

In some embodiments, the lyophilized polypeptide comprises a polypeptide that comprises, consists essentially of, or consists of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 4, 5, 6, 40, 42, 45, 46, 47, 48, 69, 74, 77, 78, 79, 138, 282, 289, 290, 291, 292, 293, 294, 295, 296, 297, 298, 299, 300, 301, 302, 303, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, and 407.

In certain embodiments of the foregoing, the lyophilized polypeptide comprises a fusion protein further comprising an Fc domain of an immunoglobulin. In certain such embodiments, the Fc domain of the immunoglobulin is an Fc domain of an IgG1 immunoglobulin. In other embodiments, the fusion protein further comprises a linker domain positioned between the polypeptide domain and the Fc domain of the immunoglobulin. In certain embodiments, the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24). In certain embodiments, the linker domain comprises TGGG (SEQ ID NO: 23).

In certain embodiments, the lyophilized polypeptide is part of a homodimer protein complex. In certain embodiments, the lyophilized polypeptide is part of a heterodimer protein complex.

In certain embodiments, the polypeptide is glycosylated.

The present disclosure provides a kit comprising a sterile powder comprising a lyophilized polypeptide as disclosed herein and an injection device. In some embodiments of the kits disclosed herein, the sterile powder comprising a lyophilized polypeptide is pre-filled in one or more containers, such as one or more vials.

In certain embodiments, the pH range for the sterile powder comprising a lyophilized polypeptide is from 7 to 8. In some embodiments, the sterile powder comprising a lyophilized polypeptide further comprises a buffering agent. In some embodiments, the buffering agent may be added in an amount of at least 10 mM. In some embodiments, the buffering agent may be added in an amount in the range of between about 10 mM to about 200 mM. In some embodiments, the buffering agent comprises citric acid monohydrate and/or trisodium citrate dehydrate.

In some embodiments, the sterile powder comprising a lyophilized polypeptide further comprises a surfactant. In some embodiments, the surfactant comprises a polysorbate. In some embodiments, the surfactant comprises polysorbate 80.

In some embodiments, the sterile powder comprising a lyophilized polypeptide further comprises a lyoprotectant. In some embodiments, the lyoprotectant comprises a sugar, such as disaccharides (e.g, sucrose). In some embodiments, the lyoprotectant comprises sucrose, trehalose, mannitol, polyvinylpyrrolidone (PVP), dextrose, and/or glycine. In some embodiments, the lyoprotectant comprises sucrose. In some embodiments, the sterile powder comprises the lyoprotectant and lyophilized polypeptide in a weight ratio of at least 1:1 lyophilized polypeptide to lyoprotectant. In some embodiments, the sterile powder comprises the lyoprotectant and lyophilized polypeptide in a weight ratio of from 1:1 to 1:10 lyophilized polypeptide to lyoprotectant. In some embodiments, the sterile powder comprises the lyoprotectant and lyophilized polypeptide in a weight ratio of 1:1, 1:2, 1:3, 1:4, 1:5, 1:6, 1:7, 1:8, 1:9, or 1:10 lyophilized polypeptide to lyoprotectant. In some embodiments, the sterile powder comprises the lyoprotectant and lyophilized polypeptide in a weight ratio of 1:6 lyophilized polypeptide to lyoprotectant. In certain embodiments of the foregoing, the sterile powder comprises lyoprotectant in an amount sufficient to stabilize the lyophilized polypeptide.

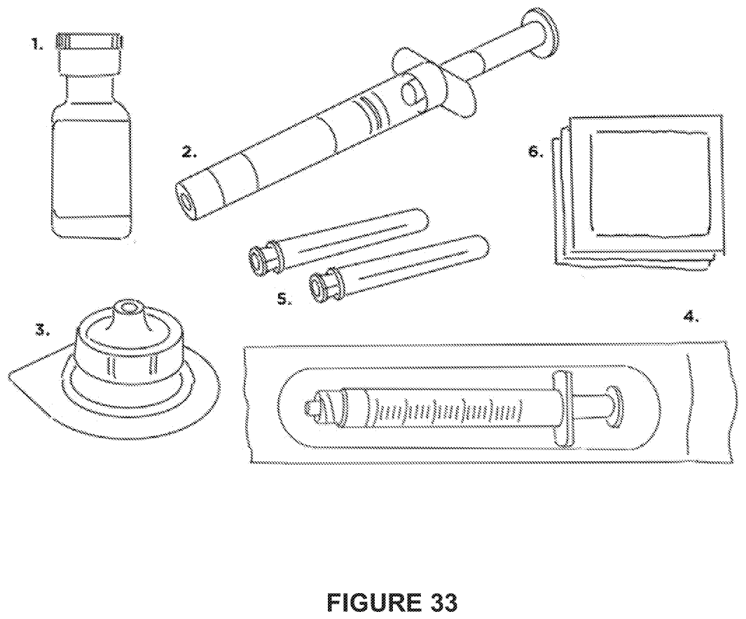

Also described herein are kits comprising a lyophilized polypeptide and one ore more of the following: an injection device, a vial (1) capable of holding a lyophilized polypeptide, reconstituted sterile injectable solution, or sterile injectable solution, a prefilled syringe (2) containing a reconstitution solution used to reconstitute lyophilized polypeptide from (1) into a sterile injectable solution, a vial adapter (3) that couples the vial (1) to the pre-filled syringe (2) via attachment to the vial at one end, and attachment to the pre-filled syringe at an opposite end, a syringe (4), a needle (5) provided for administration of sterile injectable solution, and swab wipes (6) for sterilization of individual kit components (See FIG. 33).

In certain embodiments of the kits disclosed herein, the injection device comprises a syringe. In certain such embodiments, the syringe is pre-filled with a reconstitution solution. In some embodiments, the reconstitution solution comprises a pharmaceutically acceptable carrier and/or excipient. In some embodiments, the pharmaceutically acceptable carrier comprises aqueous solutions such as water, physiologically buffered saline, or other solvents or vehicles such as glycols, glycerol, oils or injectable organic esters. In some embodiments, the pharmaceutically acceptable excipient comprises a pharmaceutically acceptable excipient selected from calcium phosphates, calcium carbonates, calcium sulfates, halites, metallic oxides, sugars, sugar alcohols, starch, glycols, povidones, mineral hydrocarbons, acrylic polymers, fatty alcohols, mineral stearates, glycerin, and/or lipids. In certain embodiments, the reconstitution solution comprises pharmaceutically acceptable sterile isotonic aqueous or nonaqueous solutions, dispersions, suspensions or emulsions. In certain such embodiments, the reconstitution solution comprises antioxidants, buffers, bacteriostats, and/or solutes which render the formulation isotonic with the blood of the intended recipient. In other embodiments, the reconstitution solution comprises suspending or thickening agents.

In certain embodiments of the kits disclosed herein, the kit further comprises a vial adapter. In some embodiments, the vial pre-filled with sterile powder comprising a lyophilized polypeptide attaches to one end of the vial adapter. In some embodiments, the syringe pre-filled with a reconstitution solution as disclosed herein attaches to an end of the vial adapter. In some embodiments, the syringe pre-filled with a reconstitution solution as disclosed herein and the vial pre-filled with sterile powder comprising a lyophilized polypeptide are attached to opposite ends of the vial adapter. In some embodiments, the reconstitution solution is transferred from the pre-filled syringe to the vial. In some embodiments, transfer of the reconstitution solution to the vial pre-filled with sterile powder comprising a lyophilized polypeptide reconstitutes the lyophilized polypeptide into a sterile injectable solution. In some embodiments, the lyophilized polypeptide is reconstituted into a sterile injectable solution. In some embodiments, the lyophilized polypeptide is reconstituted into a sterile injectable solution prior to use.

In other embodiments of the kits disclosed herein, the kit further comprises a pump apparatus. In certain embodiments, the pump apparatus comprises an electromechanical pumping assembly. In certain embodiments, the pump apparatus comprises a reservoir for holding a sterile injectable solution. In certain embodiments, the reservoir holds 1 mL of sterile injectable solution. In certain embodiments, the pump apparatus comprises one or more vials or cartridges comprising a sterile injectable solution. In certain embodiments, the vials or cartridges are prefilled with sterile injectable solution. In certain embodiments, the vials or cartridges comprise sterile injectable solution reconstituted from a lyophilized polypeptide. In certain embodiments, the reservoir is coupled to the vial or cartridge. In certain embodiments, the vial or cartridge holds 1-20 mL of sterile injectable solution. In certain embodiments, the electromechanical pumping assembly comprises a pump chamber. In certain embodiments, the electromechanical pumping assembly is coupled to the reservoir. In certain embodiments, the sterile injectable solution is received from the reservoir into the pump chamber. In some embodiments, the electromechanical pumping assembly comprises a plunger that is disposed such that sterile injectable solution in the pump chamber is in direct contact with the plunger. In certain embodiments, a sterile injectable solution is received from the reservoir into the pump chamber during a first pumping phase, and is delivered from the pump chamber to a subject during a second pumping phase. In certain embodiments, the electromechanical pumping assembly comprises control circuitry. In certain embodiments, control circuitry drives the plunger to (a) draw the sterile injectable solution into the pump chamber during the first pumping phase and (b) deliver the sterile injectable solution from the pump chamber in a plurality of discrete motions of the plunger during the second pumping phase, thereby delivering the therapeutic substance to the subject in a plurality of controlled and discrete dosages throughout the second pumping phase. In certain embodiments, a cycle of alternating the first and second pumping phases may be repeated until a desired dose is administered. In certain embodiments, the pump apparatus is coupled to a wearable patch. In certain embodiments, the pump apparatus is a wearable pump apparatus. In some embodiments, the pump apparatus administers a dose every 3 weeks. In some embodiments, the pump apparatus administers the dose via subcutaneous injection.

The present disclosure provides a kit used for reconstituting a lyophilized polypeptide into a sterile injectable solution. In certain embodiments, the resulting sterile injectable solution is useful in the methods disclosed herein.

In certain embodiments of the kits disclosed herein, the kit further comprises an injectable device for use in administering the sterile injectable solution parenterally. In some embodiments, the sterile injectable solution is administered via subcutaneous injection. In some embodiments, the sterile injectable solution is administered via intradermal injection. In some embodiments, the sterile injectable solution is administered via intramuscular injection. In some embodiments, the sterile injectable solution is administered via intravenous injection. In some embodiments, the sterile injectable solution is self-administered. In some embodiments, the sterile injectable solution comprises a therapeutically effective dose. In some embodiments, the therapeutically effective dose comprises a weight based dose. In some embodiments, the weight based dose is 0.3 mg/kg. In some embodiments, the weight based dose is 0.7 mg/kg.

In some embodiments of the kits is closed herein, the kit further comprises one or more vials or cartridges containing the lyophilized polypeptide. In some embodiments, the kit comprises at least two vials or cartridges containing the lyophilized polypeptide. In some embodiments, the kit comprises at least three vials or cartridges containing the lyophilized polypeptide. In some embodiments, the two vials can contain the same or different amounts of the lyophilized polypeptide. In some embodiments, the vials or cartridges comprise a vial or cartridge containing between 25 mg to 60 mg of lyophilized polypeptide. In some embodiments, at least one of the vials or cartridges comprise a vial or cartridge containing 60 mg of lyophilized polypeptide. In some embodiments, at least one of the vials or cartridges comprise a vial or cartridge containing 45 mg of lyophilized polypeptide. In some embodiments, at least one of the vials or cartridges comprise a vial or cartridge containing 30 mg of lyophilized polypeptide. In some embodiments, at least one of the vials or cartridges comprise a vial or cartridge containing 25 mg of lyophilized polypeptide. In some embodiments, a first vial or cartridge contains 45 mg of lyophilized polypeptide and a second vial or cartridge contains 60 mg of lyophilized polypeptide. In some embodiments, a first vial or cartridge contains 30 mg of lyophilized polypeptide and a second vial or cartridge contains 60 mg of lyophilized polypeptide. In some embodiments, a first vial or cartridge contains 30 mg of lyophilized polypeptide, a second vial or cartridge contains 45 mg of lyophilized polypeptide, and a third vial or cartridge contains 60 mg of lyophilized polypeptide. In some embodiments, a first vial or cartridge contains 25 mg of lyophilized polypeptide, a second vial or cartridge contains 45 mg of lyophilized polypeptide, and a third vial or cartridge contains 60 mg of lyophilized polypeptide. In some embodiments, the one or more vials or cartridges are refrigerated at 2-8° C.

### 7. Additional Sequences

TABLE 11

| SEQ | |
|---|---|
| ID | |
| NO | Amino Acid Sequence |
| 211 | GAILGRSETQECLFFNANWEKDRTNQTGVEPCYGDKDKRRHCFATWKNISGS |
| | IEIVKQGCWLDDINCYDRTDCVEKKDSPEVYFCCCEGNMCNEKFSYFPEMEV |
| | TQPTS |
| 284 | MEWSWVFLFFLSVTTGVHSDKTHTCPPCPAPELLGGPSVFLFPPKPKDTLMISR |
| | TPEVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSRDELT |
| | KNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 286 | GPVEVFITETPSQPNSHPIQWNAPQPSHISKYILRWRPKNSVGRWKEATIPGHL |
| | NSYTIKGLKPGVVYEGQLISIQQYGHQEVTRFDFTTTST |
| 287 | MKWVTFISLLFLFSSAYSRGVFRRDAHKSEVAHRFKDLGEENFKALVLIAFAQ |
| | YLQQCPFEDHVKLVNEVTEFAKTCVADESAENCDKSLHTLFGDKLCTVATLR |
| | ETYGEMADCCAKQEPERNECFLQHKDDNPNLPRLVRPEVDVMCTAFHDNEE |
| | TFLKKYLYEIARRHPYFYAPELLFFAKRYKAAFTECCQAADKAACLLPKLDEL |
| | RDEGKASSAKQRLKCASLQKFGERAFKAWAVARLSQRFPKAEFAEVSKLVTD |
| | LTKVHTECCHGDLLECADDRADLAKYICENQDSISSKLKECCEKPLLEKSHCI |
| | AEVENDEMPADLPSLAADFVESKDVCKNYAEAKDVFLGMFLYEYARRHPDY |
| | SVVLLLRLAKTYETTLEKCCAAADPHECYAKVFDEFKPLVEEPQNLIKQNCEL |
| | FEQLGEYKFQNALLVRYTKKVPQVSTPTLVEVSRNLGKVGSKCCKHPEAKRM |
| | PCAEDYLSVVLNQLCVLHEKTPVSDRVTKCCTESLVNRRPCFSALEVDETYVP |
| | KEFNAETFTFHADICTLSEKERQIKKQTALVELVKHKPKATKEQLKAVMDDF |
| | AAFVEKCCKADDKETCFAEEGKKLVAASQAALGL |
| 305 | ETRECIYYNANWELERTNQSGLERCYGDKDKRRHCYASWRNSSGTIELVKKG |
| | CWLDDINCYDRTDCVEKKDSPEVYFCCCEGNFCNERFTHLPEAGGPEVTYEPP |
| | PTAPT |
| 306 | ETRECIYYNANWELERTNQSGLERCYGDKDKRRHCYASWRNSSGTIELVKKG |
| | CWLDDFNCYDRTDCVEKKDSPEVYFCCCEGNFCNERFTHLPEAGGPEVTYEP |
| | PPTAPT |
| 307 | ETRECIYYNANWELERTNQSGLERCEGEQDKRLHCYASWRNSSGTIELVKKG |
| | CWLDDINCYDRTDCVEKKDSPEVYFCCCEGNFCNERFTHLPEAGGPEVTYEPP |
| | PTAPT |
| 308 | ETRECIYYNANWELERTNQSGLERCEGEQDKRLHCYASWRNSSGTIELVKKG |
| | CWLDDINCYDRTDCVEKKDSPQVYFCCCEGNFCNERFTHLPEAGGPEVTYEP |
| | PPTAPT |
| 309 | ETRECIYYNANWELERTNQSGLERCEGEQDKRLHCYASWRNSSGTIELVKKG |
| | CWLDDFNCYDRTDCVEKKDSPQVYFCCCEGNFCNERFTHLPEAGGPEVTYEP |
| | PPTAPT |
| 310 | ETRECIYYNANWELERTNQSGLERCEGEQDKRLHCYASWRNSSGTIELVKKG |
| | CWLDDFNCYDRTDCVEKKDSPEVYFCCCEGNFCNERFTHLPEAGGPEVTYEP |
| | PPTAPT |
| 311 | ETRECIYYNANWELERTNQSGLERCEGEQDKRLHCYASWRNSSGTIELVKKG |
| | CWLDDFNCYDRQECVEKKDSPEVYFCCCEGNFCNERFTHLPEAGGPEVTYEP |
| | PPTAPT |
| 312 | ETRECIYYNANWELERTNQSGLERCEGEQDKRLHCYASWRNSSGTIELVKKG |
| | CWLDDFNCYDRQECVEKKDSPQVYFCCCEGNFCNERFTHLPEAGGPEVTYEP |
| | PPTAPT |
| 313 | ETRECIYYNANWELERTNQSGLERCEGEQDKRLHCYASWRNSSGTIELVKKG |
| | CWLDDINCYDRQECVEKKDSPEVYFCCCEGNFCNERFTHLPEAGGPEVTYEPP |
| | PTAPT |
| 314 | ETRECIYYNANWELERTNQSGLERCEGEQDKRLHCYASWRNSSGTIELVKKG |
| | CWLDDFNCYDRTDCVEKKDSPEVYFCCCEGNMCNERFTHLPEAGGPEVTYEP |
| | PPTAPT |
| 315 | ETRECIYYNANWELERTNQSGLERCEGDQDKRLHCYASWRNSSGTIELVKKG |
| | CWLDDFNCYDRQECVEKKDSPEVYFCCCEGNMCNERFTHLPEAGGPEVTYEP |
| | PPTAPT |
| 316 | ETQECIYYNANWEKDRTNQTGVEPCYGDKDKRRHCYASWRNSSGTIELVKK |
| | GCWLDDFNCYDRQECVATEENPQVYFCCCEGNFCNERFTHLPEAGGPEVTYE |
| | PPPTAPT |
| 317 | ETQECIYYNANWEKDRTNQTGVEPCEGDQDKRLHCYASWRNSSGTIELVKK |
| | GCWLDDFNCYDRQECVATEENPQVYFCCCEGNFCNERFTHLPEAGGPEVTYE |
| | PPPTAPT |
| 318 | ETRECIYYNANWELERTNQSGLERCYGDKDKRRHCYASWRNSSGTIELVKKG |
| | CWLDDFNCYDRQECVATEENPQVYFCCCEGNFCNERFTHLPEAGGPEVTYEP |
| | PPTAPT |
| 319 | ETRECIYYNANWEKDRTNQTGVEPCEGDQDKRLHCYASWRNSSGTIELVKK |
| | GCWLDDFNCYDRQECVATEENPQVYFCCCEGNFCNERFTHLPEAGGPEVTYE |
| | PPPTAPT |
| 320 | ETQECIYYNANWEKDRTNQTGVEPCEGDQDKRLHCYASWRNSSGTIELVKK |
| | GCWLDDFNCYDRQECVATEENPQVYFCCCEGNFCNERFTHLPEAGGPEVTYE |
| | PPPTAPT |
| 321 | ETQECIYYNANWEKDRTNQTGLERCEGEQDKRLHCYASWRNSSGTIELVKKG |
| | CWLDDFNCYDRQECVATEENPQVYFCCCEGNFCNERFTHLPEAGGPEVTYEP |
| | PPTAPT |
| 322 | ETQECIYYNANWEKDRTNQSGLERCEGEQDKRLHCYASWRNSSGTIELVKKG |
| | CWLDDFNCYDRQECVATEENPQVYFCCCEGNFCNERFTHLPEAGGPEVTYEP |
| | PPTAPT |
| 323 | ETRECIYYNANWEKDRTNQSGLERCEGEQDKRLHCYASWRNSSGTIELVKKG |
| | CWLDDFNCYDRQECVATEENPQVYFCCCEGNFCNERFTHLPEAGGPEVTYEP |
| | PPTAPT |
| 324 | ETQECIYYNANWELERTNQSGLERCEGEQDKRLHCYASWRNSSGTIELVKKG |
| | CWLDDFNCYDRQECVATEENPQVYFCCCEGNFCNERFTHLPEAGGPEVTYEP |
| | PPTAPT |
| 325 | ETRECIYYNANWELERTNQSGLERCEGEQDKRLHCFATWRNSSGTIELVKQG |
| | CWLDDFNCYDRQECVATEENPQVYFCCCEGNFCNERFTHLPEAGGPEVTYEP |
| | PPTAPT |
| 326 | ETRECIYYNANWELERTNQSGLERCYGDKDKRRHCYASWRNSSGTIELVKKG |
| | CWLDDINCYDRQECVATEENPQVYFCCCEGNFCNERFTHLPEAGGPEVTYEP |
| | PPTAPT |
| 327 | ETRECIYYNANWELERTNQSGLERCEGEQDKRLHCYASWRNSSGTIELVKKG |
| | CWLDDFNCYDRQECVATKENPQVYFCCCEGNFCNERFTHLPEAGGPEVTYEP |
| | PPTAPT |
| 328 | ETRECIYYNANWELERTNQSGLERCEGEQDKRLHCYASWRNSSGTIELVKKG |
| | CWLDDINCYDRQECVATEENPQVYFCCCEGNFCNERFTHLPEAGGPEVTYEP |
| | PPTAPT |
| 329 | ETRECIYYNANWELERTNQSGLERCEGEQDKRLHCYASWRNSSGTIELVKKG |
| | CWLDDINCYDRQECVATKENPQVYFCCCEGNFCNERFTHLPEAGGPEVTYEP |
| | PPTAPT |
| 330 | ETRECIYYNANWELERTNQSGLERCEGDKDKRLHCYASWRNSSGTIELVKKG |
| | CWLDDINCYDRQECVATKENPQVYFCCCEGNFCNERFTHLPEAGGPEVTYEP |
| | PPTAPT |
| 331 | ETRECIYYNANWELERTNQSGLERCEGDQDKRLHCYASWRNSSGTIELVKKG |
| | CWLDDINCYDRQECVATKENPQVYFCCCEGNFCNERFTHLPEAGGPEVTYEP |
| | PPTAPT |
| 332 | ETRECIYYNANWELERTNQSGLERCEGDQDKRLHCYASWRNSSGTIELVKKG |
| | CWLDDFNCYDRQECVATKENPQVYFCCCEGNFCNERFTHLPEAGGPEVTYEP |
| | PPTAPT |
| 333 | ETRECIYYNANWELERTNQSGLERCEGDQDKRLHCYASWRNSSGTIELVKKG |
| | CWLDDFNCYDRQECVATEENPQVYFCCCEGNFCNERFTHLPEAGGPEVTYEP |
| | PPTAPT |
| 334 | ETRECIYYNANWELERTNQSGLERCYGDKDKRRHCYASWRNSSGTIELVKKG |
| | CWLDDINCYDRQECVEKKDSPEVYFCCCEGNFCNERFTHLPEAGGPEVTYEPP |
| | PTAPT |
| 335 | ETRECIFFNANWEKDRTNQTGVEPCEGEQDKRLHCYASWRNSSGTIELVKKG |
| | CWLDDFNCYDRQECVATEENPQVYFCCCEGNFCNERFTHLPEAGGPEVTYEP |
| | PPTAPT |
| 336 | ETRECIYYNANWELERTNQSGLERCEGEQDKRLHCFATWKNISGSIELVKQGC |
| | WLDDFNCYDRQECVATEENPQVYFCCCEGNFCNERFTHLPEAGGPEVTYEPP |
| | PTAPT |
| 337 | ETRECIYYNANWELERTNQSGLERCEGEQDKRLHCYASWRNSSGTIELVKKG |
| | CWLDDFNCYDRTDCVATEENPQVYFCCCEGNFCNERFTHLPEAGGPEVTYEP |
| | PPTAPT |
| 338 | ETRECIYYNANWELERTNQSGLERCEGEQDKRLHCYASWRNSSGTIELVKKG |
| | CWLDDFNCYDRQECVATEENPQVYFCCCEGNMCNERFTHLPEAGGPEVTYE |
| | PPPTAPT |
| 339 | ETRECIYYNANWELERTNQSGLERCEGDQDKRLHCYASWRNSSGTIELVKKG |
| | CWLDDINCYDRQECVATEENPQVYFCCCEGNFCNERFTHLPEAGGPEVTYEP |
| | PPTAPT |
| 340 | ETQECLFFNANWEKDRTNQSGVEPCYGDKDKRRHCYASWRNSSGTIELVKK |
| | GCWLDDFNCYDRQECVATEENPQVYFCCCEGNFCNERFTHLPEAGGPEVTYE |
| | PPPTAPT |
| 341 | ETQECLFFNANWEKDRTNQSGVEPCEGEQDKRLHCYASWRNSSGTIELVKKG |
| | CWLDDFNCYDRQECVATEENPQVYFCCCEGNFCNERFTHLPEAGGPEVTYEP |
| | PPTAPT |
| 342 | ETRECLFFNANWEKDRTNQSGVEPCEGEQDKRLHCYASWRNSSGTIELVKKG |
| | CWLDDENCYDRQECVATEENPQVYFCCCEGNFCNERFTHLPEAGGPEVTYEP |
| | PPTAPT |
| 343 | ETQECLFFNANWEKDRTNQSGVEPCYGEQDKRLHCYASWRNSSGTIELVKKG |
| | CWLDDFNCYDRQECVATEENPQVYFCCCEGNFCNERFTHLPEAGGPEVTYEP |
| | PPTAPT |
| 344 | ETRECLFFNANWEKDRTNQSGVEPCYGDKDKRRHCYASWRNSSGTIELVKK |
| | GCWLDDFNCYDRQECVATEENPQVYFCCCEGNFCNERFTHLPEAGGPEVTYE |
| | PPPTAPT |
| 345 | ETRECLFFNANWEKDRTNQTGVEPCYGDKDKRRHCFATWKNISGSIEIVKQG |
| | CWLDDFNCYDRQECVATEENPQVYFCCCEGNFCNERFTHLPEAGGPEVTYEP |
| | PPTAPT |
| 346 | ETRECLFFNANWEKDRTNQTGVEPCYGDKDKRRHCFATWKNISGSIEIVKQG |
| | CWLDDINCYDRQECVATEENPQVYFCCCEGNFCNERFTHLPEAGGPEVTYEP |
| | PPTAPT |
| 347 | ETRECLFFNANWEKDRTNQTGVEPCYGDKDKRRHCFATWKNISGSIEIVKQG |
| | CWLDDINCYDRTDCVEKKDSPEVYFCCCEGNFCNERFTHLPEAGGPEVTYEPP |
| | PTAPT |
| 348 | ETQECIYYNANWELERTNQSGLERCYGDKDKRRHCFATWKNISGSIEIVKQG |
| | CWLDDINCYDRQECVATEENPQVYFCCCEGNFCNERFTHLPEAGGPEVTYEP |
| | PPTAPT |
| 349 | ETRECIYYNANWELERTNQSGLERCYGDKDKRRHCFATWKNISGSIEIVKQGC |
| | WLDDINCYDRTDCVEKKDSPEVYFCCCEGNFCNERFTHLPEAGGPEVTYEPPP |
| | TAPT |
| 350 | ETRECIYYNANWELERTNQSGLERCYGDKDKRRHCFATWKNISGSIEIVKQGC |
| | WLDDINCYDRTDCVEKKDSPEVYFCCCEGNMCNERFTHLPEAGGPEVTYEPP |
| | PTAPT |
| 351 | ETRECIYYNANWELERTNQSGLERCEGEQDKRLHCFATWKNISGSIEIVKQGC |
| | WLDDINCYDRTDCVEKKDSPEVYFCCCEGNFCNERFTHLPEAGGPEVTYEPPP |
| | TAPT |
| 352 | ETRECIYYNANWELERTNQSGLERCEGEQDKRLHCFATWKNISGSIEIVKQGC |
| | WLDDINCYDRTDCVATEENPQVYFCCCEGNFCNERFTHLPEAGGPEVTYEPPP |
| | TAPT |
| 353 | ETRECIYYNANWELERTNQSGLERCEGEQDKRLHCFATWKNISGSIEIVKQGC |
| | WLDDFNCYDRTDCVATEENPQVYFCCCEGNFCNERFTHLPEAGGPEVTYEPP |
| | PTAPT |
| 354 | ETRECIYYNANWELERTNQSGLERCEGEQDKRLHCFATWKNISGSIEIVKQGC |
| | WLDDFNCYDRQECVATEENPQVYFCCCEGNFCNERFTHLPEAGGPEVTYEPP |
| | PTAPT |
| 355 | ETQECIYYNANWELERTNQSGLERCYGDKDKRRHCYASWRNSSGTIELVKKG |
| | CWLDDINCYDRTDCVEKKDSPEVYFCCCEGNFCNERFTHLPEAGGPEVTYEPP |
| | PTAPT |
| 356 | ETQECIYYNANWELERTNQSGLERCYGDKDKRRHCYASWRNSSGTIELVKKG |
| | CWLDDINCYDRTDCVEKKDSPEVYFCCCEGNMCNERFTHLPEAGGPEVTYEP |
| | PPTAPT |
| 357 | ETQECIYYNANWELERTNQSGLERCYGDKDKRRHCFATWKNISGSIEIVKQG |
| | CWLDDINCYDRTDCVEKKDSPEVYFCCCEGNMCNERFTHLPEAGGPEVTYEP |
| | PPTAPT |
| 358 | ETQECIYYNANWELERTNQSGLERCYGDKDKRRHCFATWKNISGSIEIVKQG |
| | CWLDDINCYDRTDCVEKKDSPEVYFCCCEGNFCNERFTHLPEAGGPEVTYEPP |
| | PTAPT |
| 359 | ETRECLFFNANWEKDRTNQTGVEPCEGEQDKRLHCFATWKNISGSIEIVKQGC |
| | WLDDINCYDRTDCVEKKDSPEVYFCCCEGNFCNERFTHLPEAGGPEVTYEPPP |
| | TAPT |
| 360 | ETRECLFFNANWEKDRTNQSGVEPCEGEQDKRLHCYASWRNSSGTIELVKKG |
| | CWLDDINCYDRTDCVEKKDSPEVYFCCCEGNFCNERFTHLPEAGGPEVTYEPP |
| | PTAPT |
| 361 | ETRECLFFNANWEKDRTNQSGVEPCYGDKDKRRHCYASWRNSSGTIELVKK |
| | GCWLDDINCYDRTDCVEKKDSPEVYFCCCEGNFCNERFTHLPEAGGPEVTYE |
| | PPPTAPT |
| 362 | ETRECIYYNANWELERTNQSGLERCYGDKDKRRHCYASWRNSSGTIELVKKG |
| | CWLDDFNCYDRTDCVATEENPQVYFCCCEGNFCNERFTHLPEAGGPEVTYEP |
| | PPTAPT |
| 363 | ETRECIYYNANWELERTNQSGLERCYGDKDKRRHCYASWRNSSGTIELVKQG |
| | CWLDDFNCYDRTDCVATEENPQVYFCCCEGNFCNERFTHLPEAGGPEVTYEP |
| | PPTAPT |
| 364 | ETRECIYYNANWELERTNQSGLERCYGDKDKRRHCYASWRNSSGTIEIVKKG |
| | CWLDDFNCYDRTDCVATEENPQVYFCCCEGNFCNERFTHLPEAGGPEVTYEP |
| | PPTAPT |
| 365 | ETRECIYYNANWELERTNQSGLERCYGDKDKRRHCYASWRNSSGSIELVKKG |
| | CWLDDFNCYDRTDCVATEENPQVYFCCCEGNFCNERFTHLPEAGGPEVTYEP |
| | PPTAPT |
| 366 | ETRECIYYNANWELERTNQSGLERCYGDKDKRRHCYASWRNSSGSIEIVKKG |
| | CWLDDFNCYDRTDCVATEENPQVYFCCCEGNFCNERFTHLPEAGGPEVTYEP |
| | PPTAPT |
| 367 | ETRECIYYNANWELERTNQSGLERCYGDKDKRRHCYASWRNSSGSIEIVKQG |
| | CWLDDFNCYDRTDCVATEENPQVYFCCCEGNFCNERFTHLPEAGGPEVTYEP |
| | PPTAPT |
| 368 | ETRECIYYNANWELERTNQSGLERCEGEQDKRLHCYASWRNSSGTIELVKKG |
| | CWLDDFNCYDRTDCVATEENPQVYFCCCEGNFCNERFTHLPEAGGPEVTYEP |
| | PPTAPT |
| 369 | ETRECIYYNANWELERTNQSGLERCEGEQDKRLHCYASWRNSSGTIELVKKG |
| | CWLDDFNCYDRQECVATEENPQVYFCCCEGNFCNEKFSYFPEMEVTQPTSNP |
| | VTPKPP |
| 370 | ETRECIYYNANWELERTNQSGLERCEGEQDKRLHCYASWRNSSGTIELVKKG |
| | CWLDDFNCYDRQECVATEENPQVYFCCCEGNFCNEKFSYFPQMEVTQPTSNP |
| | VTPKPP |
| 371 | ETRECIYYNANWELERTNQSGLERCYGDKDKRRHCYASWRNSSGTIELVKKG |
| | CWLDDINCYDRTDCVEKKDSPEVYFCCCEGNFCNEKFSYFPEMEVTQPTSNP |
| | VTPKPP |
| 372 | ETRECIYYNANWELERTNQSGLERCYGDKDKRRHCYASWRNSSGTIELVKKG |
| | CWLDDINCYDRTDCVEKKDSPEVYFCCCEGNFCNEKFSYFPQMEVTQPTSNP |
| | VTPKPP |
| 373 | ETRECIYYNANWELERTNQSGLERCYGDKDKRRHCYASWRNSSGTIELVKKG |
| | CWLDDENCYDRQECVATEENPQVYFCCCEGNFCNEKFSYFPQMEVTQPTSNP |
| | VTPKPP |
| 374 | ETRECIYYNANWELERTNQSGLERCYGDKDKRRHCYASWRNSSGTIELVKQG |
| | CWLDDFNCYDRQECVATEENPQVYFCCCEGNFCNEKFSYFPQMEVTQPTSNP |
| | VTPKPP |
| 375 | ETRECIYYNANWELERTNQSGLERCYGDKDKRRHCYASWRNSSGTIEIVKKG |
| | CWLDDFNCYDRQECVATKENPQVYFCCCEGNFCNEKFSYFPQMEVTQPTSNP |
| | VTPKPP |
| 376 | ETRECIYYNANWELERTNQSGLERCYGDKDKRRHCYASWRNSSGSIELVKKG |
| | CWLDDFNCYDRQECVATKENPQVYFCCCEGNFCNEKFSYFPQMEVTQPTSNP |
| | VTPKPP |
| 377 | ETRECIYYNANWELERTNQSGLERCEGEQDKRLHCYASWRNSSGSIELVKKG |
| | CWLDDENCYDRQECVATEENPQVYFCCCEGNFCNEKFSYFPQMEVTQPTSNP |
| | VTPKPP |
| 378 | ETRECIYYNANWELERTNQSGLERCEGEQDKRLHCYASWRNSSGTIEIVKKG |
| | CWLDDFNCYDRQECVATEENPQVYFCCCEGNFCNEKFSYFPQMEVTQPTSNP |
| | VTPKPP |
| 379 | ETRECIYYNANWELERTNQSGLERCEGEQDKRLHCYASWRNSSGTIELVKQG |
| | CWLDDFNCYDRQECVATEENPQVYFCCCEGNFCNEKFSYFPQMEVTQPTSNP |
| | VTPKPP |
| 380 | ETRECIYYNANWELERTNQSGLERCEGEQDKRLHCYASWRNSSGTIELVKKG |
| | CWLDDFNCYDRQECVETEENPQVYFCCCEGNFCNEKFSYFPQMEVTQPTSNP |
| | VTPKPP |
| 381 | ETRECIYYNANWELERTNQSGLERCEGEQDKRLHCYASWRNSSGTIELVKKG |
| | CWLDDFNCYDRQECVAKEENPQVYFCCCEGNFCNEKFSYFPQMEVTQPTSNP |
| | VTPKPP |
| 382 | ETRECIYYNANWELERTNQSGLERCEGEQDKRLHCYASWRNSSGTIELVKKG |
| | CWLDDFNCYDRQECVATKENPQVYFCCCEGNFCNEKFSYFPQMEVTQPTSNP |
| | VTPKPP |
| 383 | ETRECIYYNANWELERTNQSGLERCEGEQDKRLHCYASWRNSSGTIELVKKG |
| | CWLDDFNCYDRQECVATEDNPQVYFCCCEGNFCNEKFSYFPQMEVTQPTSNP |
| | VTPKPP |
| 384 | ETRECIYYNANWELERTNQSGLERCEGEQDKRLHCYASWRNSSGTIELVKKG |
| | CWLDDFNCYDRQECVATEESPQVYFCCCEGNFCNEKFSYFPQMEVTQPTSNP |
| | VTPKPP |
| 385 | ETRECIYYNANWELERTNQSGLERCEGEQDKRLHCYASWRNSSGTIELVKKG |
| | CWLDDFNCYDRQECVATEENPEVYFCCCEGNFCNEKFSYFPQMEVTQPTSNP |
| | VTPKPP |
| 386 | ETRECIYYNANWELERTNQSGLERCEGDKDKRLHCYASWRNSSGTIELVKKG |
| | CWLDDFNCYDRQECVETEENPQVYFCCCEGNFCNEKFSYFPQMEVTQPTSNP |
| | VTPKPP |
| 387 | ETRECIYYNANWELERTNQSGLERCEGEKDKRLHCYASWRNSSGTIELVKKG |
| | CWLDDFNCYDRQECVAKEENPQVYFCCCEGNFCNEKFSYFPQMEVTQPTSNP |
| | VTPKPP |
| 388 | ETRECIYYNANWELERTNQSGLERCEGDQDKRLHCYASWRNSSGTIELVKKG |
| | CWLDDENCYDRQECVATKENPQVYFCCCEGNFCNEKFSYFPQMEVTQPTSNP |
| | VTPKPP |
| 389 | ETRECIYYNANWELERTNQSGLERCEGDKDKRLHCYASWRNSSGTIELVKQG |
| | CWLDDFNCYDRQECVAKKENPQVYFCCCEGNFCNEKFSYFPQMEVTQPTSNP |
| | VTPKPP |
| 390 | ETRECIYYNANWELERTNQSGLERCEGDKDKRLHCYASWRNSSGTIEIVKQG |
| | CWLDDFNCYDRQECVAEKENPQVYFCCCEGNFCNEKFSYFPQMEVTQPTSNP |
| | VTPKPP |
| 391 | ETRECIYYNANWELERTNQSGLERCYGDQDKRLHCYASWRNSSGSIEIVKQG |
| | CWLDDFNCYDRQECVAKKENPQVYFCCCEGNFCNEKFSYFPQMEVTQPTSNP |
| | VTPKPP |
| 392 | ETRECIYYNANWELERTNQSGLERCEGEKDKRRHCYASWRNSSGTIEIVKKG |
| | CWLDDFNCYDRQECVATKENPQVYFCCCEGNFCNEKFSYFPQMEVTQPTSNP |
| | VTPKPP |
| 393 | ETRECIYYNANWELERTNQSGLERCYGDQDKRRHCYASWRNSSGTIELVKKG |
| | CWLDDFNCYDRQECVATEENPEVYFCCCEGNFCNEKFSYFPQMEVTQPTSNP |
| | VTPKPP |
| 394 | ETRECIYYNANWELERTNQSGLERCEGEQDKRRHCYASWRNSSGTIELVKKG |
| | CWLDDFNCYDRQECVATEENPQVYFCCCEGNFCNEKFSYFPQMEVTQPTSNP |
| | VTPKPP |
| 395 | ETRECIYYNANWELERTNQSGLERCYGEQDKRLHCYASWRNSSGSIEIVKKG |
| | CWLDDFNCYDRTDCVATEENPQVYFCCCEGNFCNEKFSYFPQMEVTQPTSNP |
| | VTPKPP |
| 396 | ETRECIYYNANWELERTNQSGLERCEGEQDKRRHCYASWRNSSGSIELVKKG |
| | CWLDDFNCYDRQECVAKEENPQVYFCCCEGNFCNEKFSYFPQMEVTQPTSNP |
| | VTPKPP |
| 397 | ETRECIYYNANWELERTNQSGLERCEGEQDKRRHCYASWRNSSGTIEIVKKG |
| | CWLDDFNCYDRQECVAKEENPQVYFCCCEGNFCNEKFSYFPQMEVTQPTSNP |
| | VTPKPP |
| 398 | ETRECIYYNANWELERTNQSGLERCEGEQDKRRHCYASWRNSSGSIEIVKKG |
| | CWLDDENCYDRQECVATKENPQVYFCCCEGNFCNEKFSYFPQMEVTQPTSNP |
| | VTPKPP |
| 399 | ETRECIYYNANWELERTNQSGLERCEGDQDKRLHCYASWRNSSGTIEIVKKG |
| | CWLDDFNCYDRQECVATKENPQVYFCCCEGNFCNEKFSYFPQMEVTQPTSNP |
| | VTPKPP |
| 400 | ETRECIYYNANWELERTNQSGLERCEGDQDKRLHCYASWRNSSGSIELVKKG |
| | CWLDDFNCYDRQECVATKENPQVYFCCCEGNFCNEKFSYFPQMEVTQPTSNP |
| | VTPKPP |
| 401 | ETRECIYYNANWELERTNQSGLERCEGDQDKRLHCYASWRNSSGTIELVKKG |
| | CWLDDFNCYDRQECVATKENPQVYFCCCEGNFCNEKFSYFPQMEVTQPTSNP |
| | VTPKPP |
| 402 | ETRECIYYNANWELERTNQSGLERCEGEQDKRLHCYASWRNSSGTIEIVKKG |
| | CWLDDFNCYDRQECVATKENPQVYFCCCEGNFCNEKFSYFPQMEVTQPTSNP |
| | VTPKPP |
| 403 | ETRECIYYNANWELERTNQSGLERCEGEQDKRLHCYASWRNSSGTIELVKKG |
| | CWLDDFNCYDRQECVATKENPQVYFCCCEGNFCNEKFSYFPQMEVTQPTSNP |
| | VTPKPP |
| 404 | ETRECIYYNANWELERTNQSGLERCEGDQDKRLHCYASWRNSSGTIEIVKKG |
| | CWLDDFNCYDRQECVAKKENPQVYFCCCEGNFCNEKFSYFPQMEVTQPTSNP |
| | VTPKPP |
| 405 | ETQECLFFNANWEKDRTNQTGVEPCYGDKDKRRHCFATWKNISGSIEIVKQG |
| | CWLDDINCYDRTDCVEKKDSPEVYFCCCEGNMCNERFTHLPEAGGPEVTYEP |
| | PPTAPT |
| 406 | ETRECIYYNANWELERTNQSGLERCYGDKDKRRHCYASWRNSSGTIELVKKG |
| | CWLDDINCYDRQECVATKENPQVYFCCCEGNFCNERFTHLPEAGGPEVTYEP |
| | PPTAPT |
| 407 | SGRGEAETRECIYYNANWELERTNQSGLERCEGEQDKRLHCYASWRNSSGTI |
| | ELVKKGCWLDDFNCYDRQECVATEENPQVYFCCCEGNFCNERFTHLPEAGGP |
| | EVTYEPPPTAPT |
| 408 | MGAATKLAFAVFLISCSSGAILGRSETQECIYYNANWEKDKTNRSGIEPCYGD |
| | KDKRRHCFATWKNISGSIEIVKQGCWLDDINCYDRNDCIEKKDSPEVFFCCCE |
| | GNMCNERFFYFPEMEVTQPTSNPVTPKPPLFNTLLYSLVPIMGIAVIVLFSFWM |
| | YRHHKLAYPPVLVPTQDPGPPPPSPLMGLKPLQLLEIKARGRFGCVWKAQLL |
| | NEYVAVKIFPIQDKQSWQNEYEIYSLPGMKHDNILQFIGAEKRGTSIDVDLWLI |
| | TAFHEKGSLTDFLKANVVSWNELCHIAQTMARGLAYLHEDIPGLKDGHKPAI |
| | SHRDIKSKNVLLKNNLTACIADFGLALKFEAGKSAGDTHGQVGTRRYMAPEV |
| | LEGAINFQRDAFLRIDMYAMGLVLWELASRCTASDGPVDEYMLPFEEEIGQH |
| | PSLEDMQEVVVHKKKRPVLRECWQKHSGMAMLCETIEECWDHDAEARLSA |
| | GCVEERIIQMQKLTNIITTEDIVTVVTMVTNVDFPPKESSL |
| 409 | AILGRSETQECLFFNANWEKDRTNQTGVEPCYGDKDKRRHCFATWKNISGSIE |
| | IVKQGCWLDDINCYDRTDCVEKKDSPEVYFCCCEGNMCNEKFSYFPEMEVTQ |
| | PTSNPVTPKPP |

### 8. Exemplification

The disclosure above will be more readily understood by reference to the following examples, which are included merely for purposes of illustration of certain embodiments of the present invention, and are not intended to be limiting.

### Example 1: ActRIIA-Fc Fusion Proteins

A soluble ActRII fusion protein was constructed that has the extracellular domain of human ActRIIA fused to a human or mouse Fc domain with a minimal linker in between. The constructs are referred to as ActRIIA-hFec and ActRIIA-mFc, respectively.

ActRIIA-hFc is shown below as purified from CHO cell lines (SEQ ID NO: 32):

ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPPTGGGTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPVPIEKTISKAKGQPREPQV
YTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
K

The ActRIIA-hFc and ActRIIA-mFc proteins were expressed in CHO cell lines. Three different leader sequences were considered:

(i) Honey bee mellitin (HBML):
(SEQ ID NO: 33)
MKFLVNVALVFMVVYISYIYA
(ii) Tissue plasminogen activator (TPA):
(SEQ ID NO: 34)
MDAMKRGLCCVLLLCGAVFVSP
(iii) Native:
(SEQ ID NO: 35)
MGAAAKLAFAVFLISCSSGA.

The selected form employs the TPA leader and has the following unprocessed amino acid sequence:

(SEQ ID NO: 36)
MDAMKRGLCCVLLLCGAVFVSPGAAILGRSETQECLFFNANWEKDRTNQ
TGVEPCYGDKDKRRHCFATWKNISGSIEIVKQGCWLDDINCYDRTDCVE
KKDSPEVYFCCCEGNMCNEKFSYFPEMEVTQPTSNPVTPKPPTGGGTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPVPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGK

This polypeptide is encoded by the following nucleic acid sequence:

(SEQ ID NO: 37)
ATGGATGCAATGAAGAGAGGGCTCTGCTGTGTGCTGCTGCTGTGTGGAG
CAGTCTTCGTTTCGCCCGGCGCCGCTATACTTGGTAGATCAGAAACTCA
GGAGTGTCTTTTTTTAATGCTAATTGGGAAAAAGACAGAACCAATCAAA
CTGGTGTTGAACCGTGTTATGGTGACAAAGATAAACGGCGGCATTGTTT
TGCTACCTGGAAGAATATTTCTGGTTCCATTGAATAGTGAAACAAGGTT
GTTGGCTGGATGATATCAACTGCTATGACAGGACTGATTGTGTAGAAAA
AAAAGACAGCCCTGAAGTATATTTCTGTTGCTGTGAGGGCAATATGTGT
AATGAAAAGTTTTCTTATTTTCCGGAGATGGAAGTCACACAGCCCACTT
CAAATCCAGTTACACCTAAGCCACCCACCGGTGGTGGAACTCACACATG
CCCACCGTGCCCAGCACCTGAACTCCTGGGGGGACCGTCAGTCTTCCTC
TTCCCCCCAAAACCCAAGGACACCCTCATGATCTCCCGGACCCCTGAGG
TCACATGCGTGGTGGTGGACGTGAGCCACGAAGACCCTGAGGTCAAGTT
CAACTGGTACGTGGACGGCGTGGAGGTGCATAATGCCAAGACAAAGCCG
CGGGAGGAGCAGTACAACAGCACGTACCGTGTGGTCAGCGTCCTCACCG
TCCTGCACCAGGACTGGCTGAATGGCAAGGAGTACAAGTGCAAGGTCTC
CAACAAAGCCCTCCCAGTCCCCATCGAGAAAACCATCTCCAAAGCCAAA
GGGCAGCCCCGAGAACCACAGGTGTACACCCTGCCCCCATCCCGGGAGG
AGATGACCAAGAACCAGGTCAGCCTGACCTGCCTGGTCAAAGGCTTCTA
TCCCAGCGACATCGCCGTGGAGTGGGAGAGCAATGGGCAGCCGGAGAAC
AACTACAAGACCACGCCTCCCGTGCTGGACTCCGACGGCTCCTTCTTCC
TCTATAGCAAGCTCACCGTGGACAAGAGCAGGTGGCAGCAGGGGAACGT
CTTCTCATGCTCCGTGATGCATGAGGCTCTGCACAACCACTACACGCAG
AAGAGCCTCTCCCTGTCTCCGGGTAAATGAGAATTC

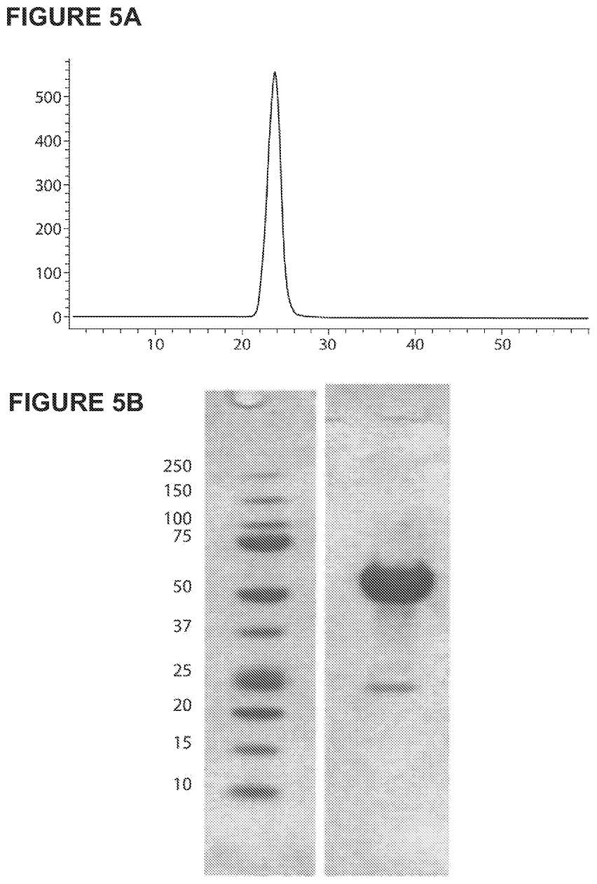

Both ActRIIA-hFc and ActRIIA-mFc were remarkably amenable to recombinant expression. As shown in FIGS. 5A and 5B, the protein was purified as a single, well-defined peak of protein. N-terminal sequencing revealed a single sequence of -ILGRSETQE (SEQ ID NO: 38). Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. The ActRIIA-hFc protein was purified to a purity of >98% as determined by size exclusion chromatography and >95% as determined by SDS PAGE.

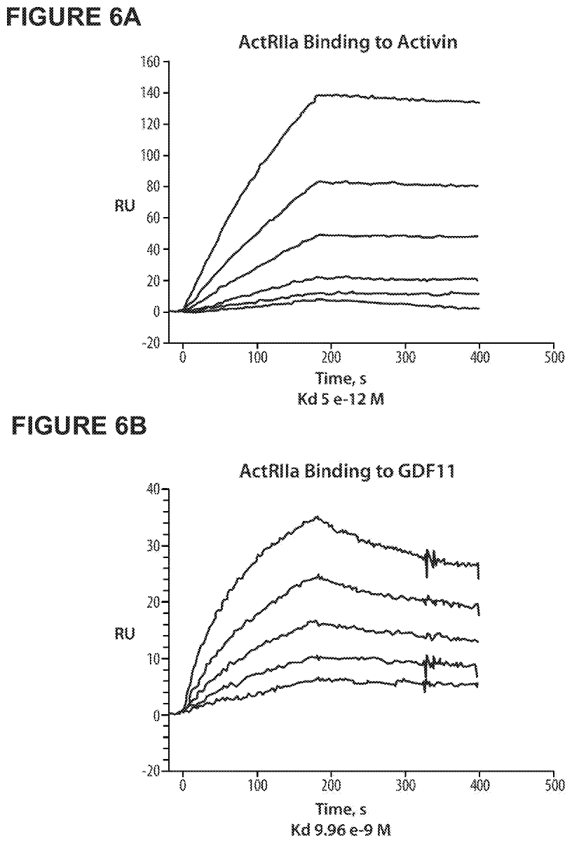

ActRIIA-hFc and ActRIIA-mFc showed a high affinity for ligands. GDF11 or activin A were immobilized on a Biacore™ CM5 chip using standard amine-coupling procedure. ActRIIA-hFec and ActRIIA-mFc proteins were loaded onto the system, and binding was measured. ActRIIA-hFc bound to activin with a dissociation constant (K_{D}) of 5×10⁻¹² and bound to GDF11 with a K_{D} of 9.96×10⁻⁹. See FIGS. 6A and 6B. Using a similar binding assay, ActRIIA-hFc was determined to have high to moderate affinity for other TGF-beta superfamily ligands including, for example, activin B, GDF8, BMP6, and BMP10. ActRIIA-mFc behaved similarly.

The ActRIIA-hFc was very stable in pharmacokinetic studies. Rats were dosed with 1 mg/kg, 3 mg/kg, or 10 mg/kg of ActRIIA-hFec protein, and plasma levels of the protein were measured at 24, 48, 72, 144 and 168 hours. In a separate study, rats were dosed at 1 mg/kg, 10 mg/kg, or 30 mg/kg. In rats, ActRIIA-hFc had an 11-14 day serum half-life, and circulating levels of the drug were quite high after two weeks (11 μg/ml, 110 μg/ml, or 304 μg/ml for initial administrations of 1 mg/kg, 10 mg/kg, or 30 mg/kg, respectively.) In cynomolgus monkeys, the plasma half-life was substantially greater than 14 days, and circulating levels of the drug were 25 μg/ml, 304 μg/ml, or 1440 μg/ml for initial administrations of 1 mg/kg, 10 mg/kg, or 30 mg/kg, respectively.

### Example 2: Characterization of an ActRIIA-hFc Protein

ActRIIA-hFc fusion protein was expressed in stably transfected CHO-DUKX B11 cells from a pAID4 vector (SV40 ori/enhancer, CMV promoter), using a tissue plasminogen leader sequence of SEQ ID NO: 34. The protein, purified as described above in Example 1, had a sequence of SEQ ID NO: 32. The Fc portion is a human IgG1 Fc sequence, as shown in SEQ ID NO: 32. Protein analysis reveals that the ActRIIA-hFc fusion protein is formed as a homodimer with disulfide bonding.

The CHO-cell-expressed material has a higher affinity for activin B ligand than that reported for an ActRIIA-hFc fusion protein expressed in human 293 cells [see, del Re et al. (2004) J Biol Chem. 279(51):53126-53135]. Additionally, the use of the TPA leader sequence provided greater production than other leader sequences and, unlike ActRIIA-Fc expressed with a native leader, provided a highly pure N-terminal sequence. Use of the native leader sequence resulted in two major species of ActRIIA-Fc, each having a different N-terminal sequence.

### Example 3: Alternative ActRIIA-Fc Proteins

A variety of ActRIIA variants that may be used according to the methods described herein are described in the International Patent Application published as WO2006/012627 (see e.g., pp. 55-58), incorporated herein by reference in its entirety. An alternative construct may have a deletion of the C-terminal tail (the final 15 amino acids of the extracellular domain of ActRIIA. The sequence for such a construct is presented below (Fc portion underlined) (SEQ ID NO: 39):

ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMTGGGTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPVPIEKTISKAKGQPREPQVYTLPPSREEMTKNQV
SLTCLVKGFYPSDIAVEWESNGQPENNYKTTPPVLDSDGSFFLYSKLTV
DKSRWQQGNVFSCSVMHEALHNHYTQKSLSLSPGK.

### Example 4: Generation of ActRIIB-Fc Fusion Proteins

Applicants constructed a soluble ActRIIB fusion protein that has the extracellular domain of human ActRIIB fused to a human or mouse Fc domain with a minimal linker in between. The constructs are referred to as ActRIIB-hFc and ActRIIB-mFc, respectively.

ActRIIB-hFc is shown below as purified from CHO cell lines (SEQ ID NO: 40):

GRGEAETRECIYYNANWELERTNQSGLERCEGEQDKRLHCYASWRNSSG
TIELVKKGCWLDDFNCYDRQECVATEENPQVYFCCCEGNFCNERFTHLP
EAGGPEVTYEPPPTAPTGGGTHTCPPCPAPELLGGPSVFLFPPKPKDTL
MISRTPEVTCVVVDVSHEDPEVKFNWYVDGVEVHNAKTKPREEQYNSTY
RVVSVLTVLHQDWLNGKEYKCKVSNKALPVPIEKTISKAKGQPREPQVY
TLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPG
K.

The ActRIIB-hFc and ActRIIB-mFc proteins were expressed in CHO cell lines. Three different leader sequences were considered: (i) Honey bee mellitin (HBML), ii) Tissue plasminogen activator (TPA), and (iii) Native: MGAAAKLAFAVFLISCSSGA (SEQ ID NO: 41).

The selected form employs the TPA leader and has the following unprocessed amino acid sequence (SEQ ID NO: 42):

MDAMKRGLCCVLLLCGAVFVSPGASGRGEAETRECIYYNANWELERTNQ
SGLERCEGEQDKRLHCYASWRNSSGTIELVKKGCWLDDENCYDRQECVA
TEENPQVYFCCCEGNFCNERFTHLPEAGGPEVTYEPPPTAPTGGGTHTC
PPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPVPIEKTISKAKGQPREPQVYTLPPSREEMTKNQVSLTCLVKGFY
PSDIAVEWESNGQPENNYKTTPPVLDSDGSFFLYSKLTVDKSRWQQGNV
FSCSVMHEALHNHYTQKSLSLSPGK

This polypeptide is encoded by the following nucleic acid sequence (SEQ ID NO: 43):

A TGGATGCAAT GAAGAGAGGG CTCTGCTGTG TGCTGCTGCT
GTGTGGAGCA GTCTTCGTTT CGCCCGGCGC CTCTGGGCGT
GGGGAGGCTG AGACACGGGA GTGCATCTAC TACAACGCCA
ACTGGGAGCT GGAGCGCACC AACCAGAGCG GCCTGGAGCG
CTGCGAAGGC GAGCAGGACA AGCGGCTGCA CTGCTACGCC
TCCTGGCGCA ACAGCTCTGG CACCATCGAG CTCGTGAAGA
AGGGCTGCTG GCTAGATGAC TTCAACTGCT ACGATAGGCA
GGAGTGTGTG GCCACTGAGG AGAACCCCCA GGTGTACTTC
TGCTGCTGTG AAGGCAACTT CTGCAACGAG CGCTTCACTC
ATTTGCCAGA GGCTGGGGGC CCGGAAGTCA CGTACGAGCC
ACCCCCGACA GCCCCCACCG GTGGTGGAAC TCACACATGC
CCACCGTGCC CAGCACCTGA ACTCCTGGGG GGACCGTCAG
TCTTCCTCTT CCCCCCAAAA CCCAAGGACA CCCTCATGAT
CTCCCGGACC CCTGAGGTCA CATGCGTGGT GGTGGACGTG
AGCCACGAAG ACCCTGAGGT CAAGTTCAAC TGGTACGTGG
ACGGCGTGGA GGTGCATAAT GCCAAGACAA AGCCGCGGGA
GGAGCAGTAC AACAGCACGT ACCGTGTGGT CAGCGTCCTC
ACCGTCCTGC ACCAGGACTG GCTGAATGGC AAGGAGTACA
AGTGCAAGGT CTCCAACAAA GCCCTCCCAG TCCCCATCGA
GAAAACCATC TCCAAAGCCA AAGGGCAGCC CCGAGAACCA
CAGGTGTACA CCCTGCCCCC ATCCCGGGAG GAGATGACCA
AGAACCAGGT CAGCCTGACC TGCCTGGTCA AAGGCTTCTA
TCCCAGCGAC ATCGCCGTGG AGTGGGAGAG CAATGGGCAG
CCGGAGAACA ACTACAAGAC CACGCCTCCC GTGCTGGACT
CCGACGGCTC CTTCTTCCTC TATAGCAAGC TCACCGTGGA
CAAGAGCAGG TGGCAGCAGG GGAACGTCTT CTCATGCTCC
GTGATGCATG AGGCTCTGCA CAACCACTAC ACGCAGAAGA
GCCTCTCCCT GTCTCCGGGT AAATGA.

N-terminal sequencing of the CHO-cell-produced material revealed a major sequence of -GRGEAE (SEQ ID NO: 44). Notably, other constructs reported in the literature begin with an -SGR . . . sequence.

Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

ActRIIB-Fc fusion proteins were also expressed in HEK293 cells and COS cells. Although material from all cell lines and reasonable culture conditions provided protein with muscle-building activity in vivo, variability in potency was observed perhaps relating to cell line selection and/or culture conditions.

Applicants generated a series of mutations in the extracellular domain of ActRIIB and produced these mutant proteins as soluble fusion proteins between extracellular ActRIIB and an Fc domain. The background ActRIIB-Fc fusion has the sequence of SEQ ID NO: 40.

Various mutations, including N- and C-terminal truncations, were introduced into the background ActRIIB-Fc protein. Based on the data presented herein, it is expected that these constructs, if expressed with a TPA leader, will lack the N-terminal serine. Mutations were generated in ActRIIB extracellular domain by PCR mutagenesis. After PCR, fragments were purified through a Qiagen column, digested with SfoI and AgeI and gel purified. These fragments were ligated into expression vector pAID4 (see WO2006/012627) such that upon ligation it created fusion chimera with human IgG1. Upon transformation into E. coli DH5 alpha, colonies were picked and DNAs were isolated. For murine constructs (mFc), a murine IgG2a was substituted for the human IgG1. Sequences of all mutants were verified. All of the mutants were produced in HEK293T cells by transient transfection. In summary, in a 500 ml spinner, HEK293T cells were set up at 6×10⁵ cells/ml in Freestyle (Invitrogen) media in 250 ml volume and grown overnight. Next day, these cells were treated with DNA:PEI (1:1) complex at 0.5 ug/ml final DNA concentration. After 4 hrs, 250 ml media was added and cells were grown for 7 days. Conditioned media was harvested by spinning down the cells and concentrated.

Mutants were purified using a variety of techniques, including, for example, a protein A column, and eluted with low pH (3.0) glycine buffer. After neutralization, these were dialyzed against PBS.

Mutants were also produced in CHO cells by similar methodology. Mutants were tested in binding assays and/or bioassays described in WO 2008/097541 and WO 2006/012627 incorporated by reference herein. In some instances, assays were performed with conditioned medium rather than purified proteins. Additional variations of ActRIIB are described in U.S. Pat. No. 7,842,663.

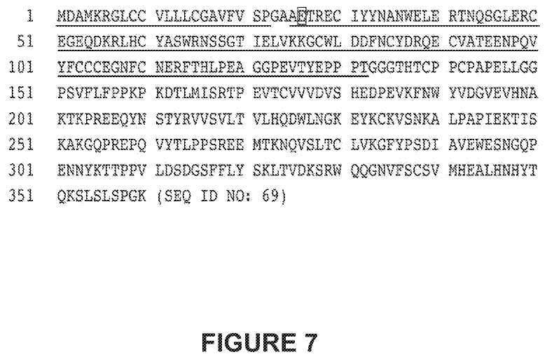

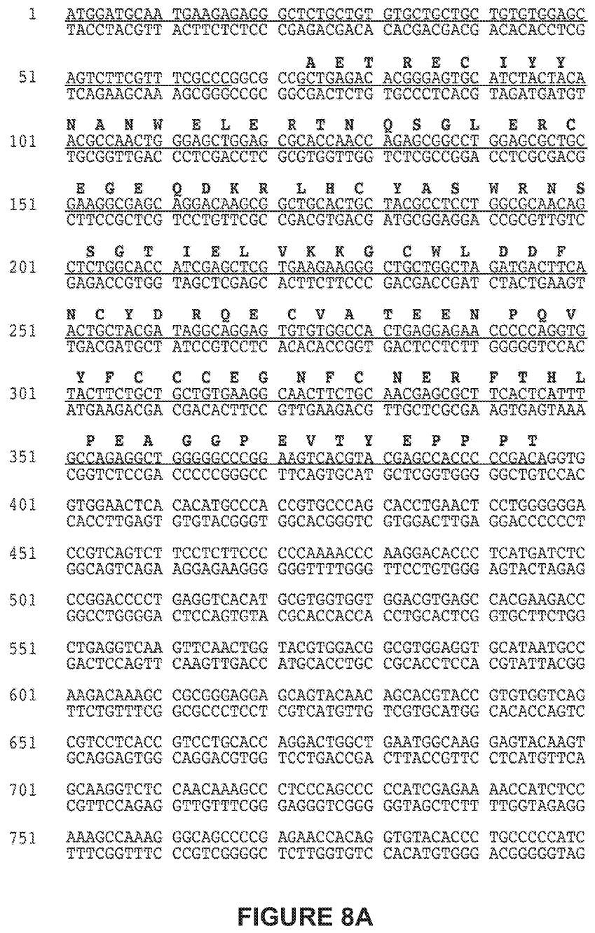

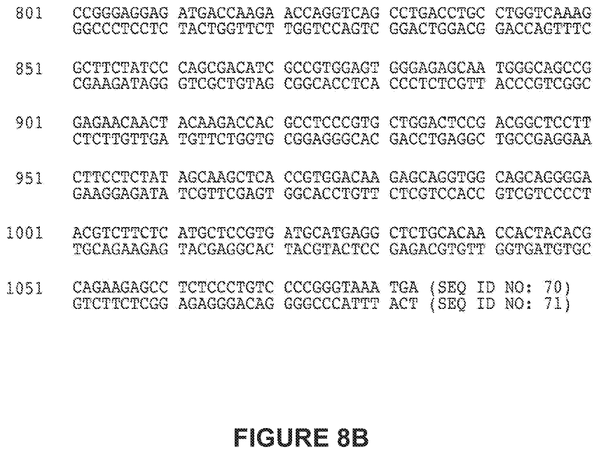

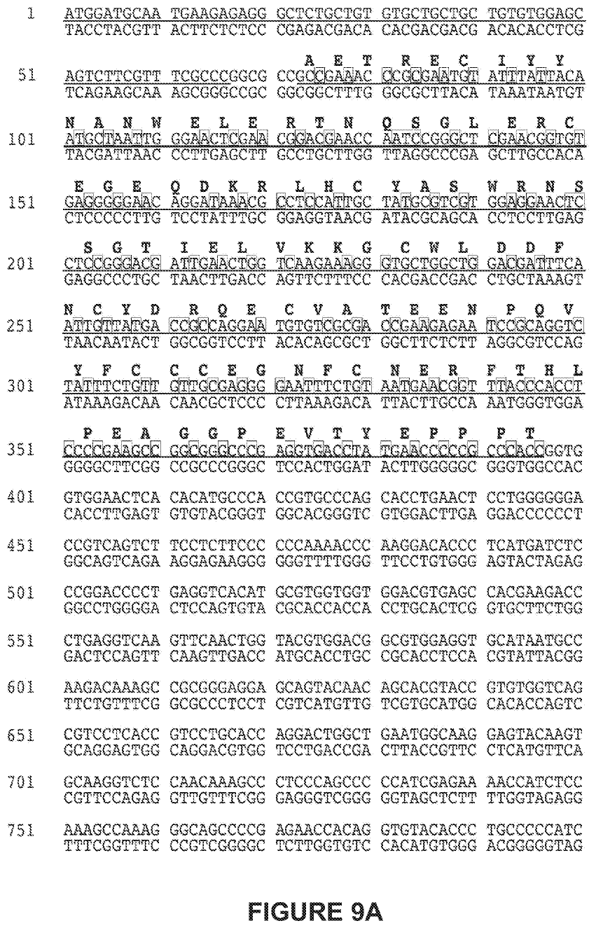

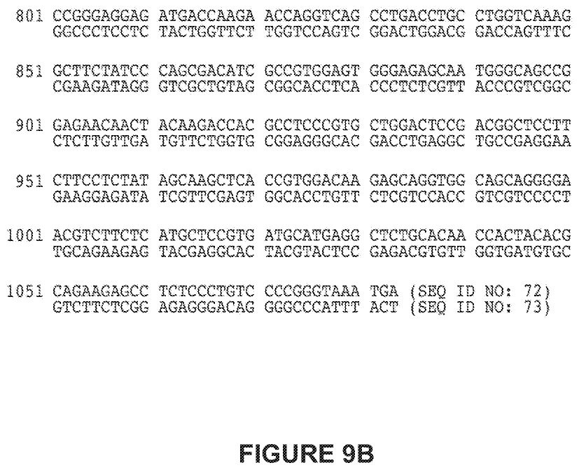

Applicant generated an ActRIIB(25-131)-hFc fusion protein, which comprises the human ActRIIB extracellular domain with N-terminal and C-terminal truncations (residues 25-131 of the native protein SEQ ID NO: 1) fused N-terminally with a TPA leader sequence substituted for the native ActRIIB leader and C-terminally with a human Fc domain via a minimal linker (three glycine residues) (FIG. 7). A nucleotide sequence encoding this fusion protein is shown in FIGS. 8A and 8B. Applicants modified the codons and found a variant nucleic acid encoding the ActRIIB(25-131)-hFc protein that provided substantial improvement in the expression levels of initial transformants (FIGS. 9A and 9B).

The mature protein has an amino acid sequence as follows (N-terminus confirmed by N-terminal sequencing)(SEQ ID NO: 45):

ETRECIYYNA NWELERTNQS GLERCEGEQD KRLHCYASWR
NSSGTIELVK KGCWLDDFNC YDRQECVATE ENPQVYFCCC
EGNFCNERFT HLPEAGGPEV TYEPPPTGGG THTCPPCPAP
ELLGGPSVFL FPPKPKDTLM ISRTPEVTCV VVDVSHEDPE
VKFNWYVDGV EVHNAKTKPR EEQYNSTYRV VSVLTVLHQD
WLNGKEYKCK VSNKALPAPI EKTISKAKGQ PREPQVYTLP
PSREEMTKNQ VSLTCLVKGF YPSDIAVEWE SNGQPENNYK
TTPPVLDSDG SFFLYSKLTV DKSRWQQGNV FSCSVMHEAL
HNHYTQKSLS LSPGK

The expressed molecule was purified using a series of column chromatography steps, including for example, three or more of the following, in any order: Protein A chromatography, Q sepharose chromatography, phenylsepharose chromatography, size exclusion chromatography and cation exchange chromatography. The purification could be completed with viral filtration and buffer exchange.

Affinities of several ligands for ActRIIB(25-131)-hFc and its full-length counterpart ActRIIB(20-134)-hFc were evaluated in vitro with a Biacore™ instrument, and the results are summarized in Table 12 below. Kd values were obtained by steady-state affinity fit due to very rapid association and dissociation of the complex, which prevented accurate determination of kₒₙ and k_{off}. ActRIIB(25-131)-hFc bound, for example, activin A, activin B, and GDF11 with high affinity.

TABLE 12
Ligand Affinities of ActRIIB-hFc Forms:

| | Activin A | Activin B | GDF11 |
|---|---|---|---|
| Fusion Construct | (e−11) | (e−11) | (e−11) |
| ActRIIB(20-134)-hFc | 1.6 | 1.2 | 3.6 |
| ActRIIB(25-131)-hFc | 1.8 | 1.2 | 3.1 |

A person of skill in the art would readily appreciate that the process outlined above could be used to produce a variant ActRII polypeptide (e.g., a variant ActRIIB polypeptide) Fc fusion protein based on the disclosure herein.

### Example 5: Generation of a ActRIIB Variant Fe Fusion Polypeptide

An ActRIIB variant Fc fusion polypeptide was constructed as follows. A polypeptide having a modified extracellular domain of ActRIIB (amino acids 20-134 of SEQ ID NO: 1 with an L79D substitution) with greatly reduced activin A binding relative to GDF11 and/or myostatin (as a consequence of a leucine-to-aspartate substitution at position 79 in SEQ ID NO: 1) was fused to a human or mouse Fc domain with a minimal linker in between. The constructs are referred to as ActRIIB(L79D 20-134)-hFc and ActRIIB(L79D 20-134)-mFc, respectively. Alternative forms with a glutamate rather than an aspartate at position 79 performed similarly (L79E). Alternative forms with an alanine rather than a valine at position 226 with respect to SEQ ID NO: 64, below were also generated and performed equivalently in all respects tested. The aspartate at position 79 (relative to SEQ ID NO: 1) is indicated with double underlining below. The valine at position 226 relative to SEQ ID NO: 64 is also indicated by double underlining below.

The ActRIIB variant Fc fusion polypeptide ActRIIB(L79D 20-134)-hFc is shown below as purified from CHO cell lines (SEQ ID NO: 46).

GRGEAETRECIYYNANWELERTNQSGLERCEGEQDKRLHCYASWRNSSG
TIELVKKGCWDDDFNCYDRQECVATEENPQVYFCCCEGNFCNERFTHLP
EAGGPEVTYEPPPTAPTGGGTHTCPPCPAPELLGGPSVFLFPPKPKDTL
MISRTPEVTCVVVDVSHEDPEVKFNWYVDGVEVHNAKTKPREEQYNSTY
RVVSVLTVLHQDWLNGKEYKCKVSNKALPVPIEKTISKAKGQPREPQVY
TLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK

The ActRIIB-derived portion of the ActRIIB variant Fc fusion polypeptide has an amino acid sequence set forth below (SEQ ID NO: 47), and that portion could be used as a monomer or as a non-Fc fusion protein as a monomer, dimer, or eater-order complex.

(SEQ ID NO: 47)
GRGEAETRECIYYNANWELERTNQSGLERCEGEQDKRLHCYASWRNSSG
TIELVKKGCWDDDFNCYDRQECVATEENPQVYFCCCEGNFCNERFTHLP
EAGGPEVTYEPPPTAPT

The ActRIIB variant Fc fusion polypeptide protein was expressed in CHO cell lines. Three different leader sequences were considered:

(i) Honey Bee Melittin (HBML), (ii) Tissue Plasminogen Activator (TPA), and (iii) Native.

The selected form employs the TPA leader and has the following unprocessed amino acid sequence:

(SEQ ID NO: 48)
MDAMKRGLCCVLLLCGAVFVSPGASGRGEAETRECIYYNANWELERTNQ
SGLERCEGEQDKRLHCYASWRNSSGTIELVKKGCWDDDFNCYDRQECVA
TEENPQVYFCCCEGNFCNERFTHLPEAGGPEVTYEPPPTAPTGGGTHTC
PPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNV
FSCSVMHEALHNHYTQKSLSLSPGK

This polypeptide is encoded by the following nucleic acid sequence (SEQ ID NO: 49):

A TGGATGCAAT GAAGAGAGGG CTCTGCTGTG TGCTGCTGCT
GTGTGGAGCA GTCTTCGTTT CGCCCGGCGC CTCTGGGCGT
GGGGAGGCTG AGACACGGGA GTGCATCTAC TACAACGCCA
ACTGGGAGCT GGAGCGCACC AACCAGAGCG GCCTGGAGCG
CTGCGAAGGC GAGCAGGACA AGCGGCTGCA CTGCTACGCC
TCCTGGCGCA ACAGCTCTGG CACCATCGAG CTCGTGAAGA
AGGGCTGCTG GGACGATGAC TTCAACTGCT ACGATAGGCA
GGAGTGTGTG GCCACTGAGG AGAACCCCCA GGTGTACTTC
TGCTGCTGTG AAGGCAACTT CTGCAACGAG CGCTTCACTC
ATTTGCCAGA GGCTGGGGGC CCGGAAGTCA CGTACGAGCC
ACCCCCGACA GCCCCCACCG GTGGTGGAAC TCACACATGC
CCACCGTGCC CAGCACCTGA ACTCCTGGGG GGACCGTCAG
TCTTCCTCTT CCCCCCAAAA CCCAAGGACA CCCTCATGAT
CTCCCGGACC CCTGAGGTCA CATGCGTGGT GGTGGACGTG
AGCCACGAAG ACCCTGAGGT CAAGTTCAAC TGGTACGTGG
ACGGCGTGGA GGTGCATAAT GCCAAGACAA AGCCGCGGGA
GGAGCAGTAC AACAGCACGT ACCGTGTGGT CAGCGTCCTC
ACCGTCCTGC ACCAGGACTG GCTGAATGGC AAGGAGTACA
AGTGCAAGGT CTCCAACAAA GCCCTCCCAG TCCCCATCGA
GAAAACCATC TCCAAAGCCA AAGGGCAGCC CCGAGAACCA
CAGGTGTACA CCCTGCCCCC ATCCCGGGAG GAGATGACCA
AGAACCAGGT CAGCCTGACC TGCCTGGTCA AAGGCTTCTA
TCCCAGCGAC ATCGCCGTGG AGTGGGAGAG CAATGGGCAG
CCGGAGAACA ACTACAAGAC CACGCCTCCC GTGCTGGACT
CCGACGGCTC CTTCTTCCTC TATAGCAAGC TCACCGTGGA
CAAGAGCAGG TGGCAGCAGG GGAACGTCTT CTCATGCTCC
GTGATGCATG AGGCTCTGCA CAACCACTAC ACGCAGAAGA
GCCTCTCCCT GTCTCCGGGT AAATGA

Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. In an example of a purification scheme, the cell culture medium is passed over a protein A column, washed in 150 mM Tris/NaCl (pH 8.0), then washed in 50 mM Tris/NaCl (pH 8.0) and eluted with 0.1 M glycine, pH 3.0. The low pH eluate is kept at room temperature for 30 minutes as a viral clearance step. The eluate is then neutralized and passed over a Q-sepharose ion-exchange column and washed in 50 mM Tris pH 8.0, 50 mM NaCl, and eluted in 50 mM Tris pH 8.0, with an NaCl concentration between 150 mM and 300 mM. The eluate is then changed into 50 mM Tris pH 8.0, 1.1 M ammonium sulfate and passed over a phenyl sepharose column, washed, and eluted in 50 mM Tris pH 8.0 with ammonium sulfate between 150 and 300 mM. The eluate is dialyzed and filtered for use.

Additional ActRIIB variant Fc fusion polypeptides (ActRIIB-Fc fusion proteins modified so as to reduce the ratio of activin A binding relative to myostatin or GDF11 binding) are described in WO 2008/097541 and WO 2006/012627, incorporated by reference herein.

### Example 6: Bioassay for GDF11- and Activin-Mediated Signaling

An A-204 reporter gene assay was used to evaluate the effects of ActRIIB-Fc proteins and ActRIIB variant Fc fusion polypeptides on signaling by GDF-11 and activin A. Cell line: human rhabdomyosarcoma (derived from muscle). Reporter vector: pGL3(CAGA)12 (described in Dennler et al, 1998, EMBO 17: 3091-3100). The CAGA12 motif is present in TGF-beta responsive genes (e.g., PAI-1 gene), so this vector is of general use for factors signaling through SMADs.
Day 1: Split A-204 cells into 48-well plate.
Day 2: A-204 cells transfected with 10 ug pGL3(CAGA)12 or pGL3(CAGA)12(10 ug)+pRLCMV (1 μg) and Fugene.
Day 3: Add factors (diluted into medium+0.1% BSA). Inhibitors need to be preincubated with factors for 1 hr before adding to cells. Six hrs later, cells were rinsed with PBS and lysed.

This is followed by a luciferase assay. In the absence of any inhibitors, activin A showed 10-fold stimulation of reporter gene expression and an ED50 ˜2 ng/ml. GDF-11: 16 fold stimulation, ED50: ˜1.5 ng/ml.

ActRIIB(20-134) is a potent inhibitor of, for example, activin A, GDF-8, and GDF-11 activity in this assay. Certain ActRIIB variants were also tested in this assay and the results are reproduced below.

### Example 7: ActRIIB-Fc Variants, Cell-Based Activity

Activity of ActRIIB-Fc proteins and ActRIIB variant Fc fusion polypeptides was tested in a cell-based assay as described above. Results are summarized in Table 13 below. Some variants were tested in different C-terminal truncation constructs. As discussed above, truncations of five or fifteen amino acids caused reduction in activity. The ActRIIB variant Fc fusion polypeptides (L79D and L79E variants) showed substantial loss of activin A inhibition while retaining almost wild-type inhibition of GDF11.

TABLE 13
Soluble ActRIIB-Fc binding to GDF11 and Activin A:

| | Portion of ActRIIB | | |
|---|---|---|---|
| ActRIIB-Fc | (corresponds to amino | GDF11 Inhibition | Activin Inhibition |
| Variations | acids of SEQ ID NO: 1) | Activity | Activity |
| R64 | 20-134 | +++ | +++ |
| | | (approx. 10⁻⁸ M K_{I}) | (approx. 10⁻⁸ M K_{I}) |
| A64 | 20-134 | + | + |
| | | (approx. 10⁻⁶ M K_{I}) | (approx. 10⁻⁶ M K_{I}) |
| R64 | 20-129 | +++ | +++ |
| R64 K74A | 20-134 | ++++ | ++++ |
| R64 A24N | 20-134 | +++ | +++ |
| R64 A24N | 20-119 | ++ | ++ |
| R64 A24N K74A | 20-119 | + | + |
| R64 L79P | 20-134 | + | + |
| R64 L79P K74A | 20-134 | + | + |
| R64 L79D | 20-134 | +++ | + |
| R64 L79E | 20-134 | +++ | + |
| R64K | 20-134 | +++ | +++ |
| R64K | 20-129 | +++ | +++ |
| R64 P129S P130A | 20-134 | +++ | +++ |
| R64N | 20-134 | + | + |

+ Poor activity (roughly 1 × 10⁻⁶ K_{I})
++ Moderate activity (roughly 1 × 10⁻⁷ K_{I})
+++ Good (wild-type) activity (roughly 1 × 10⁻⁸ K_{I})
++++ Greater than wild-type activity

The A24N variant has activity in the cell-based assay (above) and that is equivalent to the wild-type molecule. The A24N variant, and any of the other variants tested above, may be combined with the ActRIIB variant Fc fusion polypeptides, such as the L79D or L79E variants.

### Example 8: GDF11 and Activin A Binding

Binding of certain ActRIIB-Fc proteins and ActRIIB variant Fc fusion polypeptides to ligands was tested in a Biacore™ assay.

The ActRIIB-Fc variants or wild-type protein were captured onto the system using an anti-hFc antibody. Ligands were injected and flowed over the captured receptor proteins. Results are summarized in the tables below.

TABLE 14
Ligand-binding specificity IIB variants.

| Protein | Kon (1/Ms) | Koff (1/s) | KD (M) |
|---|---|---|---|
| | GDF11 | | |
| ActRIIB(20-134)-hFc | 1.34e−6 | 1.13e−4 | 8.42e−11 |
| ActRIIB(A24N 20-134)-hFc | 1.21e−6 | 6.35e−5 | 5.19e−11 |
| ActRIIB(L79D 20-134)-hFc | 6.7e−5 | 4.39e−4 | 6.55e−10 |
| ActRIIB(L79E 20-134)-hFc | 3.8e−5 | 2.74e−4 | 7.16e−10 |
| ActRIIB(R64K 20-134)-hFc | 6.77e−5 | 2.41e−5 | 3.56e−11 |
| | GDF8 | | |
| ActRIIB(20-134)-hFc | 3.69e−5 | 3.45e−5 | 9.35e−11 |
| ActRIIB(A24N 20-134)-hFc | | | |
| ActRIIB(L79D 20-134)-hFc | 3.85e−5 | 8.3e−4 | 2.15e−9 |
| ActRIIB(L79E 20-134)-hFc | 3.74e−5 | 9e−4 | 2.41e−9 |
| ActRIIB(R64K 20-134)-hFc | 2.25e−5 | 4.71e−5 | 2.1e−10 |
| ActRIIB(R64K 20-129)-hFc | 9.74e−4 | 2.09e−4 | 2.15e−9 |
| ActRIIB(P129S, P130R 20- | 1.08e−5 | 1.8e−4 | 1.67e−9 |
| 134)-hFc | | | |
| ActRIIB(K74A 20-134)-hFc | 2.8e−5 | 2.03e−5 | 7.18e−11 |
| | Activin A | | |
| ActRIIB(20-134)-hFc | 5.94e6 | 1.59e−4 | 2.68e−11 |
| ActRIIB(A24N 20-134)-hFc | 3.34e6 | 3.46e−4 | 1.04e−10 |
| ActRIIB(L79D 20-134)-hFc | | | Low binding |
| ActRIIB(L79E 20-134)-hFc | | | Low binding |
| ActRIIB(R64K 20-134)-hFc | 6.82e6 | 3.25e−4 | 4.76e−11 |
| ActRIIB(R64K 20-129)-hFc | 7.46e6 | 6.28e−4 | 8.41e−11 |
| ActRIIB(P129S, P130R 20- | 5.02e6 | 4.17e−4 | 8.31e−11 |
| 134)-hFc | | | |

These data obtained in a cell-free assay confirm the cell-based assay data, demonstrating that the A24N variant retains ligand-binding activity that is similar to that of the ActRIIB(20-134)-hFc molecule and that the L79D or L79E molecule retains myostatin and GDF11 binding but shows markedly decreased (non-quantifiable) binding to activin A.

Other variants have been generated and tested, as reported in WO2006/012627 (incorporated herein by reference in its entirety). See, e.g., pp. 59-60, using ligands coupled to the device and flowing receptor over the coupled ligands. Notably, K74Y, K74F, K741 (and presumably other hydrophobic substitutions at K74, such as K74L), and D801, cause a decrease in the ratio of activin A (ActA) binding to GDF11 binding, relative to the wild-type K74 molecule. Table 15 showing data with respect to these variants is reproduced below:

TABLE 15
Soluble ActRIIB-Fc variants binding to GDF11
and Activin A (Biacore ™ assay)

| ActRIIB | ActA | GDF11 |
|---|---|---|
| WT (64A) | KD = 1.8e−7M | KD = 2.6e−7M |
| | (+) | (+) |
| WT (64R) | na | KD = 8.6e−8M |
| | | (+++) |
| +15tail | KD ~2.6e−8M | KD = 1.9e−8M |
| | (+++) | (++++) |
| E37A | * | * |
| R40A | − | − |
| D54A | − | * |
| K55A | ++ | * |
| R56A | * | * |
| K74A | KD = 4.35e−9M | KD = 5.3e−9M |
| | +++++ | +++++ |
| K74Y | * | −− |
| K74F | * | −− |
| K74I | * | −− |
| W78A | * | * |
| L79A | + | * |
| D80K | * | * |
| D80R | * | * |
| D80A | * | * |
| D80F | * | * |
| D80G | * | * |
| D80M | * | * |
| D80N | * | * |
| D80I | * | −− |
| F82A | ++ | − |

* No observed binding
−− <⅕ WT binding
− ~½ WT binding
+ WT
++ < 2x increased binding
+++ ~5x increased binding
++++ ~10x increased binding
+++++ 40x increased binding

### Example 9: Generation of an ActRIIB Variant with Truncated ActRIIB Extracellular Domain

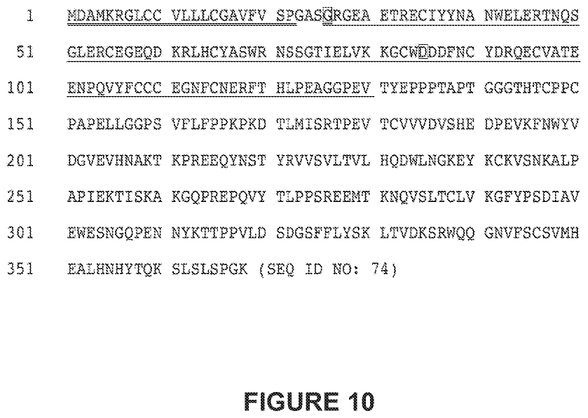

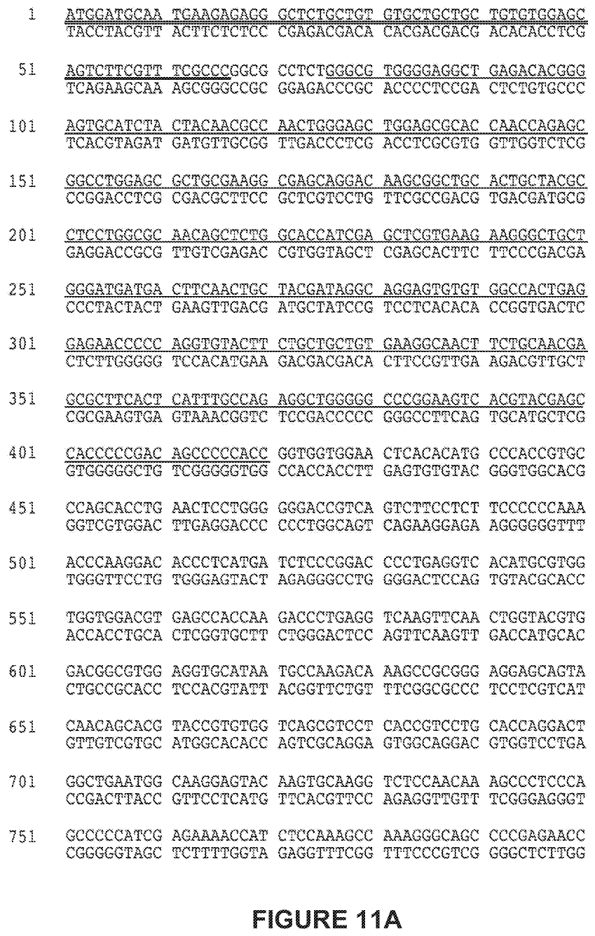

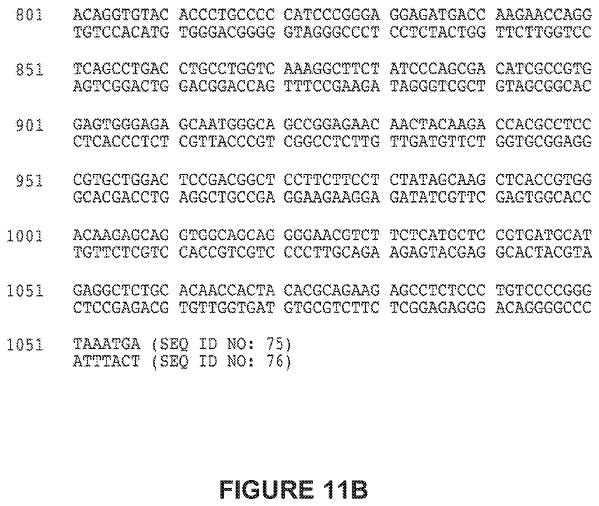

An ActRIIB variant referred to as ActRIIB(L79D 20-134)-hFc was generated by N-terminal fusion of TPA leader to the ActRIIB extracellular domain (residues 20-134 in SEQ ID NO: 1) containing a leucine-to-aspartate substitution (at residue 79 in SEQ ID NO: 1) and C-terminal fusion of human Fc domain with minimal linker (three glycine residues) (FIG. 10; SEQ ID NO: 74). A nucleotide sequence corresponding to this fusion protein is shown in FIGS. 11A and 11B (SEQ ID NO: 75, sense strand; and SEQ ID NO: 76, antisense strand).

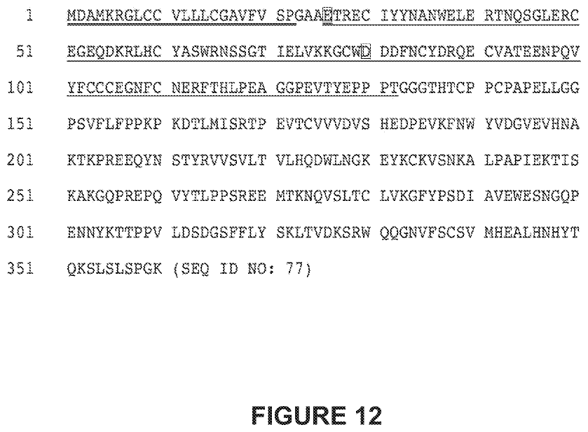

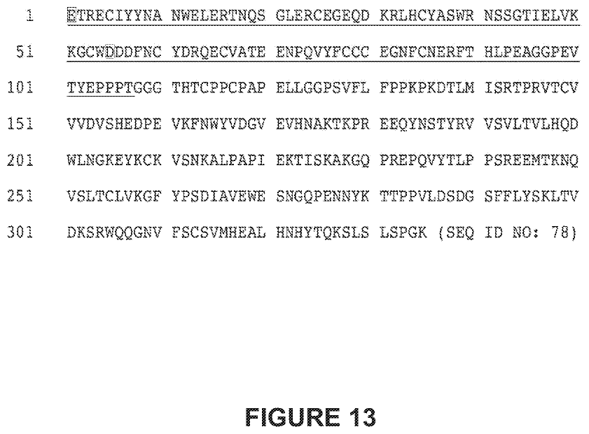

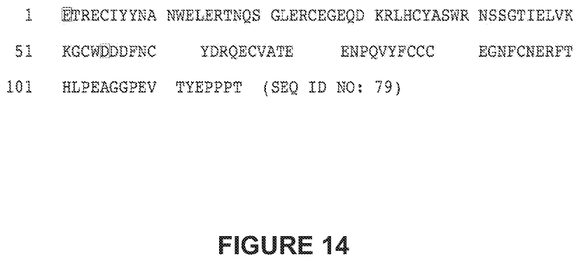

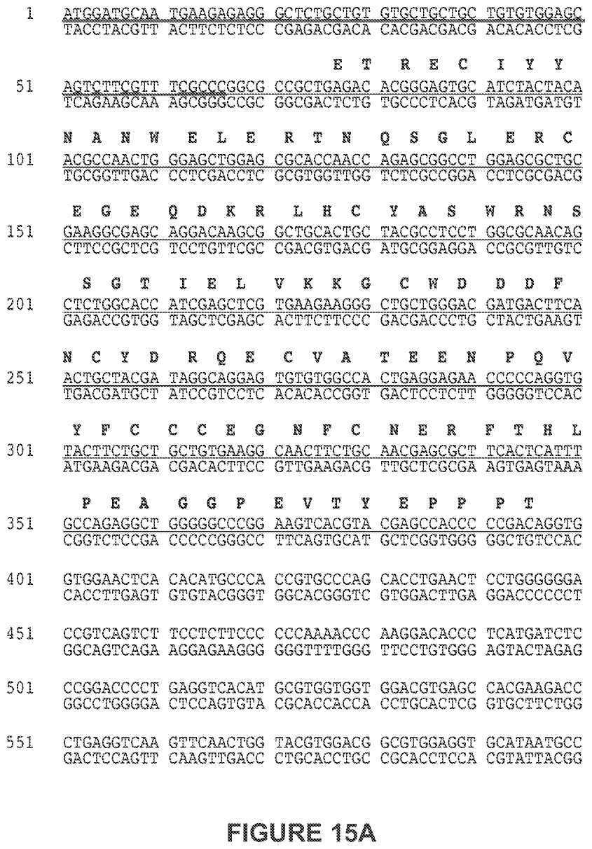

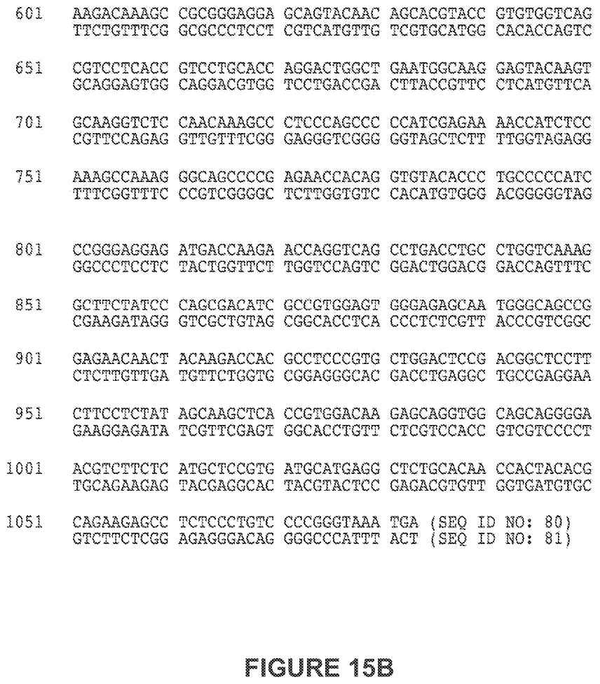

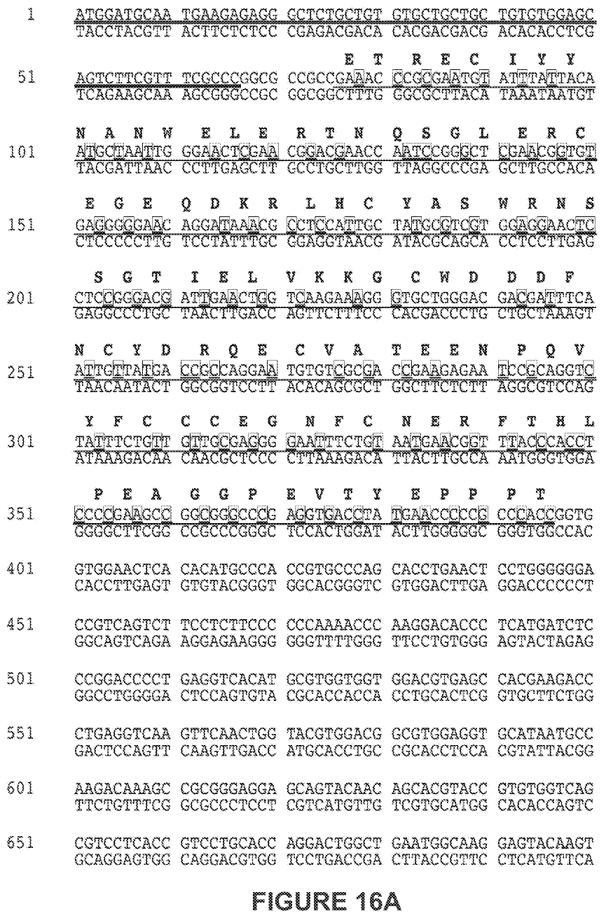

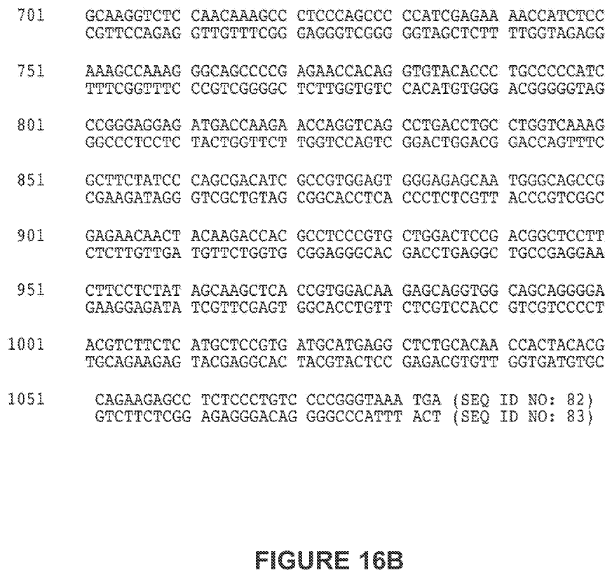

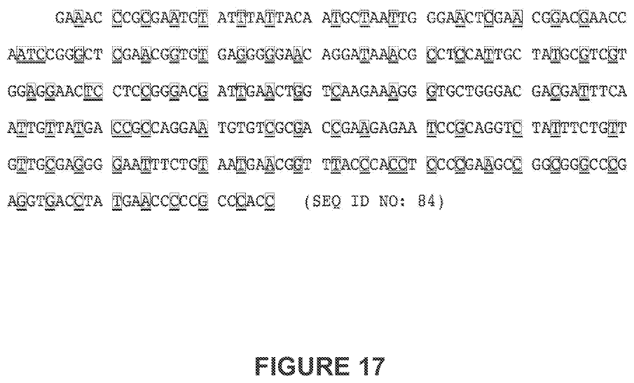

An ActRIIB variant with truncated ActRIIB extracellular domain, referred to as ActRIIB(L79D 25-131)-hFc, was generated by N-terminal fusion of TPA leader to truncated extracellular domain (residues 25-131 in SEQ ID NO:1) containing a leucine-to-aspartate substitution (at residue 79 in SEQ ID NO:1) and C-terminal fusion of human Fc domain with minimal linker (three glycine residues) (FIG. 12, SEQ ID NO: 77). The sequence of the cell purified form of ActRIIB(L79D 25-131)-hFc is presented in FIG. 13 (SEQ ID NO: 78). The sequence of the truncated ActRIIB(L79D 25-131) region without the leader, hFc domain, or linker is presented in FIG. 14 (SEQ ID NO: 79) One nucleotide sequence encoding the fusion protein is shown in FIGS. 15A and 15B (SEQ ID NO: 80) along with its complementary sequence (SEQ ID NO: 81), and an alternative nucleotide sequence encoding exactly the same fusion protein (SEQ ID NO: 82) and its complementary sequence (SEQ ID NO: 83) is shown in FIGS. 16A and 16B. An alternative nucleotide sequence (SEQ ID NO: 84) encoding only the truncated ActRIIB extracellular domain (corresponding to residues 25-131 of SEQ ID NO: 1) with the L79D substitution (SEQ ID NO: 82) is shown in FIG. 17.

### Example 10: Selective Ligand Binding by ActRIIB Variants with Double-Truncated ActRIIB Extracellular Domain

The affinity of ActRIIB variants and other ActRIIB-hFec proteins for several ligands was evaluated in vitro with a Biacore™ instrument. Results are summarized in Table 16 below. Kd values were obtained by steady-state affinity fit due to the very rapid association and dissociation of the complex, which prevented accurate determination of kₒₙ and k_{off}.

TABLE 16
Ligand Selectivity of ActRIIB-hFc Variants:

| | Activin A | Activin B | GDF11 |
|---|---|---|---|
| Fusion Construct | (Kd e−11) | (Kd e−11) | (Kd e−11) |
| ActRIIB(L79 20-134)-hFc | 1.6 | 1.2 | 3.6 |
| ActRIIB(L79D 20-134)-hFc | 1350.0 | 78.8 | 12.3 |
| ActRIIB(L79 25-131)-hFc | 1.8 | 1.2 | 3.1 |
| ActRIIB(L79D 25-131)-hFc | 2290.0 | 62.1 | 7.4 |

The ActRIIB variant with a truncated extracellular domain, ActRIIB(L79D 25-131)-hFc, equaled or surpassed the ligand selectivity displayed by the longer variant, ActRIIB(L79D 20-134)-hFc, with pronounced loss of activin A binding, partial loss of activin B binding, and nearly full retention of GDF11 binding compared to ActRIIB-hFc counterparts lacking the L79D substitution. Note that truncation alone (without L79D substitution) did not alter selectivity among the ligands displayed here [compare ActRIIB(L79 25-131)-hFc with ActRIIB(L79 20-134)-hFc]. ActRIIB(L79D 25-131)-hFc also retains strong to intermediate binding to the Smad signaling ligands GDF8, BMP6, and BMP10.

### Example 11: ActRIIB5 Variant Derived from ActRIIB5

Others have reported an alternate, soluble form of ActRIIB (designated ActRIIB5), in which exon 4, including the ActRIIB transmembrane domain, has been replaced by a different C-terminal sequence (see, e.g., WO 2007/053775).

The sequence of native human ActRIIB5 without its leader is as follows:

(SEQ ID NO: 50)
GRGEAETRECIYYNANWELERTNQSGLERCEGEQDKRLHCYASWRNSSG
TIELVKKGCWLDDFNCYDRQECVATEENPQVYFCCCEGNFCNERFTHLP
EAGGPEGPWASTTIPSGGPEATAAAGDQGSGALWLCLEGPAHE

An leucine-to-aspartate substitution, or other acidic substitutions, may be performed at native position 79 (underlined) as described to construct the variant ActRIIB5(L79D), which has the following sequence:

(SEQ ID NO: 51)
GRGEAETRECIYYNANWELERTNQSGLERCEGEQDKRLHCYASWRNSSG
TIELVKKGCWDDDFNCYDRQECVATEENPQVYFCCCEGNFCNERFTHLP
EAGGPEGPWASTTIPSGGPEATAAAGDQGSGALWLCLEGPAHE

This variant may be connected to human Fc (double underline) with a TGGG linker (SEQ ID NO: 23) (single underline) to generate a human ActRIIB5(L79D)-hFc fusion protein with the following sequence:

(SEQ ID NO: 52)
GRGEAETRECIYYNANWELERTNQSGLERCEGEQDKRLHCYASWRNSSG
TIELVKKGCWDDDFNCYDRQECVATEENPQVYFCCCEGNFCNERFTHLP
EAGGPEGPWASTTIPSGGPEATAAAGDQGSGALWLCLEGPAHETGGGTH
TCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQG
NVFSCSVMHEALHNHYTQKSLSLSPGK.

This construct may be expressed in CHO cells.

### Example 12. Effects of an ActRIIA-mFc on Group 2 Pulmonary Hypertension in a Transverse Aortic Constriction (TAC) Induced PH Mouse Model

The effects of an ActRIIA-mFc fusion protein (ActRIIA-mFc homodimer as described in Example 1) was examined in a mouse model of left ventricular systolic dysfunction (also referred to as HErEF) of pulmonary hypertension (PH). In this model, C57BL/6 mice underwent transverse aortic constriction (TAC) to induce left heart failure, and right heart and pulmonary remodeling. See, e.g., Xiong P Y, et al. Hypertension 2018, 71(1):34-55 and Chen Y, et al. Hypertension 2012, 59(6):1170-1178.

Twenty-six C57/B6 male mice (10 wks old) underwent TAC surgery and ten age-matched animals underwent a mock surgical procedure (Sham) at day 0. Two weeks after the surgery, TAC-PH mice were randomized into two groups. i) fourteen mice were injected subcutaneously with vehicle control (phosphate buffered saline (PBS)), twice weekly for 4 weeks starting from day 14 after surgery, “TAC-PH/PBS”; and a ii) twelve mice were injected subcutaneously with ActRIIA-mFc at a dose of 10 mg/kg twice weekly for 4 weeks starting from day 14 after TAC surgery, “TAC-PH/ActRII-mFc”. At the end of the study, echocardiography and pressure-volume catheter were performed to measure left and right ventricular remodeling and functional changes before animals were euthanized for heart and lung collection. Hearts and lungs of each mouse were weighed, fixed in 10% formalin, embedded in paraffin, and sectioned for Masson's trichrome stain to assess fibrosis.

Prior to euthanasia, in vivo cardiac function was assessed by transthoracic echocardiography (Acuson P300, 18 MHz transducer; Siemens) in conscious mice. From left ventricle (LV) short axis view, M-mode echocardiogram was acquired to measure left ventricle end diastolic diameter (LVEDD), and left ventricle end systolic diameter (LVESD). Fractional shortening (FS) was calculated from the end-diastolic diameter (EDD) and end-systolic diameter (ESD) using the following equation: FS=100%×[(EDD−ESD)/EDD]. Early diastolic filling peak velocity (E), early diastolic mitral annular velocity (E′), and isovolumetric relaxation time (IVRT) were measured from the medial or septal wall at the mitral valve level from tissue Doppler image. LV diastolic function was assessed by measuring the E/E′ ratio and IVRT. Three to five beats were averaged for each mouse measurement. Tricuspid annular plane systolic excursion (TAPSE), a parameter of global right ventricular function, was also measured.

On day 42, mice were anesthetized by an intraperitoneal injection of ketamine/xylazine (100/5 mg/kg) to evaluate left and right ventricular function by Millar pressure-volume conductance catheter. The respiration was supported by a small animal ventilator. Thoracotomy was made through 4-5 intercostal space, and the heart was exposed. A pressure-volume catheter (1.0-Fr, PVR-1035, Millar Instruments, Houston, TX, USA) was inserted into the left ventricle and right ventricle from the apex. Ventricular pressure and volume were calculated with LabChart 7 software. Stroke work, ejection fraction, maximum and minimum rate of pressure development (+dp/dtm, −dp/dtm) were derived.

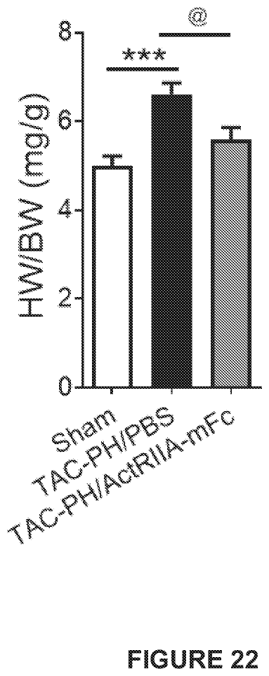

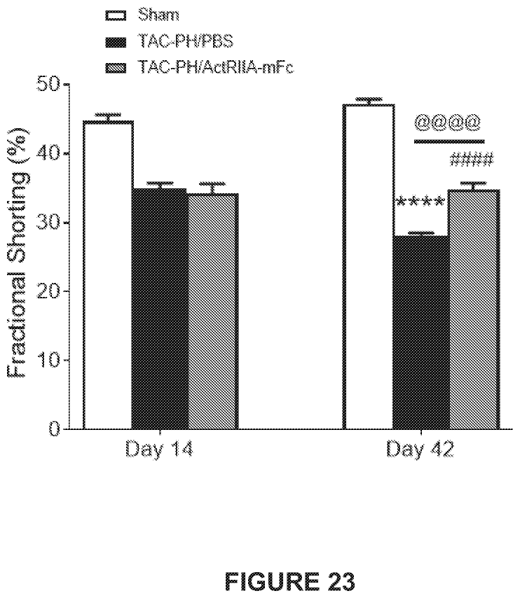

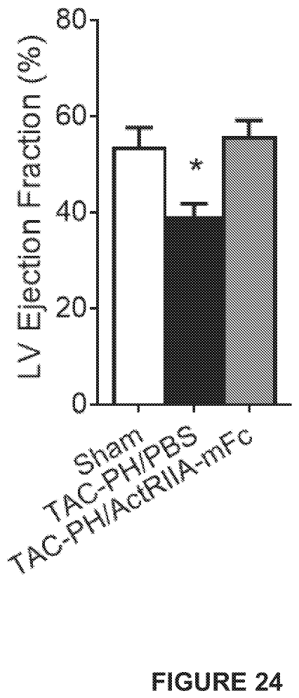

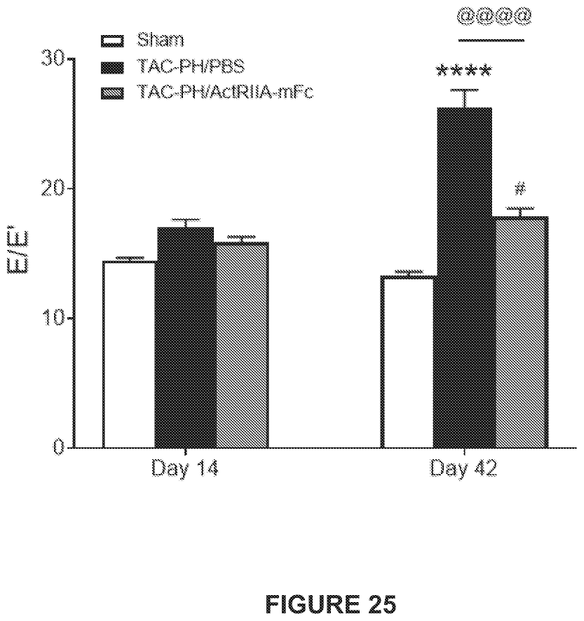

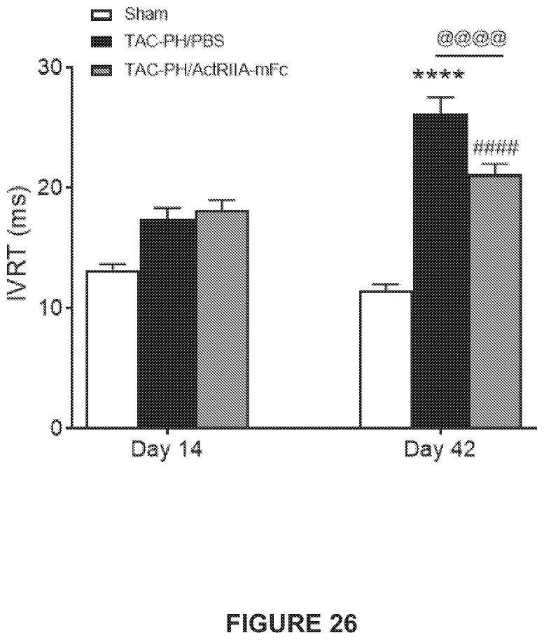

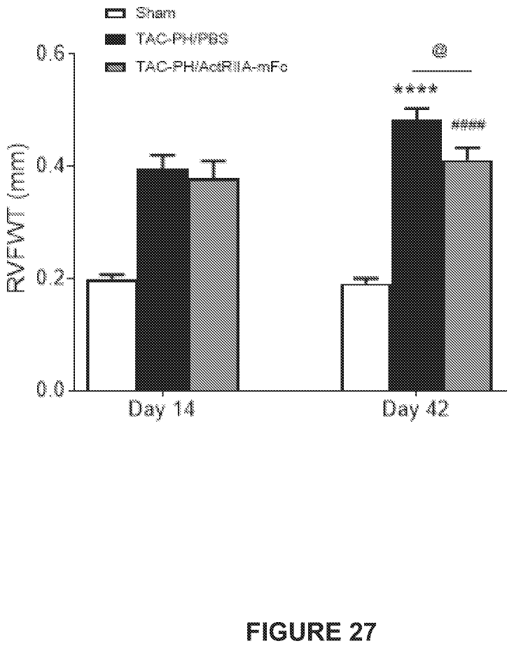

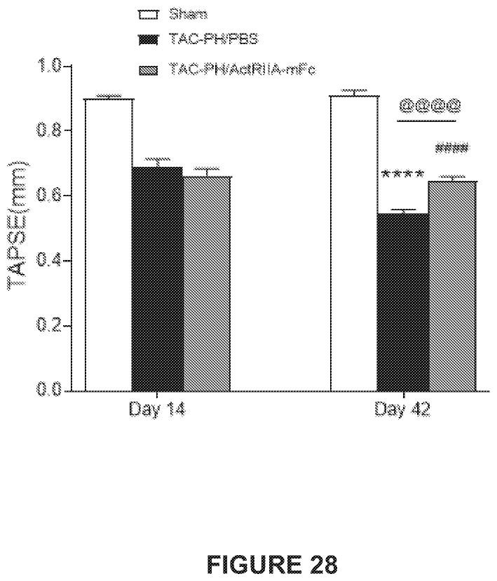

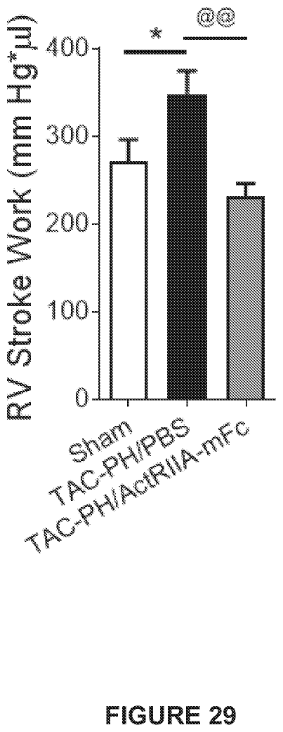

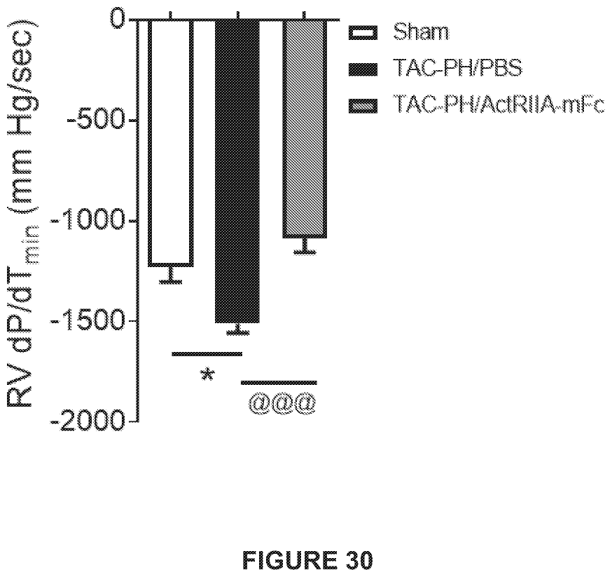

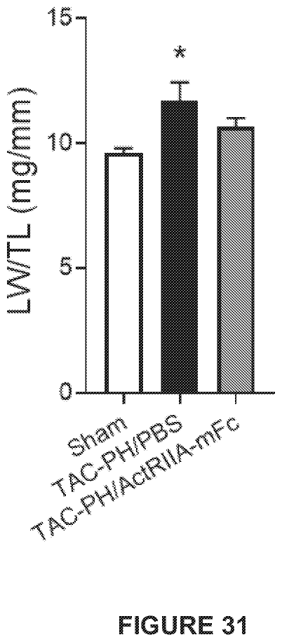

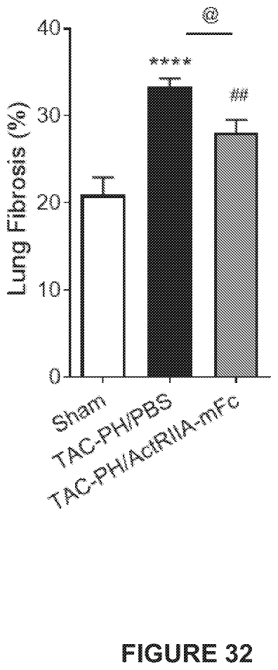

Compared to Sham control animals, TAC-PH mice in the PBS treatment group (TAC-PH/PBS) on day 42 were observed to have increased heart weight (HW/BW) (FIG. 22), reduced FS (FIG. 23), reduced LV ejection fraction (FIG. 24), increased E/E′ ratio (FIG. 25), and increased IVRT (FIG. 26), indicating cardiac hypertrophy and left heart failure. TAC mice also increased right ventricle free wall thickness (RVFWT) (FIG. 27), decreased TAPSE (FIG. 28), increased right ventricle (RV) stroke work (FIG. 29), and increased minimum rate of pressure development in RV (−dp/dTₘᵢₙ) (FIG. 30) compared to Sham control mice, suggesting the RV remodeling and RV dysfunction. In addition, increased lung weight (LW/TL) (FIG. 31) and lung fibrosis (FIG. 32) were observed in TAC-PH/PBS mice, indicating lung remodeling caused by TAC-induced left heart failure.

As shown in FIGS. 22-32, ActRIIA-mFc treatment (TAC-PH/ActRIIA-mFc) relative to PBS treatment (TAC-PH/PBS) on day 42 significantly reduced cardiac hypertrophy (FIG. 22), elevated FS (FIG. 23), restored LV ejection fraction (FIG. 24), reduced E/E′ ratio (FIG. 25), and reduced IVRT (FIG. 26). ActRIIA-mFc treatment (TAC-PH/ActRIIA-mFc) relative to PBS treatment (TAC-PH/PBS) on day 42 also significantly reduced elevated RVFWT (FIG. 27), increased reduced TAPSE (FIG. 28), reduced elevated RV stroke work (FIG. 29), and decreased increased RV −dp/dTₘᵢₙ (FIG. 30). ActRIIA-mFc treatment (TAC-PH/ActRII-mFc) relative to PBS treatment (TAC-PH/PBS) on day 42 decreased lung weight (FIG. 31) and significantly reduced lung fibrosis (FIG. 32).

Together, these data demonstrate that ActRIIA-mFc is effective in ameliorating various complications of Group 2 PH in a left heart failure-induced PH model (TAC-PH). In particular, ActRIIA-mFc had a significant effect in reducing cardiac hypertrophy, improving cardiac function, improving right heart remodeling and function, and reducing pulmonary remodeling and fibrosis.

### Example 13: Effects of an ActRIIA-mFc on Group 2 Pulmonary Hypertension in an HFpEF Induced PH Rat Model

The effects of an ActRIIA-mFc fusion protein (ActRIIA-mFc homodimer as described in Example 1) was examined in a rat model of left ventricular diastolic dysfunction (also referred to as HEpEF) group 2 (subgroup 2.2) pulmonary hypertension (PH). In this model, ZSF1-Lepr^{fa}Lepr^{cp}/Crl rats were challenged with semaxanib to induce HFpEF-PH.

Forty ZSF1 Lepr^{fa}Lepr^{cp}/Crl male mice (8 wks old) and five lean rats were subcutaneously administered with a single dose of semaxanib (100 mg/kg) at day 0, and five lean rats were included as normal control. Six weeks after semaxanib (SU5416) treatment, Thirty-six ZSF1 Lepr^{fa}Lepr^{cp}/Crl rats were randomized into four groups: i) nine rats were injected subcutaneously with vehicle control (phosphate buffered saline (PBS)), twice weekly for 8 weeks starting from day 42 after semaxanib treatment, “ZSF1-SU/PBS”; a ii) ten rats were injected subcutaneously with ActRIIA-mFc at a dose of 1 mg/kg twice weekly for 8 weeks starting from day 42 after semaxanib treatment, “ZSF1-SU/ActRIIA-mFc 1 mpk”; a iii) nine rats were injected subcutaneously with ActRIIA-mFc at a dose of 3 mg/kg twice weekly for 8 weeks starting from day 42 after semaxanib treatment, “ZSF1-SU/ActRIIA-mFc 3 mpk”; and a iv) eight rats were injected subcutaneously with ActRIIA-mFc at a dose of 10 mg/kg twice weekly for 8 weeks starting from day 42 after semaxanib treatment, “ZSF1-SU/ActRIIA-mFc 10 mpk”. At the end of the study, echocardiography and pressure-volume catheter were performed to measure left and right ventricular remodeling and functional changes before animals were euthanized for heart and lung collection. Hearts and lungs of each rat were weighed, fixed in 10% formalin, embedded in paraffin, and sectioned for Masson's trichrome stain to assess fibrosis. Serum and urine samples were collected at the end of the study.

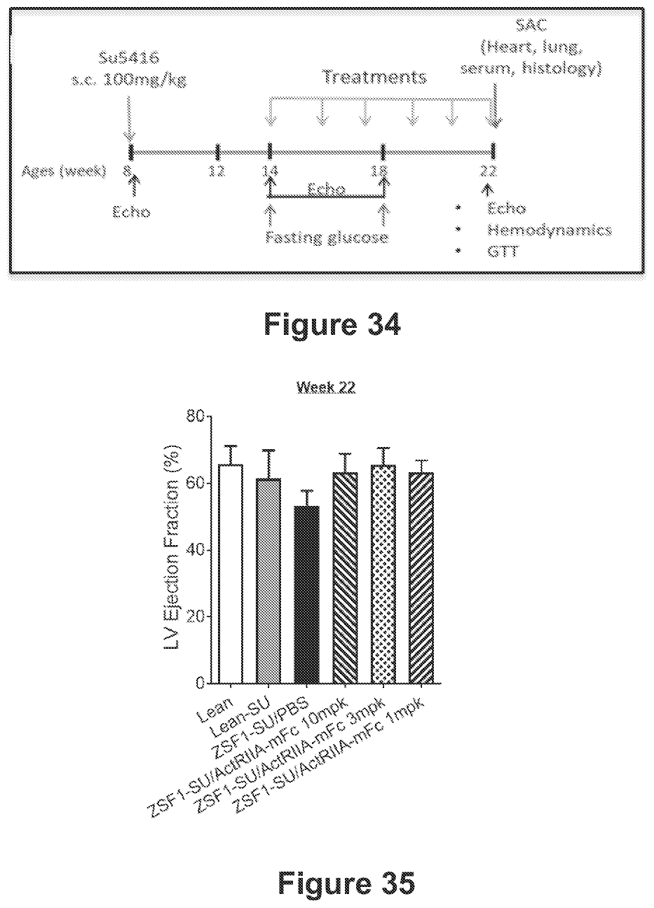

Rats were fasted overnight to measure fasting blood glucose levels at week 14 (before treatments started), week 18 (4 weeks after treatments), and week 22, and oral glucose tolerance test was performed at week 22 (see FIG. 34). Blood glucose levels were measured with a glucometer after bleeding tail vein with a 27 G needle. To prepare oral glucose tolerance test, 40% glucose stock solution run through a filter to sterilize it. After fasting overnight, rat body weight was measured. Blood glucose level was detected. Then 40% glucose solution was administered via oral gavage according to body weight (2 g/kg). Blood glucose levels were measured at 30, 60, 90, 120 minutes.

Prior to euthanization, in vivo cardiac function was assessed by transthoracic echocardiography (Acuson P300, 18 MHz linear transducer; Siemens) in lightly anesthetized rats as described. From left ventricle short axis view, M-mode echocardiogram was acquired to measure interventricular septal thickness at end diastole (IVSd), left ventricular posterior wall thickness at end diastole (LVPWd), left ventricular end diastolic diameter (LVEDD), and left ventricular end systolic diameter (LVESD). Left ventricular mass (LVM) was assessed by the equation: 1.05 [(LVEDD+LVPTD+IVSd)³−LVEDD³]. Early diastolic filling peak velocity (E), early diastolic mitral annular velocity (E′), and isovolumetric relaxation time (IVRT) were measured from the medial or septal wall at the mitral valve level from tissue Doppler image. LV diastolic function was assessed by measuring the E/E′ ratio and IVRT. Pulmonary arterial acceleration time (PAAT), a parameter of right ventricular function, was also measured.

Fourteen weeks after semaxanib treatment, rats were anesthetized with ketamine (100 mg/kg) and xylazine (5 mg/kg) at the end of the experiment to evaluate cardiac and pulmonary hemodynamics. The respiration was supported by a small animal ventilator. Thoracotomy was made through 4-5 intercostal space, and the heart was exposed. A pressure-volume catheter (2.0-Fr, SPR-869, Millar Instruments, Houston, TX, USA) was be inserted into the left ventricle and right ventricle from the apex. Ventricular pressure and volume were calculated with LabChart 7 software. Stroke work, ejection fraction, and cardiac output were derived. After finishing left ventricular measurements, the catheter was advanced to the aorta, arterial blood systolic and diastolic pressure was detected. Then the catheter returned to the left ventricle and changed the direction laterally to enter the left atrium. Similarly, right atrial pressure was measured by moving the catheter from the right ventricle into atrium. To measure pulmonary arterial pressure, the sternum was cross-sectioned at the second inter-rib space. The right ventricular outflow tract was exposed. A hole was made with 27 G needle, and then the catheter was inserted into the right ventricular outflow tract and advanced into the pulmonary artery.

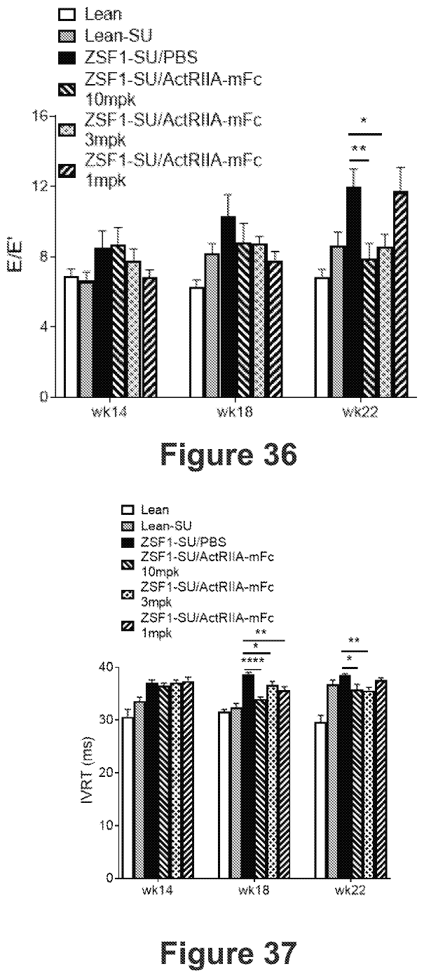

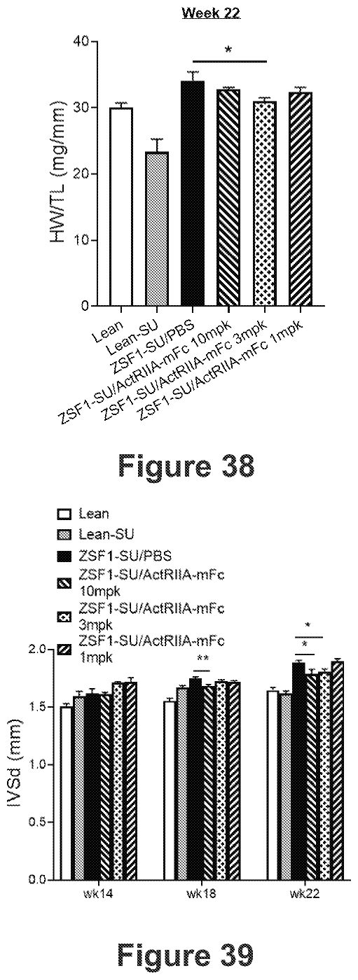

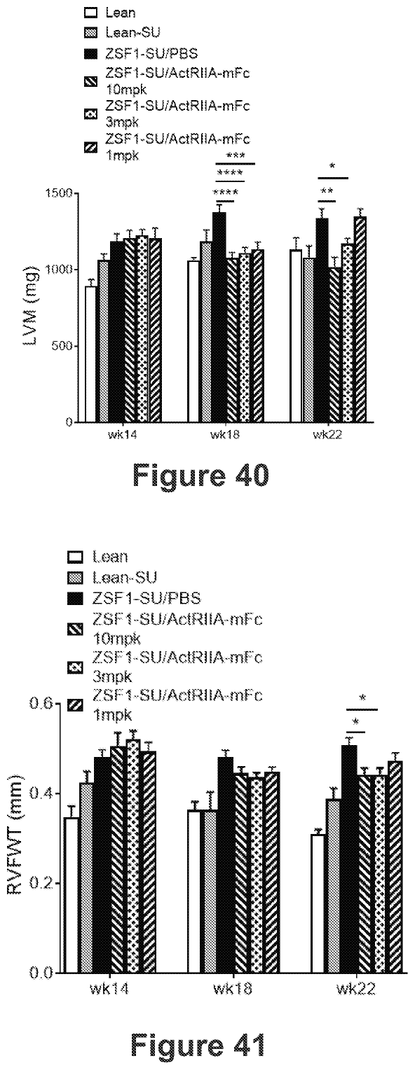

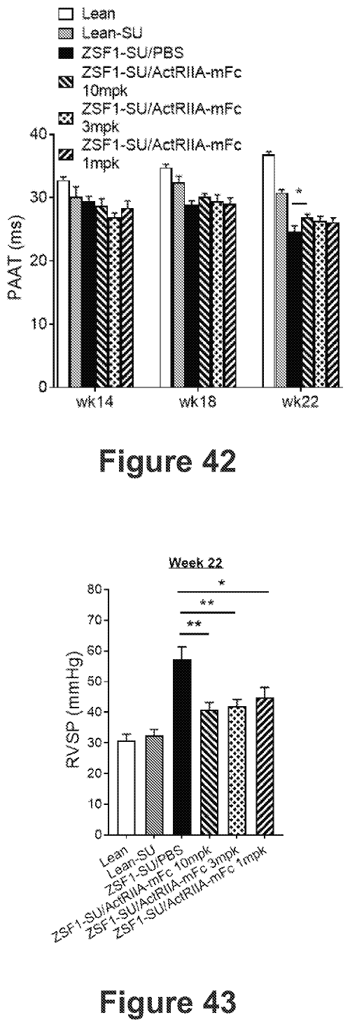

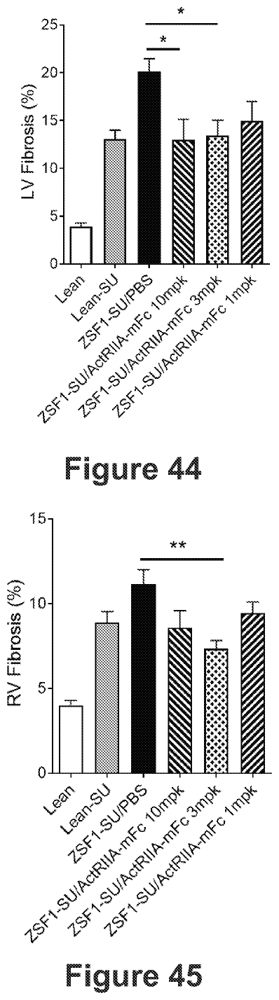

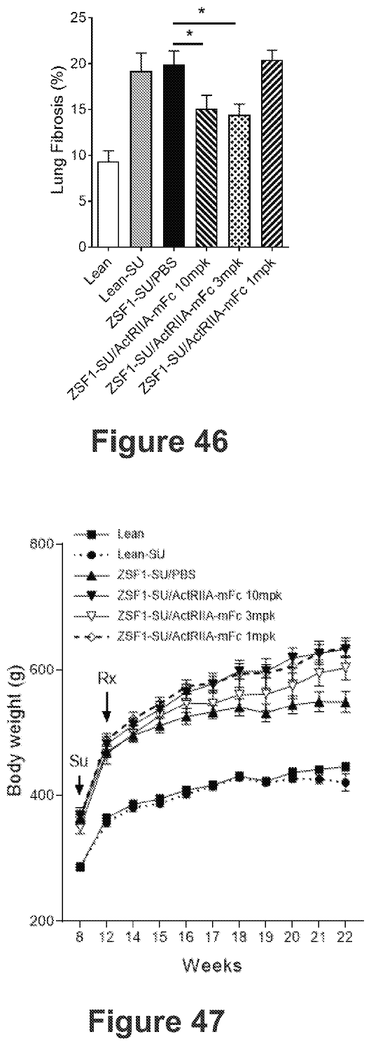

Compared to lean control animals, ZSF1-SU rats in the PBS treatment group (ZSF1-SU/PBS) 14-weeks after semaxanib treatment were observed to have increased heart weight (HW/TL) (FIG. 38), increased IVSd (FIG. 39), and increased LVM (FIG. 40), preserved LV ejection fraction (FIG. 35), increased E/E′ ratio (FIG. 36), increased IVRT (FIG. 37), indicating cardiac hypertrophy and left ventricular diastolic dysfunction. ZSF1 rats also increased right ventricle free wall thickness (RVFWT) (FIG. 41), decreased PAAT (FIG. 42), and increased RVSP (FIG. 43), compared to lean control rats, suggesting the pulmonary hypertension and RV remodeling. In addition, increased fibrosis in LV, RV and lung (FIGS. 44-46) was observed in ZSF1-SU/PBS rats.

As shown in FIGS. 38-40, ActRIIA-mFc treatment (ZSF1-SU/ActRIIA-mFc) relative to PBS treatment (ZSF1-SU/PBS) both at 3 mpk and 10 mpk significantly reduced left heart remodeling (FIGS. 38-40), and reduced E/E′ ratio (FIG. 36), and decreased IVRT (FIG. 37). ActRIIA-mFc treatment (ZSF1-SU/ActRIIA-mFc) relative to PBS treatment (ZSF1-SU/PBS) both at 3 mpk and 10 mpk also significantly reduced elevated RVFWT (FIG. 41), reduced PAAT (FIG. 42), and reduced elevated RVSP (FIG. 43). ActRIIA-mFc treatment (ZSF1-SU/ActRIIA-mFc) relative to PBS treatment (ZSF1-SU/PBS) also significantly reduced the increased fibrosis in LV, RV and lung (FIGS. 44-46).

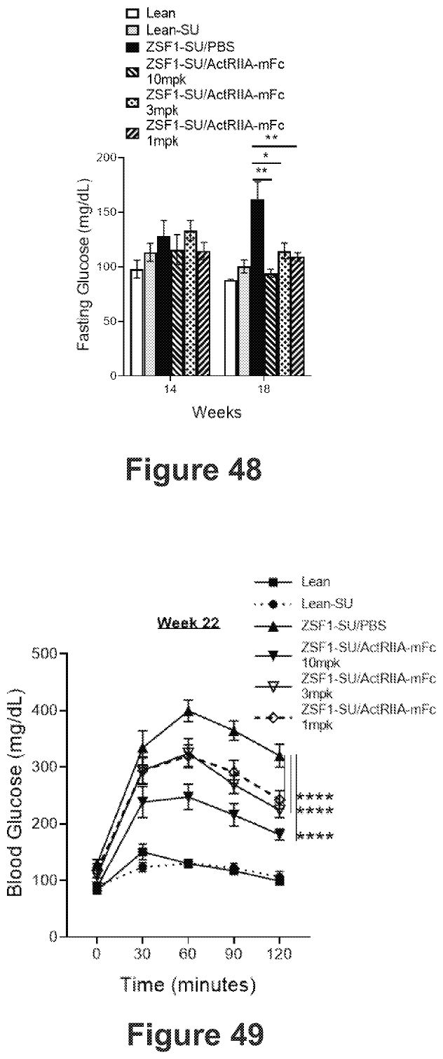

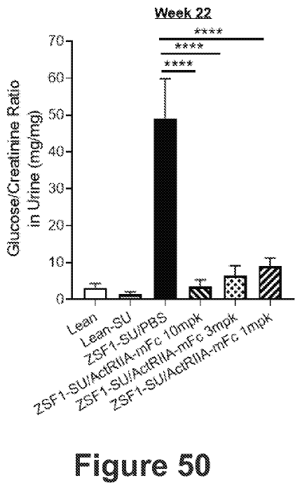

In addition, compared to lean control animals, ZSF1-SU rats in the PBS treatment group (ZSF1-SU/PBS) had elevated fasting blood glucose level and increased glucose level in urine, accompanied by glucose intolerance. ActRIIA-mFc treatment (ZSF1-SU/ActRIIA-mFc) relative to PBS treatment (ZSF1-SU/PBS) at 1 mpk, 3 mpk and 10 mpk significantly reduced fasting blood glucose, decreased glucose level in urine, and improved glucose tolerance (FIGS. 48-50). Additionally, FIG. 47 shows changes in body weight.

Together, these data demonstrate that ActRIIA-mFc is effective in ameliorating various complications of Group 2 PH in a left heart failure-induced PH model (HFpEF-PH). In particular, ActRIIA-mFc had a significant effect in reducing cardiac hypertrophy, improving diastolic function, improving right heart remodeling and function, decreasing pulmonary hypertension, and reducing cardiac and pulmonary remodeling and fibrosis. Furthermore, ActRIIA-mFc had a robust effect in reducing glucose levels and improving glucose tolerance. The data indicate that other ActRII antagonists, particularly ones having activities similar to ActRIIA-mFc, may be useful in the treatment of Group 2 PH, particularly in preventing or reducing the severity various complications of Group 2 PH.

### Example 14: Effects of an ActRIIA-hFc Polypeptide in Patients with Cpc-PH Due to HFpEF

The effects of an ActRIIA-hFec fusion protein (SEQ ID NO: 23 as described in Example 1) are examined in a double-blind, randomized, placebo-controlled study to evaluate the effects of the ActRIIA-hFc fusion protein versus placebo for the treatment of combined pre- and postcapillary pulmonary hypertension (Cpc-PH) due to heart failure with preserved ejection fraction (HFpEF).

### Patients and Trial Design

Eligible patients will have confirmed Cpc-PH due to HFpEF, Functional Class II or III as assessed by the NYHA. Additionally, eligible patients are between 18 to 85 years of age and have a six minute walk distance greater than 100 meters repeated twice during screening and both values within 15% of each other, calculated from the highest value. Patients may be receiving stable medications for heart failure or any underlying condition for at least 30 days before and throughout the study. A planned interim analysis will occur when approximately 15 participants in each of the three treatment groups have completed 24 weeks on the study. Sensitivity analysis will be performed to account for any differences in background therapy. All patients will provide informed consent.

Initially, approximately 90 eligible patients will be randomly assigned in a 1:1:1 ratio to one of three treatment groups: (1) placebo; (2) ActRIIA-hFc fusion protein 0.3 mg/kg; or (3) ActRIIA-hFc fusion protein 0.3 mg/kg then escalating to 0.7 mg/kg. ActRIIA-hFc fusion protein or placebo (saline) will be given by subcutaneous injection every 21 days for a total of 24 weeks. Safety and efficacy will be assessed at screening and every 3 weeks for 24 weeks. See, e.g., Table 17 below. Adverse events are recorded from screening until the end of primary treatment study visit, 8 weeks after the last dose of study drug. An interim analysis will occur when approximately 15 participants in each of the 3 treatment groups have completed 24 weeks of treatment in the placebo-controlled treatment period.

Participants who have not discontinued early from the placebo-controlled treatment period and have had the 24-week PVR assessment will continue into the 18-month extension period and will be treated as follows: Placebo participants will be re-randomized in a 1:1 ratio to one of the two ActRIIA-hFc fusion protein treatment groups utilized in the placebo-controlled treatment period to receive either (1) ActRIIA-hFc fusion protein SC at a dose level of 0.3 mg/kg every 21 days for up to 18 months in the Extension Period or (2) ActRIIA-hFc fusion protein SC at a starting dose of 0.3 mg/kg plus background therapy, then escalate to 0.7 mg/kg at Visit 12 and every 21 days for up to 18 months in the Extension Period.

TABLE 17
Efficacy Endpoints

| Type | End points |
|---|---|
| Primary | Change in pulmonary vascular resistance from baseline |
| end point | to 24 weeks |
| Key | Change in 6-minute walk distance from baseline to |
| secondary | 24 weeks |
| end point | |
| Other | Clinical Worsening |
| secondary | Number of Clinical Worsening events, defined as follows, |
| end points | at 24 and 48 weeks: |
| | The occurrence of any 1 of the following clinical |
| | worsening events: hospitalization due to a cardiopulmonary |
| | indication (a non-elective hospitalization lasting at |
| | least 24 hours in duration caused by clinical conditions |
| | directly related to PH and/or heart failure), |
| | administration of IV diuretics, death (all causes), |
| | decrease in 6MWD >15% from Baseline (or the subject |
| | was too ill to walk, and the cause was directly |
| | related to the disease under study) at 2 consecutive |
| | visits on different days (except Week 24) |
| | Number of Participants with first Clinical Worsening |
| | event, defined as above, at 24 and 48 weeks |
| | Time to Clinical Worsening, defined as above |
| | Change in dyspnea score (assessed by Borg CR10 |
| | scale ®) at Week 24 from baseline |
| | Change in hemodynamic and ECHO parameters, |
| | including but not limited to mPAP, PCWP, TAPSE, |
| | RVFAC, and LVEF at 24 weeks from baseline |
| | Change in NT-proBNP at 24 weeks from baseline |
| | Change in NYHA FC at 24 weeks from baseline |
| | Change in 6MWD at 48 weeks from baseline |
| | Change in hemodynamic and ECHO parameters, including |
| | but not limited to PVR, mPAP, PCWP, TAPSE, RVFAC, |
| | and LVEF, at 48 weeks from baseline |
| | Change in NT-proBNP at 48 weeks from baseline |
| | Change in NYHA FC at 48 weeks from baseline |
| | Change in PVR, 6MWD and NYHA FC at week 48 from |
| | baseline in the extension in the Placebo-Crossed |
| | treatment group |
| | Change in PVR, 6MWD and NYHA FC from week 24 to |
| | week 48 in the extension in the Placebo-Crossed |
| | treatment group |
| Exploratory | Placebo-Controlled Treatment and Extension Periods |
| end points | Changes in Kansas City Cardiomyopathy Questionnaire |
| | (KCCQ) and EQ-5D-5L scores |
| | Change from baseline in disease-related biomarkers |
| | at 24 weeks and 48 weeks |
| | Correlation of clinical efficacy vs. genetic phenotype |

### Example 15: Effects of an ActRIIA-mFc on Group 2 Pulmonary Hypertension in a Transverse Aortic Constriction (TAC) Induced PH Mouse Model

The effects of an ActRIIA-mFc fusion protein (ActRIIA-mFc homodimer as described in Example 1) was examined in a mouse model of left ventricular systolic dysfunction (also referred to as HErEF) of pulmonary hypertension (PH) and valvular heart disease. In this model, BALB/cJ mice underwent transverse aortic constriction (TAC) to induce left heart failure, and right heart and pulmonary remodeling. See, e.g., Xiong P Y, et al. Hypertension 2018, 71(1):34-55 and Chen Y, et al. Hypertension 2012, 59(6):1170-1178.

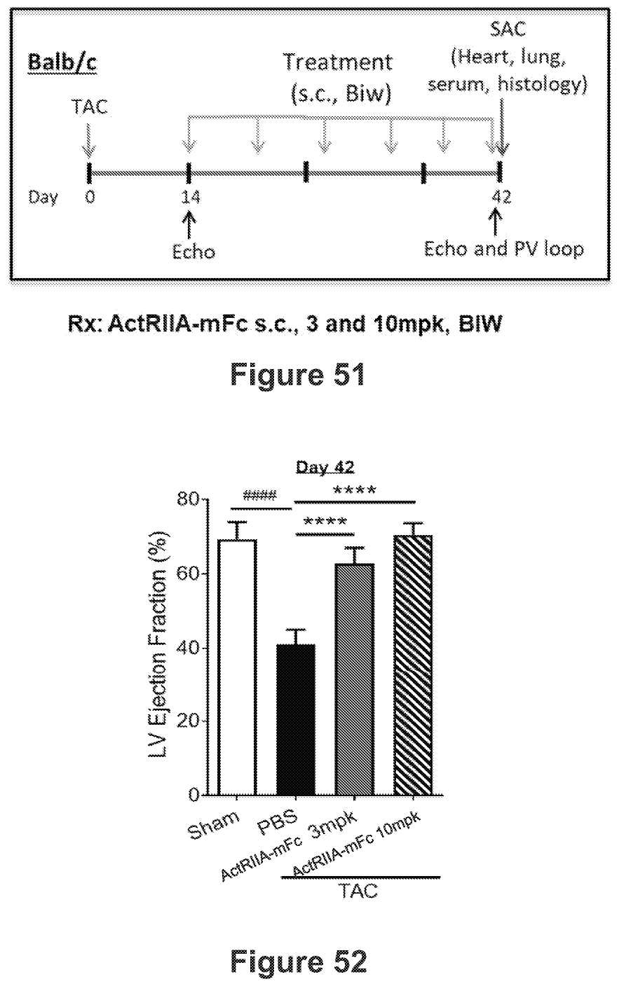

The experimental strategy used to test the preventative effects of ActRIIA-mFc in the rat model of HErEF is shown in FIG. 51. Forty-four BALB/cJ male mice (10 wks old) underwent TAC surgery and fourteen age-matched animals underwent a mock surgical procedure (Sham) at day 0. Two weeks after the surgery, TAC-PH mice were randomized into three groups. i) fourteen mice were injected subcutaneously with vehicle control (phosphate buffered saline (PBS)), twice weekly for 4 weeks starting from day 14 after surgery, “TAC PBS”; ii) fifteen mice were injected subcutaneously with ActRIIA-mFc at a dose of 3 mg/kg twice weekly for 4 weeks starting from day 14 after TAC surgery, “TAC ActRIIA-mFc 3 mpk”; and iii) fifteen mice were injected subcutaneously with ActRIIA-mFc at a dose of 10 mg/kg twice weekly for 4 weeks starting from day 14 after TAC surgery, “TAC ActRIIA-mFc 10 mpk.” At the end of the study, echocardiography and pressure-volume catheter were performed to measure left and right ventricular remodeling and functional changes before animals were euthanized for heart and lung collection. Hearts and lungs of each mouse were weighed, fixed in 10% formalin, embedded in paraffin, and sectioned for Masson's trichrome stain to assess fibrosis.

Prior to euthanasia, in vivo cardiac function was assessed by transthoracic echocardiography (Acuson P300, 18 MHz transducer; Siemens) in conscious mice. From left ventricle (LV) short axis view, M-mode echocardiogram was acquired to measure left ventricle end diastolic diameter (LVEDD), and left ventricle end systolic diameter (LVESD). Fractional shortening (FS) was calculated from the end-diastolic diameter (EDD) and end-systolic diameter (ESD) using the following equation: FS=100%×[(EDD−ESD)/EDD]. Early diastolic filling peak velocity (E), early diastolic mitral annular velocity (E′), and isovolumetric relaxation time (IVRT) were measured from the medial or septal wall at the mitral valve level from tissue Doppler image. LV diastolic function was assessed by measuring the E/E′ ratio and IVRT. Three to five beats were averaged for each mouse measurement. RV free wall thickness (RVFWT) was measured using M-mode in a modified parasternal long-axis view through the aortic valve. Pulmonary artery acceleration time (PAAT) was measured as the time from start to peak velocity of blood flow in the lumen of the main pulmonary artery distal to the pulmonary valve as obtained from the pulse-wave doppler recording.

On day 42, mice were anesthetized by an intraperitoneal injection of ketamine/xylazine (100/5 mg/kg) to evaluate left and right ventricular function by Millar pressure-volume conductance catheter. The respiration was supported by a small animal ventilator. Thoracotomy was made through 4-5 intercostal space, and the heart was exposed. A pressure-volume catheter (1.0-Fr, PVR-1035, Millar Instruments, Houston, TX, USA) was inserted into the left ventricle and right ventricle from the apex. Ventricular pressure and volume were calculated with LabChart 7 software. Ejection fraction was derived. Afterwards, animals were euthanized for heart and lung collection. Cardiac hypertrophy was measured by heart weight (HW) normalized by tibial length (TL). Left ventricle (LV), right ventricle (RV) and lung of each mouse were separated, fixed in 10% formalin, embedded in paraffin, and sectioned for Masson's trichrome stain to assess fibrosis. Serum and urine samples were collected at the end of the study.

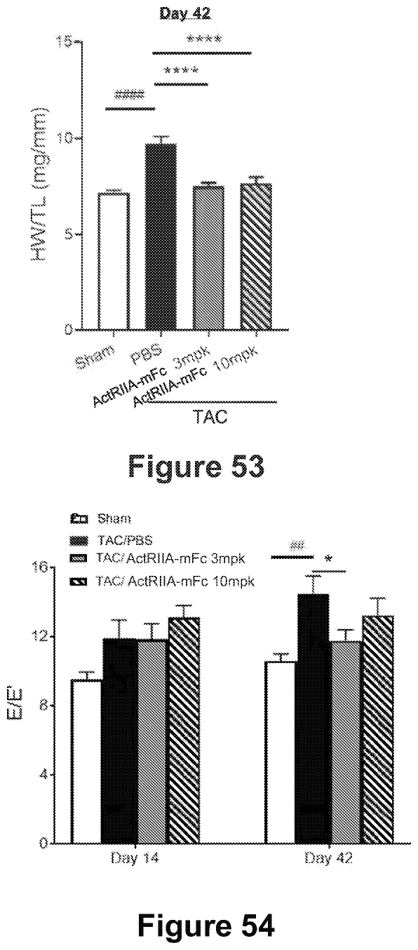

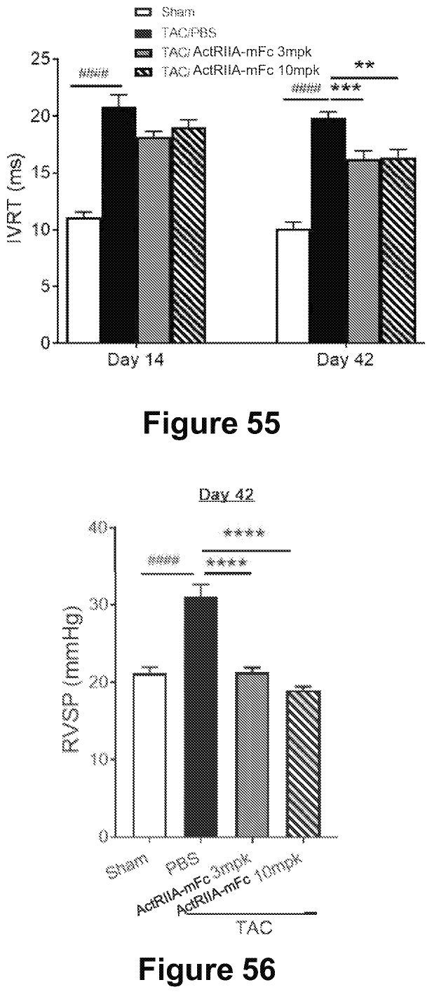

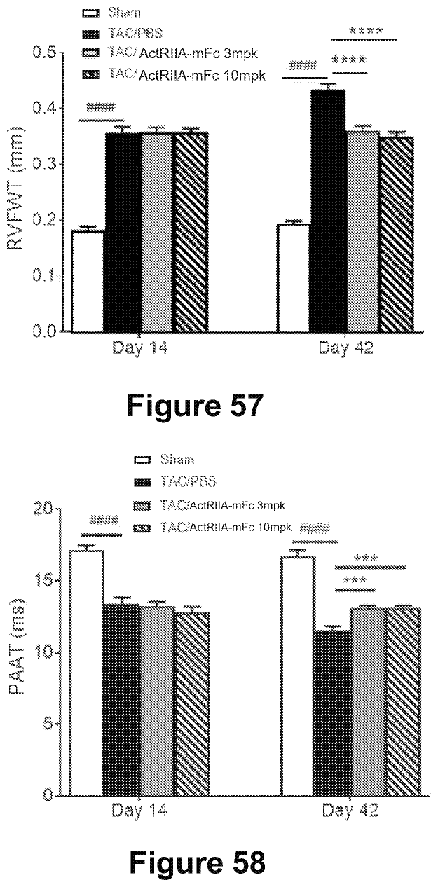

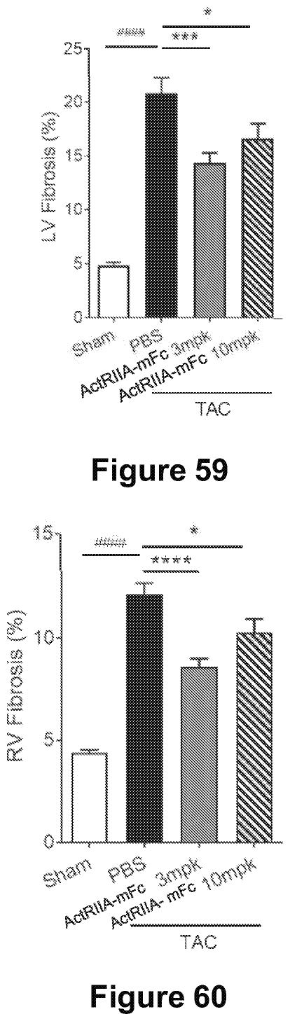

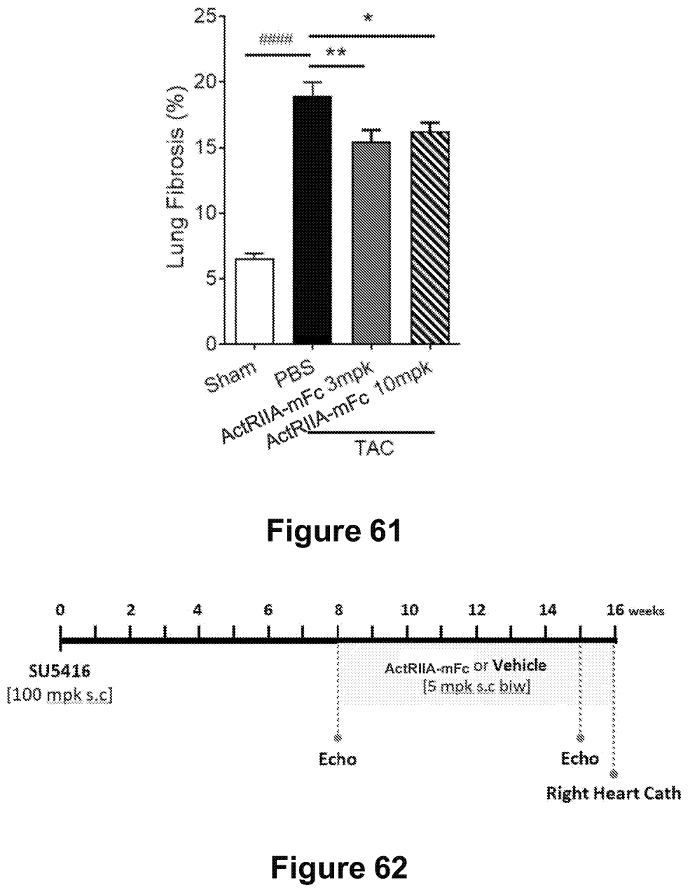

Compared to Sham control animals, TAC-PH mice in the PBS treatment group (TAC PBS) on day 42 were observed to have decreased left ventricle ejection fraction (FIG. 52), increased heart weight (HW/TL) (FIG. 53), increased E/E′ ratio (FIG. 54), increased isovolumic relaxation time (IVRT) (FIG. 55), and increased left ventricle fibrosis (FIG. 59), indicating cardiac hypertrophy and left heart failure. TAC mice also had increased right ventricle free wall thickness (RVFWT) (FIG. 57), decreased PAAT (FIG. 58), and increased right ventricle fibrosis (FIG. 60) compared to Sham control mice, suggesting the RV remodeling and RV dysfunction. In addition, increased RVSP (FIG. 56) and increased lung fibrosis (FIG. 61) were observed in TAC-PH/PBS mice, indicating pulmonary hypertension and lung remodeling caused by TAC-induced left heart failure.

As shown in FIGS. 52-61, ActRIIA-mFc treatment (TAC ActRIIA-mFc 3 mpk or TAC ActRIIA-mFc 10 mpk) relative to PBS treatment (TAC PBS) on day 42 significantly reduced cardiac hypertrophy (FIG. 53), restored LV ejection fraction (FIG. 52), reduced E/E′ ratio at 3 mpk (FIG. 54), and reduced IVRT (FIG. 55). ActRIIA-mFc treatment (TAC ActRIIA-mFc 3 mpk or TAC ActRIIA-mFc 10 mpk) relative to PBS treatment (TAC PBS) on day 42 also significantly reduced elevated RVFWT (FIG. 57), reduced RVSP (FIG. 56), (FIG. 57), and increased PAAT (FIG. 58). ActRIIA-mFc treatment (TAC ActRIIA-mFc 3 mpk or TAC ActRIIA-mFc 10 mpk) relative to PBS treatment (TAC PBS) on day 42 significantly reduced lung fibrosis (FIG. 42), LV fibrosis (FIG. 59), and RV fibrosis (FIG. 60).

Together, these data demonstrate that ActRIIA-mFc is effective in ameliorating various complications of Group 2 PH in a left heart failure-induced PH model (TAC PH). In particular, ActRIIA-mFc had a significant effect in reducing cardiac hypertrophy, improving cardiac function, improving right heart remodeling and function, improving LV function, and reducing pulmonary remodeling and fibrosis.

### Example 16: Effects of an ActRIIA-mFc on Group 2 Pulmonary Hypertension in an HFpEF Induced PH Rat Model

The effects of an ActRIIA-mFc fusion protein (ActRIIA-mFc homodimer as described in Example 1) was examined in a rat model of left ventricular diastolic dysfunction (also referred to as HFpEF) group 2 (subgroup 2.2) pulmonary hypertension (PH). The experimental strategy used to test the preventative effects of an ActRIIA-mFc fusion protein in the rat model of HEpEF is shown in FIG. 62. In this model, ZSF1-Lepr^{fa}Lepr^{cp}/Crl rats were challenged with semaxanib (SU5416) to induce HFpEF-PH.

Twenty ZSF1 Lepr^{fa}Lepr^{cp}/Crl male mice (8 wks old) and ten lean rats were subcutaneously administered with a single dose of semaxanib (100 mg/kg) at day 0, and ten lean rats were included as normal control. Eight weeks after semaxanib (SU5416) treatment, twenty ZSF1 Lepr^{fa}Lepr^{cp}/Crl rats were randomized into two groups: i) ten rats were injected subcutaneously with vehicle control (phosphate buffered saline (PBS)), twice weekly for 8 weeks starting from day 64 after semaxanib treatment, “Obese ZSF1 Veh”; and ii) ten rats were injected subcutaneously with ActRIIA-mFc at a dose of 5 mpk twice weekly for 8 weeks starting from day 64 after semaxanib treatment, “Obese ZSF1 ActRIIA-mFc.” At the end of the study, echocardiography and pressure-volume catheter were performed to measure left and right ventricular remodeling and functional changes before animals were euthanized for heart and lung collection.

Prior to euthanization, in vivo cardiac function was assessed by transthoracic echocardiography (Acuson P300, 18 MHz linear transducer; Siemens) in lightly anesthetized rats as described. Echocardiographic assessments were conducted at week 8 (before therapy with ActRIIA-mFc or vehicle) and week 15 (after therapy) in each rat. From left ventricle short axis view, M-mode echocardiogram was acquired to measure interventricular septal thickness at end diastole (IVSd), left ventricular posterior wall thickness at end diastole (LVPWd), left ventricular end diastolic diameter (LVEDD), and left ventricular end systolic diameter (LVESD). Left ventricular mass (LVM) was assessed by the equation: 1.05 [(LVEDD+LVPTD+IVSd)³−LVEDD³]. Early diastolic filling peak velocity (E), early diastolic mitral annular velocity (E′), and isovolumetric relaxation time (IVRT) were measured from the medial or septal wall at the mitral valve level from tissue Doppler image. LV diastolic function was assessed by measuring the E/E′ ratio and IVRT. Pulmonary arterial acceleration time (PAAT), a parameter of right ventricular function, was measured. Tricuspid annular plane systolic excursion (TAPSE), a parameter of global right ventricular function, was also measured. RV free wall thickness (RVFWT) was measured using M-mode in a modified parasternal long-axis view through the aortic valve. Pulmonary artery acceleration time (PAAT) was measured as the time from start to peak velocity of blood flow in the lumen of the main pulmonary artery distal to the pulmonary valve as obtained from the pulse-wave doppler recording.

Sixteen weeks after semaxanib treatment, rats were anesthetized with ketamine (100 mg/kg) and xylazine (5 mg/kg) at the end of the experiment to evaluate cardiac and pulmonary hemodynamics. The respiration was supported by a small animal ventilator. Thoracotomy was made through 4-5 intercostal space, and the heart was exposed. A pressure-volume catheter (2.0-Fr, SPR-869, Millar Instruments, Houston, TX, USA) was inserted into the left ventricle and right ventricle from the apex. Ventricular pressure and volume were calculated with LabChart 7 software. Stroke work, ejection fraction, and cardiac output were derived. After finishing left ventricular measurements, the catheter was advanced to the aorta, arterial blood systolic and diastolic pressure was detected. Then the catheter returned to the left ventricle and changed the direction laterally to enter the left atrium. Similarly, right atrial pressure was measured by moving the catheter from the right ventricle into atrium. To measure pulmonary arterial pressure, the sternum was cross-sectioned at the second inter-rib space. The right ventricular outflow tract was exposed. A hole was made with 27 G needle, and then the catheter was inserted into the right ventricular outflow tract and advanced into the pulmonary artery.

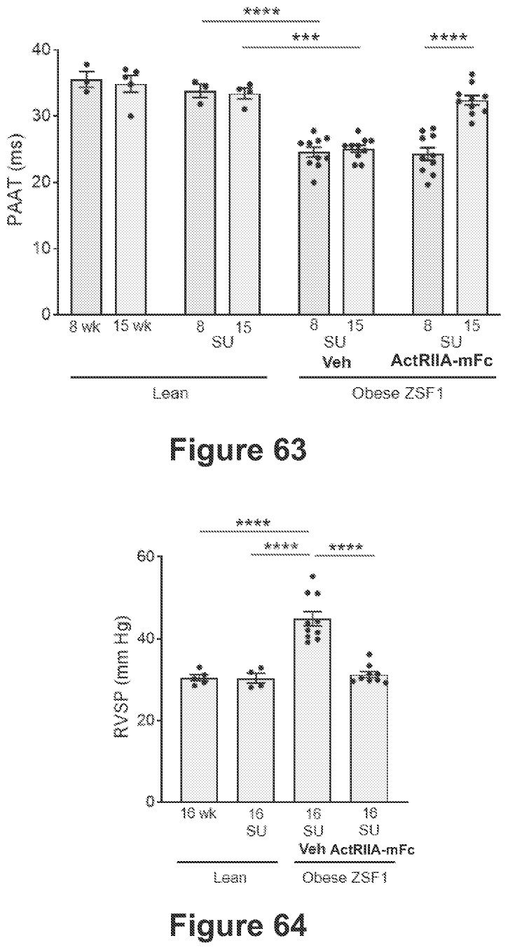

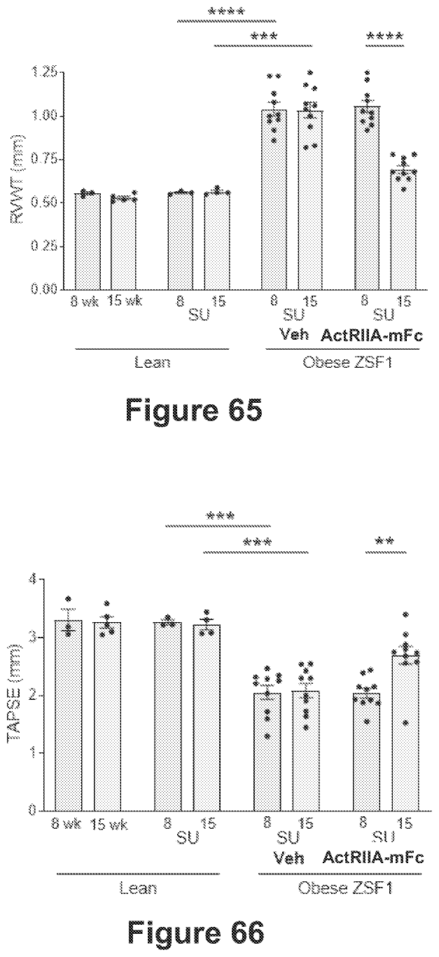

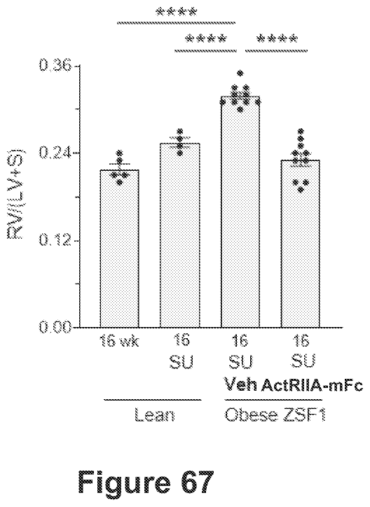

Compared to lean control animals, Obese ZSF1-SU rats in the PBS treatment group (Obese ZSF1 SU/Veh) 16-weeks after semaxanib treatment were observed to have decreased pulmonary artery acceleration time (PAAT) (FIG. 63), increased RVSP (FIG. 64), increased right ventricle free wall thickness (RVFWT) (FIG. 65), decreased tricuspid annular plane systolic excursion (TAPSE) (FIG. 66), and increased Fulton Index, calculated as the ratio of right ventricular weight (RV) to weight of the combined left ventricle and septum (LV+S) (FIG. 67).

As shown in FIGS. 63 and 64, ActRIIA-mFc treatment (Obese ZSF1-SU/ActRIIA-mFc) relative to PBS treatment (Obese ZSF1-SU/Veh) at 5 mpk normalized cardiopulmonary function as shown by the significantly increased PAAT (FIG. 63) and significantly reduced right ventricular systolic pressure (RVSP) (FIG. 64). ActRIIA-mFc treatment (Obese ZSF1-SU/ActRIIA-mFc) relative to PBS treatment (Obese ZSF1-SU/Veh) at 5 mpk also normalized right ventricular structure and function as shown by the significantly reduced elevated RVWT (FIG. 65), increased TAPSE (FIG. 66), and the decreased Fulton index (FIG. 67).

Together, these data demonstrate that ActRIIA-mFc is effective in ameliorating various complications of Group 2 PH in a left heart failure-induced PH model (HFpEF-PH). In particular, ActRIIA-mFc had a significant effect in normalizing cardiopulmonary function and in normalizing right ventricular structure and function.

1-130. (canceled)

131. A method of treating post-capillary pulmonary hypertension (PcPH), comprising administering to a patient in need thereof an effective amount of a fusion protein comprising:
(i) an ActRII variant polypeptide comprising an amino acid sequence that is at least 90% identical to any of SEQ ID NOs: 299, 300, 301, 302, 207, and 196;
(ii) an Fc domain of an immunoglobulin;
(iii) a linker domain positioned between the ActRII polypeptide domain and the Fc domain of the immunoglobulin.

132. The method of claim 131, wherein the PcPH is isolated post-capillary pulmonary hypertension (IpcPH).

133. The method of claim 131, wherein the PcPH is combined post- and pre-capillary PH (CpcPH).

134. The method of claim 131, wherein the Fc domain of the immunoglobulin is at least 90% identical to the amino acid sequence of SEQ ID NO: 14, 15, 16, 17, 18, 133, 134, 135, 136, 233, or 284.

135. The method of claim 131, wherein the Fc domain of the immunoglobulin is at least 95% identical to the amino acid sequence of SEQ ID NO: 14, 15, 16, 17, 18, 133, 134, 135, 136, 233, or 284.

136. The method of claim 131, wherein the Fc domain of the immunoglobulin is at least 99% identical to the amino acid sequence of SEQ ID NO: 14, 15, 16, 17, 18, 133, 134, 135, 136, 233, or 284.

137. The method of claim 131, wherein the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24).

138. The method of claim 131, wherein the linker domain is an amino acid spacer selected from the group consisting of GGG, GGGA (SEQ ID NO: 234), GGGG (SEQ ID NO: 20), GGGAG (SEQ ID NO: 263), GGGAGG (SEQ ID NO: 264), or GGGAGGG (SEQ ID NO: 265).

139. The method of claim 131, wherein the ActRII variant polypeptide comprises an amino acid sequence that is at least 95% identical to the amino acid sequence of SEQ ID NO: 299, 300, 301, 302, 207, and 196.

140. The method of claim 131, wherein the ActRII variant polypeptide comprises an amino acid sequence that is at least 99% identical to the amino acid sequence of SEQ ID NO: 299, 300, 301, 302, 207, and 196.

141. The method of claim 131, wherein the ActRII variant polypeptide comprises an amino acid sequence that is at least 99% identical to the amino acid sequence of SEQ ID NO: 207 or 196.

142. The method of claim 131, wherein the ActRII variant polypeptide comprises an amino acid sequence that is at least 99% identical to the amino acid sequence of SEQ ID NO: 299, 300, 301, or 302.

143. The method of claim 131, wherein the patient has Group 2 pulmonary hypertension as recognized by the World Health Organization (WHO).

144. The method of claim 131, wherein the patient has pulmonary hypertension due to heart failure with preserved left ventricular ejection fraction (LVEF).

145. The method of claim 131, wherein the method decreases PVR in the patient.

146. The method of claim 131, wherein the method prevents the progression of IpcPH to CpcPH.

147. The method of claim 131, wherein the method reduces the development of a pre-capillary component of PH.

148. The method of claim 131, wherein the method increases the patient's 6-minute walk distance.

149. The method of claim 131, wherein the method delays clinical worsening of PcPH in accordance with the World Health Organization's functional classification system for pulmonary hypertension.

150. The method of claim 131, wherein the method reduces the risk of hospitalization for one or more complications associated with PcPH.
